(19) United States
(12) Patent Application Publication
(10) Pub. No.: US 2022/0389007 A1
(43) Pub. Date: Dec. 8, 2022

(54) INHIBITORS OF HUMAN IMMUNODEFICIENCY VIRUS REPLICATION

(71) Applicant: VIIV HEALTHCARE UK (No. 5) LIMITED, Brentford, Middlesex (GB)

(72) Inventors: Michael S. BOWSHER, Wallingford, CT (US); Eric P. GILLIS, Branford, CT (US); Christiana IWUAGWU, Branford, PA (US); B Narasimhulu NAIDU, Branford, CT (US); Kyle E. PARCELLA, Branford, CT (US); Manoj PATEL, Branford, CT (US)

(21) Appl. No.: 17/763,133

(22) PCT Filed: Sep. 29, 2020
(86) PCT No.: PCT/IB2020/059103

§ 371 Date: Mar. 23, 2022

Related U.S. Application Data
(60) Provisional application No. 62/908,715, filed on Oct. 1, 2019.
(60) Provisional application No. 62/925,300, filed on Oct. 24, 2019.

(51) Int. Cl. C07D 471/04; C07D 277/08; A61P 31/18

(52) U.S. Cl. CPC C07D 471/04; C07D 277/08; A61P 31/18

(57) ABSTRACT

Compounds of Formula I, including pharmaceutically acceptable salts thereof, and compositions and methods for treating human immunodeficiency virus (HIV) infection are set forth:

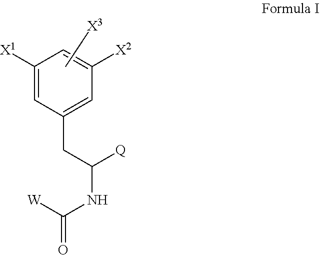

### FIELD OF THE INVENTION

The invention relates to compounds, compositions, and methods for the treatment of human immunodeficiency virus (HIV) infection. More particularly, the invention provides novel Capsid inhibitors, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. HIV drug resistance—an emerging threat to epidemic control. N. Engl. J. Med. 2017, 377, 1605-1607; Gupta, R. K., Gregson J., et al. HIV-1 drug resistance before initiation or re-initiation of first-line antiretroviral therapy in low-income and middle-income countries: a systematic review and meta-regression analysis. Lancet Infect. Dis. 2017, 18, 346-355; Zazzi, M., Hu, H., Prosperi, M. The global burden of HIV-1 drug resistance in the past 20 years. PeerJ. 2018, DOI 10.7717/peerj.4848). As a result, new drugs are needed that are easier to take, have high genetic barriers to the development of resistance and have improved safety over current agents. In this panoply of choices, novel mechanisms of action (MOAs) that can be used as part of the preferred antiretroviral therapy (ART) can still have a major role to play since they should be effective against viruses resistant to current agents.

Certain potentially therapeutic compounds have now been described in the art and set forth in Blair, Wade S. et.al. Antimicrobial Agents and Chemotherapy (2009), 53(12), 5080-5087, Blair, Wade S. et al. PLoS Pathogens (2010), 6(12), e1001220, Thenin-Houssier, Suzie; Valente, Susana T. Current HIV Research, 2016, 14, 270-282, and PCT Patent applications with the following numbers: WO 2012065062, WO 2013006738, WO 2013006792, WO 2014110296, WO 2014110297, WO 2014110298, WO 2014134566, WO 2015130964, WO2015130966, WO 2016033243, W02018035359, W02018203235, WO 2019161017, and WO 2019161280.

What is now needed in the art are additional compounds which are novel and useful in the treatment of HIV. Additionally, these compounds should provide advantages for pharmaceutical uses, for example, with regard to one or more of their mechanisms of action, binding, inhibition efficacy, target selectivity, solubility, safety profiles, bioavailability or reduced frequency of dosing. Also needed are new formulations and methods of treatment which utilize these compounds.

### SUMMARY OF THE INVENTION

Briefly, in one aspect, the present invention discloses a compound of Formula I, or a pharmaceutically acceptable salt thereof:

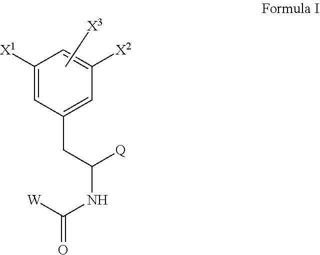

wherein:
X¹ and X² are independently selected from H, F, Cl or —CH₃ and X³ is H, F, Cl, —CH_{3,} —OCH₃, —OCHF₂, or —OCF₃ with the proviso that within the group X¹, X², and X³ the substituent Cl is not used more than twice and the substituent —CH₃ is not used more than twice;
Q is selected from:

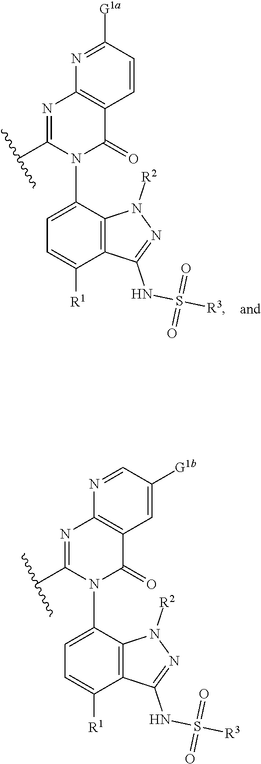

R¹ is H, Cl, or CH₃;
R² is H, C₁-C₃alkyl optionally substituted with 1-3 fluorines, or C₃-C₆cycloalkyl optionally substituted with 1-2 fluorines;
R³ is C₁-C₃alkyl or C₃-C₄cycloalkyl;
G^{1a} is phenyl, pyridine, pyrazine, or pyrimidine, each of which is substituted with —SF₅, or G^{1a} is selected from:

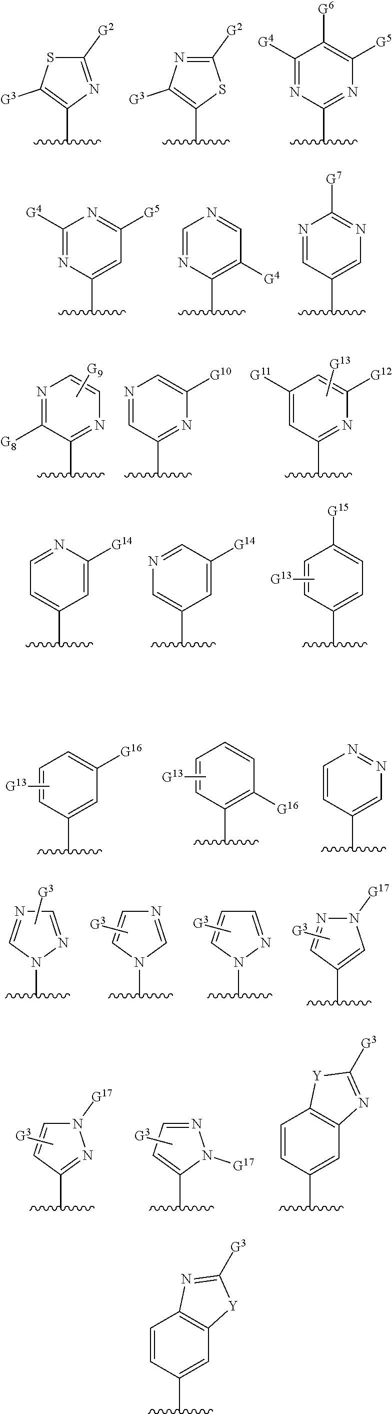

G² is C₁-C₃alkyl, —O(C₁-C₃alkyl), —S(O₂)CH₃, or —C(CH₃)₂OH wherein C₁-C₃alkyl is optionally substituted with 1-3 fluorines;
G³ is H, or methyl optionally substituted with 1-3 fluorines;
G⁴ and G⁵ are independently selected from H, —O(C₁-C₃alkyl), or C₁-C₂alkyl optionally substituted with 1-3 fluorines;

### G⁶ is H, Cl, or F;

G⁷ is H, —OCH₃, or —S(O₂)CH₃;
G⁸ is H, methyl, ethyl, or Cl;

### G⁹ is H or Cl;

G¹⁰ is H, C₁-C₂alkyl, —OCH₃, or —SF₅ where C₁-C₂alkyl is optionally substituted with 1-3 fluorines;
G¹¹ and G¹² are independently selected from H, F, Cl or C₁-C₂alkyl wherein C₁-C₂alkyl are optionally substituted with 1-3 fluorines;

### G¹³ is H or F;

G¹⁴ is H, methyl, Cl, —OCH₃;
G¹⁵ is —O(C₁-C₂alkyl) substituted with 1-3 fluorines, or —S(O₂)CH₃;
G¹⁶ is C₁-C₂alkyl substituted with 1-3 fluorines, or —O(C₁-C₂alkyl) wherein substituted with 1-3 fluorines;
G¹⁷ is H, cPr, —CH2cPr, or C₁-C₄alkyl wherein C₁-C₄alkyl is optionally substituted with 1-5 fluorines;

### Y is O, S or N;

G^{1b} is pyridine, pyrimidine, pyrazine, or phenyl, each of which is substituted once from the group F, Cl, or C₁-C₂alkyl wherein C₁-C₂alkyl is optionally substituted with 1-3 fluorines;
W is selected from:

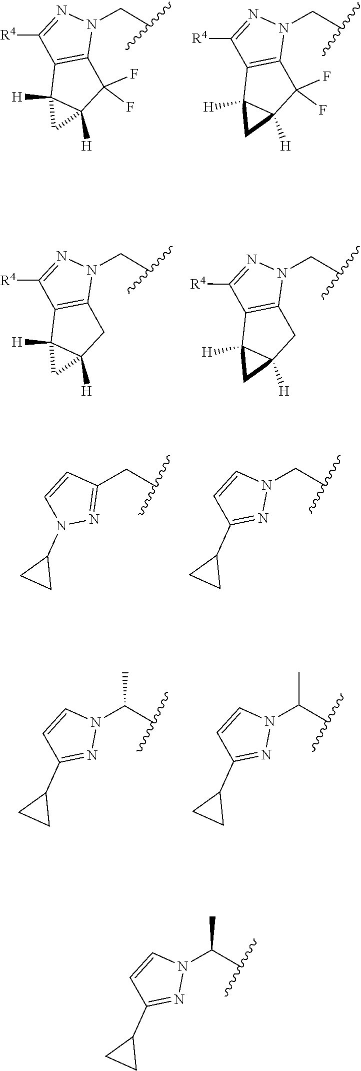

wherein R⁴ is methyl optionally substituted with 1-3 fluorines.

In another aspect, the present invention discloses a composition comprising a compound of Formula I or a pharmaceutically acceptable salt thereof.

In another aspect, the present invention discloses a method of treating HIV infection in a human comprising administering a compound of Formula I or a pharmaceutically acceptable salt thereof to a patient.

In another aspect, the present invention discloses a compound of Formula I or pharmaceutically acceptable salt thereof for use in therapy.

In another aspect, the present invention discloses a compound of Formula I or pharmaceutically acceptable salt thereof for use in treating HIV infection in a human. In another aspect, the present invention discloses the use of a compound of Formula I or pharmaceutically acceptable salt thereof in the manufacture of a medicament for the treatment of HIV infection in a human.

### DETAILED DESCRIPTION OF THE INVENTION

In one embodiment, the present invention discloses compounds of Formula I and pharmaceutically acceptable salts thereof wherein Q is:

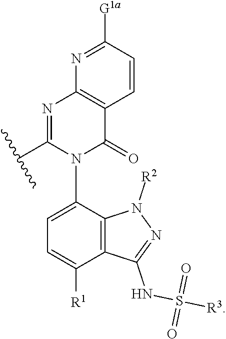

In one embodiment, the present invention discloses compounds of Formula I and pharmaceutically acceptable salts thereof wherein Q is:

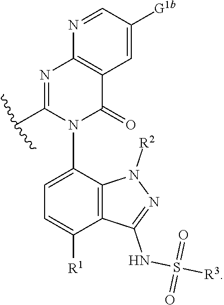

In one embodiment, the present invention discloses compounds of Formula I and pharmaceutically acceptable salts thereof wherein W is:

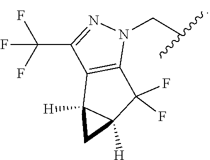 [SMILES: *Cn1nc(C(F)(F)F)c2c1C(F)(F)[C@H]1C[C@@H]21]

In one embodiment, the present invention discloses compounds of Formula I and pharmaceutically acceptable salts thereof wherein W is:

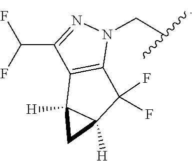 [SMILES: *Cn1nc(C(F)F)c2c1C(F)(F)[C@H]1C[C@@H]21]

In one embodiment, the present invention discloses compounds of Formula I and pharmaceutically acceptable salts thereof wherein W is the following:

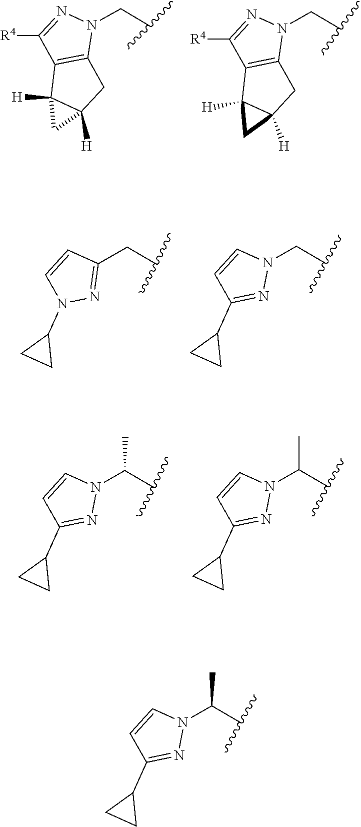

wherein R⁴ is methyl optionally substituted with 1-3 fluorines.

In one embodiment, the present invention discloses compounds of Formula I and pharmaceutically acceptable salts thereof wherein R¹ is Cl; R² is methyl, 2,2-difluoroethyl, or 2,2,2-trifluoroethyl; and R³ is methyl or cyclopropyl.

In one embodiment, the present invention discloses compounds of Formula I and pharmaceutically acceptable salts thereof wherein R¹ is Cl; R² is methyl; and R³ is methyl.

In one embodiment, the present invention discloses compounds of Formula I and pharmaceutically acceptable salts thereof wherein X³ is H. In another embodiment, the present invention discloses compounds of Formula I and pharmaceutically acceptable salts thereof wherein X¹ is F, X² is F, and X³ is H. In another embodiment, the present invention discloses compounds of Formula I and pharmaceutically acceptable salts thereof wherein if X³ is H then at least one of X¹ and X² is other than F.

In one embodiment, the present invention discloses compounds of Formula I and pharmaceutically acceptable salts thereof wherein G^{1a} is one of the following:

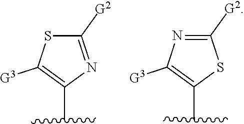

In one embodiment, the present invention discloses compounds of Formula I and pharmaceutically acceptable salts thereof wherein G^{1a} is one of the following:

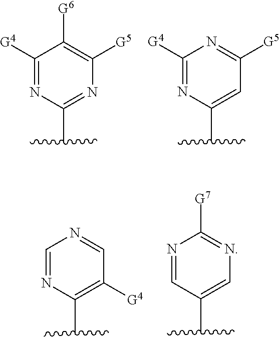

In one embodiment, the present invention discloses compounds of Formula I and pharmaceutically acceptable salts thereof wherein G^{1a} is one of the following:

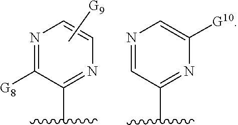

In one embodiment, the present invention discloses compounds of Formula I and pharmaceutically acceptable salts thereof wherein G^{1a} is one of the following:

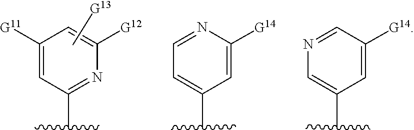

In one embodiment, the present invention discloses compounds of Formula I and pharmaceutically acceptable salts thereof wherein G^{1a} is one of the following:

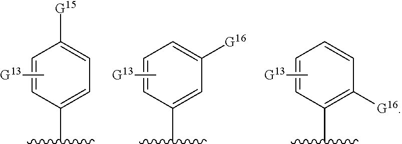

In one embodiment, the present invention discloses compounds of Formula I and pharmaceutically acceptable salts thereof wherein G^{1a} is:

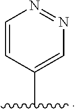 [SMILES: *c1ccnnc1]

In one embodiment, the present invention discloses compounds of Formula I and pharmaceutically acceptable salts thereof wherein G^{1a} is:

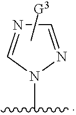

In one embodiment, the present invention discloses compounds of Formula I and pharmaceutically acceptable salts thereof wherein G^{1a} is one of the following:

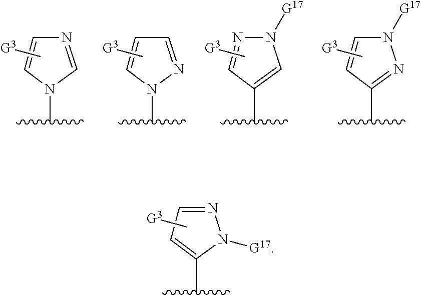

In one embodiment, the present invention discloses compounds of Formula I and pharmaceutically acceptable salts thereof wherein G^{1a} is one of the following:

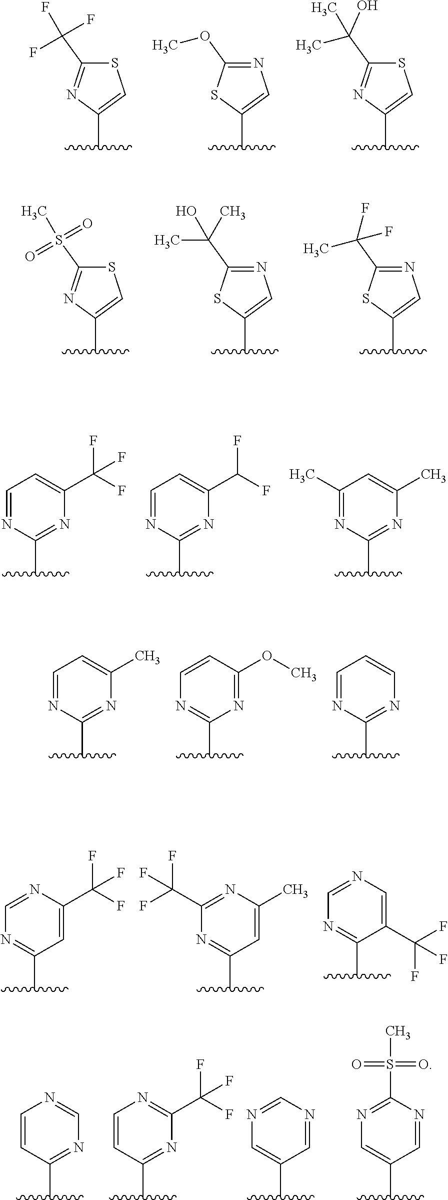

In one embodiment, the present invention discloses compounds of Formula I and pharmaceutically acceptable salts thereof wherein G^{1a} is one of the following:

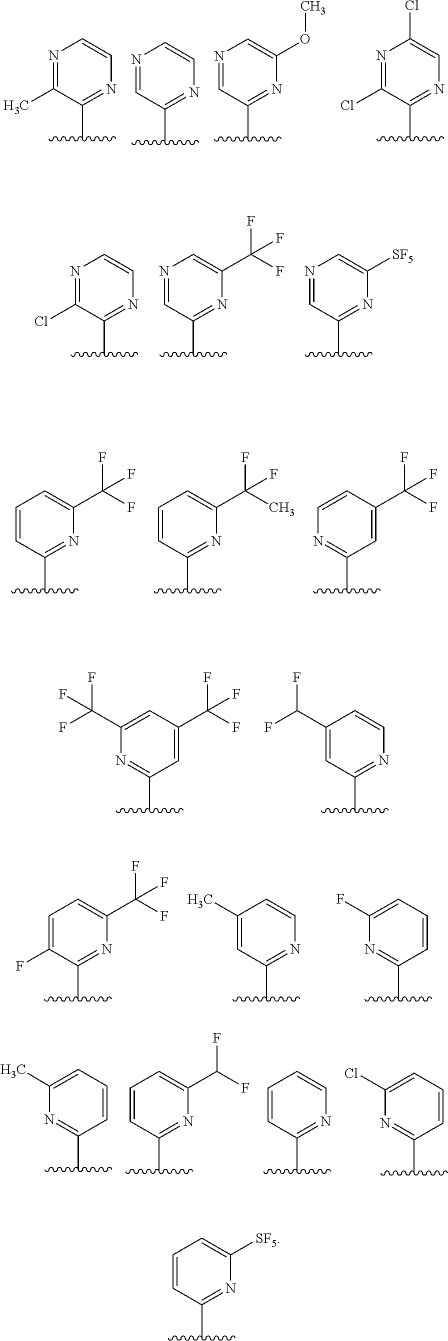

In one embodiment, the present invention discloses compounds of Formula I and pharmaceutically acceptable salts thereof wherein G^{1a} is one of the following:

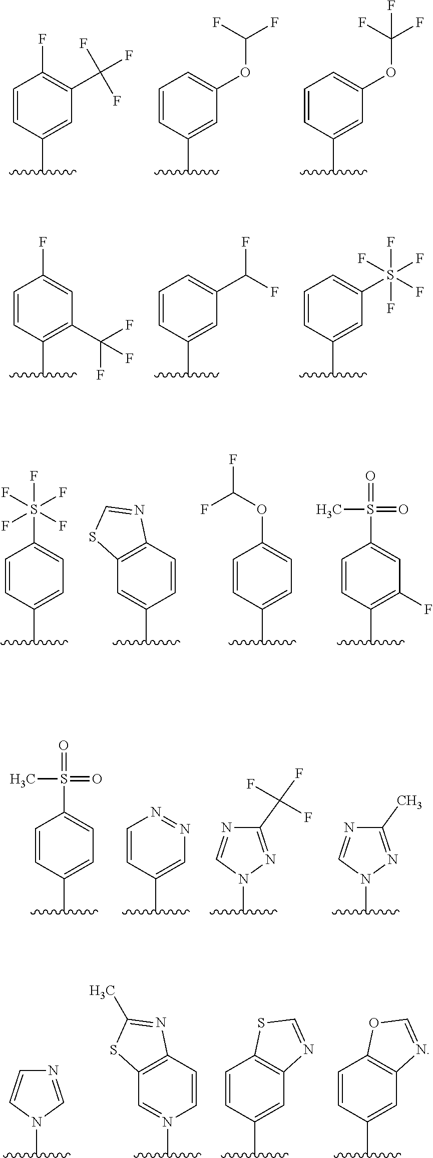

In one embodiment, the present invention discloses compounds of Formula I and pharmaceutically acceptable salts thereof wherein G^{1a} is one of the following:

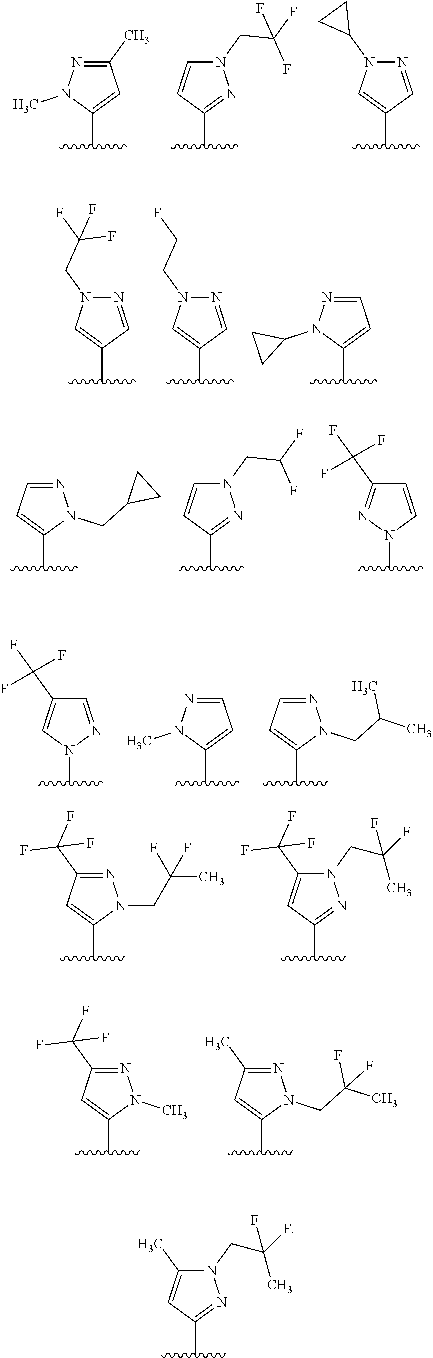

In one embodiment, the present invention discloses compounds of Formula I and pharmaceutically acceptable salts thereof wherein G^{1b} is one of the following:

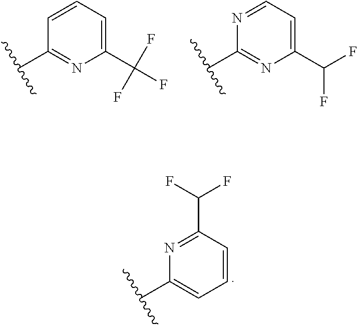 [SMILES: *c1cccc(C(F)(F)F)n1 ; *c1nccc(C(F)F)n1 ; *c1cccc(C(F)F)n1]

In one embodiment, the present invention discloses compounds of Formula I and pharmaceutically acceptable salts thereof wherein G^{1a} or G^{1b} contains 2-3 fluorines.

In one embodiment, the present invention discloses compounds of Formula I and pharmaceutically acceptable salts thereof wherein the chemical formula of G^{1a} or G^{1b} is C₍₄₋₆₎H₍₂₋₃₎F₍₂₋₃₎N₍₁₋₂₎.

In one embodiment, the present invention discloses compounds of Formula I and pharmaceutically acceptable salts thereof wherein the stereochemistry is as depicted below:

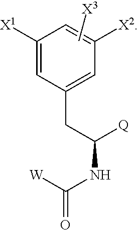

In one embodiment, the present invention discloses compounds of Formula I and pharmaceutically acceptable salts thereof wherein the stereochemistry is as depicted below:

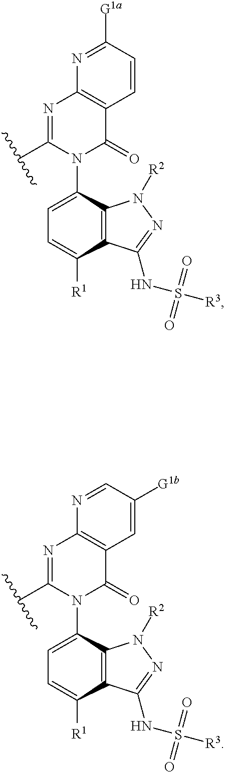

In one embodiment, the present invention discloses compounds and salts selected from the group consisting of:

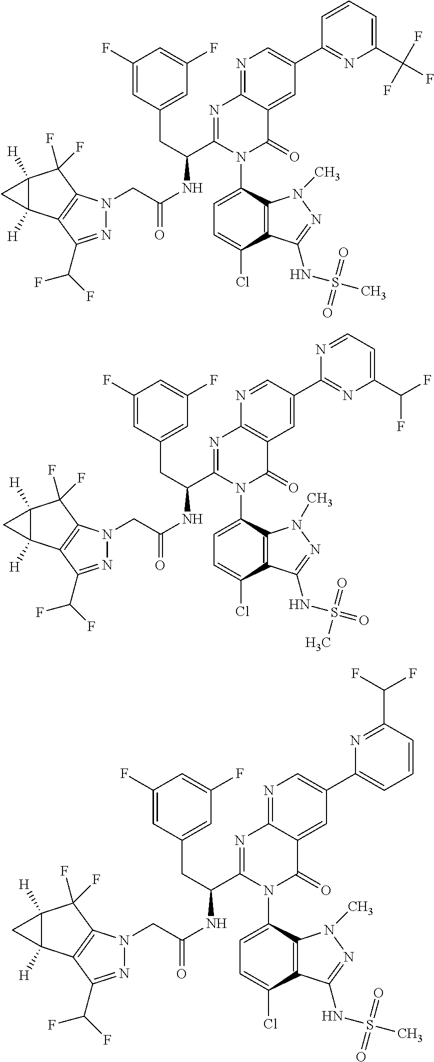

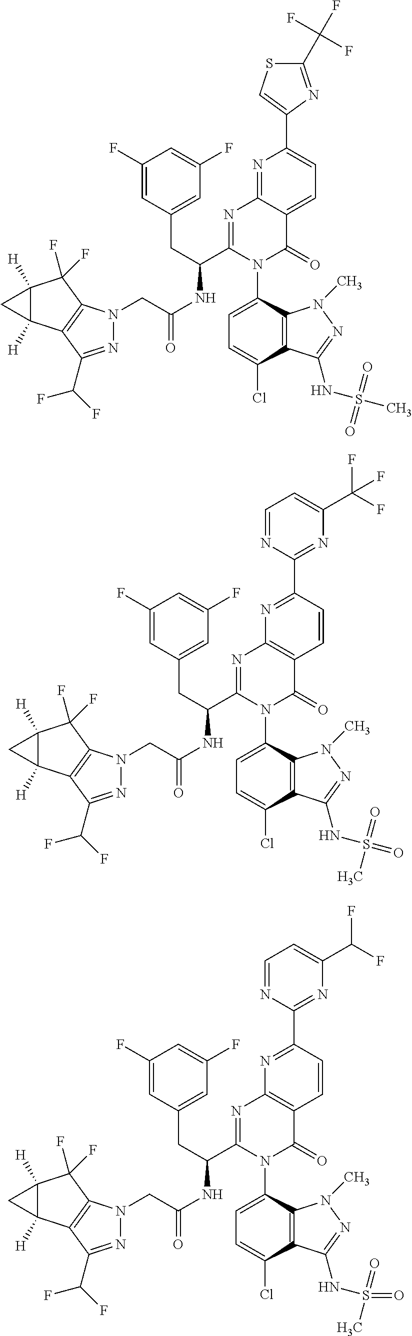

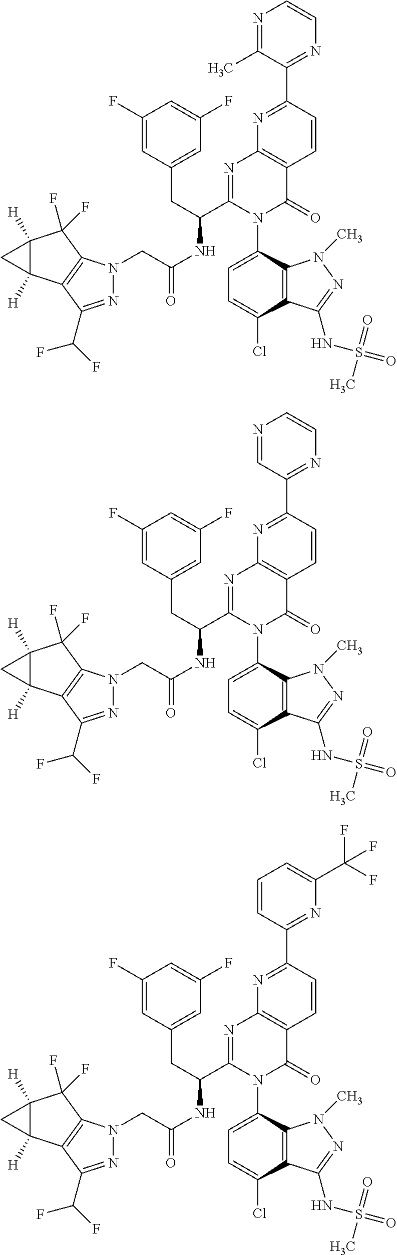

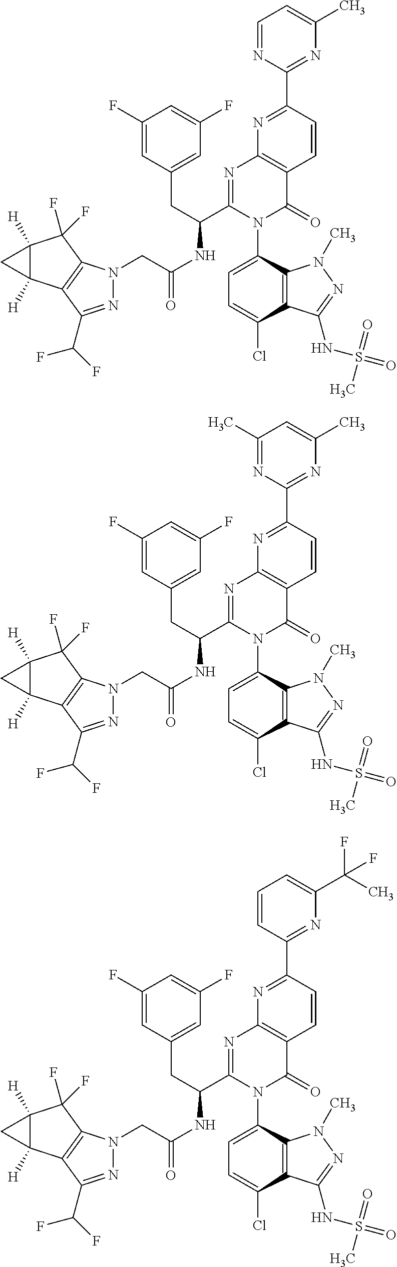

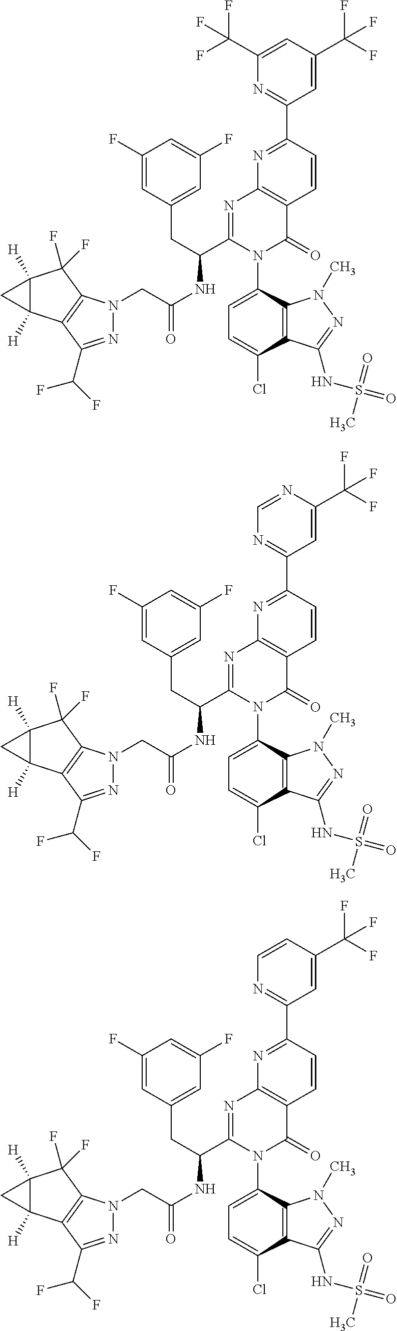

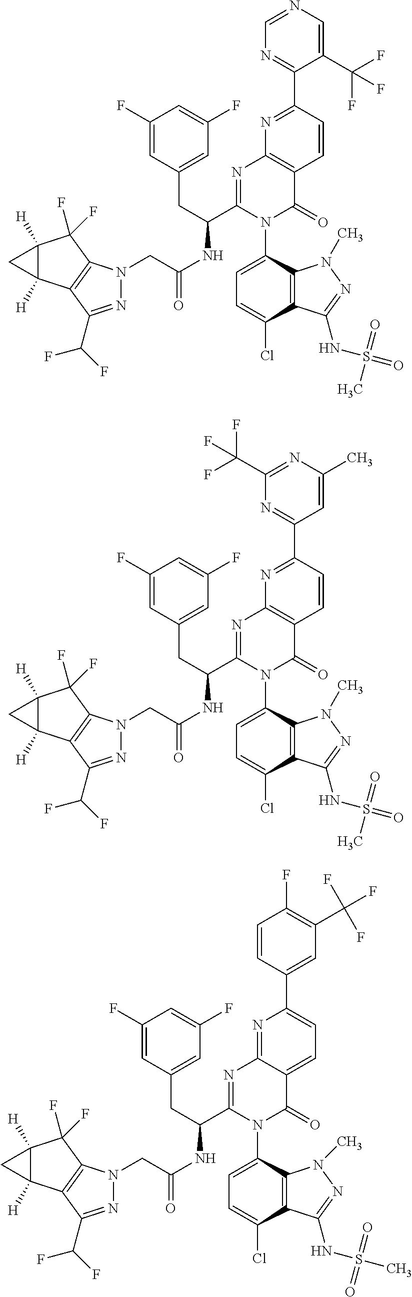

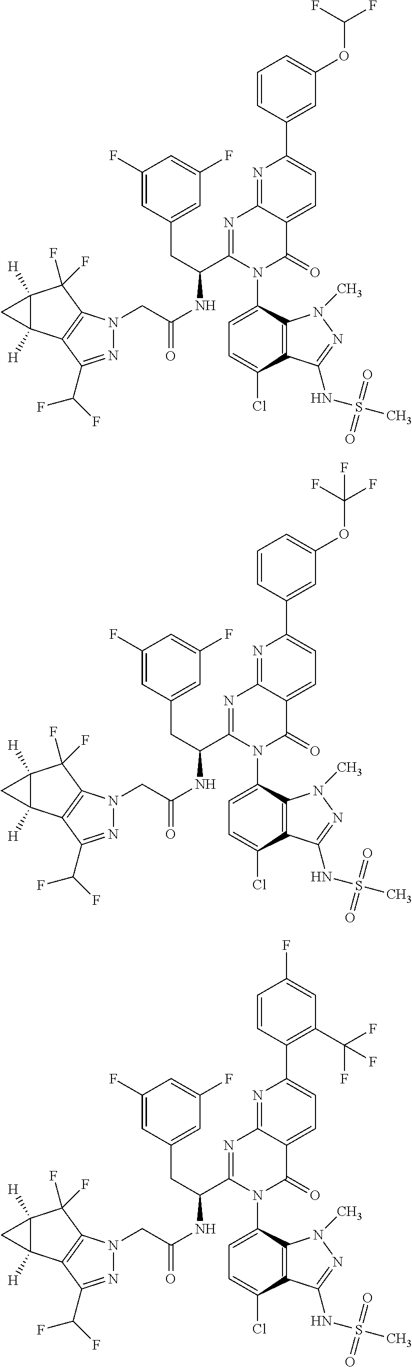

and pharmaceutically acceptable salts thereof.

In one embodiment, the present invention discloses compounds and salts selected from the group consisting of:

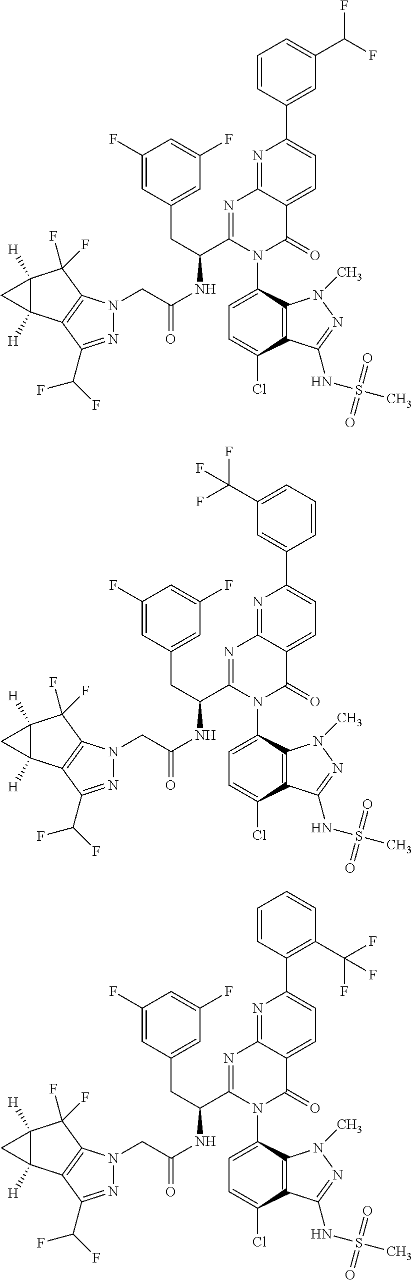

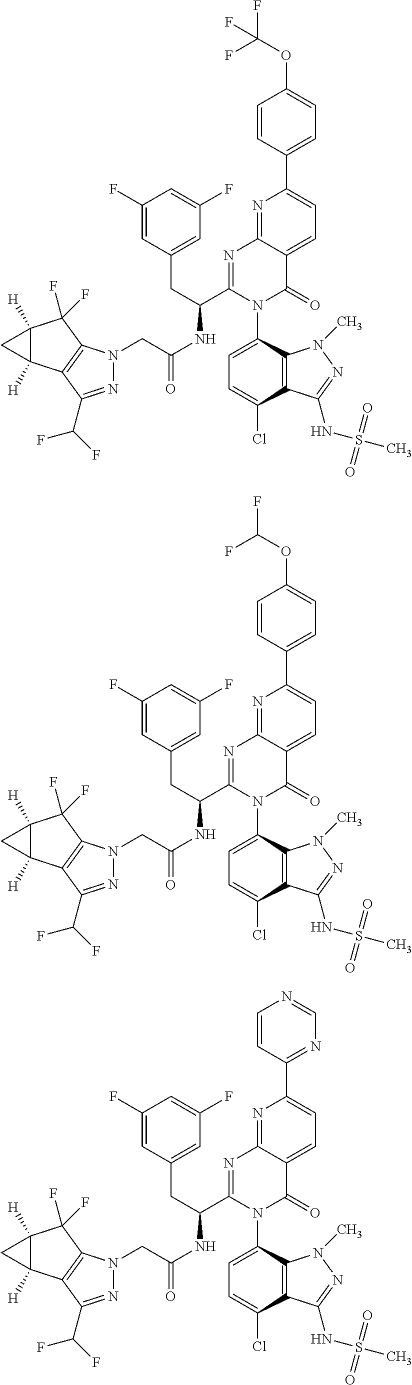

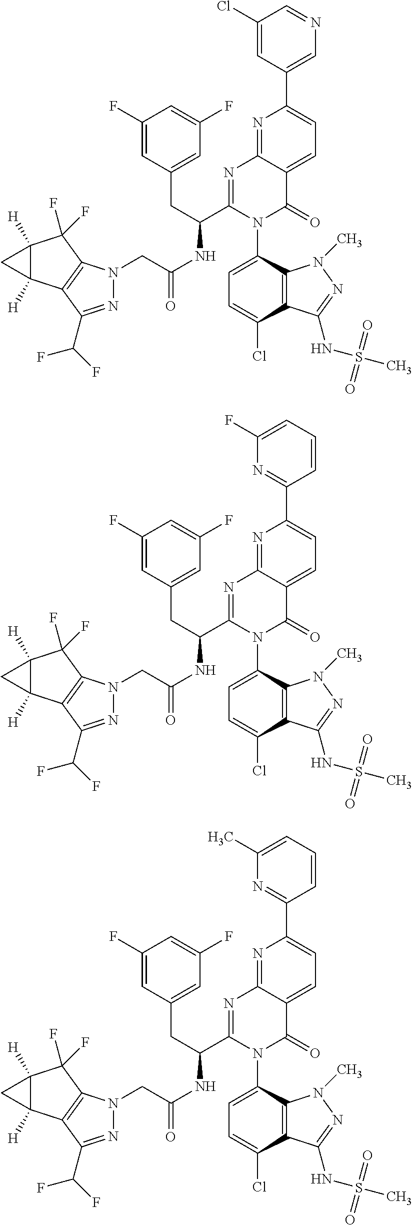

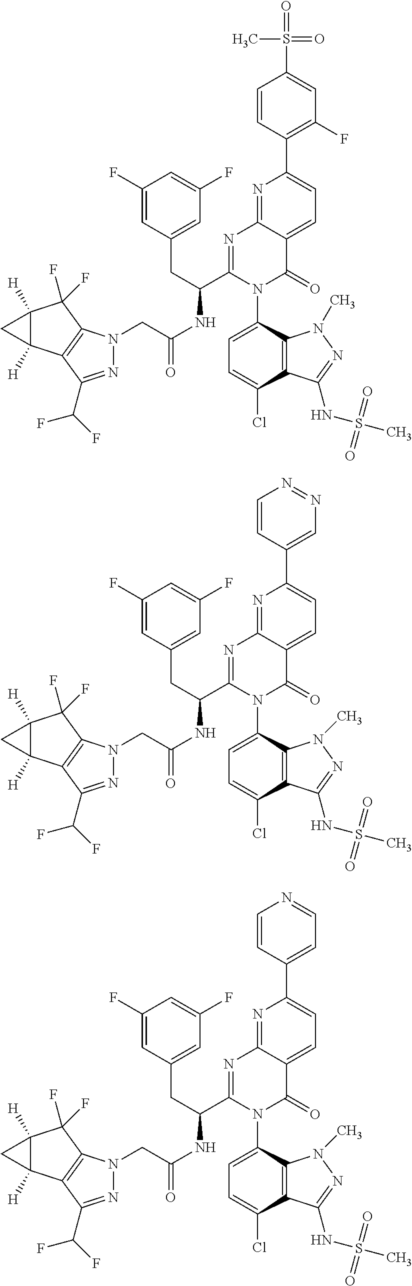

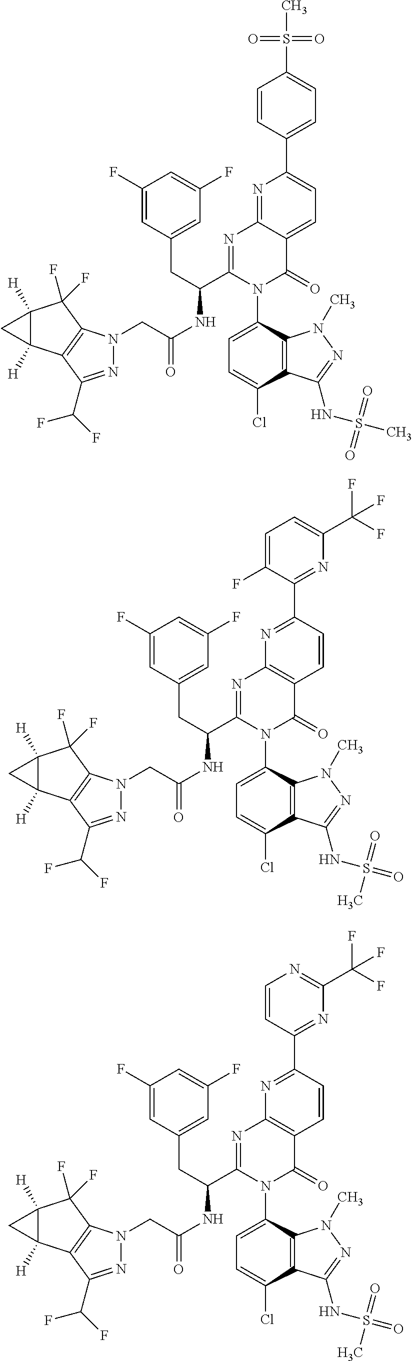

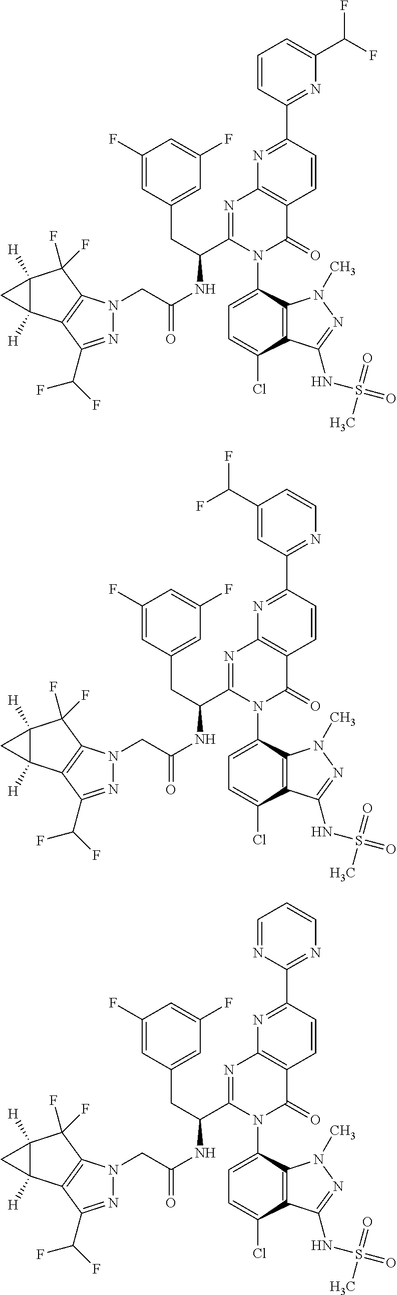

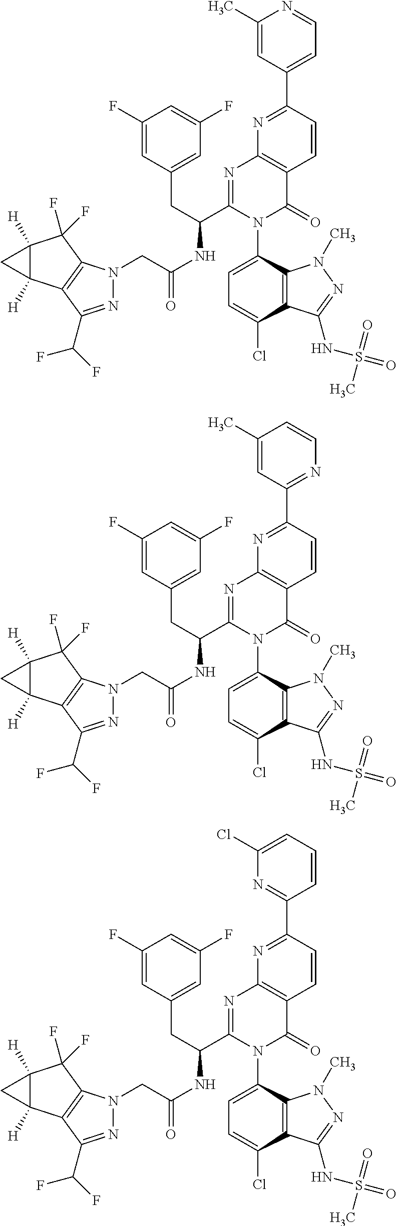

and pharmaceutically acceptable salts thereof.

In one embodiment, the present invention discloses compounds and salts selected from the group consisting of:

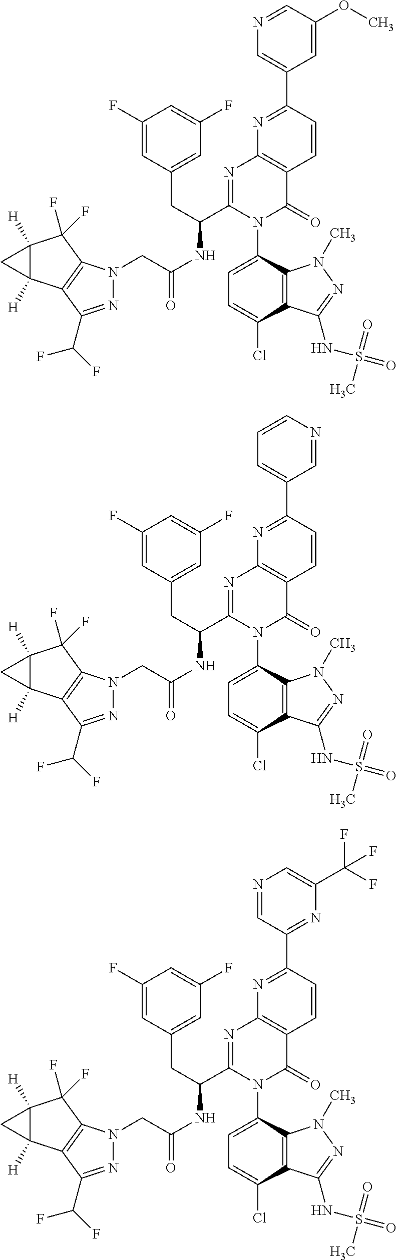

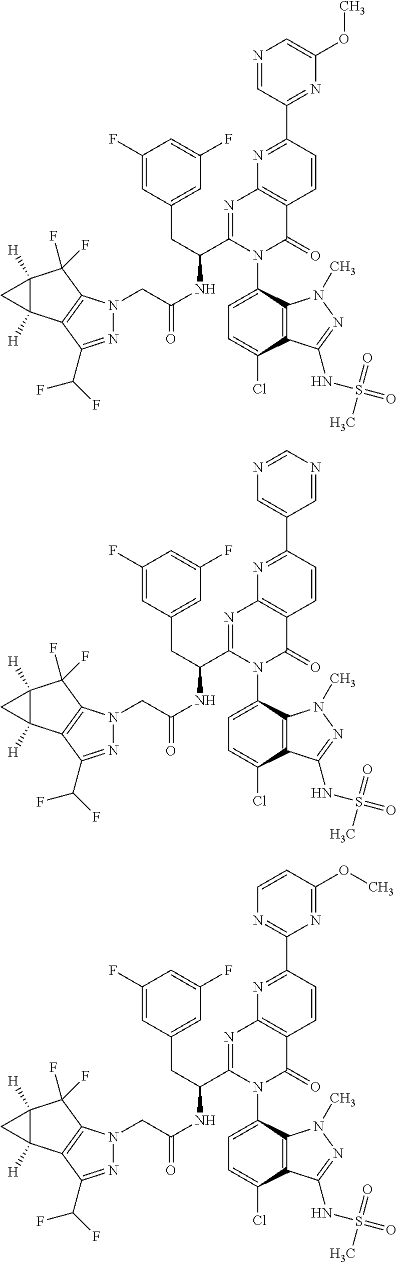

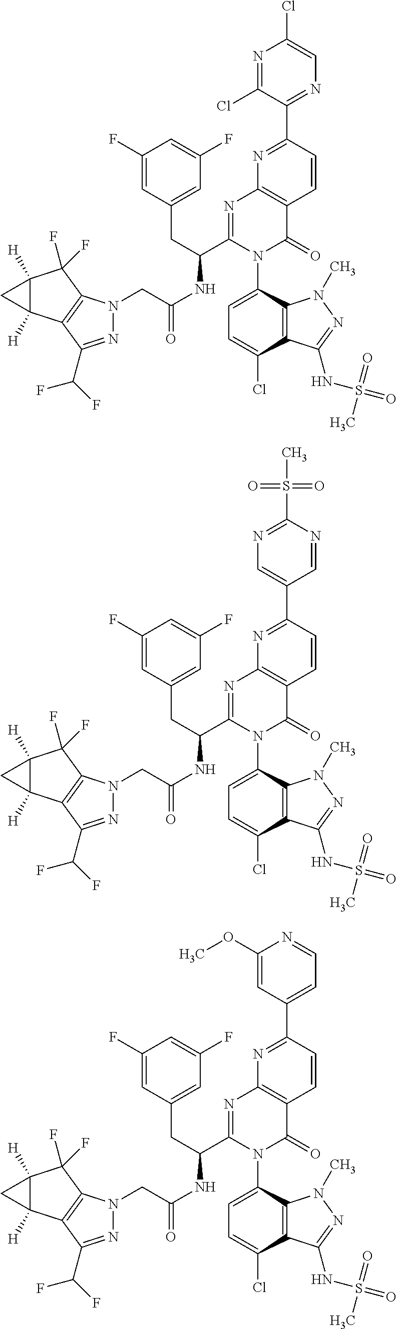

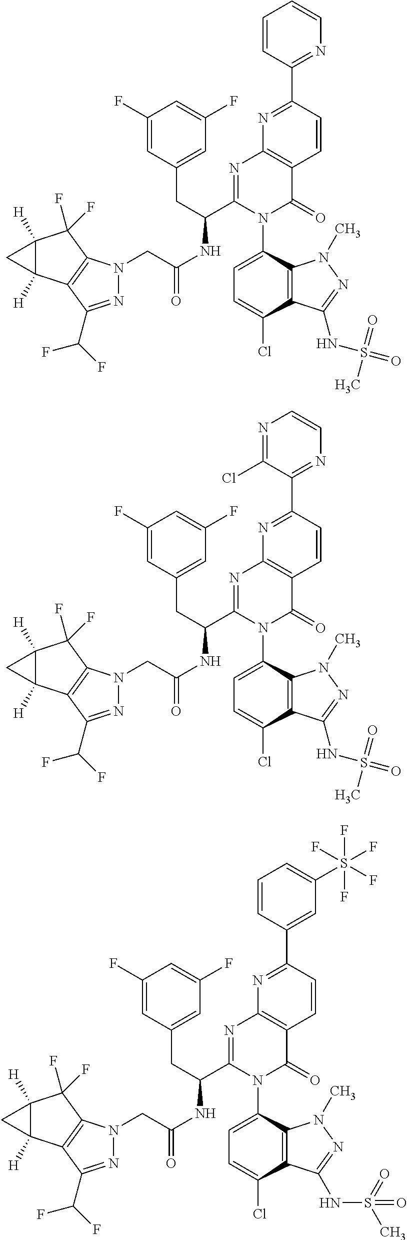

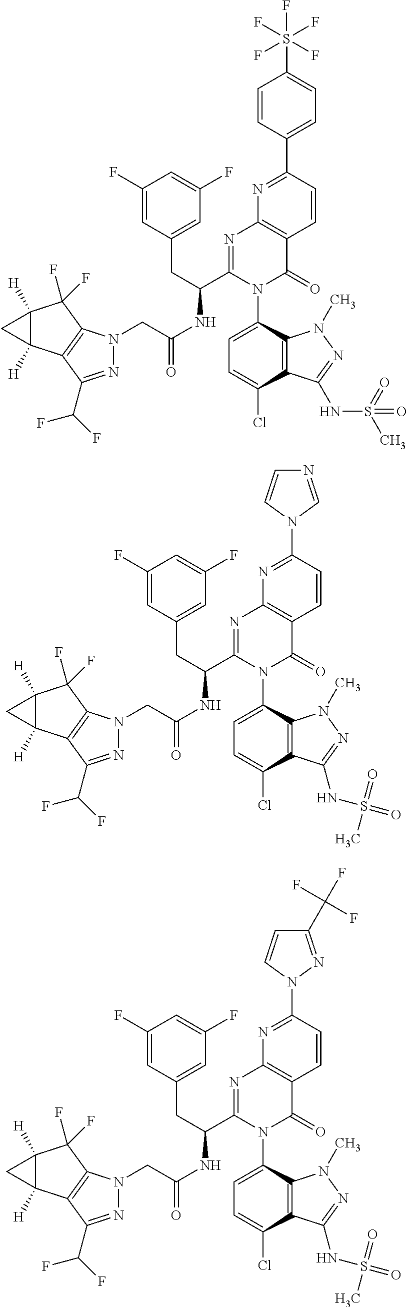

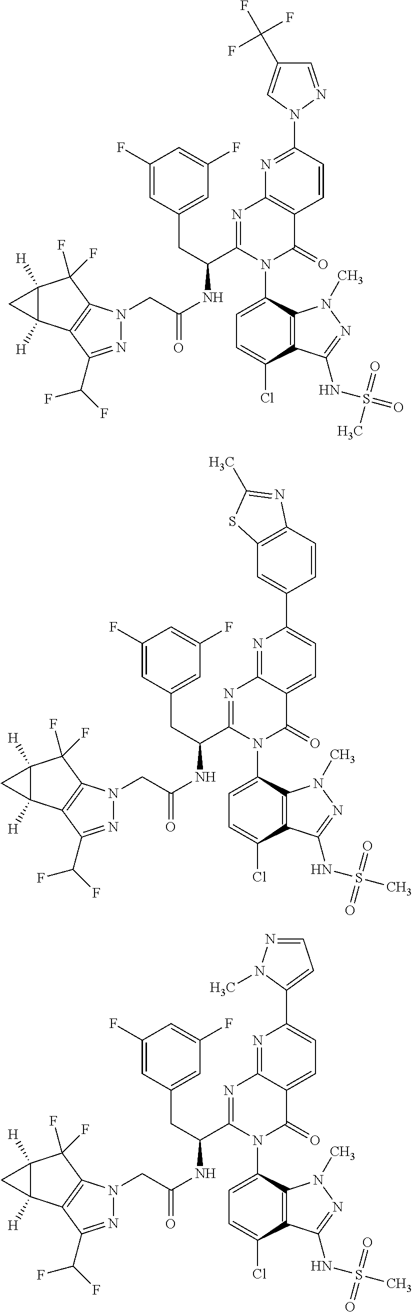

and pharmaceutically acceptable salts thereof.

In one embodiment, the present invention discloses compounds and salts selected from the group consisting of:

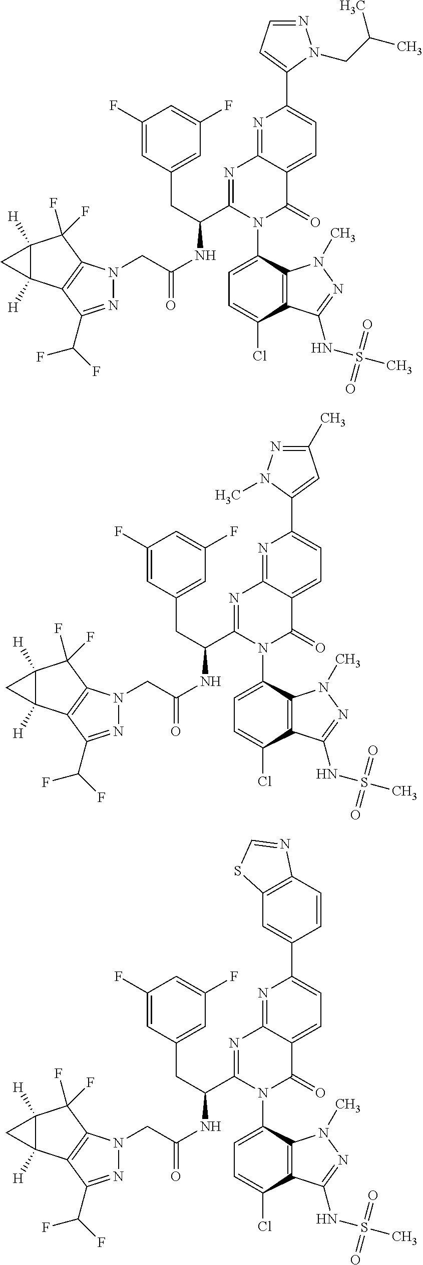

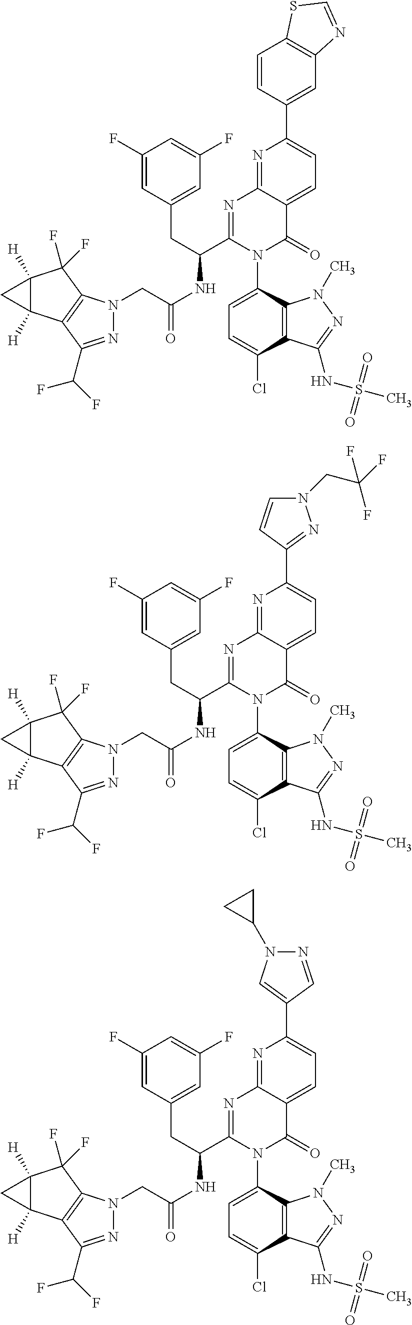

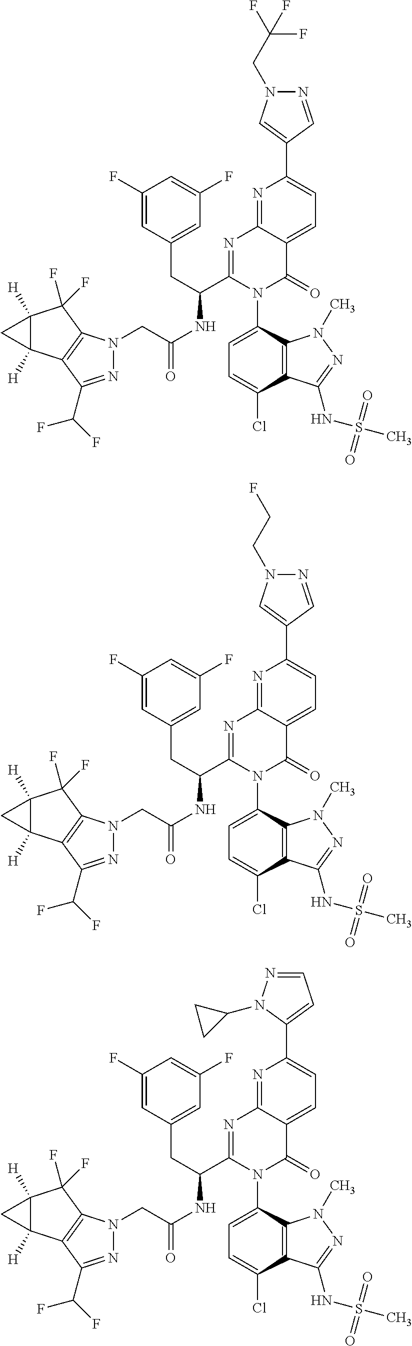

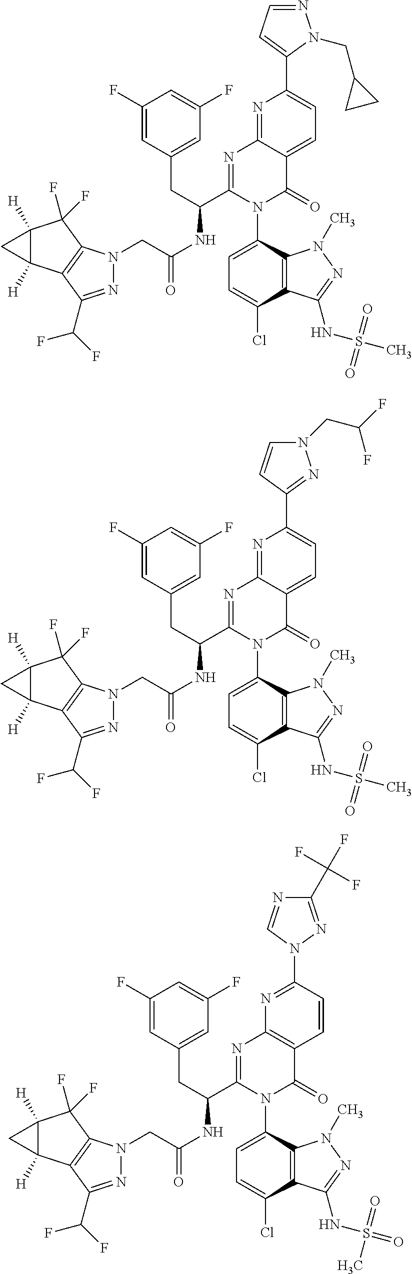

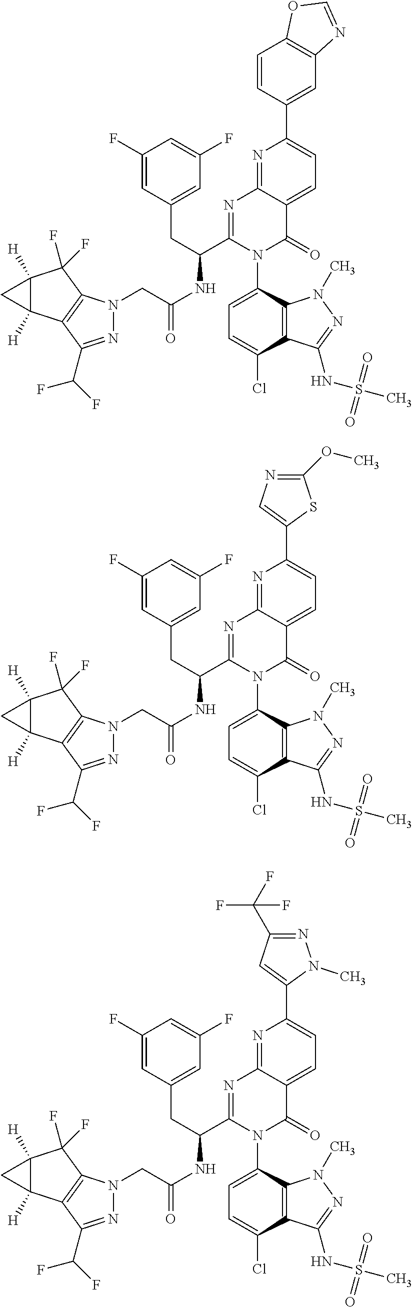

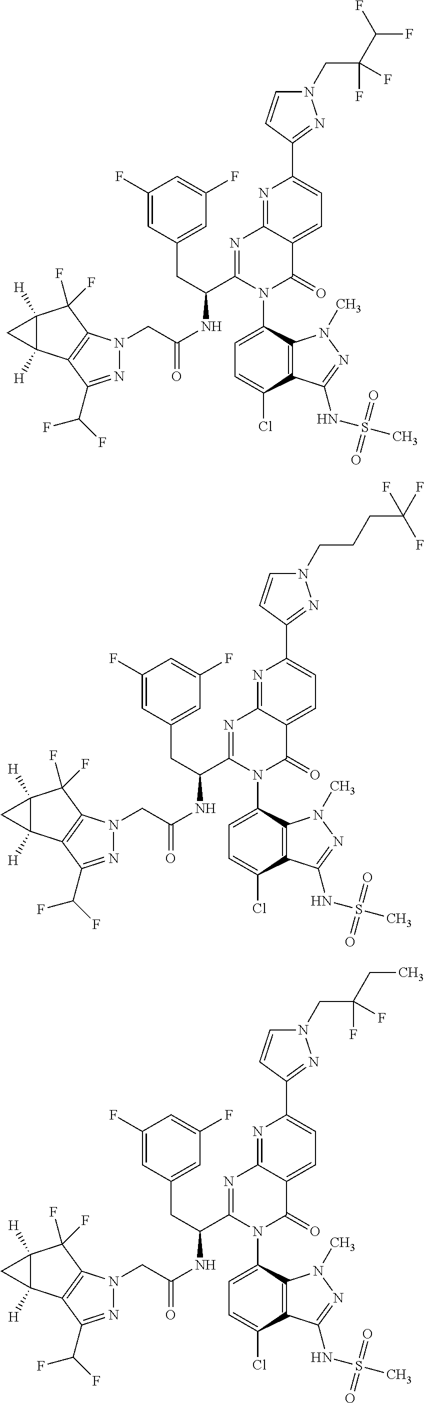

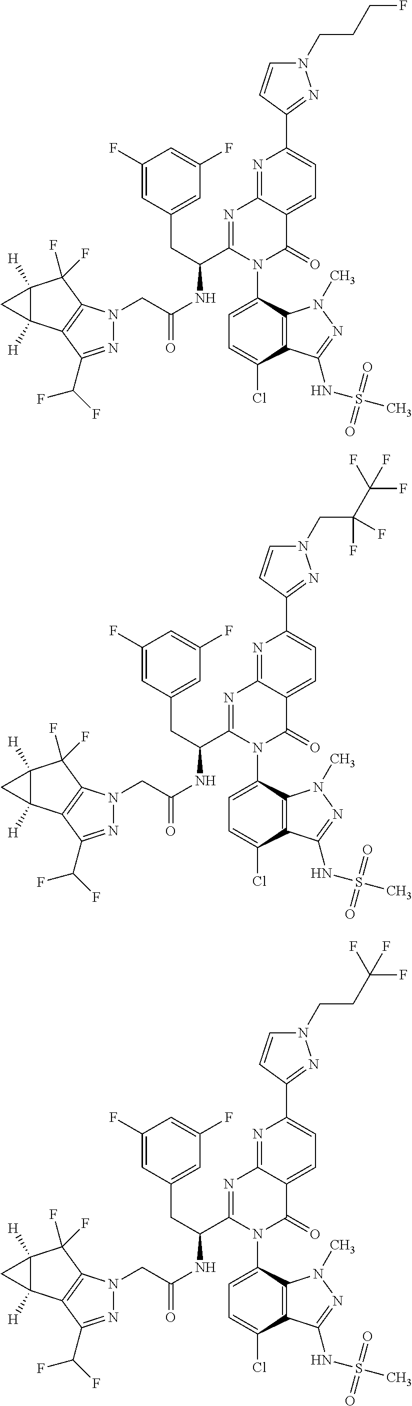

and pharmaceutically acceptable salts thereof.

In one embodiment, the present invention discloses compounds and salts selected from the group consisting of:

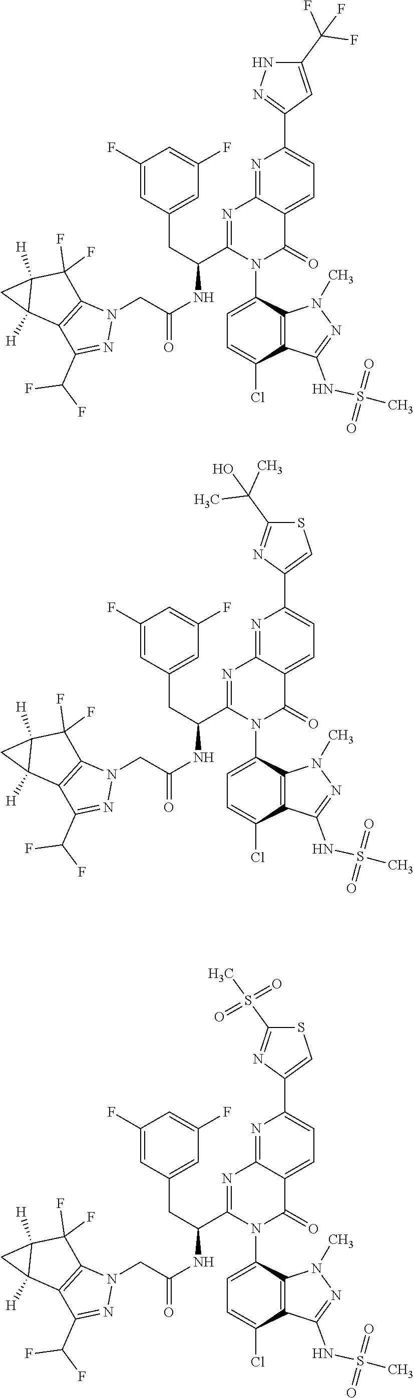

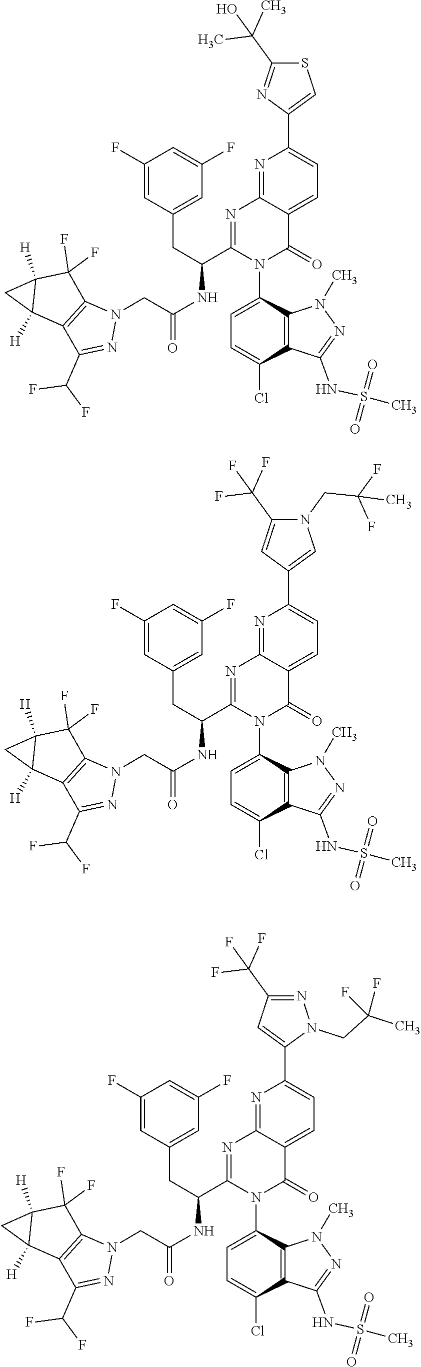

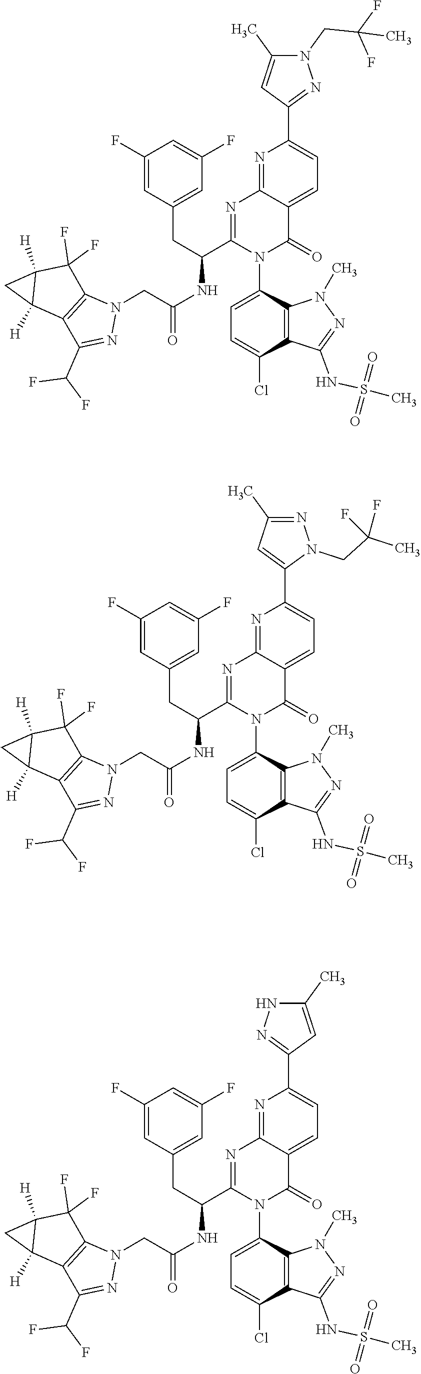

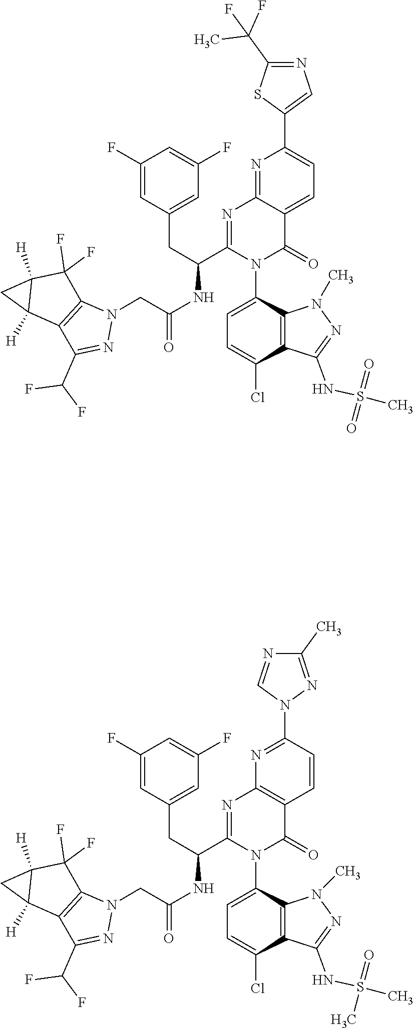

and pharmaceutically acceptable salts thereof.

The salts of the invention are pharmaceutically acceptable. Such salts may be acid addition salts or base addition salts. For a review of suitable pharmaceutically acceptable salts see, for example, Berge et al, J. Pharm, Sci., 66, 1-19, 1977.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In one embodiment, the compositions of this invention further comprise a pharmaceutically acceptable excipient. In the method of this invention, preferred routes of administration are oral and by injection to deliver subcutaneously or intramuscularly. Therefore, preferred pharmaceutical compositions include compositions suitable for oral administration (for example tablets) and compositions suitable for subcutaneous or intramuscular injection.

In another aspect the present invention discloses methods of preventing HIV infection in a human or reducing the risk of infection, comprising administering a compound or salt of this invention. Pre-exposure prophylaxis (or PrEP) is when people at risk for HIV infection take daily medicine to lower their chances of getting HIV infection. PrEP has been shown to be effective in reducing the risk of infection. As used herein, “HIV” or “Human Immunodeficiency Virus” refers to HIV-1 and/or to HIV-2.

The compounds and salts of this invention are believed to have as their biological target the HIV capsid and thus their mechanism of action is to modify in one or more ways the function of the HIV capsid.

The compounds and salts of the present invention may be employed alone or in combination with other therapeutic agents. Combination therapies according to the present invention thus comprise the administration of at least one compound or salt of the invention, and the administration of at least one other agent which may be useful in the treatment of HIV infection. A compound or salt of the present invention, and the other agent may be formulated and administered together in a single pharmaceutical composition or may be formulated and administered separately. When formulated and administered separately, administration may occur simultaneously or sequentially in any order. Suitable other agents include, for example, abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, the antibody N6LS, GSK3739937/VH3739937 and GSK4000422/VH4000422, indinavir, lamivudine, lopinavir, maraviroc, nelfinavir, nevirapine, raltegravir, rilpiverine, ritonavir, saquinavir, slatravir, stavudine, tipranavir, tenofovir, tenofovir alafenamide, tenofovir disoproxil fumarate, zalcitabine, zidovudine, and S-648414. Preferred agents include, for example, bictegravir, cabotegravir, dolutegravir, fostemsavir, islatravir, and lamivudine. Particularly preferred agents include, for example, bictegravir, cabotegravir, dolutegravir, fostemsavir, and lamivudine.

### EXAMPLES

### General Procedures

### General Procedure B:

A 5 mL microwave vial was charged with (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (0.047-0.093 mmol, 1 equiv.), the indicated stannane (1.0-1.2 equiv), and Pd(PPh₃)₄ (0.10 equiv.). To the mixture was added degassed (sparged with nitrogen gas for 2 minutes) N,N-Dimethylformamide (1 mL). The mixture was stirred and purged with nitrogen for 2 min. The vial was capped and the mixture was then heated at 100° C. for 18 h. The reaction mixture was diluted with water and extracted with EtOAc, dried over Na₂SO₄ and concentrated in vacuo. The resulting residue was subjected to silica gel chromatography (12 g or 24 g column) using 5-100% ethyl acetate in hexanes and then 100% ethyl acetate. The desired fractions were pooled and then concentrated under reduced pressure to afford a solid (typically purple or yellow). The solid was taken up in DCM (1 mL): TFA (0.5 mL) and to the solution was added triflic acid (3 equiv.). The resultant purple solution was stirred at RT for 30-60 min and then was concentrated in vacuo. The residue was taken up in ethyl acetate. The pH was adjusted to pH>7 using aq. 1 N NaOH. The mixture was dried over Na₂SO₄, filtered, and then concentrated in vacuo. The resulting residue was dissolved in DMF, the mixture was filtered, and the filtrate was subjected to prep-HPLC purification to afford the indicated product.

### General Procedure E:

To 5 mL vial equipped with a stir bar was added a solution of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (30 mg, 0.031 mmol) in THF (1.0 mL), a solution of K₃PO₄ (0.025 g, 0.094 mmol) in water (0.25 mL), dichloro[9,9-dimethyl-4,5-bis(diphenylphosphino)xanthene]palladium(II) (2.377 mg, 3.14 μmol), and the appropriate boronic acid (0.094 mmol). The vial was degassed (the flask was evacuated and the atmosphere replaced with Ar; this process repeated three time) and then maintained under Ar atmosphere. The mixture was stirred at rt for 16 h. To the mixture was added 2 M ammonia in methanol (1 mL). The mixture was stirred for 2 h and then concentrated under reduced pressure. The resulting residue was dissolved in DMF, the solution was filtered, and the filtrate was subjected to prep-HPLC purification to afford the product as indicated.

### General Procedure F:

A 5 mL microwave vial was charged with N-((S)-1-((3P)-3-(4-chloro-3-(N-(4-methoxybenzyl)methylsulfonamido)-1-methyl-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.042 g, 0.035 mmol), the appropriate halide (0.104 mmol), copper(I) iodide (0.658 mg, 3.46 μmol) and Pd(PPh₃)₄ (3.99 mg, 3.46 μmol). To the mixture was added degassed (bubbled with nitrogen gas for 2 minutes) N,N-Dimethylformamide (1 mL). The mixture was stirred and purged with nitrogen for 2 min. The vial was capped and heated at 100° C. for 18 h. The reaction mixture was diluted with water and extracted with EtOAc, dried over Na₂SO₄ and concentrated under reduced pressure. The resulting residue was purified by silica gel chromatography (24 g RediSep Gold column) using 0-70% ethyl acetate in hexanes over 10 CV, then at 70% EtOAc in hexanes over 5 CV. The desired fractions were pooled and concentrated to afford a yellow solid. The solid was taken up in DCM (1 mL):TFA (0.5 mL) and to the solution was added triflic acid (0.016 mL, 0.182 mmol). The resulting purple solution was stirred for 30 min and then concentrated in vacuo. The residue was taken up in ethyl acetate and the pH was then adjusted to pH>7 by the addition of aq. 1N NaOH. The mixture was dried over Na₂SO₄, filtered, and concentrated in vacuo. The resulting residue was dissolved in DMF, the solution was filtered, and the filtrate was subjected to prep-HPLC purification to afford the indicated product.

### General Procedure G:

A 5 mL microwave vial was charged with the indicated stannane (0.068 mmol)), (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-y)acetamido)-2-(3,5-difluorophenyl)ethyl)-4-oxo-3,4-dihydropyrido[2,3-d]pyrimidin-7-yl trifluoromethanesulfonate (0.05 g, 0.052 mmol), and Pd(PPh₃)₄ (6.06 mg, 5.24 μmol). To the mixture was added degassed (bubbled with nitrogen gas for 2 minutes) N,N-Dimethylformamide (1 mL). The mixture was stirred and purged with nitrogen for 2 min. The vial was capped and heated at 100° C. for 18 h. The reaction mixture was diluted with DMF(1 mL), filtered, and the filtrate was subjected to prep-HPLC purification to afford the indicated product.

### General Procedure H:

In a 5 mL microwave vial equipped with a stir bar were combined N-((S)-1-((3P)-3-(4-chloro-1-methyl-3-(methylsulfonamido)-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1 equiv, typically 0.032-0.041 mmol), Pd(PPh₃)₄ (0.1 equiv), copper(I) iodide (0.1 equiv), the indicated halo-heterocycle (3 equiv), and DMF (0.03M relative to stannane). The vial was sealed with a septum cap and then was placed under Ar atmosphere (vacuum evacuation followed by refill with Ar, repeated 3 times). The vial was placed in a 100° C. heating block upon which the yellow solution quickly turned brown and then black. The mixture was stirred at 100° C. for 30-60 min. The reaction mixture was diluted with DMF (up to 2 mL), then filtered, and the filtrate was subjected to HPLC purification to afford the indicated product.

### General Procedure J:

In a 5 mL microwave vial was combined (3P)-3-(4-chloro-1-methyl-3-(methylsulfonamido)-1H -indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (1 equiv, typically 0.031-0.037 mmol), copper(I) iodide (0.1 equiv), Pd(PPh₃)₄ (0.1 equiv), the indicated stannane (3 equiv) and DMF (0.1M relative to trifluoromethanesulfonate). The vial was purged with N₂ gas and then was capped with a septum cap. The vial was placed in a 100° C. reaction block with stirring for 15-60 min (reaction progress monitored by LCMS). The reaction solution was cooled to r.t. and then was subjected to HPLC purification to afford the indicated product.

### General Procedure K:

In a dry 1 dram vial equipped with a stir bar was combined (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (or other coupling partner as indicated) (1 equiv, typically 0.028-0.037 mmol), tribasic potassium phosphate (3 equiv), dichloro[9,9-dimethyl-4,5-bis(diphenylphosphino)xanthene]palladium(II) (0.05-0.1 equiv) and the indicated boronic acid or boronic ester (2-3 equiv). The vial was purged with argon and then was sealed with a septum cap. To the vial was added THF:water (4:1, 0.05M relative to trifluoromethanesulfonate). The mixture was stirred at either ambient temperature or 60° C. for 1-18 h (typically 18 h). Upon cooling to ambient temperature, the reaction was concentrated and the residue was subjected to HPLC purification to afford the indicated product.

### General Procedure M:

To a solution of N-((S)-1-((3P)-3-(4-chloro-3-(N-(4-methoxybenzyl)methylsulfonamido)-1-methyl-1H-indazol-7-yl)-4-oxo-7-(1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (or other pyrazole as indicated) (25 mg, 0.025 mmol) and the indicated triflate (0.076 mmol) in acetonitrile (1 mL) was added cesium carbonate (12.31 mg, 0.038 mmol). The resulting mixture was heated at 50° C. for 1 h, then the mixture was then cooled to room temperature, filtered and concentrated under reduced pressure. The resulting residue was taken up in DCM (0.5 mL) and to the mixture was added TFA (1 mL) and then triflic acid (0.05 mL). The mixture was stirred at rt for 1 h and then was concentrated in vacuo. The resulting residue was then taken up in DMF (2 mL), filtered, and the filtrate was subjected to HPLC purification to afford the indicated product.

### General LCMS Analysis Methods

### LCMS Method A:

Column=Acquity UPLC BEH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.6 min, then a 0.25 min hold at 95% B. Detection=215 nm.

### LCMS Method B:

Column=Acquity BEH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### LCMS Method D:

Column: Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min.; Start % B=0, Final % B=100; Gradient Time=3.5 min, then a 1 min hold at 100% B. Detection=220 and 254 nm.

### LCMS Method G:

Column=XBridge C18 2.1×50 mm, 3.5 μm particles; Solvent A=95:5 Water:MeCN w/ 10 mM NH₄OAc; Solvent B=5:95 Water:MeCN 10 mM NH₄OAc; Flow Rate=1.0 mL/min.; Start % B=0, Final % B=100; Gradient Time=3 min, then a 1 min hold at 100% B. Detection=220 nm and 254 nm.

### LCMS Method H:

Column=Acquity CSH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### General HPLC Purification Conditions:

HPLC purification was performed using one of the conditions indicated below, optionally followed by a second HPLC purification using a different condition indicated below. Based on analytical HPLC data obtained on the crude reaction mixture, the purification condition was optimized for each target compound by modifying the initial Solvent A:Solvent B ratio, the gradient time, the final Solvent A:Solvent B ratio, and the hold time at the final Solvent A:Solvent B concentration.

HPLC Condition A: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Wavelength=215 and 254 nm. ESI+Range: 150 to 1500 dalton.

HPLC Condition B: Column: Sunfire prep C18 OBD, 30×100 mm, 5 μm particles; Solvent A: water:MeCN 95:5 w/ 0.1% TFA, Solvent B: MeCN:water 95:5 w/ 0.1% TFA. Flow Rate=42 mL/min. Wavelength=220 and 254 nm.

HPLC Condition C: Column: Waters Xterra C18, 19×100 mm, 10 μm particles; Solvent A=0.1% NH4OH in 100% Water. Solvent B=Acetonitrile. Flow Rate=40 mL/min. Wavelength=215 and 254 nm. ESI+Range: 150 to 1500 dalton.

HPLC Condition D: Column: Waters XSelect CSH C18, 19×100 mm, 5 μm particles; Solvent A=0.1% Formic Acid in 100% Water. Solvent B=Acetonitrile. Flow Rate=40 mL/min. Wavelength=215 and 254 nm. ESI+Range: 150 to 1500 dalton.

### Preparation of bicyclo[3.1.0]hexan-3-ol

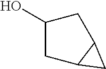

To a stirred solution of cyclopent-3-enol (130 g, 1545 mmol) in DCM (1200 mL) under N₂ atmosphere at 0-5° C. was added dropwise a solution of diethyl zinc in hexane (1.0 M, 3091 mL, 3091 mmol) over a period of 3 h. To the solution at 0° C. was added dropwise a solution of diiodomethane (249 mL, 3091 mmol) in DCM (300 mL) over a period of 1 h. The reaction mixture was allowed to warm to 27° C. upon which formation of a white precipitation was observed. The mixture stirred for 16 h. Progress of the reaction was monitored by TLC (SiO₂, 20% EtOAc/pet, Rf=0.3, UV-inactive, PMA-active). The reaction mixture was quenched via the careful addition of aq. saturated NH₄Cl solution (1.5 L). The mixture was filtered through pad of Celite. The aqueous layer was extracted with DCM (2×1L). The combined organic layers were dried over anhydrous Na₂SO₄, filtered and then concentrated under reduced pressure to afford crude bicyclo[3.1.0]hexan-3-ol as red liquid, 180 g. ¹H NMR (400 MHz, CDCl₃)δ=4.41-4.35 (m, 1H), 2.18-2.05 (m, 2H), 1.73 (d, J=13.9 Hz, 2H), 1.35-1.25 (m, 2H), 1.21-1.14 (m, 1H), 0.57-0.43 (m, 2H). GCMS: m/z=98.1).

### Preparation of bicyclo[3.1.0]hexan-3-one

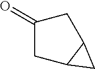

To a stirred solution of bicyclo[3.1.0]hexan-3-ol (210 g, 2054 mmol) in DCM (5000 mL) under N₂ atmosphere at 0° C. was added portion-wise Dess-Martin periodinane (954 g, 225 mmol). The mixture was allowed to warm to 27° C. and was then stirred for 16 h. Progress of the reaction was monitored by TLC (SiO₂, 20% Acetone/Hex, Rf=0.3, UV in-active, PMA-active). The reaction mixture was filtered through pad of Celite and the filtrate was washed with aq. NaOH (1N, 8×1 L). The combined aqueous phases were extracted with DCM (5×1 L). The combined organic layers were dried over anhydrous Na₂SO₄, filtered, and then concentrated under reduced pressure (bath temperature: 20° C.) to afford crude bicyclo[3.1.0]hexan-3-one as brown liquid. The liquid was further purified by downward distillation at 70° C. to afford bicyclo[3.1.0]hexan-3-one as a pale yellow viscous liquid, 125 g (62%). ¹H NMR (400 MHz, CDCl₃)δ=2.61-2.54 (m, 2H), 2.17-2.12 (m, 2H), 1.54-1.46 (m, 2H), 0.92-0.86 (m, 1H), −0.01-−0.08 (m, 1H); GCMS: M/Z=96.1.

### Preparation of 2-(2,2-difluoroacetyl)bicyclo[3.1.0]hexan-3-one

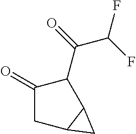 [SMILES: O=C1CC2CC2C1C(=O)C(F)F]

To a stirred solution of bicyclo[3.1.0]hexan-3-one (125 g, 1274 mmol) in THF (1500 mL) under N₂ atmosphere at −78° C. was added LDA (2.0 M in THF, 0.701 L, 1402 mmol). The solution was stirred for 1 h at −78° C. To the solution was added slowly over 30 minutes a solution of ethyldifluoroacetate (174 g, 1402 mmol) in THF (300 mL) maintaining a temperature of −78° C. The reaction mixture was allowed to warm to 27° C. and was then stirred for 1 h. Progress of the reaction was monitored by TLC (SiO₂, 20% Acetone/Hexane, Rf=0.3, UV-active). The reaction mixture was quenched via the addition of aq. HCl (1N, 2000 mL). The mixture was stirred for 30 min. and then was extracted with EtOAc (3×1000 mL). The combined organic layers were washed with brine (1000 mL), dried over anhydrous Na₂SO₄ and filtered. The filtrate was concentrated under reduced pressure to afford 2-(2,2-difluoroacetyl)bicyclo[3.1.0]hexan-3-one as a pale yellow viscous liquid, 180 g (71%). ¹H NMR (400 MHz, CDCl₃)δ=6.18 (t, J=54.8 Hz, 1H), 2.70-2.62 (m, 1H), 2.35 (d, J=19.4 Hz, 1H), 2.14 (br s, 1H), 1.26-1.21 (m, 1H), 1.04-1.03 (m, 1H), 0.22-0.21 (m, 1H), LCMS: M/Z=173.17).

### Preparation of ethyl 2-(3-(difluoromethyl)-3b,4,4a,5-tetrahydro-1H-cyclopropa[3,4]cyclopenta[1,2-c]pyrazol-1-yl)acetate.

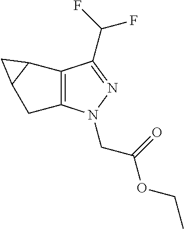 [SMILES: CCOC(=O)Cn1nc(C(F)F)c2c1CC1CC21]

To a stirred solution of 2-(2,2-difluoroacetyl)bicyclo[3.1.0]hexan-3-one (180 g, 910 mmol) in ethanol (2 L) under N₂ atmosphere at 27° C. was added ethyl 2-hydrazinylacetate hydrochloride (422 g, 2729 mmol) followed by sulfuric acid (20 mL, 375 mmol). The mixture was stirred for 30 min. and then was heated to 100° C. and stirred for 16 h. Progress of the reaction was monitored by TLC (SiO₂, 20% Acetone/Hexane, Rf=0.3, UV-active). The reaction mixture was concentrated under reduced pressure. The residue was dissolved in EtOAc (2000 mL) and was washed with water (2×1 L), brine (1.0 L), dried over anhydrous Na₂SO₄, filtered, and then was concentrated under reduced pressure. The resulting residue was subjected to silica gel column chromatography (pet.:acetone 100:0→98:2) to afford ethyl 2-(3-(difluoromethyl)-3b,4,4a,5-tetrahydro-1H-cyclopropa[3,4]cyclopenta[1,2-c]pyrazol-1-yl)acetate as an off-white solid, 110 g (46%). ¹H NMR (400 MHz, DMSO-d₆) δ=6.86 (t, J=54.8 Hz, 1H), 4.93 (s, 2H), 4.14 (q, J=7.2 Hz, 2H), 2.88-2.79 (m, 1H), 2.76-2.68 (m, 1H), 2.14-2.04 (m, 2H), 1.19 (t, J=7.2 Hz, 3H), 1.10-1.03 (m, 1H), 0.14 (q, J=4.3 Hz, 1H).

### Preparation of ethyl 2-(3-(difluoromethyl)-5-oxo-3b,4,4a,5-tetrahydro-1H-cyclopropa[3,4]cyclopenta[1,2-c]pyrazol-1-yl)acetate.

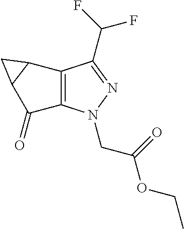 [SMILES: CCOC(=O)Cn1nc(C(F)F)c2c1C(=O)C1CC21]

To a stirred solution of ethyl 2-(3-(difluoromethyl)-3b,4,4a,5-tetrahydro-1H-cyclopropa[3,4]cyclopenta[1,2-c]pyrazol-1-yl)acetate (110 g, 422 mmol) and Celite (395 g) in cyclohexane (3.5 L) at 0° C. was added portionwise pyridinium dichromate (794 g, 2110 mmol). To the mixture under nitrogen atmosphere was added dropwise tert-butyl hydroperoxide (355 mL, 2130 mmol) over a period of 10 min. The reaction mixture was warmed to 27° C. and was then stirred at that temperature for 48 h. Progress of the reaction was monitored by TLC (SiO₂, 30% Acetone/pet, Rf=0.4, UV-active). The reaction mixture was filtered, and the filter cake was extracted with EtOAc (1000 mL). The filtrate was washed with saturated aq. Na₂S₂O₃ (2×500 mL); saturated aq. FeSO₄ (300 mL); and then brine (500 mL). The organic layer was dried over anhydrous Na₂SO₄, filtered and concentrated under reduced pressure to obtain the crude title compound (150 g).

### Preparation of ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate.

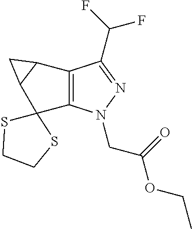 [SMILES: CCOC(=O)Cn1nc(C(F)F)c2c1C1(SCCS1)C1CC21]

To a stirred solution of ethyl 2-(3-(difluoromethyl)-5-oxo-3b,4,4a,5-tetrahydro-1H-cyclopropa[3,4]cyclopenta[1,2-c]pyrazol-1-yl)acetate (75 g, 269 mmol) in DCM (1500 mL) at 27° C. under nitrogen atmosphere was added ethane-1,2-dithiol (43.0 mL, 511 mmol) followed by the addition of boron trifluoride acetic acid (72.6 mL, 511 mmol). The solution was stirred for 16 h. Progress of the reaction was monitored by TLC (SiO₂, 20% Acetone/Pet, Rf=0.35, UV-active). After completion, the reaction mixture was cooled to 0° C. and quenched via the addition of aq. saturated NaHCO₃ (500 mL). The mixture was extracted with DCM (2×1000 mL). The combined organics were washed with brine (1000 mL), dried over anhydrous Na₂SO₄, filtered and concentrated under reduced pressure to obtain a brown liquid. This material was subjected to silica gel column chromatography (Pet.:EtOAc 95:5→90:10) to afford ethyl 2-(3-(difluoromethyl)-4,4a-dihydrospiro[cyclopropa[3,4]cyclopenta[1,2-c]pyrazole -5,2′-[1,3]dithiolane]-1(3bH)-yl)acetate as an off-white solid, 80 g (74%). ¹H-NMR (400 MHz, CDCl₃) δ=6.61 (t, J=55.2 Hz, 1H), 5.00-4.85 (m, 2H), 4.29-4.19 (m, 2H), 3.55-3.46 (m, 4H), 2.63-2.53 (m, 1H), 2.49-2.38 (m, 1H), 1.30-1.24 (m, 4H), 0.65-0.60 (m, 1H). LCMS M+H=346.9.

### Preparation of ethyl 2-(3-(difluoromethyl)-5,5-difluoro-3b,4,4a,5-tetrahydro-1H-cyclopropa[3,4]cyclopenta[1,2-c]pyrazol-1-yl)acetate

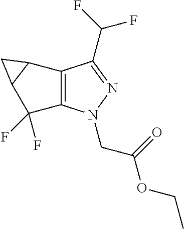 [SMILES: CCOC(=O)Cn1nc(C(F)F)c2c1C(F)(F)C1CC21]

To a stirred solution of 1,3-dibromo-5,5-dimethylimidazolidine-2,4-dione (26.3 g, 92 mmol) in DCM (20 mL) at −70° C. under N₂ atmosphere was added HF-pyridine (2.460 g, 24.83 mmol). The solution was for 30 min. To the solution was added a solution of ethyl 2-(3-(difluoromethyl)-4,4a-dihydrospiro[cyclopropa[3,4]cyclopenta[1,2-c]pyrazole-5,2′-1,3]dithiolane]-1(3bH)-yl)acetate (10 g, 25 mmol) in DCM (20 mL). The reaction mixture was allowed to warm to -40° C. and then was stirred at that temperature for 1 h. Progress of the reaction was monitored by TLC (SiO2, 30% EtOAc/Pet, Rf=0.3, UV in-active). The reaction mixture was quenched via the addition of aq. sat. NaHCO₃ (200 mL). The mixture was warmed to room temperature and was then extracted with EtOAc (2×100 mL). The combined organics were washed with brine (50 mL); dried over anhydrous Na₂SO₄; filtered; and were concentrated under reduced pressure to afford a brown solid. This material was subjected to silica gel column chromatography (Pet.:EtOAc 100:0→75-25) to afford ethyl 2-(3-(difluoromethyl)-5,5-difluoro-3b,4,4a,5-tetrahydro-1H-cyclopropa[3,4]cyclopenta[1,2-c]pyrazol-1-yl)acetate as a pale yellow solid, 8.5 g (91%). ¹H NMR (400 MHz, CDCl₃) δ=6.62 (t, J=55.2 Hz, 1H), 4.82 (s, 2H), 4.30-4.18 (m, 2H), 2.51-2.37 (m, 2H), 1.42-1.35 (m, 1H), 1.31-1.23 (m, 3H), 1.14-1.08 (m, 1H). LCMS M+H=293.07.

### Preparation of 2-(3-(difluoromethyl)-5,5-difluoro-3b,4,4a,5-tetrahydro-1H-cyclopropa[3,4]cyclopenta[1,2-c]pyrazol-1-yl)acetic acid

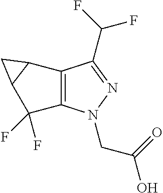 [SMILES: O=C(O)Cn1nc(C(F)F)c2c1C(F)(F)C1CC21]

To a stirred solution of ethyl 2-(3-(difluoromethyl)-5,5-difluoro-3b,4,4a,5-tetrahydro-1H-cyclopropa[3,4]cyclopenta[1,2-c]pyrazol-1-yl)acetate (15 g, 50 mmol) in THF (17 mL) and MeOH (66 mL) at 0° C. under N₂ atmosphere was added a solution of LiOH (1.788 g, 74.7 mmol) in water (66 mL). The reaction mixture was allowed to warm to 27° C. and was then stirred for 3 h at that temperature. Progress of the reaction was monitored by TLC (SiO₂, 5% MeOH/DCM, Rf=0.2, UV Active). After completion, the reaction mixture was concentrated under reduced pressure; diluted with water (50 mL); and washed with EtOAc (2×250 mL) to remove impurities. The aqueous layer was adjusted to pH 2-3 using aq. HCl (1M), then was extracted with EtOAc (3×1000 mL). The combined organics were dried over anhydrous Na₂SO₄; filtered; and concentrated under reduced pressure to afford 2-(3-(difluoromethyl)-5,5-difluoro-3b,4,4a,5-tetrahydro-1H-cyclopropa[3,4]cyclopenta[1,2-c]pyrazol-1-yl)acetic acid as an off white solid, 14 g (98%). LCMS M+H=265.15.

### Separation affording 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid and 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid

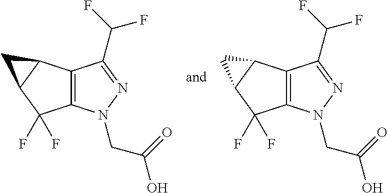 [SMILES: O=C(O)Cn1nc(C(F)F)c2c1C(F)(F)[C@H]1C[C@@H]21 ; O=C(O)Cn1nc(C(F)F)c2c1C(F)(F)[C@@H]1C[C@H]21]

2-(3-(difluoromethyl)-5,5-difluoro-3b,4,4a,5-tetrahydro-1H-cyclopropa[3,4]cyclopenta[1,2-c]pyrazol-1-yl)acetic acid (5.5 g) was dissolved in isopropanol (20 mL). The solution was subjected portion-wise to SFC chiral separation as follows: Instrument=Thar 80; column=Chiralpak IC 30×250 mm, 5 micron; solvent A=super critical CO₂; solvent B=isopropanol with 0.5% isopropylamine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afford the separated enantiomer in 80-90% recovery.

### Preparation of N-(7-amino-4-chloro-1-methyl-1H-indazol-3-yl)-N-(4-methoxybenzyl)methanesulfonamide.

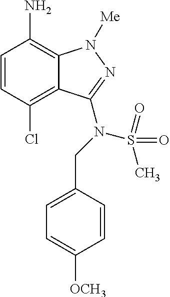 [SMILES: COc1ccc(CN(c2nn(C)c3c(N)ccc(Cl)c23)S(C)(=O)=O)cc1]

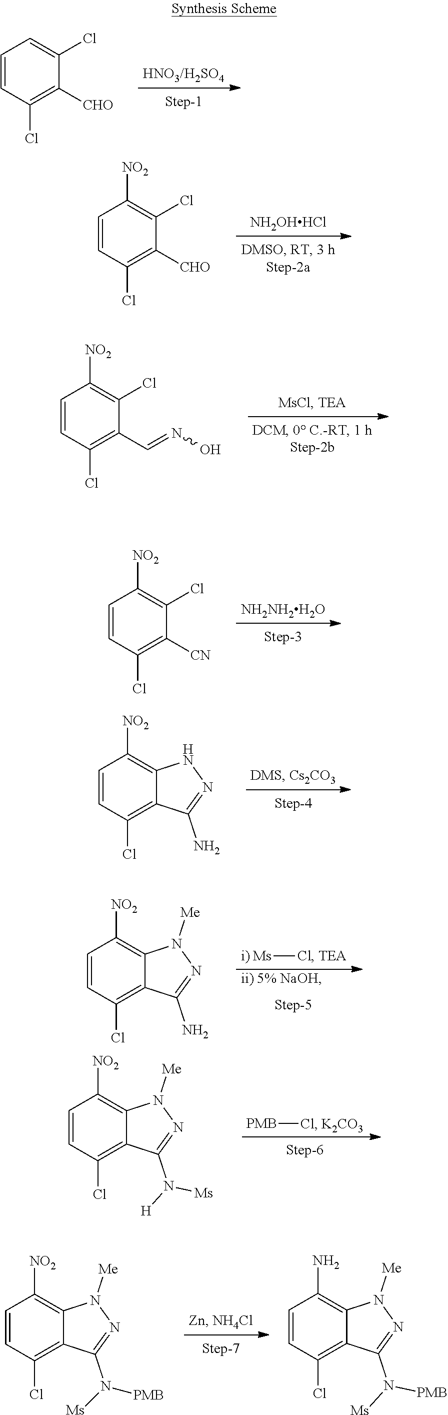

### Step 1: Preparation of 2,6-dichloro-3-nitrobenzaldehyde

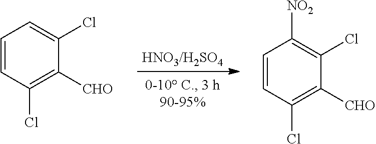 [SMILES: O=Cc1c(Cl)cccc1Cl ; O=Cc1c(Cl)ccc([N+](=O)[O-])c1Cl]

To a solution of sulfuric acid (H₂SO₄) (5.63 L, 4.5 V) in a round-bottom flask at 0-5° C. was added 2,6-dichlorobenzaldehyde (1.25 kg, 7.10 mol, 1.0 equiv.) in portions at below 15° C. The reaction mass was stirred at 0-5° C. for 30 min. A solution of freshly prepared nitration mixture [Prepared from Conc. H₂SO₄ (0.425 L, 0.34 V) and 70% HNO₃ (0.85 kg, 13.49 mol, 1.30 equiv.) at 0° C.] was added to the above reaction mixture at below 10° C. [Note: Reaction is slightly exothermic (3-6° C.); so that addition is preferred at lower temperature]. The reaction mixture was stirred at 5-10° C. for 2-3 h. After completion of the reaction (monitored by TLC), it was quenched with ice cold water (18.75 L, 15 V) at below 25° C. Then the reaction mass was allowed warm to room temperature and stirred for 2 h. The solids were isolated by filtration and then were washed with water (2.5 L, 2.0 V). Bulk residual water was removed from the solids by maintaining vacuum filtration for 60-90 min. The crude wet solid was initially dried under air atmosphere; then in a hot air oven at 50-55° C. for 10-12 h (until moisture content is not more than 5.0%) to get the dried title product, 2,6-dichloro-3-nitrobenzaldehyde (1.44 kg, 92% yield) as a yellow solid. ¹H NMR (400 MHz, CDCl₃): δ10. 44 (s, 1H), 7.88 (d, J=8.4 Hz, 1H), 7.56 (d, J=8.8 Hz, 1H).

### Step 2: Preparation of 2,6-dichloro-3-nitrobenzonitrile

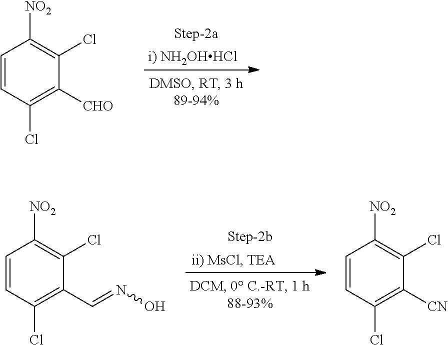

(Step-2a) To a solution of DMSO (5.9 L, 5.0 V)) in a round-bottom flask was added 2,6-dichloro-3-nitrobenzaldehyde (1.17 kg, 5.31 mol, 1.0 equiv.) at room temperature. After being stirred for 30 min at room temperature, hydroxylamine hydrochloride (0.63 kg, 9.04 mol, 1.70 equiv.) was added and the reaction mass was stirred at room temperature for 3 h. After completion of the reaction (monitored by TLC), the reaction mass was quenched by the addition of ice-cold water (18.0 L, 15.0 V) added at a rate sufficient to maintain the temperature below 30° C. (Observation: Solids formed upon water addition). The reaction mass was stirred at room temperature for 60-90 min. The solids were isolated by filtration; washed with water (2.5 L, 2.0 V); followed by washing with a mixture of acetone and hexanes (6.0 L, 1:1 ratio). Bulk residual water was removed from the solids by maintaining vacuum filtration for 60-90 min. The wet solid was initially air dried and then finally dried in a hot air oven at 50-55° C. for 10-12 h (until moisture content was not more than 1.0%) to get the dried target product, 2,6-dichloro-3-nitrobenzaldehyde oxime (1.22 kg, 92% yield) as an off-white solid. The crude product (which contains 10-20% of 2,6-dichloro-3-nitrobenzonitrile) was used directly in the next step without further purification.

(Step-2b) To a stirred solution of the crude oxime (preparation described above, 1.13 kg, 4.80 mol, 1.0 equiv.) in DCM (9.04 L, 8.0 V) at 0-5° C. was added triethylamine (“TEA”, 1.02 kg, 10.09 mol, 2.1 equiv.). After being stirred for 5 min, methanesulfonyl chloride (0.60 kg, 5.29 mol, 1.1 equiv.) was added (Observation: An exotherm is noted during the addition) slowly at 15° C. Then the reaction mass was stirred at room temperature for 30-45 min. After completion of the reaction (progress of reaction was monitored by TLC; mobile phase: 20% ethyl acetate in hexanes), the reaction mass was diluted with water (6.78 L, 6.0 V); the organic layer was separated; and the aqueous layer was extracted with DCM (3.4 L, 3.0 V). The combined organic layers were washed with brine (5.65 L, 5.0 V); dried over Na₂SO₄; and concentrated under vacuum. The resulting crude solids were triturated with hexanes (4.50 L, 4.0 V) at room temperature. The wet material was dried in a hot air oven at 50-55° C. for 5-6 h to get the dried product, 2,6-dichloro-3-nitrobenzonitrile (0.95 kg, 91% yield) as a yellow solid. ¹H NMR (400 MHz, CDCl₃): δ8.07 (d, J=8.8 Hz, 1H), 7.63 (d, J=8.8 Hz, 1H).

### Step 3: Preparation of 4-chloro-7-nitro-1H-indazol-3-amine

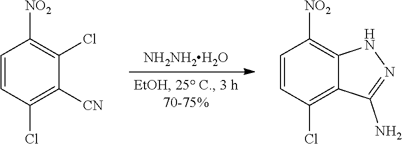 [SMILES: N#Cc1c(Cl)ccc([N+](=O)[O-])c1Cl ; "NH2NH2·H2O" ; Nc1n[nH]c2c([N+](=O)[O-])ccc(Cl)c12]

To a stirred solution of 2,6-dichloro-3-nitrobenzonitrile (750.0 g, 3.45 mol, 1.0 equiv.) in ethanol (7.5 L, 10.0 V) at 15-20° C. was slowly added hydrazine hydrate (519.0 g, 10.36 mol, 3.0 equiv.) while maintaining the reaction mass below 25° C. (Observation: Addition is slightly exothermic and solid formation will begin upon addition). The reaction mixture temperature was slowly raised to room temperature and then the mixture was stirred for 3 h (Observation: the quantity of solids will increase during this time). After completion of the reaction (monitored by TLC), the mixture was diluted with water (7.5 L, 10.0 V) and further stirred for 1 h at room temperature. The solids were isolated via filtration and then were washed with water (2.25 L, 3.0 V). The wet solid was washed with a 1:1 ratio mixture of acetone (1.875 L, 2.5 V) and hexanes (1.875 L, 2.5 V). Bulk residual water was removed from the solids by maintaining vacuum filtration for 60-90 min. The wet solid was finally dried in a hot air oven for 7-8 h at 50° C. (until moisture content reaches below 1.5%) to get the dried product, 4-chloro-7-nitro-1H-indazol-3-amine (549.0 g, 75% yield) as a brick red-colored solid. ¹H NMR (400 MHz, CDCl₃): δ10.36 (bs, 1H), 8.20 (d, J=8.4 Hz, 1H), 7.07 (d, J=8.40 Hz, 1H), 4.73 (bs, 2H).

### Step 4: Preparation of 4-chloro-1-methyl-7-nitro-1H-indazol-3-amine

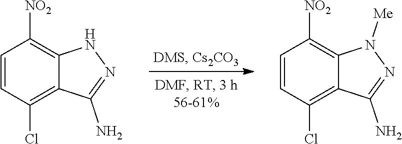

To a stirred solution of 4-chloro-7-nitro-1H-indazol-3-amine (500 g, 0.42 mol, 1.0 equiv.) in DMF (5.0 L, 10.0 V) at 5-10° C. was slowly added cesium carbonate (Cs₂CO₃) (1.91 kg, 5.88 mol, 2.5 equiv.) while maintaining the reaction mass below 10° C. After being stirred for 5-10 min, dimethyl sulphate (326.3 g, 2.59 mol, 1.1 equiv.) was added while maintaining the reaction mass below 10° C. (Note: Slow addition is preferred for obtaining more favorable regio-selectivity). Then, the reaction temperature was slowly raised to room temperature and stirring was continued an additional 2 h at the same temperature. After completion of the reaction (monitored by TLC), the reaction mass was quenched by the addition of ice-cold water (15.0 L, 30.0 V) and the resulting mixture was then stirred for 6-8 h at room temperature. The solids were isolated via filtration and were then washed with water (1.5 L, 3.0 V). The wet solid was washed with IPA (1.5 L, 3.0 V) followed by hexanes (1.0 L, 2.0 V). Bulk residual water was removed from the solids by maintaining vacuum filtration for 60-90 min. The wet solid was dried in a hot air oven for 7-8 h at 50° C. (until moisture content is below 1.0%). The isolated material, 4-chloro-1-methyl-7-nitro-1H-indazol-3-amine (319.0 g, 60% yield), was used in the next step without further purification. ¹H NMR (400 MHz, CDCl₃): δ7.97 (d, J=8.32 Hz, 1H), 6.97 (d, J=8.24 Hz, 1H), 4.63 (bs, 2H), 3.96 (s, 3H).

### Step 5: Preparation of N-(4-chloro-1-methyl-7-nitro-1H-indazol-3-yl)methanesulfonamide

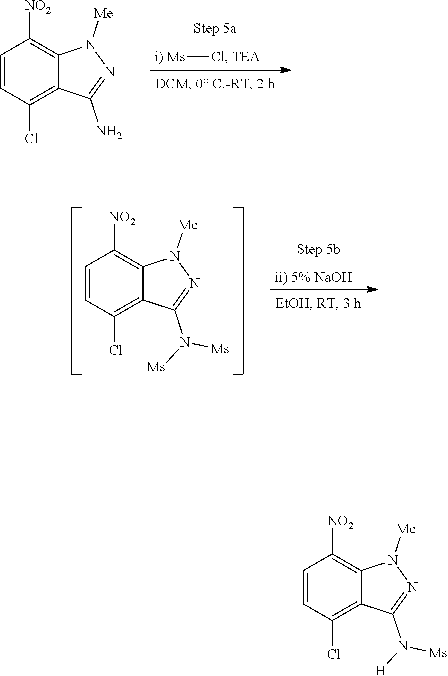

(Step 5a) To a solution of 4-chloro-1-methyl-7-nitro-1H-indazol-3-amine (625.0 g, 2.76 mol, 1.0 equiv.) in DCM (6.25 L, 10.0 V) at 0-5° C. was added triethylamine (TEA) (837.0 g, 8.27 mol, 3.0 equiv.); followed by the addition of 4-dimethylaminopyridine (DMAP) (20.60 g, 0.165 mol, 0.06 equiv.). The reaction mass was stirred for 5-10 min., then methanesulfonyl chloride (MSCl) (790.0 g, 6.89 mol, 2.5 equiv.) added slowly while maintaining the reaction mass below 10° C. The reaction mixture was allowed to warm to room temperature and was then stirred for 1.5-2.0 h. After completion of the reaction (monitored by TLC), the mixture was diluted with water (6.25 L, 10.0 V) and then stirred at room temperature for 15 min. The organic layer was separated, and the aqueous layer was extracted with DCM (6.25 L, 10.0 V). The combined organic layers were washed with brine (1.25 L, 2.0 V), dried over Na₂SO₄ and concentrated to get the crude solids. The solids were triturated with hexanes (1.25 L, 2.0 V) at room temperature to obtain the intermediate, N-(4-chloro-1-methyl-7-nitro-1H-indazol-3-yl)-N-(methylsulfonyl)methanesulfonamide, which was used directly in the next step.

(ii) To a stirred solution of N-(4-chloro-1-methyl-7-nitro-1H-indazol-3-yl)-N-(methylsulfonyl)methanesulfonamide (prepared above) in ethanol (10.5 L, 20.0 V) at room temperature was added slowly an aq. 5% NaOH solution (4.38 L, 7.0 V) [Note: Slow addition is preferred via dropping funnel]. The reaction mass was stirred at the same temperature for 3 h. After completion of the reaction (monitored by TLC) [Sample preparation for TLC analysis: ˜1.0 ml of sample acidified with aq. 2.0 N HCl to reach the pH: 2-3, extract it with ethyl acetate and analyze the organic layer by TLC], the reaction mass was cooled to 0-5° C. and the pH was adjusted to 2-3 by the addition of aq. 2.0 N HCl (3.13 L, 5.0 V) while maintain the reaction temperature below 10° C. [Note: Precipitation occurred upon addition of HCl and increased with stirring]. The reaction mixture was warmed to room temperature and then stirred for 1.5-2.0 h. Solids obtained were isolated via filtration and were then washed with water (1.25 L, 2.0 V); followed by washing with hexanes (1.25 L, 2.0 V). Bulk residual water was removed from the solids by maintaining vacuum filtration for 60-90 min. The wet material was dried in a hot air oven at 50° C. for 6-7 h (Until the moisture content is below 1.0%) to get the dried product, N-(4-chloro-1-methyl-7-nitro-1H-indazol-3-yl)methanesulfonamide (640.0 g, 76%) as a yellow solid. ¹H NMR (400 MHz, CDCl₃): δ8.05 (d, J=8.32 Hz, 1H), 7.32 (bs, 1H), 7.17 (d, J=8.28 Hz, 1H), 4.15 (s, 3H), 3.45 (s, 3H).

### Step 6: Preparation of N-(4-chloro-1-methyl-7-nitro-1H-indazol-3-yl)-N-(4-methoxybenzyl)methanesulfonamide

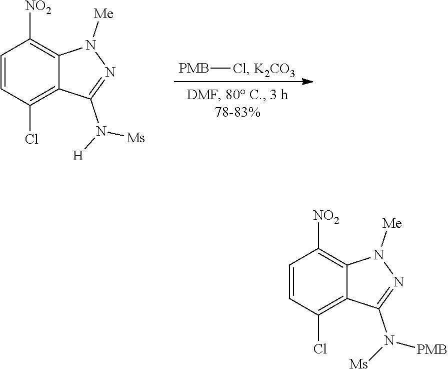

To a mixture of N-(4-chloro-1-methyl-7-nitro-1H-indazol-3-yl)methanesulfonamide (635.0 g, 2.08 mol, 1.0 equiv.) and 1-(chloromethyl)-4-methoxybenzene (359.0 g, 2.30 mol, 1.1 equiv.) in DMF (6.35 L, 10.0 V) at room temperature was added potassium carbonate (374.7 g, 2.70 mol, 1.3 equiv.). The reaction mixture was heated to 80-90° C. and maintained at that temperature for 3 h. After completion of the reaction (monitored by TLC), the mixture was poured into ice cold water (19.05 L, 30.0 V) [Note: Slow quenching with vigorous stirring is preferred to avoid clumping as the product precipitates]. The resulting solids were isolated via filtration and washed with water (1.90 L, 3.0 V); then the solids were washed with hexanes (1.27 L, 2.0 V). Bulk residual water was removed from the solids by maintaining vacuum filtration for 60-90 min. The isolated solid was dissolved in Ethyl acetate (12.7 L, 20.0 V) and charcoal was added (63.5 g). The mixture was heated to 60-70° C. and then stirred for 30-45 min. at that temperature. The mixture was filtered while still hot (40-50° C.) through a pad of Celite and the Celite pad was then extracted with ethyl acetate (3.17 L, 5.0 V). The combined filtrates were concentrated to dryness under reduced pressure at below 50° C. Ethyl acetate (0.635 L, 1.0 V) was added to the solids at room temperature. The resultant solid suspension was stirred for 30 min. The solids were isolated via filtration and then were washed with hexanes (1.27 L, 2.0 V). Residual water was removed from the solids by maintaining vacuum filtration for 45-60 min. to afford the product N-(4-chloro-1-methyl-7-nitro-1H-indazol-3-yl)-N-(4-methoxybenzyl) methane sulfonamide (705.0 g, 80% yield) as a yellow solid. ¹H NMR (400 MHz, CDCl₃): δ7.99 (d, J=8.24 Hz, 1H), 7.27 (d, J=8.68 Hz, 2H), 7.19 (d, J=8.24 Hz, 1H), 6.80 (d, J=8.44 Hz, 2H), 4.95-4.76 (m, 2H), 4.17 (s, 3H), 3.76 (s, 3H), 3.01 (s, 3H).

### Step 7: Preparation of N-(7-Amino-4-chloro-1-methyl-1H-indazol-3-yl)-N-(4-methoxybenzyl)methanesulfonamide

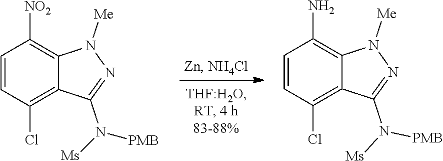

To a stirred suspension of zinc powder (540.0 g, 8.23 mol, 10.0 equiv.) in a mixture of THF (3.50 L, 10.0 V) and water (7.0 L, 20.0 V) at room temperature was added ammonium chloride (NH₄Cl) (449.0 g, 8.23 mol, 10.0 equiv.). To the mixture was added N-(4-chloro-1-methyl-7-nitro-1H-indazol-3-yl)-N-(4-methoxybenzyl)methanesulfonamide (350 g, 0.823 mol, 1.0 equiv.) in THF (7.0 L, 20.0 V). The reaction mixture was stirred at room temperature for 3-4 h. After completion of the reaction (monitored by in-process TLC/HPLC), the mixture was diluted with ethyl acetate (3.5 L, 10.0 V) and water (1.12 L, 2.5 V). The mixture was stirred for 15 min. The reaction mass was filtered through a pad of Celite bed washing with ethyl acetate (1.75 L, 5.0 V). The bi-phasic filtrate was collected, and the phases were separated. The aqueous layer was extracted with ethyl acetate (3.50 L, 10.0 V). The combined organic layers were washed with brine (3.50 L, 10 V), dried over Na₂SO₄, and then concentrated in vacuo to afford a crude solid. To the crude product was added MTBE (3.25 L, 10 V) and the suspension was stirred for 30 min at room temperature. The solids were isolated by filtration. Bulk residual water was removed from the solids by maintaining vacuum filtration for 30-45 min. The wet product was dried in a hot air oven (50° C.) for 2 h to afford the title product, N-(7-amino-4-chloro-1-methyl-1H-indazol-3-yl)-N-(4-methoxybenzyl)methanesulfonamide (276.0 g, 85% yield) as off-white solid. ¹H NMR (400 MHz, CDCl₃): δ7.29-7.26 (m, 2H), 6.86-6.79 (m, 2H), 6.42 (d, J=7.80 Hz, 1H), 4.99-4.70 (m, 2H), 4.25 (s, 3H), 3.77 (s, 5H), 2.98 (s, 3H).

### Preparation of 2-amino-6-(benzyloxy)nicotinic acid

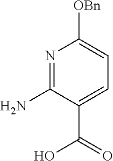 [SMILES: Nc1nc(OCc2ccccc2)ccc1C(=O)O]

A solution of 2-amino-6-chloronicotinic acid (5 g, 29 mmol) and potassium tert -butoxide (9.75 g, 87 mmol) in benzyl alcohol (97 mL) was stirred at 120° C. for 3 h. After cooling to ambient temperature, the very dark reaction mixture was diluted with water and then washed with ether (x3). The aqueous layer was then acidified with 0.5 M citric acid. The tan precipitate was isolated by filtration to provide the product (4.4 g, 62%) which was used in the next reaction without further purification. ¹H NMR (500 MHz, DMSO-d6) δ12.40 (br s, 1H), 7.94 (d, J=8.55 Hz, 1H), 7.06-7.52 (m, 5H), 6.04 (d, J=8.24 Hz, 1H), 5.33 (s, 2H). LC/MS: m/z=245.15 [M+1 ]⁺.

### Preparation of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide

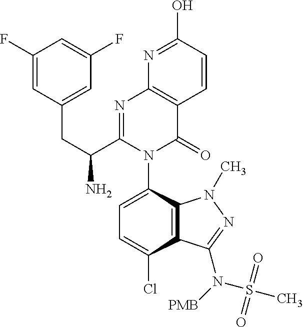

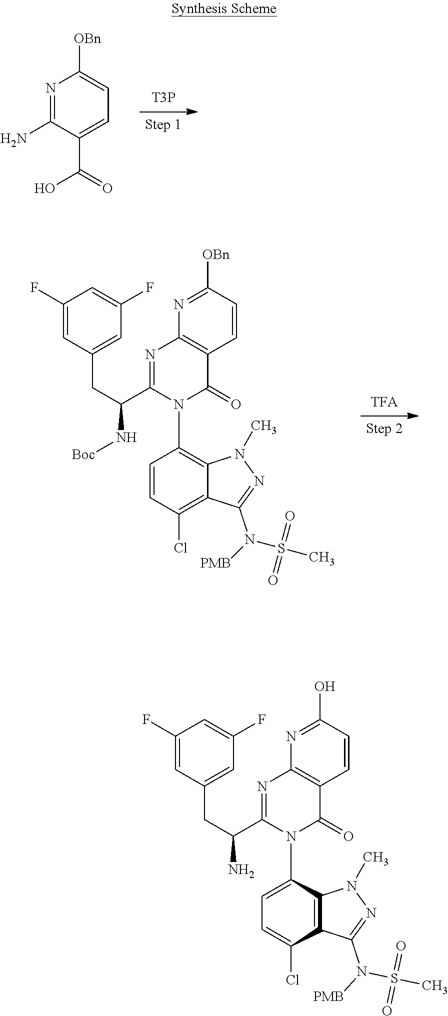

### Step 1:

To a suspension of (S)-2-((tert-butoxycarbonyl)amino)-3-(3,5-difluorophenyl)propanoic acid (5.49 g, 18.23 mmol) and 2-amino-6-(benzyloxy)nicotinic acid (4.45 g, 18.23 mmol) in acetonitrile (92 mL) (yellow solution) at −25° C. was added pyridine (9.83 mL, 122 mmol) followed by 2,4,6-tripropyl-1,3,5,2,4,6-trioxatriphosphinane 2,4,6-trioxide (“T3P”, 45.2 ml, 76 mmol). The reaction mixture (became a clear solution after T₃P addition) was stirred at −25° C. to 10° C. over 4.5 h, then N-(7-amino-4-chloro-1-methyl-1H-indazol-3-yl)-N-(4-methoxybenzyl)methanesulfonamide (6 g, 15.19 mmol) was added and the mixture was stirred for 18 h while warming to rt. The reaction mixture was diluted with ethyl acetate, washed with 1N NaOH, then water, then 0.5 M citric acid, then water, then dried over Na₂SO₄ and concentrated in vacuo. The resulting residue was purified on silica (330 g RediSep Gold column) using 0-60% ethyl acetate in hexanes over 15 CV, then holding at 60% EtOAc for 10 CV. The desired fractions were pooled and concentrated to afford a pale yellow solid (8.1 g, 9.14 mmol, 60.1% yield), a mixture of tert-butyl N-[(1S)-1-[(3P,3P)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (major) and tert-butyl N-[(1S)-1-[(3M,3M)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (minor). LC/MS: m/z=886.25[M+1]⁺.

### Step 2:

TFA (21.1 mL, 274 mmol) was added to a solution of tert-butyl (S)-(1-(7-(benzyloxy)-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate (Product from Step 1, 8.1 g, 9.14 mmol) in dichloromethane (45.7 mL). The mixture was stirred at rt for 2 h. The resultant pale yellow solution was concentrated. The residue was taken up in ethyl acetate, then washed three times with 1 N NaOH, then dried over Na2SO4 and then concentrated in vacuo to afford an oily residue. The residue was purified on silica gel (330 g RediSep Gold column) by a gradient method of Solvent A:Solvent B 65:35→0:100 (2 CV), then 0:100 (9 CV); Solvent A=hexanes; Solvent B=9:9:2 hexanes:ethyl acetate:MeOH. The first eluting isomer (major) was collected and concentrated in vacuo to afford N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (4.1 g, 5.89 mmol, 64.5% yield). ¹H NMR (500 MHz, DMSO-d6) δ 7.86-7.98 (m, 1H) 7.15-7.37 (m, 4H) 6.97-7.06 (m, 1H) 6.70-6.89 (m, 4H) 6.40-6.48 (m, 1H) 4.70-4.88 (m, 2H) 3.41-3.81 (m, 7H) 3.20-3.28 (m, 1H) 3.08-3.12 (m, 3H) 2.71-2.79 (m, 1H) 1.69-2.00 (m, 2H). LC/MS: m/z=696.20[M+1]⁺.

### Preparation of N-((S)-1-((3P)-3-(4-chloro-3-(N-(4-methoxybenzyl)methylsuIfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

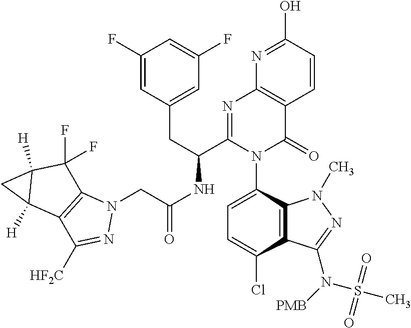

To a stirred solution of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (0.926 g, 1.330 mmol) in DMF (13 ml) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.351 g, 1.330 mmol), 2-(3H-[1,2,3]triazolo[4,5-b]pyridin-3-yl)-1,1,3,3-tetramethylisouronium hexafluorophosphate(V) (“HATU”, 0.531 g, 1.397 mmol), and DIPEA (0.581 ml, 3.33 mmol). The reaction mixture was stirred for 2 h after which the reaction mixture was diluted with water and extracted with ethyl acetate. The combined EtOAc extractions were washed with brine, dried over Na₂SO₄, and concentrated in vacuo. The crude product was purified via silica gel flash chromatography using 10-100% ethyl acetate in hexanes to provide N-((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.1 g, 88%) as an off-white foamy solid. LC/MS: m/z=942.25 [M+1]⁺.

### Preparation of N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

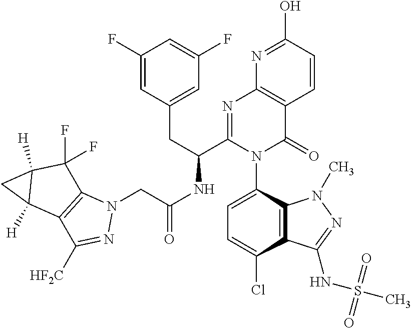

To a solution of N-((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.05 g, 0.053 mmol) in DCM (1 mL) and TFA (0.250 mL) was added triflic acid (0.014 mL, 0.159 mmol). The resultant purple solution was stirred for 1 h and then concentrated in vacuo. The crude residue was taken up in ethyl acetate and washed with saturated aqueous NaHCO3. The organic layer was concentrated in vacuo and then purified HPLC to afford the title compound, N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. ¹H NMR (500 MHz, METHANOL-d4) δ 8.09-8.17 (m, 1H) 7.27-7.32 (m, 1H) 7.16-7.21 (m, 1H) 6.58-6.85 (m, 5H) 4.81-4.83 (m, 2H) 4.42-4.47 (m, 2H) 3.65-3.70 (m, 3H) 3.43-3.49 (m, 1H) 3.23-3.27 (m, 3H) 3.06-3.14 (m, 1H) 2.41-2.50 (m, 2H) 1.35-1.41 (m, 1H) 0.96-1.02 (m, 1H). LCMS Method A: retention time=1.15 min; observed ion=822.6 [M+H]⁺

### Preparation of N-((S)-1-((3P)-3-(4-chloro-1-methyl-3-(N-(methylsulfonyl)acetamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

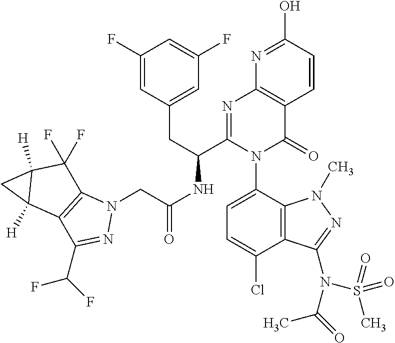 [SMILES: CC(=O)N(c1nn(C)c2c(-n3c([C@H](Cc4cc(F)cc(F)c4)NC(=O)Cn4nc(C(F)F)c5c4C(F)(F)[C@@H]4C[C@H]54)nc4nc(O)ccc4c3=O)ccc(Cl)c12)S(C)(=O)=O]

To a stirred solution of N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2 g, 2.433 mmol) in N,N-Dimethylformamide (12 mL) were added Acetic acid (0.84 mL, 14.60 mmol), 2-(3H-[1,2,3]triazolo[4,5-b]pyridin-3-yl)-1,1,3,3-tetramethylisouronium hexafluorophosphate(V) (“HATU”, 1.295 g, 3.41 mmol) and DIPEA (1.3 mL, 7.30 mmol). The reaction mixture was stirred for 2 days at rt. The mixture was diluted with ethyl acetate (200 mL), washed with water, brine, dried over Na₂SO₄, filtered, concentrated and the residue was purified by silica gel chromatography (120 g RediSep Gold column) using 10-80% ethyl acetate in hexanes over 15 CV, then at 80% ethyl acetate in hexanes for 10 CV. The desired fractions were pooled and then concentrated to afford N-((S)-1-((3P)-3-(4-chloro-1-methyl-3-(N -(methylsulfonyl)acetamido)-1H-indazol-7-yl)-7-hydroxy-4-oxo-3,4-dihydropyrido[2,3- d]pyrimidin-2-yl)-2-(3,5-difluorophenyl)ethyl)-2-((3bS,4aR)-3-(difluoromethyl)-5,5-difluoro -3b,4,4a,5-tetrahydro-1H-cyclopropa[3,4]cyclopenta[1,2-c]pyrazol-1-yl)acetamide (1.89 g, 90%) as a pale yellow solid. LC/MS: m/z=864.05[M+1]⁺.

### Preparation of N-((S)-1-((3P)-7-chloro-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

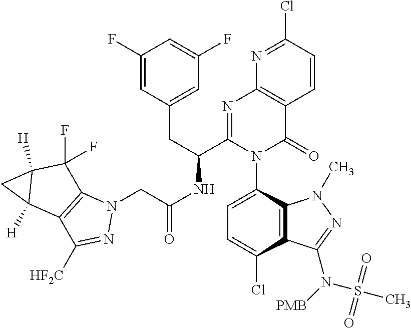

N-(1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.1 g, 0.106 mmol) in phosphorus oxychloride (0.53 mL) was heated at 80° C. for 3 h and then was concentrated in vacuo. The residue was taken up in ethyl acetate, washed with sat. NaHCO₃, brine, dried over Na₂SO₄ and concentrated under reduced pressure to give a yellow solid (used as is in the next step). LC/MS: m/z=960.15[M+1]⁺.

### Preparation of N-((S)-1-(7-chloro-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

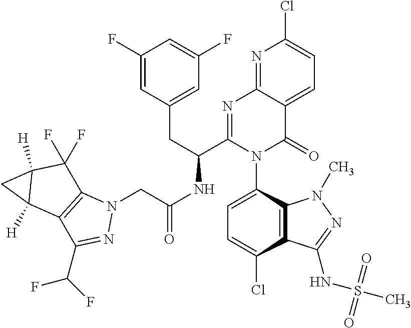

A solution of N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.0 g, 1.2 mmol) in POCl₃ (12 mL) was stirred overnight (app 18 h) at 40° C. The solution was poured over of ice (100 g) and then was diluted with EtOAc and stirred until the mixture had reached room temperature. The organic phase was isolated and dried over Na₂SO₄, filtered, and concentrated in vacuo to afford an orange oil (˜15 g). The material was adsorbed onto Celite and the resulting powder was subjected to silica gel purification (40 g, eluted with 30-100% ethyl acetate in hexanes over 5 CVs and then 100% ethyl acetate over 5 CVs). Fractions containing the desired product were pooled and then concentrated in vacuo. TLC analysis indicated that an impurity co-eluted with the desired product, suggesting that the quench of the POCl₃ had been incomplete. The isolated material (˜13 g) was stirred with water and then was extracted with ethyl acetate. The organic phase was washed with sat. aq. sodium carbonate upon which the organic phase turned from a colorless to a yellow solution and the aqueous phase tested as pH>7. The organic phase was isolated and then concentrated in vacuo to afford N-((S)-1-(7-chloro-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (865 mg, 1.029 mmol, 85% yield). LCMS Method D: retention time=2.961 min.; observed ion=841.95 (M+H). ¹H NMR (500 MHz, METHANOL-d₄) δ ppm 0.99 (td, J=3.73, 2.09 Hzz, 1H) 1.34 (br dd, J=7.60, 1.94 Hz, 1H) 2.37-2.46 (m, 2H) 3.04-3.13 (m, 5H) 3.43 (d, J=4.17 Hz, 1 H) 3.45-3.46 (m, 1H) 3.53 (s, 3H) 4.90 (s, 1H) 6.54-6.79 (m, 4H) 7.16 (q, J=7.75 Hz, 2 H) 7.69-7.72 (m, 1H) 8.63-8.66 (m, 1H). The LCMS and NMR data indicated that the sample contained approximately about 20% mol of N-((R)-1-(7-chloro-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (the diastereomer) that had formed in the reaction.

### Preparation of N-((S)-1-(7-bromo-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

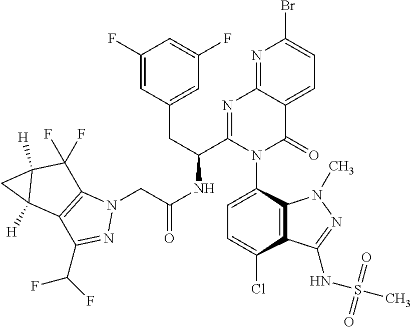

To a solution of N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.0 g, 1.2 mmol) in 1,2-Dichloroethane (DCE) (12 ml) was added phosphoryl tribromide (1.7 g, 6.1 mmol) and the solution was then stirred at 60° C. for 2 hrs. To the solution was added phosphoryl tribromide (1.7 g, 6.1 mmol) the solution was stirred at 60° C. for 3 hrs. The solution was cooled to room temperature and then was poured over ice (100 g). The mixture was diluted with EtOAc and then stirred until the mixture warmed to room temperature (approximately 15 minutes). The organic phase was washed with sat. sodium carbonate until the pH was >7 upon which the color of the organic phase changed from colorless to yellow. The organic phase was concentrated in vacuo and adsorbed onto Celite. The resulting powder was subjected to silica gel chromatography (40 g column, ethyl acetate and hexanes as eluent) to afford N-((S)-1-(7-bromo-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (932 mg, 1.05 mmol, 87% yield). LCMS Method D: retention time=2.987 min; observed ion=885.80 (M+H). ¹H NMR (500 MHz, METHANOL-d₄) δ ppm 0.99 (br d, J=2.98 Hz, 1H) 1.35 (s, 1H) 2.41 (br dd, J=6.56, 4.17 Hz, 2H) 3.04-3.15 (m, 4H) 3.54 (s, 2H) 4.57 (d, J=14.60 Hz, 1H) 4.77 (s, 1H) 4.79-4.83 (m, 2H) 6.41-6.84 (m, 4H) 7.12-7.21 (m, 2H) 7.85 (s, 1H) 7.86 (t, J=3.98 Hz, 1H) 8.50-8.53 (m, 1H). The LCMS and NMR data indicated that the sample contained approximately about 30% mol of N-((R)-1-(7-bromo-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (the diastereomer) that had formed in the reaction.

### Preparation of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

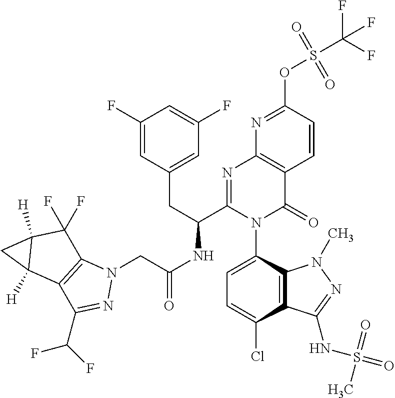

To a solution of N-((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.12 g, 2.58 mmol) and 1,1,1-trifluoro-N-(pyridin-2-yl)-N-((trifluoromethyl)sulfonyl)methanesulfonamide (1.940 g, 5.42 mmol) in dichloromethane (12.9 mL) was added triethylamine (0.76 mL, 5.42 mmol) and the mixture was stirred at rt for 18 h. The reaction mixture was then directly subjected to silica gel chromatography (120 g RediSep column) eluting with 0-60% ethyl acetate in hexanes over 10 CV, then at 60% ethyl acetate in hexanes for 8 CV. The desired fractions were pooled and then concentrated under reduced pressure to afford (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (1.6 g, 1.677 mmol, 65.0% yield) as an off-white solid foam. LC/MS: m/z=955.95 [M+1]⁺.

### Preparation of (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

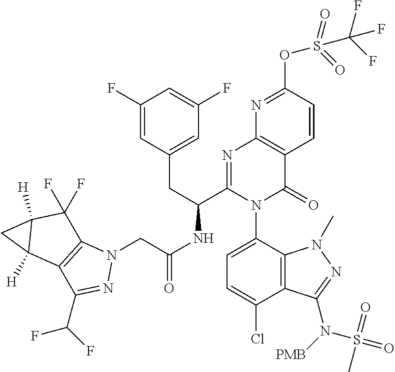

To a solution of N-((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.2 g, 1.273 mmol) and 1,1,1-trifluoro-N-(pyridin-2-yl)-N-((trifluoromethyl)sulfonyl)methanesulfonamide (0.958 g, 2.67 mmol) in Dichloromethane (DCM) (10 mL) was added triethylamine (0.373 mL, 2.67 mmol) and the mixture was then stirred at rt for 18 h. The reaction mixture was loaded directly onto a silica gel column (220 g RediSep) and purified using 0-80% ethyl acetate in hexanes as eluent. The desired fractions were concentrated to afford (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (900 mg, 0.838 mmol, 65.8% yield). LCMS (M+H)+=1074.05

### Preparation of (3P)-3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1 H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

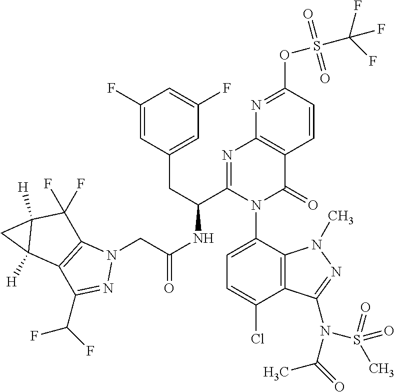 [SMILES: CC(=O)N(c1nn(C)c2c(-n3c([C@H](Cc4cc(F)cc(F)c4)NC(=O)Cn4nc(C(F)F)c5c4C(F)(F)[C@@H]4C[C@H]54)nc4nc(OS(=O)(=O)C(F)(F)F)ccc4c3=O)ccc(Cl)c12)S(C)(=O)=O]

To a solution of N-((S)-1-((3P)-3-(4-chloro-1-methyl-3-(N-(methylsulfonyl)acetamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (3.3 g, 3.82 mmol) and 1,1,1-trifluoro-N-(pyridin-2-yl)-N-((trifluoromethyl)sulfonyl)methanesulfonamide (3.42 g, 9.55 mmol) in Dichloromethane (DCM) (20 mL) was added triethylamine (1.331 mL, 9.55 mmol) and the mixture was stirred at rt for 18 h. The reaction mixture was then loaded directly on silica gel column (330 g isco) and purified using 0-80% ethyl acetate in hexanes gradient. The desired fractions were pooled and then concentrated in vacuo to afford (3P)-3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (2.5 g, 2.509 mmol, 65.7% yield) as an off-white foamy solid. LCMS (M+H)=996.00

### Preparation of N-((S)-1-((3P)-3-(4-chloro-3-(N-(4-methoxybenzyl)methylsuIfonamido)-1-methyl-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

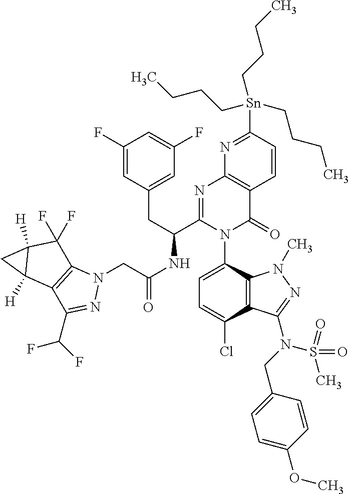

A 5 mL microwave vial was charged with 1,1,1,2,2,2-hexabutyldistannane (0.19 mL, 0.375 mmol), N-((S)-1-((3P)-7-chloro-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.3 g, 0.312 mmol) and Pd(PPh₃)₄ (0.036 g, 0.031 mmol). To the mixture was added degassed (bubbled with Nitrogen for 2 minutes) N,N-Dimethylformamide (3.12 mL). The mixture was stirred and purged with nitrogen for 2 min. The vial was capped and heated at 110° C. for 18 h. The reaction mixture was diluted with ethyl acetate, washed with water, brine, dried over Na₂SO₄ and concentrated in vacuo. The resulting residue was purified by silica gel chromatography (24 g RediSep Gold column) using 0-60% ethyl acetate in hexanes over 20 CV. The desired fractions were pooled and then concentrated under reduced pressure to afford N-((S)-1-((3P)-3-(4-chloro-3-(N-(4-methoxybenzyl)methylsulfonamido)-1-methyl-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as a clear oil (0.11 g, 0.093 mmol, 30% yield). LC/MS: m/z=1216.2 [M+1]⁺.

### Preparation of N-((S)-1-((3P)-3-(4-chloro-1-methyl-3-(methylsulfonamido)-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1 H-cyclopropa[3,4]cyclopenta[1,2-c]pyrazol-1-yl)acetamide

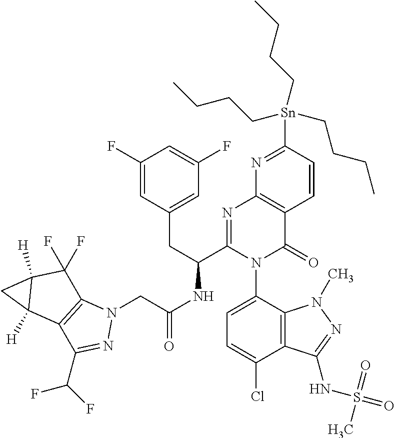 [SMILES: CCC[CH2][Sn]([CH2]CCC)([CH2]CCC)[c]1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2n1]

A 5 mL microwave vial was charged with 1,1,1,2,2,2-hexabutyldistannane (0.75 ml, 1.5 mmol), N-((S)-1-(7-chloro-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (500 mg, 0.595 mmol), Pd(PPh₃)₄ (69 mg, 0.059 mmol) and N,N-Dimethylformamide (DMF) (5.9 ml). The vial was capped and the mixture was degassed (brief vacuum evacuation followed by refill with Ar, repeated 3 times). The mixture was stirred under Ar atmosphere at 100° C. overnight (app 18 hrs). The reaction mixture was diluted with ethyl acetate, washed with water and then brine, dried over Na₂SO₄, filtered and then concentrated in vacuo. The resulting residue was purified by silica gel chromatography (40 g RediSep Gold column) using 0-100% ethyl acetate in hexanes over 10 CV and then 100% ethyl acetate for 5 CVs. Fractions containing the desired product were pooled and then concentrated in vacuo to afford N-((S)-1-((3P)-3-(4-chloro-1-methyl-3-(methylsulfonamido)-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (45 mg, 0.041 mmol, 6.91% yield). LCMS Method D: retention time=4.166 min; observed ion=1095.90 (M+H). ¹H NMR (500 MHz, METHANOL-d₄) δ ppm 1.19-1.23 (m, 2H) 1.27-1.43 (m, 19H) 1.62-1.69 (m, 8H) 3.24 (s, 3H) 3.62 (s, 2H) 4.54-4.58 (m, 8H) 6.52-6.82 (m, 4H) 7.19-7.33 (m, 2H) 7.84-7.87 (m, 1H) 8.36-8.53 (m, 1H)

### Alternate Preparation of N-((S)-1-((3P)-3-(4-chloro-1-methyl-3-(methylsulfonamido)-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

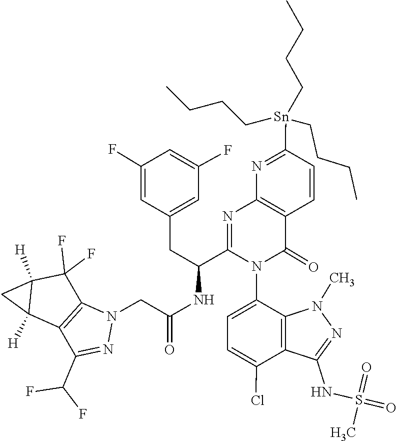 [SMILES: CCC[CH2][Sn]([CH2]CCC)([CH2]CCC)[c]1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2n1]

A 5 mL microwave vial was charged with 1,1,1,2,2,2-hexabutyldistannane (1.0 mL, 2.0 mmol), N-((S)-1-(7-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (710 mg, 0.802 mmol), Pd(PPh₃)₄ (93 mg, 0.080 mmol) and N,N-Dimethylformamide (DMF) (8.0 mL). The vial was capped and the mixture was degassed (brief vacuum evacuation followed by refill with argon, repeated 3 times). The mixture was stirred under argon atmosphere at 120° C. overnight (app 18 hrs). The mixture was concentrated in vacuo and the residue was adsorbed onto Celite. The resulting powder was subjected to silica gel chromatography (0-100% ethyl acetate in hexanes over 10 CVs) to afford N-((S)-1-((3P)-3-(4-chloro-1-methyl-3-(methylsulfonamido)-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (233 mg, 0.213 mmol, 26.5% yield). LCMS Method D: retention time=4.166 min; observed ion=1096.10 (M+H).

### Preparation of (S)-tert-butyl (1-(6-bromo-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate

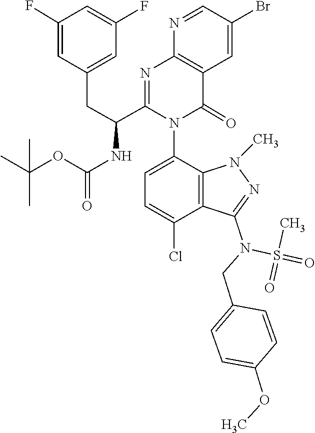 [SMILES: COc1ccc(CN(c2nn(C)c3c(-n4c([C@H](Cc5cc(F)cc(F)c5)NC(=O)OC(C)(C)C)nc5ncc(Br)cc5c4=O)ccc(Cl)c23)S(C)(=O)=O)cc1]

To a mixture of (S)-2-((tert-butoxycarbonyl)amino)-3-(3,5-difluorophenyl)propanoic acid (11.45 g, 38.0 mmol) and 2-amino-5-bromonicotinic acid (9.07 g, 41.8 mmol) in acetonitrile (200 mL) (a white slurry) was added pyridine (7.37 ml, 91 mmol) followed by dropwise addition over 5 minutes of 2,4,6-tripropyl-1,3,5,2,4,6-trioxatriphosphinane 2,4,6-trioxide (50% in EtOAc, 113 mL, 190 mmol) upon which the mixture slowly turned to a brown color. The mixture was stirred at 40° C. for 4 h. To the mixture was added N-(7-amino-4-chloro-1-methyl-1H-indazol-3-yl)-N-(4-methoxybenzyl)methanesulfonamide (15 g, 38.0 mmol) and the mixture was stirred at 40° C. for 18 h. The mixture was cooled to room temperature and then was filtered. The filtrate was concentrated under reduced pressure and the residue was subjected to silica gel chromatography (330 g RediSep column, 10-90% EtOAc in hexanes) to afford the product tert-butyl (S)-(1-(6-bromo-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate (4.85 g, 5.65 mmol, 14.86% yield) as a light brown foam. LCMS Method D: retention time=3.35 min.; observed ion=802.0/803.9 (M−tBu+H); RT (min)=3.35.

### Preparation of (S)-N-((6P)-7-(2-(1-amino-2-(3,5-difluorophenyl)ethyl)-6-bromo-4-oxopyrido[2,3-d]pyrimidin-3(4H)-yl)-4-chloro-1-methyl-1H-indazol-3-yl)-N-(4-methoxybenzyl)methanesulfonamide

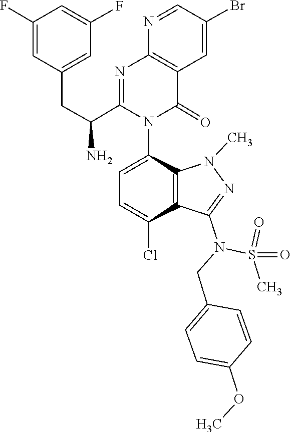

To a stirred solution of tert-butyl (S)-(1-(6-bromo-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate (4.85 g, 5.65 mmol) in Dichloromethane (DCM) (28.2 ml) was added TFA (8.70 ml, 113 mmol). The solution was stirred at RT for 1 hr. The solution was concentrated under reduced pressure. The dark yellow oily residue was dissolved in EtOAc (50 mL) and washed with 1M NaOH (20 mL). The organic layer was washed with brine, dried (MgSO₄), filtered and concentrated under reduced pressure to afford the crude residue which was purified by silica gel chromatography (330 g RediSep Gold column, 20-100% Solvent B in hexanes, Solvent B=9:18:3 Hexane:EtOAc:MeOH). This purification afforded two peaks containing the product mass (atropisomers). Fractions of the first peak to elute were pooled and concentrated in vacuo to afford the product (S)-N-((6P)-7-(2-(1-amino-2-(3,5-difluorophenyl)ethyl)-6-bromo-4-oxopyrido[2,3-d]pyrimidin-3(4H)-yl)-4-chloro-1-methyl-1H-indazol-3-yl)-N-(4-methoxybenzyl)methanesulfonamide (2.58 g, 3.40 mmol, 60.2% yield) as a white foam. LCMS Method D: retention time=2.34 min.; observed ion=757.95/759.65 (M+H).

### Preparation of N-((S)-1-((3P)-6-bromo-3-(4-chloro-3-(N-(4- methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

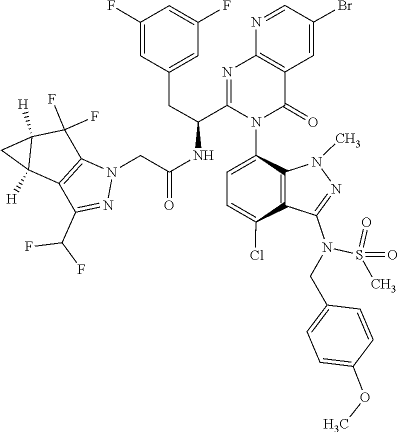

To a stirred solution of (S)-N-((6P)-7-(2-(1-amino-2-(3,5-difluorophenyl)ethyl)-6-bromo-4-oxopyrido[2,3-d]pyrimidin-3(4H)-yl)-4-chloro-1-methyl-1H-indazol-3-yl)-N-(4-methoxybenzyl)methanesulfonamide (2.58 g, 3.40 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.988 g, 3.74 mmol), and HATU (1.551 g, 4.08 mmol) in Tetrahydrofuran (THF) (34.0 mL) was added diisopropylethylamine (1.781 mL, 10.20 mmol). The resulting mixture was stirred at RT for 2 hrs. The mixture was then concentrated in vacuo and the residue was dissolved in EtOAc (100 mL). The organic solution was washed with aq. 1 M HCl (100 mL), then water (100 mL), then brine. The organic solution was dried (MgSO₄), filtered and then concentrated in vacuo to afford N-((S)-1-((3P)-6-bromo-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (3.42 g, 3.40 mmol, 100% yield) as a light brown solid. LCMS Method D: retention time=3.29 min.; observed ion=1004.0/1005.9 (M+H).

### Preparation of N-((S)-1-((3P)-6-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

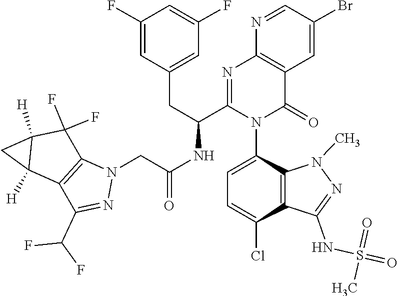

To a stirred solution of N-((S)-1-((3P)-6-bromo-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (3.42 g, 3.40 mmol) in Dichloromethane (DCM) (17.01 ml) was added TFA (7.86 ml, 102 mmol) followed by triflic acid (0.906 ml, 10.21 mmol). The solution was stirred at RT for 1 hr. The solution was concentrated under reduced pressure. The dark red oily residue was dissolved in EtOAc (75 mL) and washed with 1M NaOH (60 mL). The organic layer was washed with brine, dried (MgSO₄), filtered and concentrated under reduced pressure to afford the crude residue which was purified by silica gel chromatography (120 g RediSep Gold column, 10-75% EtOAc in hexanes) to afford the product N-((S)-1-((3P)-6-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.76 g, 3.12 mmol, 92% yield) as a white solid. LCMS Method D: retention time=2.95 min.; observed ion=884.0/885.9 (M+H).

### Preparation of N-((S)-1-(3-(4-chloro-1-methyl-3-(methylsulfonamido)-1H-indazol-7-yl)-4-oxo-6-(4,4,5,5-tetramethyl-1,3,2-dioxaborolan-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

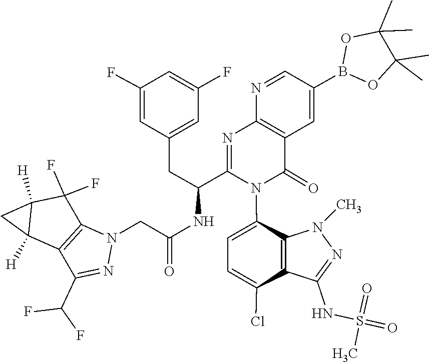

To a mixture of N-((S)-1-((3P)-6-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.0 g, 1.130 mmol), 4,4,4′,4′,5,5,5′,5′-octamethyl-2,2′-bi(1,3,2-dioxaborolane) (0.301 g, 1.186 mmol), PdCl₂(dppf) (0.041 g, 0.056 mmol), and potassium acetate (0.333 g, 3.39 mmol) was added degassed (bubbled with nitrogen for 5 min.) 1,4-Dioxane (11.30 mL). The mixture stirred under nitrogen atmosphere at 80° C. for 2 hrs. The mixture was cooled to ambient temperature and then was diluted with EtOAc. The organic solution was washed with water, the brine, dried (MgSO₄), filtered and concentrated under reduced pressure to afford the product N-((S)-1-((3P)-3-(4- chloro-1-methyl-3-(methylsulfonamido)-1H-indazol-7-yl)-4-oxo-6-(4,4,5,5-tetramethyl-1,3,2-dioxaborolan-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.05 g, 1.13 mmol, 100% yield). LCMS Method D: retention time=2.59 min; observed ion=850.0 (M+H).

### Preparation of N-((S)-1-((3P)-3-(4-chloro-3-(N-(4-methoxybenzyl)methylsuIfonamido)-1-methyl-1H-indazol-7-yl)-4-oxo-7-(1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

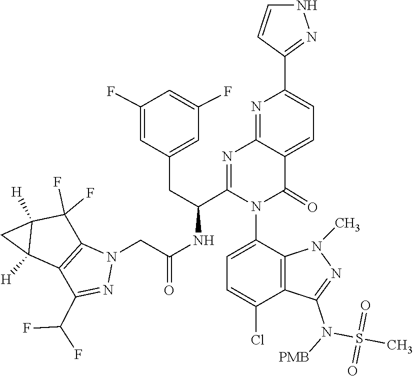

To a solution of (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (400 mg, 0.372 mmol), (1H-pyrazol-3-yl)boronic acid (125 mg, 1.117 mmol) and K₃PO₄ (237 mg, 1.117 mmol) in Tetrahydrofuran (THF) (4 mL)/Water (1.0 mL) was added Dichloro[9,9-dimethyl-4,5-bis(diphenylphosphino)xanthene]palladium(II) (28.1 mg, 0.037 mmol) and the resulting mixture was heated at 50° C. for 2 h. LCMS at this point indicated the desired product as a major peak. The mixture was cooled to room temp, diluted with ethyl acetate and washed with water. The organic solution was dried (Na₂SO₄), filtered and concentrated. The resulting residue was purified by silica gel chromatography (5-100% EtOAC in hexanes) to afford N-((S)-1-((3P-3-(4-chloro-3-(N-(4-methoxybenzyl)methylsulfonamido)-1-methyl-1H-indazol-7-yl)-4-oxo-7-(1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (290 mg, 0.292 mmol, 78% yield). LCMS (M+H)+=992.10

### Preparation of N-((S)-1-((3P)-3-(4-chloro-3-(N-(4-methoxybenzyl)methylsulfonamido)-1-methyl-1H-indazol-7-yl)-4-oxo-7-(5-(trifluoromethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

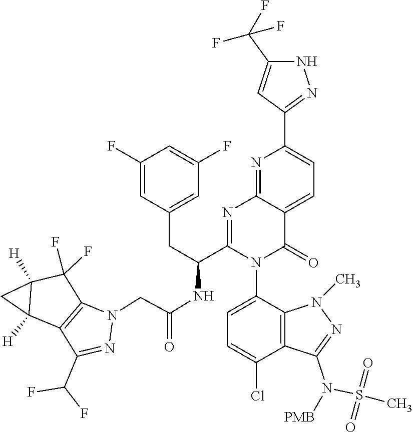

To a solution of (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (120 mg, 0.112 mmol), (5-(trifluoromethyl)-1H-pyrazol-3-yl)boronic acid (60.3 mg, 0.335 mmol) and K₃PO₄ (71.1 mg, 0.335 mmol) in Tetrahydrofuran (THF) (2 mL)/Water (0.500 mL) was added Dichloro[9,9-dimethyl-4,5-bis(diphenylphosphino)xanthene]palladium(II) (8.44 mg, 0.011 mmol) and the resulting mixture was heated at 50° C. for 2 h. LCMS at this point indicated the desired product as the major peak. The mixture was then cooled to room temp, diluted with ethyl acetate and washed with water. The organic phase was dried (Na₂SO₄), filtered and concentrated in vacuo. The resulting residue was then purified by silica gel chromatography (5-100% EtOAC in hexanes) to afford N-((S)-1-((3P)-3-(4-chloro-3-(N-(4-methoxybenzyl)methylsulfonamido)-1-methyl-1H-indazol-7-yl)-4-oxo-7-(5-(trifluoromethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. LCMS (M+H)+=1060.10

### Preparation of N-((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

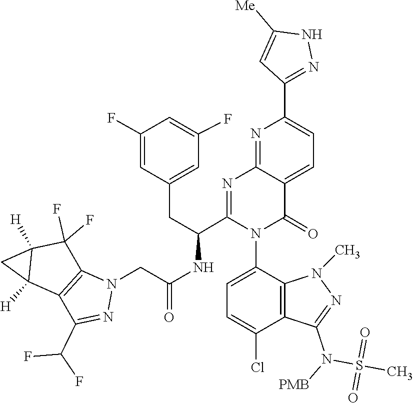

To a solution of (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (120 mg, 0.112 mmol), (5-methyl-1H-pyrazol-3-yl)boronic acid (42.2 mg, 0.335 mmol) and K₃PO₄ (71.1 mg, 0.335 mmol) in Tetrahydrofuran (THF) (2 mL)/Water (0.500 mL) was added dichloro[9,9-dimethyl-4,5-bis(diphenylphosphino)xanthene]palladium(II) (8.44 mg, 0.011 mmol) and the resulting mixture was heated at 50° C. for 2 h. LCMS at this point indicated the desired product was the major peak. The mixture was then cooled to room temp, diluted with ethyl acetate and washed with water. The organic phase was dried (Na₂SO₄), filtered and concentrated. The resulting residue was then purified by silica gel chromatography (5-100% EtOAC in hexanes) to afford N-((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (90 mg, 0.089 mmol, 80% yield). LCMS (M+H)+=1006.0

### Preparation of Example 1: N-((S)-1-((3P)-3-(4-chloro-1-methyl-3-(methylsulfonamido)-1H-indazol-7-yl)-4-oxo-6-(6-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

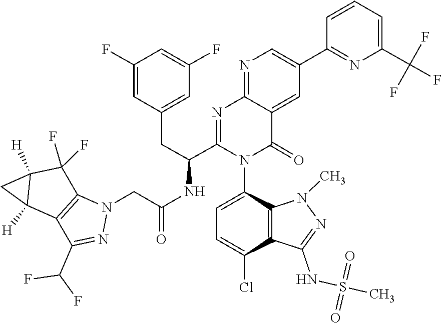

To a 1 dram vial charged with N-((S)-1-(6-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (35 mg, 0.040 mmol), 2-(4,4,5,5-tetramethyl-1,3,2-dioxaborolan-2-yl)-6-(trifluoromethyl)pyridine (21.60 mg, 0.079 mmol), Dichloro[9,9-dimethyl-4,5-bis(diphenylphosphino)xanthene]palladium(II) (2.99 mg, 3.95 μmol) and K₃PO₄ (25.2 mg, 0.119 mmol) was added degassed (Nitrogen bubbling for 1 min) Tetrahydrofuran (THF) (1 mL):Water (0.25 mL) and the resulting mixture was stirred at room temp for 16 h under an atmosphere of nitrogen. The LCMS indicated the reaction was complete. The reaction mass was transferred to a 20 mL scintillation vial. To the mixture was added EtOAc (5 mL) and aqueous 1 M HCl (5 mL). The vial was sealed and shaken. The organic layer was pipetted away and concentrated in vacuo. The resulting residue was dissolved in DMF (2 mL) and was then subjected to HPLC purification to afford the title compound, N-((S)-1-((3P)-3-(4-chloro-1-methyl-3-(methylsulfonamido)-1H-indazol-7-yl)-4-oxo-6-(6-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.46 min.; observed ion=951.1 (M+H). 1H NMR (500 MHz, METHANOL-d4) δ ppm 9.86 (d, J=2.38 Hz, 1H), 9.40 (d, J=2.38 Hz, 1H), 8.46 (d, J=7.75 Hz, 1H), 8.24 (t, J=7.75 Hz, 1H), 7.91 (d, J=7.75 Hz, 1H), 7.30-7.37 (m, 2H), 6.57-6.84 (m, 4H), 4.57 (d, J=3.58 Hz, 2H), 3.68 (s, 3H), 3.53 (dd, J=14.01, 4.47 Hz, 1H), 3.26 (s, 3 H), 3.17 (dd, J=14.16, 9.69 Hz, 1H), 2.39-2.47 (m, 2H), 1.33-1.39 (m, 1H), 1.01 (dtd, J=5.66, 3.80, 3.80, 2.24 Hz, 1H)

### Preparation of Example 2: N-((S)-1-((3P)-3-(4-chloro-1-methyl-3-(methylsulfonamido)-1H-indazol-7-yl)-6-(4-(difluoromethyl)pyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

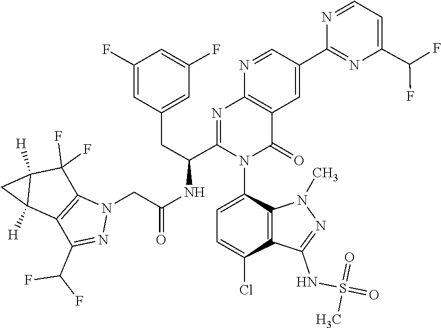

To a 7 mL vial was charged with N-((S)-1-(6-bromo-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (45mg, 0.051 mmol), 4-(difluoromethyl)-2-(tributylstannyl)pyrimidine (32.0 mg, 0.076 mmol) ,copper(I) iodide (0.968 mg, 5.08 μmol) and Pd(PPh₃)₄ (5.88 mg, 5.08 μmol) was added degassed (bubbled with Nitrogen for 2 minutes) N,N-Dimethylformamide (DMF) (0.5 mL). The mixture was stirred and purged with nitrogen for 2 min. The vial was capped and then heated at 100° C. for 18 h. The LCMS indicated the reaction was complete. The reaction mixture was diluted with DMF (1.5 mL) and the resulting solution was subjected to HPLC purification to afford the title compound, N-((S)-1-((3P)-3-(4-chloro-1-methyl-3-(methylsulfonamido)-1H-indazol-7-yl)-6-(4-(difluoromethyl)pyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.4 min.; observed ion=934.2 (M+H). 1H NMR (500 MHz, METHANOL-d4) δ ppm 10.11 (d, J=2.38 Hz, 1H), 9.68 (d, J=2.38 Hz, 1H), 9.20 (d, J=5.07 Hz, 1H), 7.79 (d, J=5.07 Hz, 1H), 7.35 (d, J=2.09 Hz, 2H), 6.55-7.02 (m, 5H), 4.57 (d, J=4.47 Hz, 2H), 3.68 (s, 3H), 3.50-3.56 (m, 1H), 3.26 (s, 3H), 3.15-3.20 (m, 1H), 2.40 -2.46 (m, 2H), 1.34-1.39 (m, 1H), 0.98-1.03 (m, 1H)

### Preparation of Example 3: N-((S)-1-((3P)-3-(4-chloro-1-methyl-3-(methylsulfonamido)-1H-indazol-7-yl)-6-(6-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

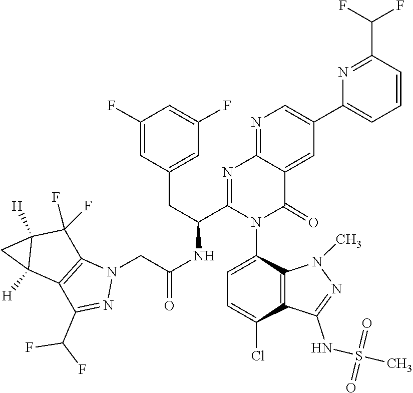

To a 7 mL vial charged with N-((S)-1-(3-(4-chloro-1-methyl-3-(methylsulfonamido)-1H-indazol-7-yl)-4-oxo-6-(4,4,5,5-tetramethyl-1,3,2-dioxaborolan-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (105 mg, 0.113 mmol), 2-chloro-6-(difluoromethyl)pyridine (20.27 mg, 0.124 mmol), SPhos-Pd-G3 (8.79 mg, 0.011 mmol) and K₃PO₄ (71.7 mg, 0.338 mmol) was added degassed (Nitrogen bubbling for 1 min) 1,4-Dioxane (845 μl):Water (282 μl) and the resulting mixture was stirred at 60° C. for 1 h under an atmosphere of nitrogen. The LCMS indicated the reaction was complete. The reaction mass was transferred to a 20 mL scintillation vial. To the mixture was added EtOAc (5 mL) and aqueous 1 M HCl (5 mL). The vial was sealed and shaken. The organic layer was pipetted away and concentrated in vacuo. The residue was dissolved in DMF (2 mL) and subjected to HPLC purification to afford the title compound, N-((S)-1-((3P)-3-(4-chloro-1-methyl-3-(methylsulfonamido)-1H-indazol-7-yl)-6-(6-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.42 min.; observed ion=933 (M+H). 1H NMR (500 MHz, METHANOL-d4) δ ppm 9.73 (d, J=2.68 Hz, 1H), 9.26 (d, J=2.38 Hzz, 1H), 8.22 (dd, J=8.05, 0.89 Hz, 1H), 8.06 (t, J=7.90 Hz, 1H), 7.67 (d, J=8.05 Hz, 1H), 7.22 (q, J=8.05 Hz, 2H), 6.45-6.88 (m, 5H), 4.45 (d, J=3.28 Hz, 2H), 3.56 (s, 3H), 3.41 (dd, J=14.16, 4.62 Hz, 1H), 3.14 (s, 3H), 3.02-3.09 (m, 1H), 2.28-2.34 (m, 2H), 1.23-1.27 (m, 1H), 0.86-0.92 (m, 1H)

### Preparation of Example 4: N-((S)-1-((3P)-3-(4-chloro-1-methyl-3-(methylsulfonamido)-1H-indazol-7-yl)-4-oxo-7-(2-(trifluoromethyl)thiazol-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

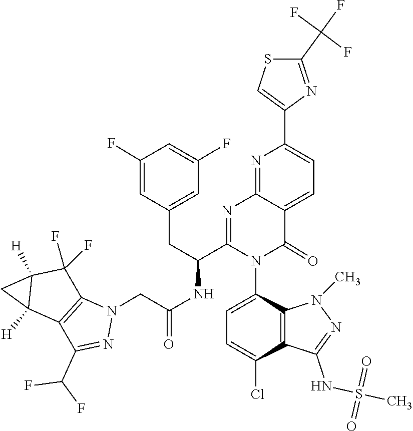

To a 1 dram vial charged with N-((S)-1-(7-chloro-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (35 mg, 0.042 mmol), 4-(4,4,5,5-tetramethyl-1,3,2-dioxaborolan-2-yl)-2-(trifluoromethyl)thiazole (23.24 mg, 0.083 mmol), SPhos-Pd-G3 (3.25 mg, 4.16 μmol) and K3PO4 (26.5 mg, 0.125 mmol) was added degassed (nitrogen bubbling for 1 min) 1,4-Dioxane (666 μl):Water (167 μl) and the resulting mixture was stirred at 60° C. for 48 h under an atmosphere of nitrogen. The LCMS showed a product peak but the reaction did not go to completion. The reaction mixture was cooled to ambient temp, transferred to a 20 mL scintillation vial, and was diluted with EtOAc (5 mL) and aq. HCl (1 M, 5 mL). The biphasic mixture was shaken. The organic layer was pipetted out and concentrated under reduced pressure. The residue was dissolved in DMF (2 mL) and subjected to HPLC purification to afford the title compound, N-((S)-1-((3P)-3-(4-chloro-1-methyl-3-(methylsulfonamido)-1H-indazol-7-yl)-4-oxo-7-(2-(trifluoromethyl)thiazol-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.52 min.; observed ion=957.3 (M+H). 1H NMR (500 MHz, METHANOL-d4) δ ppm 8.94 (s, 1 H), 8.84 (d, J=8.35 Hz, 1H), 8.49 (d, J=8.05 Hz, 1H), 7.27-7.34 (m, 2H), 6.57-6.83 (m, 4 H), 4.92-4.94 (m, 1H), 4.56 (d, J=4.47Hz, 2H), 3.67 (s, 3H), 3.54 (dd, J=14.31, 4.77 Hz, 1H), 3.25 (s, 3H), 3.16-3.21 (m, 1H), 2.43 (ddd, J=11.40, 7.53, 4.32 Hz, 2H), 1.35-1.39 (m, 1H), 0.99-1.02 (m, 1H)

### Preparation of Example 5: N-((S)-1-((3P)-3-(4-chloro-1-methyl-3-(methylsulfonamido)-1H-indazol-7-yl)-4-oxo-7-(4-(trifluoromethyl)pyrimidin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

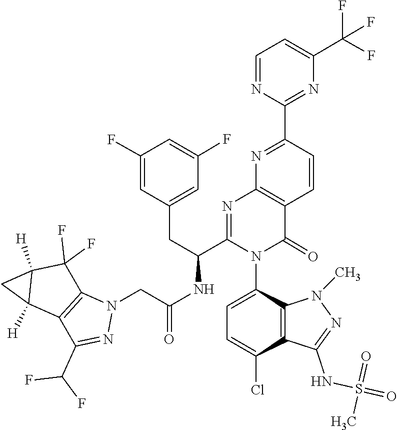

The title compound was prepared according to General Procedure B using 2-(tributylstannyl)-4-(trifluoromethyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(4-(trifluoromethyl)pyrimidin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=1.45 min.; observed ion=952.2 (M+H). 1H NMR (500 MHz, METHANOL-d4) δ ppm 9.38-9.45 (m, 1H) 8.89-8.96 (m, 1H) 8.79-8.85 (m, 1H) 8.01 -8.09 (m, 1H) 7.22-7.34 (m, 2H) 6.54-6.81 (m, 4H) 4.89-4.92 (m, 1H) 4.52-4.65 (m, 2 H) 3.61-3.68 (m, 3H) 3.47-3.53 (m, 1H) 3.20-3.24 (m, 3H) 3.13-3.19 (m, 1H) 2.35 -2.45 (m, 2H) 1.30-1.37 (m, 1H) 0.95-1.01 (m, 1H)

### Preparation of Example 6: N-((S)-1-((3P)-3-(4-chloro-1-methyl-3-(methylsulfonamido)-1H-indazol-7-yl)-7-(4-(difluoromethyl)pyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

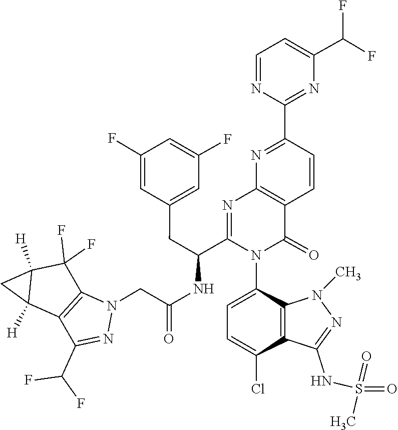

The title compound was prepared according to General Procedure B using 4-(difluoromethyl) -2-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-7-(4-(difluoromethyl)pyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=1.37 min.; observed ion=934.1 (M+H). 1H NMR (500 MHz, METHANOL-d4) δ ppm 9.29-9.36 (m, 1H) 8.88-8.93 (m, 1H) 8.80-8.85 (m, 1H) 7.89 -7.95 (m, 1H) 7.21-7.34 (m, 2H) 6.56-7.05 (m, 5H) 4.89-4.93 (m, 1H) 4.54-4.62 (m, 2 H) 3.61-3.65 (m, 3H) 3.47-3.54 (m, 1H) 3.22-3.24 (m, 3H) 3.13-3.19 (m, 1H) 2.36 -2.44 (m, 2H) 1.30-1.37 (m, 1H) 0.96-1.01 (m, 1H)

### Preparation of Example 7: N-((S)-1-((3P)-3-(4-chloro-1-methyl-3-(methylsulfonamido)-1H-indazol-7-yl)-7-(3-methylpyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

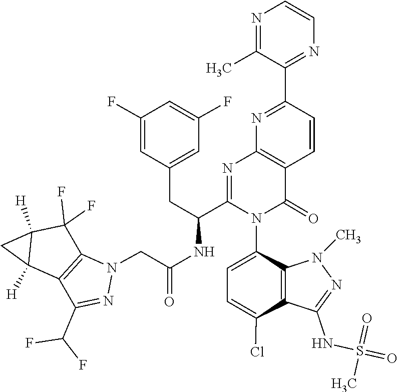

The title compound was prepared according to General Procedure B using 2-methyl-3-(tributylstannyl)pyrazine as the coupling partner. The experiment afforded the title compound, N-((S)-1-((3P)-3-(4-chloro-1-methyl-3-(methylsulfonamido)-1H-indazol-7-yl)-7-(3-methylpyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=1.34 min.; observed ion=898.2 (M+H). 1H NMR (500 MHz, METHANOL-d4) δ ppm 8.80-8.89 (m, 1H) 8.62-8.69 (m, 2H) 8.19-8.26 (m, 1H) 7.23 -7.35 (m, 2H) 6.55-7.09 (m, 5H) 4.88-4.91 (m, 1H) 4.54-4.58 (m, 2H) 3.62-3.67 (m, 3 H) 3.47-3.52 (m, 1H) 3.23-3.25 (m, 3H) 2.89-2.92 (m, 3H) 2.38-2.43 (m, 2H) 1.31 -1.37 (m, 1H) 0.95-1.01 (m, 1H)

### Preparation of Example 8: N-((S)-1-((3P)-3-(4-chloro-1-methyl-3-(methylsulfonamido)-1H-indazol-7-yl)-4-oxo-7-(pyrazin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

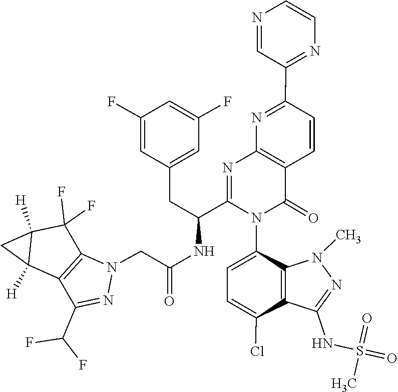

The title compound was prepared according to General Procedure B using 2-(tributylstannyl)pyrazine as the coupling partner. The experiment afforded the title compound, N-((S)-1-((3P)-3-(4-chloro-1-methyl-3-(methylsulfonamido)-1H-indazol-7-yl)-4-oxo-7-(pyrazin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=1.35 min.; observed ion=884.3 (M+H). 1H NMR (500 MHz, METHANOL-d4) δ ppm 9.74 -9.86 (m, 1H) 8.66-8.89 (m, 4H) 7.25-7.34 (m, 2H) 6.52-6.82 (m, 4H) 4.52-4.63 (m, 3 H) 3.62-3.68 (m, 3H) 3.47-3.55 (m, 1H) 3.22-3.24 (m, 3H) 3.14-3.20 (m, 1H) 2.37 -2.44 (m, 2H) 1.30-1.37 (m, 1H) 0.96-1.01 (m, 1H)

### Preparation of Example 9: N-((S)-1-((3P)-3-(4-chloro-1-methyl-3-(methylsulfonamido)-1H-indazol-7-yl)-4-oxo-7-(6-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

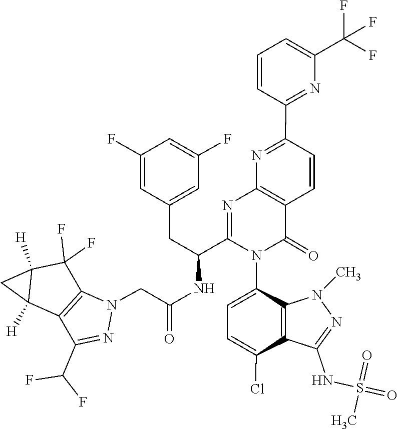

The title compound was prepared according to General Procedure B using 2-(tributylstannyl)-6-(trifluoromethyl)pyridine as the coupling partner. The experiment afforded the title compound, N-((S)-1-((3P)-3-(4-chloro-1-methyl-3-(methylsulfonamido)-1H-indazol-7-yl)-4-oxo-7-(6-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=951 (M+H). 1H NMR (500 MHz, METHANOL-d4) δ ppm 8.91-8.94 (m, 1H) 8.85-8.89 (m, 1H) 8.75-8.81 (m, 1H) 8.29-8.36 (m, 1H) 7.98-8.02 (m, 1H) 7.29-7.37 (m, 2H) 6.56-6.84 (m, 4H) 4.90-4.95 (m, 1 H) 4.53-4.64 (m, 2H) 3.66-3.69 (m, 3H) 3.50-3.57 (m, 1H) 3.26-3.27 (m, 3H) 3.18 -3.23 (m, 1H) 2.37-2.48 (m, 2H) 1.34-1.40 (m, 1H) 0.97-1.05 (m, 1H)

### Preparation of Example 10: N-((S)-1-((3P)-3-(4-chloro-1-methyl-3-(methylsulfonamido)-1H-indazol-7-yl)-7-(4-methylpyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

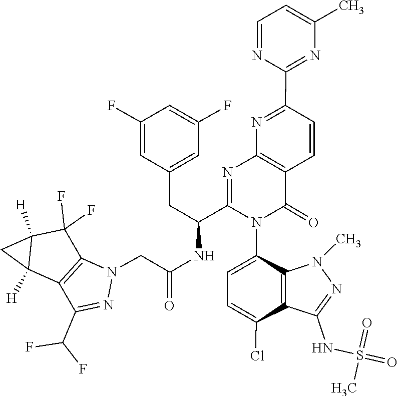

The title compound was prepared according to General Procedure B using 4-methyl-2-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-7-(4-methylpyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.33 min.; observed ion=898.2 (M+H). 1H NMR (500 MHz, METHANOL-d4) δ ppm 8.92-8.95 (m, 1H) 8.87-8.90 (m, 1H) 8.78-8.81 (m, 1H) 7.54 -7.56 (m, 1H) 7.32-7.34 (m, 1H) 7.23-7.26 (m, 1H) 6.58-6.83 (m, 4H) 4.91-4.96 (m, 1 H) 4.55-4.65 (m, 2H) 3.63-3.68 (m, 3H) 3.49-3.56 (m, 1H) 3.26-3.28 (m, 3H) 3.14 -3.19 (m, 1H) 2.73-2.77 (m, 3H) 2.39-2.46 (m, 2H) 1.34-1.39 (m, 1H) 0.99-1.04 (m, 1 H)

### Preparation of Example 11: N-((S)-1-((3P)-3-(4-chloro-1-methyl-3-(methylsulfonamido)-1H-indazol-7-yl)-7-(4,6-dimethylpyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

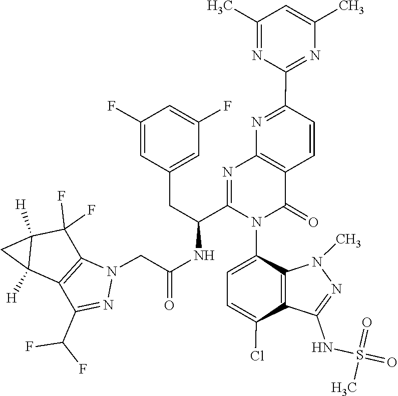

The title compound was prepared according to General Procedure B using 4,6-dimethyl-2-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-7-(4,6-dimethylpyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.38 min.; observed ion=912.2 (M+H). 1H NMR (500 MHz, METHANOL-d4) δ ppm 8.84-8.89 (m, 1H) 8.72-8.77 (m, 1H) 7.41-7.44 (m, 1H) 7.31 -7.35 (m, 1H) 7.21-7.27 (m, 1H) 6.57-6.83 (m, 4H) 4.91-4.96 (m, 1H) 4.55-4.63 (m, 2 H) 3.63-3.69 (m, 3H) 3.49-3.57 (m, 1H) 3.24-3.27 (m, 3H) 3.15-3.21 (m, 1H) 2.66 -2.71 (m, 6H) 2.38-2.46 (m, 2H) 1.33-1.39 (m, 1H) 0.98-1.04 (m, 1H)

### Preparation of Example 12: N-((S)-1-((3P)-3-(4-chloro-1-methyl-3-(methylsulfonamido)-1H-indazol-7-yl)-7-(6-(1,1-difluoroethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

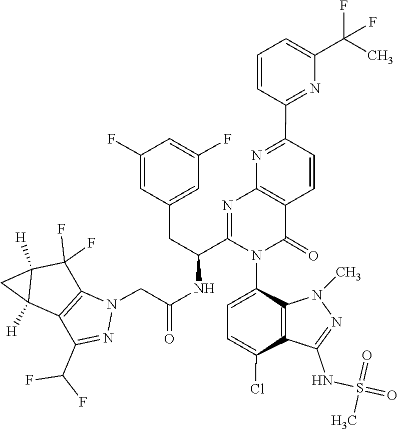

To a 5 mL microwave vial charged with N-((S)-1-(3-(4-chloro-3-(N-(4-methoxybenzyl)methylsulfonamido)-1-methyl-1H-indazol-7-yl)-4-oxo-7-(tributylstannyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.113 g, 0.093 mmol), 2-bromo-6-(1,1-difluoroethyl)pyridine (0.062 g, 0.279 mmol), copper(I) iodide (1.771 mg, 9.30 μmol) and Pd(PPh₃)₄ (10.74 mg, 9.30 μmol) was added degassed (bubbled with nitrogen for 2 minutes) N,N-Dimethylformamide (1 mL). The mixture was stirred and purged with nitrogen for 2 min. The vial was capped and the mixture was heated at 100° C. for 18 h. The reaction was diluted with water and extracted with EtOAc. The organic phase was dried over Na₂SO₄, filtered, and concentrated in vacuo. The resulting residue was purified by silica gel chromatography (12 g RediSep Gold column) using 0-70% ethyl acetate in hexanes over 10 CV, then 70% ethyl acetate in hexanes over 5 CV. The desired fractions were pooled and then concentrated to afford a yellow solid. The solid was dissolved up in DCM (1 mL) and TFA (0.5 mL), and to the solution was added triflic acid (0.025 ml, 0.279 mmol). The resulting purple solution was stirred for 30 min and then was concentrated in vacuo. The residue was taken up in ethyl acetate and the pH was adjusted to pH>7 using aq. 1 N NaOH. The mixture was dried over Na₂SO₄ and then concentrated in vacuo. The resulting residue was dissolved in DMF, the solution was filtered, and the filtrate was subjected to prep-HPLC purification to afford the title compound, N-((S)-1-((3P)-3-(4-chloro-1-methyl-3-(methylsulfonamido)-1H-indazol-7-yl)-7-(6-(1,1-difluoroethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=947.2 (M+H). 1H NMR (500 MHz, METHANOL-d4) δ ppm 8.23-8.29 (m, 1H) 7.41-7.46 (m, 1H) 7.30-7.36 (m, 1H) 7.24 -7.28 (m, 1H) 7.10-7.16 (m, 1H) 6.66-6.81 (m, 3H) 5.93-5.97 (m, 1H) 4.92-4.99 (m, 1 H) 4.30-4.39 (m, 1H) 3.69-3.79 (m, 2H) 3.41-3.45 (m, 1H) 3.34-3.36 (m, 3H) 3.30 -3.30 (m, 3H) 3.13-3.19 (m, 1H) 2.75-2.81 (m, 2H) 1.83-1.94 (m, 1H) 1.66-1.80 (m, 1 H) 1.48-1.62 (m, 1H) 0.96-1.06 (m, 1H) 0.60-0.70 (m, 2H)

### Preparation of Example 13: N-((S)-1-((3P)-3-(4-chloro-1-methyl-3-(methylsulfonamido)-1H-indazol-7-yl)-4-oxo-7-(6-(trifluoromethyl)pyrazin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

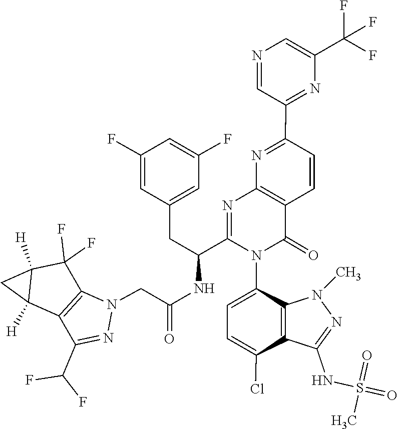

The title compound was prepared according to General Procedure F using 2-chloro-6-(trifluoromethyl)pyrazine as the coupling partner. The experiment afforded the title compound, N-((S)-1-((3P)-3-(4-chloro-1-methyl-3-(methylsulfonamido)-1H-indazol-7-yl)-4-oxo-7-(6-(trifluoromethyl)pyrazin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=952.2 (M+H). 1H NMR (500 MHz, METHANOL-d4) δ ppm 9.92-9.99 (m, 1H) 9.11-9.16 (m, 1H) 8.75-8.82 (m, 1H) 8.58 -8.65 (m, 1H) 7.19-7.27 (m, 2H) 6.43-6.74 (m, 4H) 4.81-4.83 (m, 1H) 4.41-4.53 (m, 2 H) 3.54-3.58 (m, 3H) 3.40-3.45 (m, 1H) 3.14-3.16 (m, 3H) 3.06-3.11 (m, 1H) 2.27 -2.34 (m, 2H) 1.23-1.27 (m, 1H) 0.87-0.92 (m, 1H).

### Preparation of Example 14: N-((S)-1-(7-(4,6-bis(trifluoromethyl)pyridin-2-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

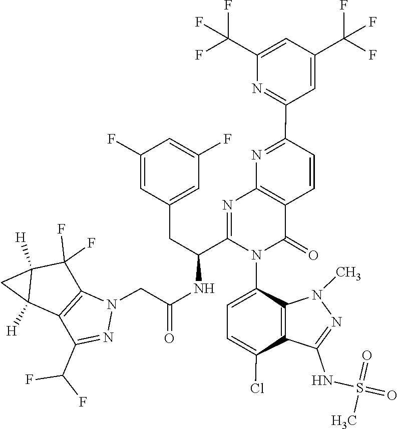

The title compound was prepared according to General Procedure F using 2-chloro-4,6- bis(trifluoromethyl)pyridine as the coupling partner. The experiment afforded the title compound, N-((S)-1-(7-(4,6-bis(trifluoromethyl)pyridin-2-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.65 min.; observed ion=1019.1 (M+H). 1H NMR (500 MHz, METHANOL-d4) δ ppm 9.20-9.27 (m, 1H) 8.90-8.95 (m, 1H) 8.77-8.84 (m, 1H) 8.31-8.37 (m, 1H) 7.31-7.38 (m, 2H) 6.55-6.84 (m, 4H) 4.92 (br d, J=4.47 Hz, 1H) 4.53-4.66 (m, 2H) 3.66-3.70 (m, 3H) 3.51-3.57 (m, 1H) 3.27 (s, 3H) 3.18-3.23 (m, 1 H) 2.39-2.46 (m, 2H) 1.34-1.39 (m, 1H) 0.99-1.04 (m, 1H)

### Preparation of Example 15: N-((S)-1-((3P)-3-(4-chloro-1-methyl-3-(methylsulfonamido)-1H-indazol-7-yl)-4-oxo-7-(6-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

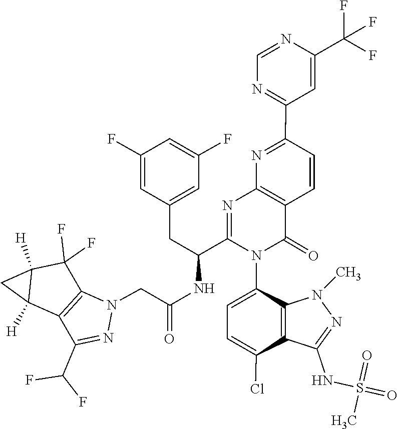

The title compound was prepared according to General Procedure F using 4-chloro-6-(trifluoromethyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(6-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=952.2 (M+H). 1H NMR (500 MHz, METHANOL-d4) δ ppm 9.59-9.65 (m, 1H) 9.00-9.06 (m, 1H) 8.92-8.97 (m, 1H) 8.85 -8.89 (m, 1H) 7.32-7.41 (m, 2H) 6.56-6.85 (m, 4H) 4.92-4.94 (m, 1H) 4.53-4.66 (m, 2 H) 3.65-3.72 (m, 3H) 3.50-3.57 (m, 1H) 3.27 (br s, 3H) 3.17-3.21 (m, 1H) 2.39-2.46 (m, 2H) 1.34-1.39 (m, 1H) 0.98-1.03 (m, 1H)

### Preparation of Example 16: N-((S)-1-((3P)-3-(4-chloro-1-methyl-3-(methylsulfonamido)-1H-indazol-7-yl)-4-oxo-7-(4-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

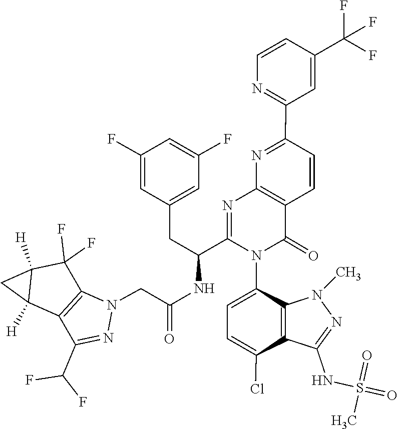

The title compound was prepared according to General Procedure F using 2-chloro-4-(trifluoromethyl)pyridine as the coupling partner. The experiment afforded the title compound, N-((S)-1-((3P)-3-(4-chloro-1-methyl-3-(methylsuIfonamido)-1H-indazol-7-yl)-4-oxo-7-(4-(trifluoromethyl)pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=951.3 (M+H). H NMR (500 MHz, METHANOL-d4) δ ppm 9.05-9.08 (m, 1H) 8.98 (s, 1H) 8.86-8.89 (m, 1H) 8.79-8.82 (m, 1H) 7.86-7.91 (m, 1H) 7.32-7.38 (m, 2H) 6.56-6.84 (m, 4H) 4.93-4.94 (m, 1H) 4.55 -4.68 (m, 2H) 3.67-3.70 (m, 3H) 3.52-3.56 (m, 1H) 3.26-3.27 (m, 3H) 3.17-3.21 (m, 1 H) 2.38-2.47 (m, 2H) 1.35-1.39 (m, 1H) 0.98-1.04 (m, 1H)

### Preparation of Example 17: N-((S)-1-((3P)-3-(4-chloro-1-methyl-3-(methylsulfonamido)-1H-indazol-7-yl)-4-oxo-7-(5-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

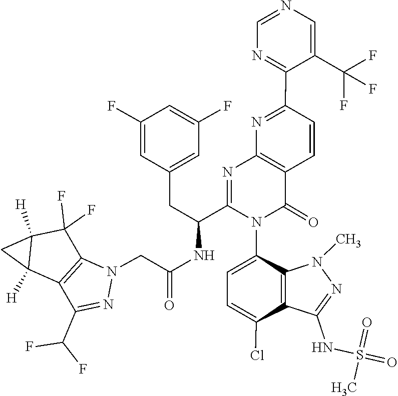

The title compound was prepared according to General Procedure F using 4-chloro-5-(trifluoromethyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(5-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.4 min.; observed ion=952.4 (M+H). 1H NMR (500 MHz, METHANOL-d4) δ ppm 8.88-9.09 (m, 3H) 8.07-8.13 (m, 1H) 7.31-7.38 (m, 2H) 6.54 -6.82 (m, 4H) 4.93-4.94 (m, 1H) 4.49-4.60 (m, 2H) 3.68-3.72 (m, 3H) 3.49-3.55 (m, 1 H) 3.26-3.28 (m, 3H) 3.12-3.17 (m, 1H) 2.37-2.47 (m, 2H) 1.34-1.40 (m, 1H) 0.97 -1.03 (m, 1H)

### Preparation of Example 18: N-((S)-1-((3P)-3-(4-chloro-1-methyl-3-(methylsulfonamido)-1H-indazol-7-yl)-7-(6-methyl-2-(trifluoromethyl)pyrimidin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3 b,4,4a,5-tetrahydro-1H-cyclopropa[3,4]cyclopenta[1,2-c]pyrazol-1-yl)acetamide.

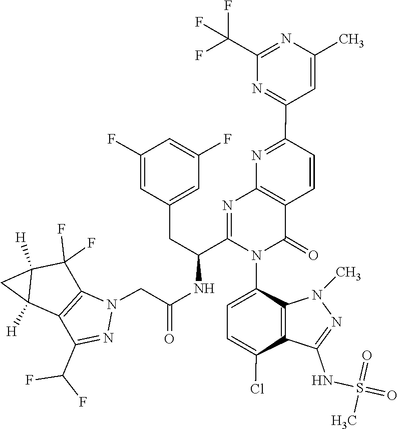

The title compound was prepared according to General Procedure F using 4-chloro-6-methyl-2-(trifluoromethyl)pyrimidine as the coupling partner. The experiment afforded the title compound, N-((S)-1-((3P)-3-(4-chloro-1-methyl-3-(methylsulfonamido)-1H-indazol-7-yl)-7-(6-methyl-2-(trifluoromethyl)pyrimidin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.51 min.; observed ion=did not ionize ( ). 1H NMR (500 MHz, METHANOL-d4) δ ppm 8.89-8.98 (m, 1H) 8.71-8.82 (m, 2H) 7.30-7.39 (m, 2H) 6.55-6.84 (m, 4H) 4.92-4.93 (m, 1H) 4.52-4.61 (m, 2H) 3.66-3.70 (m, 3H) 3.55 (dd, J=14.46, 4.32 Hz, 1H) 3.27 (s, 3H) 3.18-3.21 (m, 1H) 2.81-2.85 (m, 3H) 2.39-2.46 (m, 2H) 1.34-1.39 (m, 1H) 0.99-1.04 (m, 1H)

### Preparation of Example 19: N-((S)-1-((3P)-3-(4-chloro-1-methyl-3-(methylsulfonamido)-1H-indazol-7-yl)-7-(4-fluoro-3-(trifluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

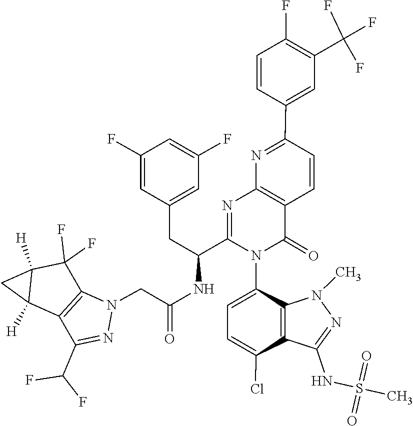

The title compound was prepared according to General Procedure E using (4-fluoro-3-(trifluoromethyl)phenyl)boronic acid as the coupling partner. The experiment afforded the title compound, N-((S)-1-((3P)-3-(4-chloro-1-methyl-3-(methylsuIfonamido)-1H-indazol-7-yl)-7-(4-fluoro-3-(trifluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=1.61 min.; observed ion=968.2 (M+H). 1H NMR (500 MHz, METHANOL-d4) δ ppm 8.66-8.76 (m, 1H) 8.28-8.39 (m, 2H) 8.11-8.19 (m, 1H) 7.77 -7.88 (m, 2H) 7.13-7.27 (m, 2H) 6.41-6.74 (m, 4H) 4.40-4.52 (m, 2H) 3.53-3.58 (m, 3 H) 3.38-3.43 (m, 1H) 3.14-3.15 (m, 3H) 3.03-3.09 (m, 1H) 2.26-2.34 (m, 2H) 1.21 -1.27 (m, 1H) 0.86-0.91 (m, 1H)

### Preparation of Example 20: N-((S)-1-((3P)-3-(4-chloro-1-methyl-3-(methylsulfonamido)-1H-indazol-7-yl)-7-(3-(difluoromethoxy)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

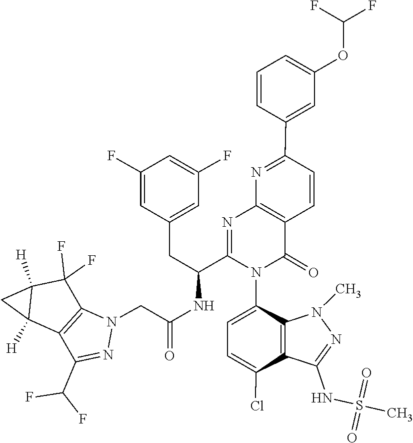

The title compound was prepared according to General Procedure E using (3-(difluoromethoxy)phenyl)boronic acid as the coupling partner. The experiment afforded the title compound, N-((S)-1-((3P)-3-(4-chloro-1-methyl-3-(methylsulfonamido)-1H-indazol-7-yl)-7-(3-(difluoromethoxy)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=948.4 (M+H). 1H NMR (500 MHz, METHANOL-d4) δ ppm 8.78 (d, J=8.35 Hz, 1H) 8.20-8.27 (m, 1H) 8.10-8.17 (m, 2H) 7.62-7.70 (m, 1H) 7.38-7.43 (m, 1H) 7.28-7.37 (m, 2H) 6.54-7.17 (m, 5H) 4.54-4.67 (m, 2H) 3.63-3.69 (m, 3H) 3.49-3.56 (m, 1H) 3.25-3.27 (m, 3H) 3.15-3.21 (m, 1H) 2.38-2.46 (m, 2H) 1.33-1.39 (m, 1H) 0.98-1.06 (m, 1H) 0.51-0.58 (m, 1H).

### Preparation of Example 21: N-((S)-1-((3P)-3-(4-chloro-1-methyl-3-(methylsulfonamido)-1H-indazol-7-yl)-4-oxo-7-(3-(trifluoromethoxy)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

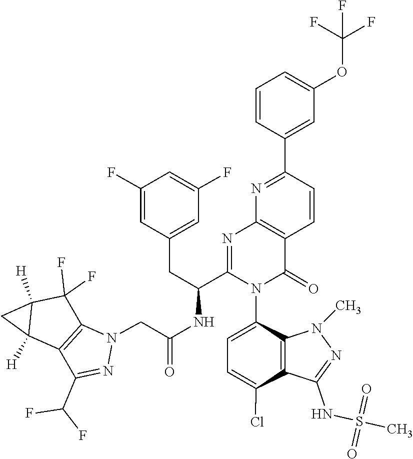

The title compound was prepared according to General Procedure E using (3-(trifluoromethoxy)phenyl)boronic acid as the coupling partner. The experiment afforded the title compound, N-((S)-1-((3P)-3-(4-chloro-1-methyl-3-(methylsulfonamido)-1H-indazol-7-yl)-4-oxo-7-(3-(trifluoromethoxy)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.6 min.; observed ion=966.3 (M+H). 1H NMR (500 MHz, METHANOL-d4) δ ppm 8.77-8.85 (m, 1H) 8.22-8.32 (m, 3H) 7.70-7.78 (m, 1H) 7.51 -7.58 (m, 1H) 7.26-7.38 (m, 2H) 6.52-6.85 (m, 4H) 4.61 (d, J=14.60 Hz, 2H) 3.61-3.70 (m, 3H) 3.52 (dd, J=14.16, 4.62 Hz, 1H) 3.26 (s, 3H) 3.19-3.23 (m, 1H) 2.38-2.48 (m, 2 H) 1.33-1.40 (m, 1H) 0.98-1.05 (m, 1H)

### Preparation of Example 22: N-((S)-1-((3P)-3-(4-chloro-1-methyl-3-(methylsulfonamido)-1H-indazol-7-yl)-7-(4-fluoro-2-(trifluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

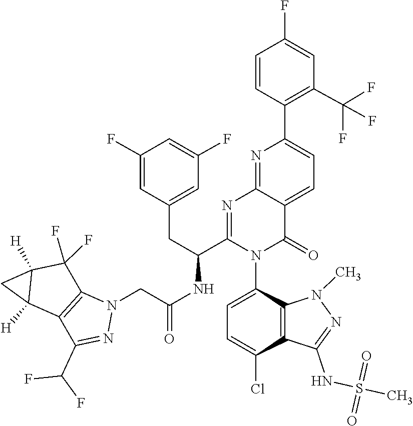

The title compound was prepared according to General Procedure E using (4-fluoro-2-(trifluoromethyl)phenyl)boronic acid as the coupling partner. The experiment afforded the title compound, N-((S)-1-((3P)-3-(4-chloro-1-methyl-3-(methylsuIfonamido)-1H-indazol-7-yl)-7-(4-fluoro-2-(trifluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.51 min.; observed ion=968.4 (M+H). 1H NMR (500 MHz, METHANOL-d4) δ ppm 8.65-8.75 (m, 1H) 7.49-7.72 (m, 4H) 7.20-7.27 (m, 2H) 6.43 -6.71 (m, 4H) 4.41-4.53 (m, 2H) 3.56-3.59 (m, 3H) 3.35-3.41 (m, 1H) 3.15-3.17 (m, 3 H) 2.99-3.05 (m, 1H) 2.27-2.33 (m, 2H) 1.23-1.27 (m, 1H) 0.86-0.91 (m, 1H)

### Preparation of Example 23: N-((S)-1-((3P)-3-(4-chloro-1-methyl-3-(methylsulfonamido)-1H-indazol-7-yl)-7-(3-(difluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

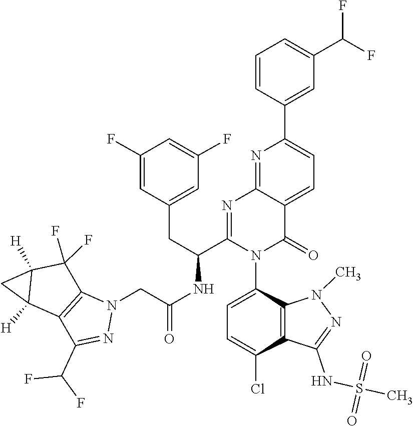

The title compound was prepared according to General Procedure E using (3-(difluoromethyl)phenyl)boronic acid as the coupling partner. The experiment afforded the title compound, N-((S)-1-((3P)-3-(4-chloro-1-methyl-3-(methylsulfonamido)-1H-indazol-7-yl)-7-(3-(difluoromethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.51 min.; observed ion=932.4 (M+H). 1H NMR (500 MHz, METHANOL-d4) δ ppm 8.74-8.86 (m, 1H) 8.48-8.55 (m, 1H) 8.39-8.46 (m, 1H) 8.22 -8.31 (m, 1H) 7.73-7.84 (m, 2H) 7.26-7.38 (m, 2H) 6.53-7.10 (m, 5H) 4.52-4.68 (m, 2 H) 3.65-3.69 (m, 3H) 3.50-3.55 (m, 1H) 3.25-3.27 (m, 3H) 3.18-3.21 (m, 1H) 2.39 -2.46 (m, 2H) 1.34-1.39 (m, 1H) 0.98-1.05 (m, 1H)

### Preparation of Example 24: N-((S)-1-((3P)-3-(4-chloro-1-methyl-3-(methylsulfonamido)-1H-indazol-7-yl)-4-oxo-7-(3-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

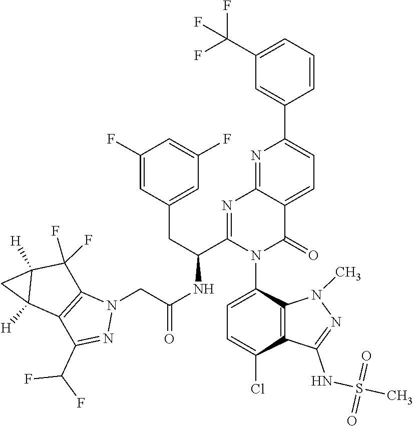

The title compound was prepared according to General Procedure E using (3-(trifluoromethyl)phenyl)boronic acid as the coupling partner. The experiment afforded the title compound, N-((S)-1-((3P)-3-(4-chloro-1-methyl-3-(methylsulfonamido)-1H-indazol-7-yl)-4-oxo-7-(3-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.58 min.; observed ion=950.4 (M+H). 1H NMR (500 MHz, METHANOL-d4) δ ppm 8.76-8.85 (m, 1H) 8.65-8.70 (m, 1H) 8.49-8.56 (m, 1H) 8.27 -8.34 (m, 1H) 7.90-7.95 (m, 1H) 7.81-7.88 (m, 1H) 7.29-7.38 (m, 2H) 6.55-6.84 (m, 4 H) 4.54-4.67 (m, 2H) 3.64-3.69 (m, 3H) 3.49-3.57 (m, 1H) 3.26-3.28 (m, 3H) 3.15 -3.20 (m, 1H) 2.39-2.47 (m, 2H) 1.34-1.39 (m, 1H) 0.98-1.05 (m, 1H).

### Preparation of Example 25: N-((S)-1-((3P)-3-(4-chloro-1-methyl-3-(methylsulfonamido)-1H-indazol-7-yl)-4-oxo-7-(2-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

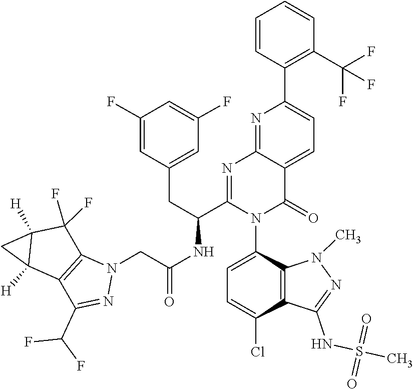

The title compound was prepared according to General Procedure E using (2-(trifluoromethyl)phenyl)boronic acid as the coupling partner. The experiment afforded the title compound, N-((S)-1-((3P)-3-(4-chloro-1-methyl-3-(methylsulfonamido)-1H-indazol-7-yl)-4-oxo-7-(2-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=950.4 (M+H). 1H NMR (500 MHz, METHANOL-d4) δ ppm 8.75-8.87 (m, 1H) 7.93-7.99 (m, 1H) 7.84-7.90 (m, 1H) 7.76 -7.83 (m, 2H) 7.66-7.71 (m, 1H) 7.32-7.38 (m, 2H) 6.55-6.84 (m, 4H) 4.52-4.65 (m, 2 H) 3.66-3.71 (m, 3H) 3.46-3.53 (m, 1H) 3.25-3.28 (m, 3H) 3.09-3.17 (m, 1H) 2.37 -2.46 (m, 2H) 1.33-1.40 (m, 1H) 0.98-1.04 (m, 1H)

### Preparation of Example 26: N-((S)-1-((3P)-3-(4-chloro-1-methyl-3-(methylsulfonamido)-1H-indazol-7-yl)-4-oxo-7-(4-(trifluoromethoxy)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

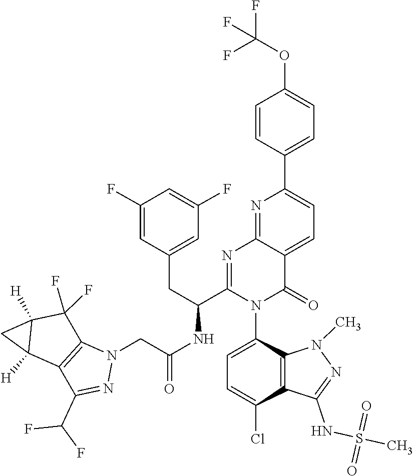

The title compound was prepared according to General Procedure E using (4-(trifluoromethoxy)phenyl)boronic acid as the coupling partner. The experiment afforded the title compound, N-((S)-1-((3P)-3-(4-chloro-1-methyl-3-(methylsulfonamido)-1H-indazol-7-yl)-4-oxo-7-(4-(trifluoromethoxy)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.6 min.; observed ion=966.4 (M+H). 1H NMR (500 MHz, METHANOL-d4) δ ppm 8.75-8.83 (m, 1H) 8.37-8.46 (m, 2H) 8.18-8.28 (m, 1H) 7.54 (d, J=8.05 Hz, 2H) 7.25-7.38 (m, 2H) 6.52-6.88 (m, 4H) 4.91 (br d, J=4.47 Hz, 1H) 4.53-4.68 (m, 2H) 3.63-3.72 (m, 3H) 3.53 (dd, J=14.01, 4.47 Hz, 1H) 3.25-3.28 (m, 3H) 3.15-3.21 (m, 1H) 2.39-2.46 (m, 2H) 1.33-1.39 (m, 1H) 0.98-1.04 (m, 1H)

### Preparation of Example 27: N-((S)-1-((3P)-3-(4-chloro-1-methyl-3-(methylsulfonamido)-1H-indazol-7-yl)-7-(4-(difluoromethoxy)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

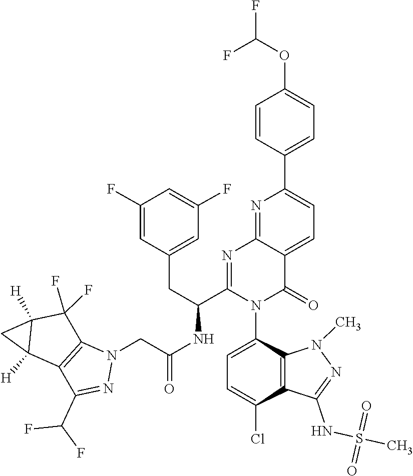

The title compound was prepared according to General Procedure E using (4-(difluoromethoxy)phenyl)boronic acid as the coupling partner. The experiment afforded the title compound, N-((S)-1-((3P)-3-(4-chloro-1-methyl-3-(methylsulfonamido)-1H-indazol-7-yl)-7-(4-(difluoromethoxy)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=948.3 (M+H). 1H NMR (500 MHz, METHANOL-d4) δ ppm 8.70-8.80 (m, 1H) 8.32-8.39 (m, 2H) 8.18-8.23 (m, 1H) 7.62 -7.69 (m, 2H) 7.37-7.42 (m, 2H) 6.52-6.87 (m, 5H) 4.52-4.66 (m, 2H) 3.61-3.69 (m, 3 H) 3.53 (br dd, J=14.16, 4.62 Hz, 1H) 3.26 (s, 3H) 3.18-3.21 (m, 1H) 2.36-2.48 (m, 2H) 1.35-1.41 (m, 1H) 1.01 (br d, J=3.87 Hz, 1H)

### Preparation of Example 28: N-((S)-1-((3P)-3-(4-chloro-1-methyl-3-(methylsulfonamido)-1H-indazol-7-yl)-4-oxo-7-(pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

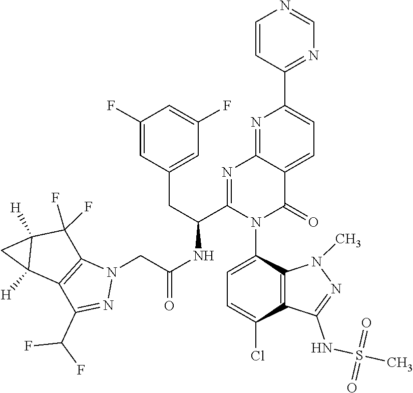

The title compound was prepared according to General Procedure G using 4-(dibutyl(pyrimidin-4-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-4-oxo-7-(pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.32 min.; observed ion=884.4 (M+H). 1H NMR (500 MHz, METHANOL-d4) δ ppm 9.34-9.47 (m, 1H) 9.07 (d, J=5.07 Hz, 1H) 8.85-8.91 (m, 1H) 8.79 (d, J=8.35 Hz, 1H) 8.65 (dd, J=5.36, 1.49 Hz, 1H) 7.26-7.34 (m, 2H) 6.54-6.82 (m, 4H) 4.88-4.92 (m, 1H) 4.51-4.61 (m, 2H) 3.62-3.66 (m, 3H) 3.51 (dd, J=14.01, 4.47 Hz, 1H) 3.21-3.25 (m, 3H) 3.14-3.20 (m, 1H) 2.36-2.44 (m, 2H) 1.30-1.37 (m, 1H) 0.95-1.01 (m, 1H)

### Preparation of Example 29: N-((S)-1-((3P)-3-(4-chloro-1-methyl-3-(methylsulfonamido)-1H-indazol-7-yl)-7-(5-chloropyridin-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

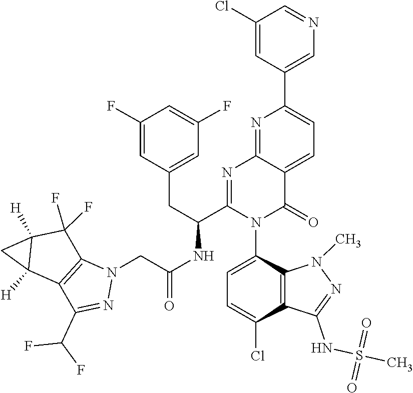

The title compound was prepared according to General Procedure G using 4-(dibutyl(5-chloropyridin-3-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-7-(5-chloropyridin-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.41 min.; observed ion=917.4 (M+H). 1H NMR (500 MHz, METHANOL-d4) δ ppm 9.30-9.44 (m, 1H) 8.74-8.87 (m, 3H) 8.22-8.35 (m, 1H) 7.20 -7.36 (m, 2H) 6.49-6.85 (m, 4H) 4.53-4.63 (m, 2H) 3.63 (s, 3H) 3.50 (dd, J=14.31, 3.87 Hz, 1H) 3.22 (s, 3H) 3.12-3.18 (m, 1H) 2.37-2.45 (m, 2H) 1.30-1.37 (m, 1H) 0.96 -1.01 (m, 1H)

### Preparation of Example 30: N-((S)-1-((3P)-3-(4-chloro-1-methyl-3-(methylsulfonamido)-1H-indazol-7-yl)-7-(6-fluoropyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

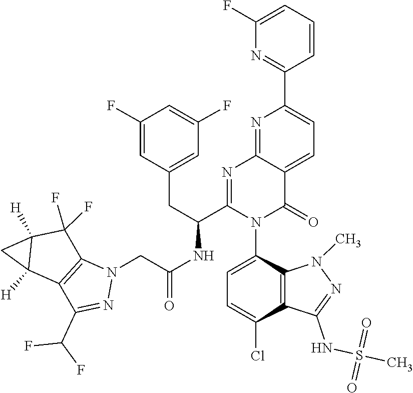

The title compound was prepared according to General Procedure G using 4-(dibutyl(6-fluoropyridin-2-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-7-(6-fluoropyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.47 min.; observed ion=901.4 (M+H). 1H NMR (500 MHz, METHANOL-d4) δ ppm 8.77-8.85 (m, 1H) 8.53-8.64 (m, 2H) 8.11-8.24 (m, 1H) 7.20 -7.35 (m, 3H) 6.53-6.82 (m, 4H) 4.51-4.62 (m, 2H) 3.60-3.68 (m, 3H) 3.48-3.53 (m, 1 H) 3.22-3.23 (m, 3H) 3.13-3.17 (m, 1H) 2.34-2.44 (m, 2H) 1.30-1.37 (m, 1H) 0.93 -1.01 (m, 1H)

### Preparation of Example 31: N-((S)-1-((3P)-3-(4-chloro-1-methyl-3-(methylsulfonamido)-1H-indazol-7-yl)-7-(6-methylpyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

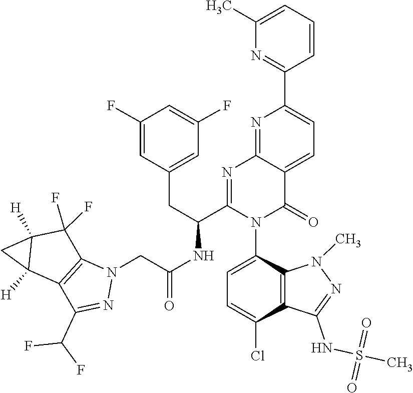

The title compound was prepared according to General Procedure G using 4-(dibutyl(6-methylpyridin-2-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-7-(6-methylpyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.44 min.; observed ion=897.4 (M+H). 1H NMR (500 MHz, METHANOL-d4) δ ppm 8.75-8.83 (m, 1H) 8.65-8.72 (m, 1H) 8.39-8.46 (m, 1H) 7.88 -7.98 (m, 1H) 7.41-7.45 (m, 1H) 7.22-7.34 (m, 2H) 6.52-6.82 (m, 4H) 4.88-4.92 (m, 1 H) 4.50-4.63 (m, 2H) 3.59-3.67 (m, 3H) 3.48-3.54 (m, 1H) 3.23 (s, 3H) 3.13-3.19 (m, 1H) 2.67-2.69 (m, 3H) 2.36-2.44 (m, 2H) 1.31-1.36 (m, 1H) 0.96-1.01 (m, 1H)

### Preparation of Example 32: N-((S)-1-((3P)-3-(4-chloro-1-methyl-3-(methylsulfonamido)-1H-indazol-7-yl)-7-(2-fluoro-4-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

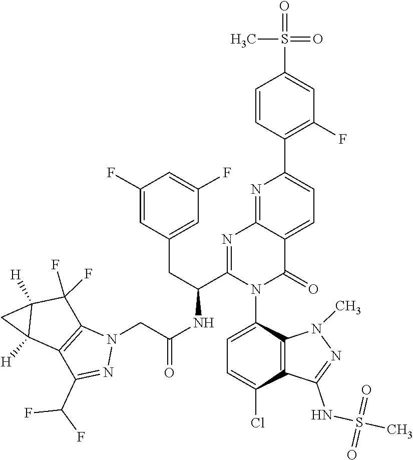

The title compound was prepared according to General Procedure G using (2-methyl-4-(methylsulfonyl)phenyl)boronic acid as the coupling partner. The experiment afforded the title compound, N-((S)-1-((3P)-3-(4-chloro-1-methyl-3-(methylsuIfonamido)-1H-indazol-7-yl)-7-(2-fluoro-4-(methylsuIfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.39 min.; observed ion=978.4 (M+H). 1H NMR (500 MHz, METHANOL-d4) δ ppm 8.78-8.89 (m, 1H) 8.31-8.41 (m, 1H) 8.14 (dd, J=8.34, 2.09 Hz, 1 H) 7.92-8.07 (m, 2H) 7.23-7.38 (m, 2H) 6.52-6.84 (m, 4H) 4.90 (br s, 1H) 4.47-4.64 (m, 2H) 3.59-3.68 (m, 3H) 3.45-3.54 (m, 1H) 3.26 (s, 3H) 3.22 (s, 3H) 3.13-3.18 (m, 1H) 2.37-2.47 (m, 2H) 1.31-1.38 (m, 1H) 0.96-1.01 (m, 1H)

### Preparation of Example 33: N-((S)-1-((3P)-3-(4-chloro-1-methyl-3-(methylsulfonamido)-1H-indazol-7-yl)-4-oxo-7-(pyridazin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

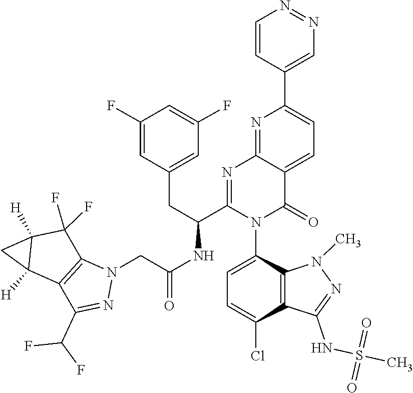

The title compound was prepared according to General Procedure G using 4-(dibutyl(pyridazin-4-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-4-oxo-7-(pyridazin-4-yl)-3,4-dihydropyrido[2,3-d]pyrimidin-2-yl)-2-(3,5- difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.23 min.; observed ion=884.4 (M+H). 1H NMR (500 MHz, METHANOL-d4) δ ppm 9.99-10.15 (m, 1H) 9.41-9.56 (m, 1H) 8.81-8.96 (m, 1H) 8.48 -8.58 (m, 1H) 8.39-8.44 (m, 1H) 7.25-7.39 (m, 2H) 6.48-6.84 (m, 4H) 4.49-4.66 (m, 2 H) 3.60-3.71 (m, 3H) 3.47-3.54 (m, 1H) 3.24 (s, 3H) 3.14-3.20 (m, 1H) 2.37-2.44 (m, 2H) 1.37-1.41 (m, 1H) 0.96-1.01 (m, 1H)

### Preparation of Example 34: N-((S)-1-((3P)-3-(4-chloro-1-methyl-3-(methylsulfonamido)-1H-indazol-7-yl)-4-oxo-7-(pyridin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

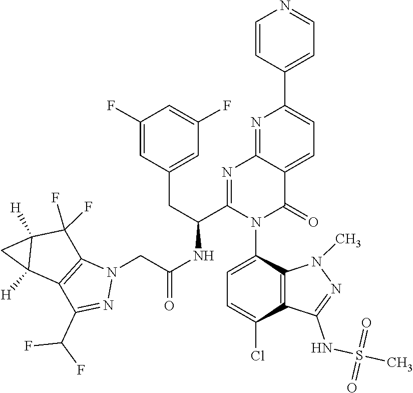

The title compound was prepared according to General Procedure G using 4-(dibutyl(pyridin-4-yl)stannyl)butan-1-ylium as the coupling partner. The experiment afforded the title compound, N-((S)-1-((3P)-3-(4-chloro-1-methyl-3-(methylsulfonamido)-1H-indazol-7-yl)-4-oxo-7-(pyridin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.2 min.; observed ion=883.4 (M+H). 1H NMR (500 MHz, METHANOL-d4) δ ppm 8.75-8.86 (m, 3H) 8.26-8.33 (m, 3H) 7.25-7.33 (m, 2H) 6.51-6.82 (m, 4H) 4.89-4.91 (m, 1H) 4.51-4.64 (m, 2H) 3.60-3.71 (m, 3H) 3.45-3.57 (m, 1H) 3.23 (s, 3H) 3.12-3.20 (m, 1 H) 2.36-2.46 (m, 2H) 1.31-1.36 (m, 1H) 0.95-1.01 (m, 1H).

### Preparation of Example 35: N-((S)-1-((3P)-3-(4-chloro-1-methyl-3-(methylsulfonamido)-1H-indazol-7-yl)-7-(4-(methylsuIfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

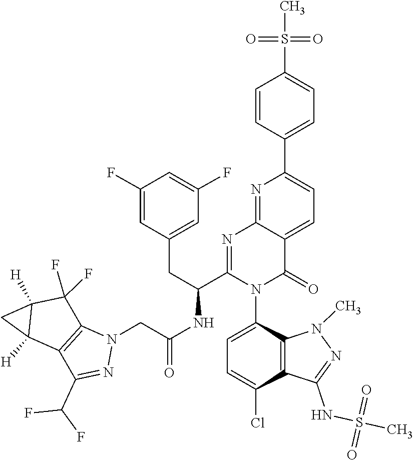

The title compound was prepared according to General Procedure E using (4-(methylsulfonyl)phenyl)boronic acid as the coupling partner. The experiment afforded the title compound, N-((S)-1-((3P)-3-(4-chloro-1-methyl-3-(methylsulfonamido)-1H-indazol-7-yl)-7-(4-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=2.82 min.; observed ion=960 (M+H). 1H NMR (500 MHz, METHANOL-d4) δ ppm 8.77-8.84 (m, 1H) 8.48-8.55 (m, 2H) 8.26-8.31 (m, 1H) 8.15 -8.22 (m, 2H) 7.24-7.32 (m, 2H) 6.52-6.82 (m, 4H) 4.51-4.63 (m, 2H) 3.60-3.66 (m, 3 H) 3.47-3.54 (m, 1H) 3.20-3.25 (m, 6H) 3.12-3.19 (m, 1H) 2.35-2.44 (m, 2H) 1.31 -1.38 (m, 1H) 0.96-1.02 (m, 1H)

### Preparation of Example 36: N-((S)-1-((3P)-3-(4-chloro-1-methyl-3-(methylsulfonamido)-1H-indazol-7-yl)-7-(3-fluoro-6-(trifluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[ 1,2-c]pyrazol-1-yl)acetamide.

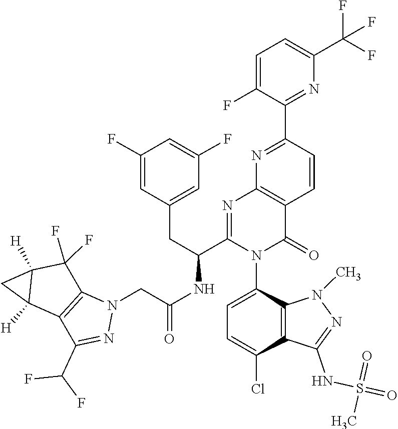

The title compound was prepared according to General Procedure H using 2-chloro-3-fluoro-6-(trifluoromethyl)pyridine as the coupling partner. The experiment afforded the title compound, N-((S)-1-((3P)-3-(4-chloro-1-methyl-3-(methylsulfonamido)-1H-indazol-7-yl)-7-(3-fluoro-6-(trifluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.477 min.; observed ion=969.2 (M+H). 1H NMR (500 MHz, CD3OD, 303 K) Shift (ppm)=8.92-8.86 (m, 1H), 8.33 (d, J=8.0 Hz, 1H), 8.10 (d, J=6.3 Hz, 2H), 7.34-7.27 (m, 2H), 6.77 (br d, J=5.4 Hz, 4H), 4.95-4.92 (m, 1H), 4.61-4.54 (m, 2H), 3.65 (s, 3H), 3.54-3.43 (m, 1H), 3.23 (s, 3H), 2.43-2.37 (m, 2H), 1.42-1.28 (m, 2H), 1.02-0.96 (m, 1H)

### Preparation of Example 37: N-((S)-1-((3P)-3-(4-chloro-1-methyl-3-(methylsulfonamido)-1H-indazol-7-yl)-4-oxo-7-(2-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

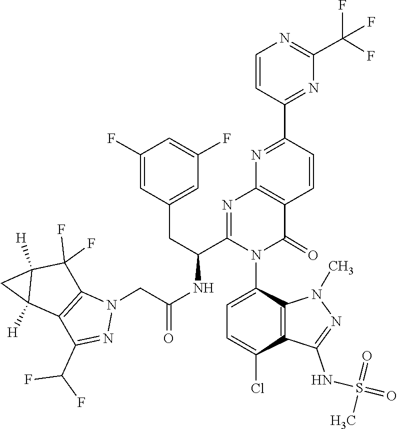

The title compound was prepared according to General Procedure H using 4-chloro-2-(trifluoromethyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(2-(trifluoromethyl)pyrimidin-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.471 min.; observed ion=952.2 (M+H). 1H NMR (500 MHz, CD3OD, 303 K) Shift (ppm)=9.27 (d, J=4.8 Hz, 1H), 8.92 (d, J=7.9 Hz, 1H), 8.84 -8.79 (m, 2H), 7.34-7.29 (m, 2H), 6.79-6.76 (m, 1H), 6.67-6.59 (m, 3H), 4.84-4.81 (m, 1H), 4.55 (d, J=6.9 Hz, 1H), 3.65 (s, 3H), 3.55-3.49 (m, 1H), 3.23 (s, 3H), 3.18-3.16 (m, 1H), 2.44-2.37 (m, 2H), 1.37-1.30 (m, 2H), 1.01-0.96 (m, 1H)

### Preparation of Example 38: N-((S)-1-((3P)-3-(4-chloro-1-methyl-3-(methylsulfonamido)-1H-indazol-7-yl)-7-(6-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

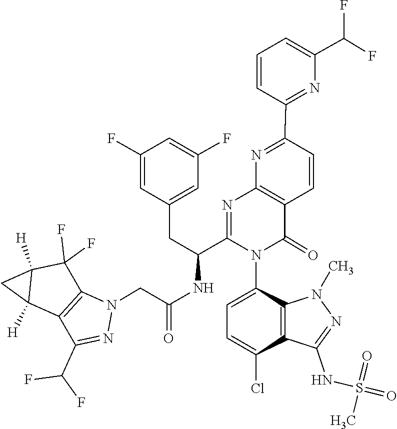

The title compound was prepared according to General Procedure H using 2-bromo-6-(difluoromethyl)pyridine as the coupling partner. The experiment afforded the title compound, N-((S)-1-((3P)-3-(4-chloro-1-methyl-3-(methylsulfonamido)-1H-indazol-7-yl)-7-(6-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.464 min.; observed ion=933.2 (M+H). 1H NMR (500 MHz, METHANOL-d4) δ ppm 0.01-0.07 (m, 1H) 0.97-1.00 (m, 1H) 1.34 (br d, J=5.96 Hz, 1H) 2.40 (br dd, J=6.26, 3.58 Hz, 2H) 3.14-3.20 (m, 1H) 3.23 (s, 3H) 3.47-3.56 (m, 1H) 3.65 (s, 3H) 4.49-4.62 (m, 2H) 6.54-7.00 (m, 5H) 7.27-7.32 (m, 2H) 7.87 (d, J=7.75 Hz, 1H) 8.24 (t, J=7.75 Hz, 1H) 8.75-8.79 (m, 2H) 8.82 (t, J=8.20 Hz, 1H)

### Preparation of Example 39: N-((S)-1-((3P)-3-(4-chloro-1-methyl-3-(methylsulfonamido)-1H-indazol-7-yl)-7-(4-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

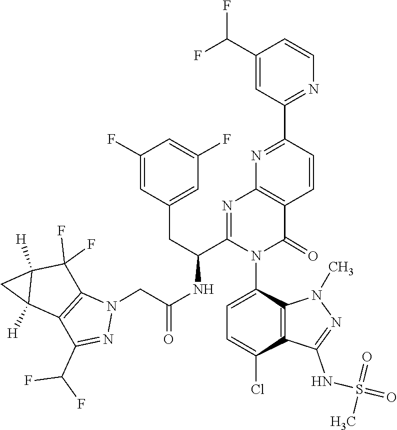

The title compound was prepared according to General Procedure H using 2-chloro-4-(difluoromethyl)pyridine as the coupling partner. The experiment afforded the title compound, N-((S)-1-((3P)-3-(4-chloro-1-methyl-3-(methylsulfonamido)-1H-indazol-7-yl)-7-(4-(difluoromethyl)pyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.448 min.; observed ion=933.2 (M+H). 1H NMR (500 MHz, METHANOL-d4) δ ppm 0.97-1.00 (m, 1H), 1.31-1.36 (m, 1H), 2.37-2.43 (m, 2H), 3.14-3.21 (m, 1H), 3.23 (s, 3H), 3.46-3.55 (m, 1H), 3.65 (s, 3H), 4.50-4.63 (m, 2H), 4.91-4.93 (m, 1H), 6.53-6.79 (m, 4H), 7.02 (t, J=55.43 Hz, 1H), 7.28-7.33 (m, 2H), 7.71 (d, J=5.09 Hz, 1H), 8.70-8.86 (m, 3H), 8.93 (dd, J=5.07, 0.60 Hz, 1H)

### Preparation of Example 47: N-((S)-1-((3P)-3-(4-chloro-1-methyl-3-(methylsulfonamido)-1H-indazol-7-yl)-4-oxo-7-(pyrimidin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

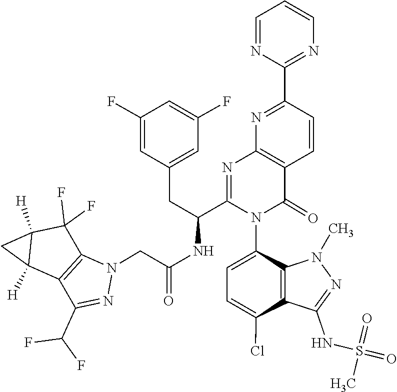

The title compound was prepared according to General Procedure J using 2-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(pyrimidin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.295 min.; observed ion=884.4 (M+H). 1H NMR (500 MHz, CD3OD, 303 K) Shift (ppm)=9.12 (d, J=4.8 Hz, 2H), 8.90 (d, J=8.0 Hz, 1H), 8.83 (d, J=8.0 Hz, 1H), 7.68 (t, J=4.9 Hz, 1H), 7.35 (d, J=7.7 Hz, 1H), 7.27 (d, J=7.7 Hz, 1H), 6.83-6.77 (m, 1H), 6.69-6.62 (m, 2H), 6.69 (br t, J=54.7 Hz, 1H), 4.94-4.91 (m, 1H), 4.65-4.57 (m, 2H), 3.67 (s, 3H), 3.55-3.47 (m, 1H), 3.28-3.24 (m, 3H), 3.21-3.14 (m, 1H), 2.46-2.39 (m, 2H), 1.33-1.25 (m, 1H), 1.04-0.99 (m, 1H)

### Preparation of Example 48: N-((S)-1-((3P)-3-(4-chloro-1-methyl-3-(methylsulfonamido)-1H-indazol-7-yl)-7-(2-methylpyridin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

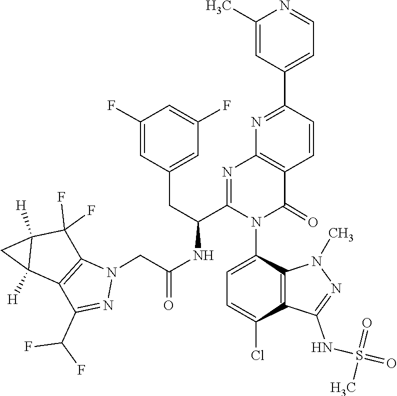

The title compound was prepared according to General Procedure J using 2-methyl-4-(tributylstannyl)pyridine as the coupling partner. The experiment afforded the title compound, N-((S)-1-((3P)-3-(4-chloro-1-methyl-3-(methylsulfonamido)-1H-indazol-7-yl)-7-(2-methylpyridin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.111 min.; observed ion=895.4 (M-H). 1H NMR (500 MHz, CDCl3, 303 K) Shift (ppm)=8.77 (d, J=8.3 Hz, 1H), 8.74 (d, J=4.8 H, 1H), 8.03 (d, J=8.3 Hz, 1H), 7.97 (s, 1H), 7.83 (dd, J=1.6, 5.2 Hz, 1H), 7.13 (d, J=7.7 Hz, 1H), 6.98 (d, J=8.3 Hz, 1H), 6.74-6.69 (m, 1H), 6.65 (t, J=54.8 Hz, 1H), 6.52 (d, J=7.7 Hz, 1H), 6.42 (d, J=6.3 Hz, 2H), 4.88 (q, J=7.7 Hz, 1H), 4.61 (s, 2H), 3.60 (s, 3H), 3.39-3.33 (m, 4H), 3.02 (dd, J=7.9, 13.9 Hz, 1H), 2.74 (s, 3H), 2.48-2.38 (m, 2H), 1.47-1.32 (m, 2H), 1.16 -1.09 (m, 1H)

### Preparation of Example 49: N-((S)-1-((3P)-3-(4-chloro-1-methyl-3-(methylsulfonamido)-1H-indazol-7-yl)-7-(4-methylpyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

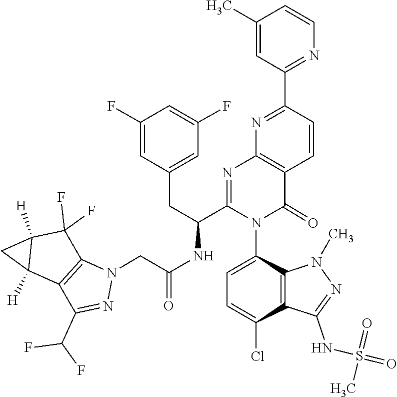

The title compound was prepared according to General Procedure J using 4-methyl-2-(tributylstannyl)pyridine as the coupling partner. The experiment afforded the title compound, N-((S)-1-((3P)-3-(4-chloro-1-methyl-3-(methylsuIfonamido)-1H-indazol-7-yl)-7-(4-methylpyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1 H-cyclopropa[3,4]cyclopenta[1,2-c]pyrazol-1-yl)acetamide. The sample was analyzed using LCMS Method B: retention time=1.413 min.; observed ion=897.4 (M+H). 1H NMR (500 MHz, CDCl3, 303 K) Shift (ppm)=8.76-8.71 (m, 2H), 8.65 (d, J=5.1 Hz, 1H), 8.53 (s, 1H), 7.31-7.28 (m, 1H), 7.14-7.11 (m, 1H), 7.05-6.99 (m, 1H), 6.80-6.55 (m, 3H), 6.41 (br d, J=5.7 Hz, 2H), 4.91-4.86 (m, 1H), 4.61 (s, 2H), 3.60 (s, 3H), 3.40-3.32 (m, 4H), 3.02 (dd, J=8.2, 13.9 Hz, 1H), 2.54 (s, 3H), 2.47-2.36 (m, 2H), 1.42-1.35 (m, 1H), 1.30-1.22 (m, 1H), 1.15-1.08 (m, 1H)

### Preparation of Example 50: N-((S)-1-((3P)-3-(4-chloro-1-methyl-3-(methylsulfonamido)-1H -indazol-7-yl)-7-(6-chloropyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

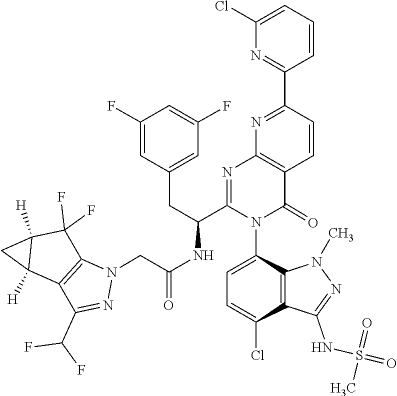

The title compound was prepared according to General Procedure J using 2-chloro-6-(tributylstannyl)pyridine as the coupling partner. The experiment afforded the title compound, N-((S)-1-((3P)-3-(4-chloro-1-methyl-3-(methylsulfonamido)-1H-indazol-7-yl)-7-(6-chloropyridin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.497 min.; observed ion=917.4 (M+H). 1H NMR (500 MHz, CDCl3, 303 K) Shift (ppm)=8.89 (d, J=8.3 Hz, 1H), 8.79 (d, J=8.3 Hz, 1H), 8.76 (dd, J=1.0, 7.9 Hz, 1H), 8.63-8.57 (m, 2H), 8.09 (t, J=7.9 Hz, 1H), 7.88 (t, J=7.7 Hz, 1H), 7.42 (dd, J=0.9, 7.7 Hz, 1H), 7.14 (d, J=8.0 Hz, 1H), 6.99 (d, J=8.6 Hz, 1H), 6.79 -6.54 (m, 3H), 6.43 (dd, J=1.9, 7.3 Hz, 2H), 4.89 (dd, J=1.0, 8.5 Hz, 1H), 4.62 (s, 2H), 3.62 (s, 3H), 3.39-3.34 (m, 4H), 3.04 (dd, J=8.0, 14.0 Hz, 1H), 2.48-2.38 (m, 2H), 1.38 (br d, J=7.2 Hz, 1H), 1.25 (s, 1H), 1.14-1.09 (m, 1H)

### Preparation of Example 51: N-((S)-1-((3P)-3-(4-chloro-1-methyl-3-(methylsulfonamido)-1H-indazol-7-yl)-7-(5-methoxypyridin-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

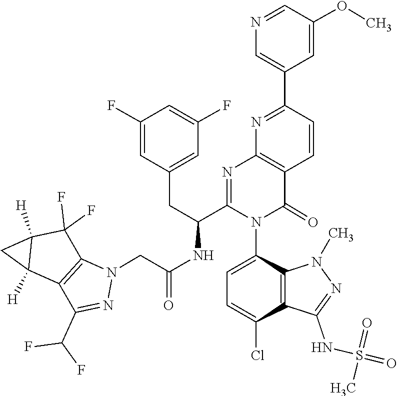

The title compound was prepared according to General Procedure J using 3-methoxy-5-(tributylstannyl)pyridine as the coupling partner. The experiment afforded the title compound, N-((S)-1-((3P)-3-(4-chloro-1-methyl-3-(methylsulfonamido)-1H-indazol-7-yl)-7-(5-methoxypyridin-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.313 min.; observed ion=913.4 (M+H). 1H NMR (500 MHz, CDCl3, 303 K) Shift (ppm)=8.96 (d, J=1.5 Hz, 1H), 8.75 (d, J=8.3 Hz, 1H), 8.50 (d, J=2.7 Hzz, 1H), 8.06 (dd, J=1.9, 2.8 Hz, 1H), 8.02 (d, J=8.3 Hz, 1H), 7.12 (d, J=8.0 Hz, 1H), 6.95 (br d, J=8.0 Hz, 1H), 6.76-6.50 (m, 3H), 6.43 (d, J=6.1 Hz, 2H), 4.87 (d, J=6.9 Hz, 1H), 4.61 (s, 2H), 4.03 (s, 3H), 3.61 (s, 3H), 3.40-3.33 (m, 4H), 3.02 (dd, J=7.9, 13.6 Hz, 1H), 2.44 (br d, J=4.2 Hz, 2H), 1.42-1.35 (m, 1H), 1.25 (s, 1H), 1.14-1.09 (m, 1H)

### Preparation of Example 52: N-((S)-1-((3P)-3-(4-chloro-1-methyl-3-(methylsulfonamido)-1H-indazol-7-yl)-4-oxo-7-(pyridin-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

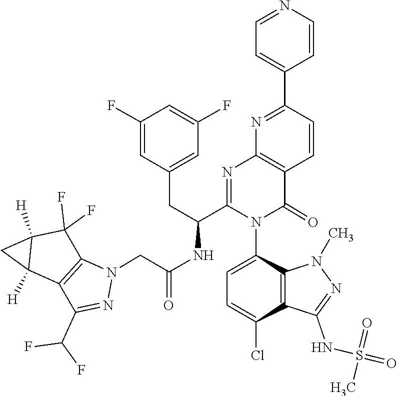

The title compound was prepared according to General Procedure J using 3-(tributylstannyl)pyridine as the coupling partner. The experiment afforded the title compound, N-((S)-1-((3P)-3-(4-chloro-1-methyl-3-(methylsulfonamido)-1H-indazol-7-yl)-4-oxo-7-(pyridin-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.247 min.; observed ion=883.4 (M+H). 1H NMR (500 MHz, CDCl3, 303 K) Shift (ppm)=9.41 (d, J=2.1 Hz, 1H), 8.81 (dd, J=1.5, 4.8 Hz, 1H), 8.76 (d, J=8.3 Hz, 1H), 8.54 (td, J=2.0, 8.0 Hz, 1H), 8.03 (d, J=8.0 Hz, 1H), 7.54 (dd, J=5.1, 8.0 Hz, 1H), 7.13 (d, J=7.7 Hz, 1H), 6.95 (d, J=8.9 Hz, 1H), 6.75-6.69 (m, 1H), 6.63 (br t, J=54.8 Hz, 1H), 6.52 (d, J=7.7 Hz, 1H), 6.44-6.40 (m, 2H), 4.87 (d, J=7.5 Hz, 1H), 4.61 (s, 2H), 3.61 (s, 3H), 3.39 -3.33 (m, 4H), 3.03 (dd, J=7.9, 13.6 Hz, 1H), 2.44 (br dd, J=4.5, 8.3 Hz, 2H), 1.39 (br d, J=6.9 Hz, 1H), 1.25 (s, 1H), 1.14-1.10 (m, 1H)

### Preparation of Example 54: N-((S)-1-((3P)-3-(4-chloro-1-methyl-3-(methylsulfonamido)-1H-indazol-7-yl)-7-(6-methoxypyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

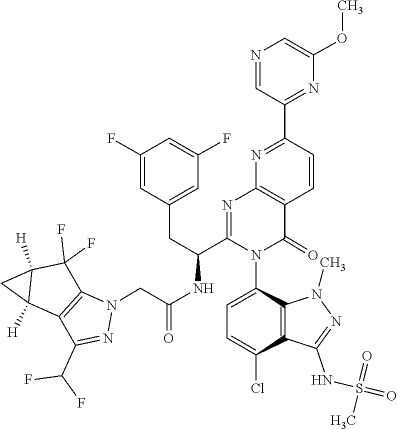

The title compound was prepared according to General Procedure J using 2-methoxy-6-(tributylstannyl)pyrazine as the coupling partner. The experiment afforded the title compound, N-((S)-1-((3P)-3-(4-chloro-1-methyl-3-(methylsulfonamido)-1H-indazol-7-yl)-7-(6-methoxypyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.41 min.; observed ion=914.4 (M+H). 1H NMR (500 MHz, CDCl3, 303 K) Shift (ppm)=9.47 (s, 1H), 8.77 (d, J=8.3 Hz, 1H), 8.62 (d, J=8.3 Hz, 1H), 8.42 (d, J=0.6 Hz, 1H), 7.40-7.30 (m, 1H), 7.13 (d, J=7.7 Hz, 1H), 6.94 (d, J=8.6 Hz, 1H), 6.75-6.69 (m, 1H), 6.56 (d, J=7.7 Hz, 1H), 6.64 (t, J=54.8 Hz, 1H), 6.44-6.40 (m, 2H), 4.87 (dt, J=6.6, 8.2 Hz, 1H), 4.62 (s, 2H), 4.15 (s, 3H), 3.61 (s, 3H), 3.40-3.33 (m, 4H), 3.03 (dd, J=7.9, 13.9 Hz, 1H), 2.51-2.41 (m, 2H), 1.43-1.38 (m, 1H), 1.14 (dt, J=1.8, 4.0 Hz, 1H)

### Preparation of Example 55: N-((S)-1-((3P)-3-(4-chloro-1-methyl-3-(methylsulfonamido)-1H-indazol-7-yl)-4-oxo-7-(pyrimidin-5-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

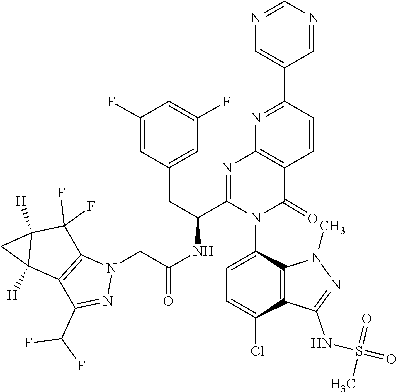

The title compound was prepared according to General Procedure J using 5-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-4-oxo-7-(pyrimidin-5-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.363 min.; observed ion=884.2 (M+H). 1H NMR (500 MHz, CDCl3, 303 K) Shift (ppm)=8.85-8.80 (m, 1H), 8.77 (br d, J=5.4 Hz, 1H), 8.73 (d, J=8.3 Hz, 1H), 7.32 (br s, 1H), 7.14-7.04 (m, 1H), 6.95-6.88 (m, 1H), 6.76-6.65 (m, 1H), 6.74 (br t, J=55.6 Hz, 1H), 6.46-6.31 (m, 3H), 4.92-4.85 (m, 1H), 4.65-4.55 (m, 2H), 4.19 (s, 3H), 3.57 (s, 2H), 3.39-3.30 (m, 4H), 3.03-2.95 (m, 1H), 2.46-2.36 (m, 2H), 1.41-1.30 (m, 1H), 1.08 (br d, J=1.5 Hz, 1H)

### Preparation of Example 56: N-((S)-1-((3P)-3-(4-chloro-1-methyl-3-(methylsulfonamido)-1H-indazol-7-yl)-7-(4-methoxypyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

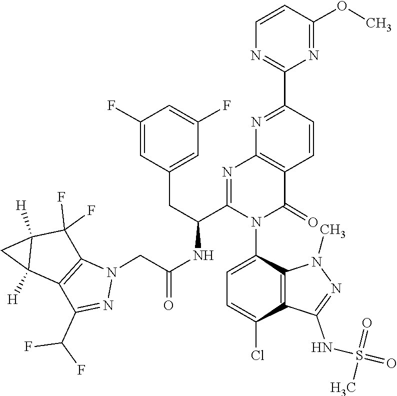

The title compound was prepared according to General Procedure J using 4-methoxy-2-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-7-(4-methoxypyrimidin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.262 min.; observed ion=914.2 (M+H). 1H NMR (500 MHz, CDCl3, 303 K) Shift (ppm)=9.54 (s, 2H), 9.41 (s, 1H), 8.81 (d, J=8.0 Hz, 1H), 8.03 (d, J=8.3 Hz, 1H), 7.36-7.33 (m, 1H), 7.14 (d, J=7.7 Hz, 1H), 6.90 (d, J=8.6 Hz, 1H), 6.75-6.50 (m, 3H), 6.42 (d, J=6.7 Hz, 2H), 4.86 (d, J=6.9 Hz, 1H), 4.61 (d, J=1.5 Hz, 2H), 3.62 (s, 3H), 3.42-3.33 (m, 4H), 3.03 (dd, J=7.9, 13.9 Hz, 1H), 2.46 (td, J=4.2, 8.3 Hz, 2H), 1.43-1.39 (m, 1H), 1.27-1.24 (m, 1H), 1.20-1.10 (m, 1H), 0.87-0.80 (m, 1H)

### Preparation of Example 57: N-((S)-1-((3P)-3-(4-chloro-1-methyl-3-(methylsulfonamido)-1H-indazol-7-yl)-7-(3,5-dichloropyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

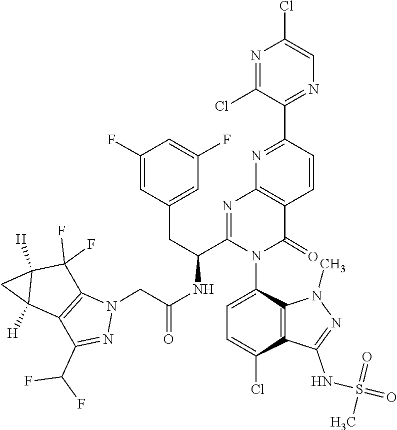

The title compound was prepared according to General Procedure J using 3,5-dichloro-2-(tributylstannyl)pyrazine as the coupling partner. The experiment afforded the title compound, N-((S)-1-((3P)-3-(4-chloro-1-methyl-3-(methylsulfonamido)-1H-indazol-7-yl)-7-(3,5-dichloropyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.468 min.; observed ion=952.1 (M+H). 1H NMR (500 MHz, CDCl3, 303 K) Shift (ppm)=8.83 (d, J=8.0 Hz, 1H), 8.72 (s, 1H), 8.08 (d, J=8.3 Hz, 1H), 7.35-7.31 (m, 1H), 7.13 (d, J=8.0 Hz, 1H), 6.92 (d, J=8.6 Hz, 1H), 6.77-6.51 (m, 3H), 6.42 (d, J=6.8 Hz, 2H), 4.90 (d, J=7.5 Hz, 1H), 4.58 (s, 2H), 3.62 (s, 3H), 3.40-3.31 (m, 4H), 3.01 (dd, J=7.6, 13.9 Hz, 1H), 2.47-2.38 (m, 2H), 1.25 (br d, J=1.2 Hz, 1H), 1.11-1.07 (m, 1H)

### Preparation of Example 58: N-((S)-1-((3P)-3-(4-chloro-1-methyl-3-(methylsulfonamido)-1H-indazol-7-yl)-7-(2-(methylsulfonyl)pyrimidin-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

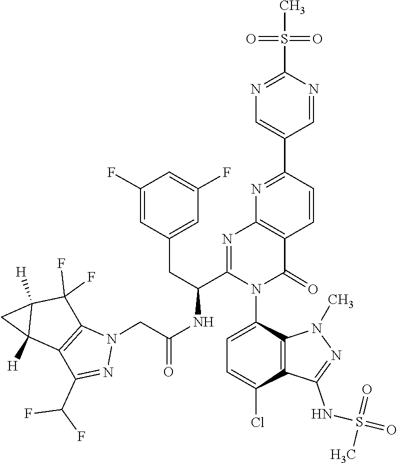

The title compound was prepared according to General Procedure J using 2-(methylsulfonyl)-5-(tributylstannyl)pyrimidine as the coupling partner. The experiment afforded the title compound, N-((S)-1-((3P)-3-(4-chloro-1-methyl-3-(methylsulfonamido)-1H-indazol-7-yl)-7-(2-(methylsulfonyl)pyrimidin-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.29 min.; observed ion=962.2 (M+H). 1H NMR (500 MHz, CDCl3, 303 K) Shift (ppm)=9.69 (s, 2H), 8.87 (d, J=8.0 Hz, 1H), 8.22 (s, 2H), 8.07 (d, J=8.0 Hz, 1H), 7.34 (br s, 1H), 6.92-6.87 (m, 1H), 6.74-6.69 (m, 1H), 6.60-6.56 (m, 1H), 6.42 (d, J=6.6 Hz, 2H), 4.88-4.80 (m, 1H), 4.62 (d, J=1.8 Hz, 2H), 3.63 (s, 3H), 3.48 (s, 3H), 3.41-3.35 (m, 4H), 3.06-3.00 (m, 1H), 1.45-1.40 (m, 2H), 1.14 (br s, 1H), 1.04 -1.02 (m, 1H)

### Preparation of Example 59: N-((S)-1-((3P)-3-(4-chloro-1-methyl-3-(methylsulfonamido)-1H-indazol-7-yl)-7-(2-methoxypyridin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

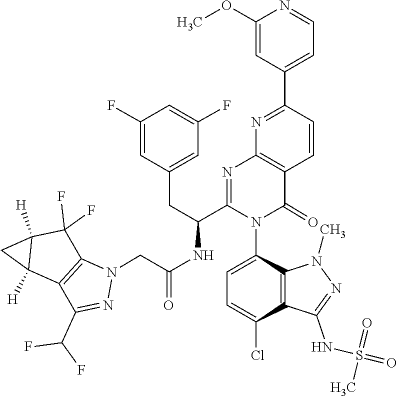

The title compound was prepared according to General Procedure J using 2-methoxy-4-(tributylstannyl)pyridine as the coupling partner. The experiment afforded the title compound, N-((S)-1-((3P)-3-(4-chloro-1-methyl-3-(methylsulfonamido)-1H-indazol-7-yl)-7-(2-methoxypyridin-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.407 min.; observed ion=913.4 (M+H). 1H NMR (500 MHz, CD3OD, 303 K) Shift (ppm)=8.82 (d, J=8.0 Hz, 1H), 8.39 (d, J=5.4 Hz, 1H), 8.26 (d, J=8.3 Hz, 1H), 7.79 (dd, J=1.5, 5.4 Hz, 1H), 7.67 (s, 1H), 7.36-7.30 (m, 2H), 6.83 -6.78 (m, 1H), 6.64 (dd, J=1.9, 7.9 Hz, 2H), 6.67 (t, J=54.8 Hz, 1H), 4.97-4.91 (m, 1H), 4.64-4.58 (m, 2H), 4.05 (s, 3H), 3.66 (s, 3H), 3.52 (dd, J=4.6, 14.2 Hz, 1H), 3.26 (s, 3H), 3.21-3.15 (m, 1H), 2.45-2.39 (m, 2H), 1.39-1.33 (m, 1H), 1.02-0.96 (m, 1H)

### Preparation of Example 60: N-((S)-1-((3P)-3-(4-chloro-1-methyl-3-(methylsulfonamido)-1H-indazol-7-yl)-4-oxo-7-(pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

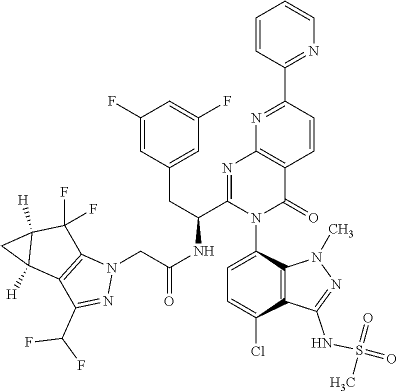

The title compound was prepared according to General Procedure J using 2-(tributylstannyl)pyridine as the coupling partner. The experiment afforded the title compound, N-((S)-1-((3P)-3-(4-chloro-1-methyl-3-(methylsulfonamido)-1H-indazol-7-yl)-4-oxo-7-(pyridin-2-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.389 min.; observed ion=883.4 (M+H). 1H NMR (500 MHz, CD3OD, 303 K) Shift (ppm)=8.84-8.81 (m, 2H), 8.71-8.65 (m, 2H), 8.11-8.09 (m, 1H), 7.60 (t, J=6.3 Hz, 1H), 7.36 -7.30 (m, 2H), 6.71 (s, 4H), 4.87-4.86 (m, 1H), 4.59 (d, J=8.6 Hz, 2H), 3.67 (s, 3H), 3.56 -3.51 (m, 1H), 3.26 (s, 3H), 3.20-3.18 (m, 1H), 2.45-2.39 (m, 2H), 1.36 (br d, J=6.9 Hz, 1H), 1.01 (s, 1H)

### Preparation of Example 61: N-((S)-1-((3P)-3-(4-chloro-1-methyl-3-(methylsulfonamido)-1H-indazol-7-yl)-7-(3-chloropyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

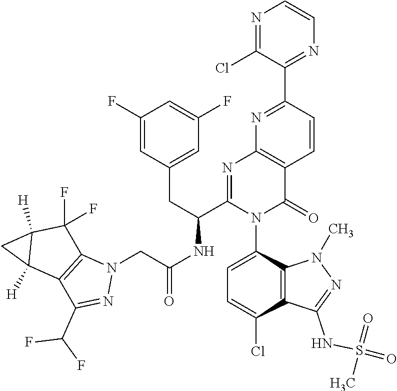

The title compound was prepared according to General Procedure J using 2-chloro-3-(tributylstannyl)pyrazine as the coupling partner. The experiment afforded the title compound, N-((S)-1-((3P)-3-(4-chloro-1-methyl-3-(methylsulfonamido)-1H-indazol-7-yl)-7-(3-chloropyrazin-2-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.36 min.; observed ion=918.2 (M+H). 1H NMR (500 MHz, CD3OD, 303 K) Shift (ppm)=8.90 (d, J=8.3 Hz, 1H), 8.82 (d, J=2.4 Hz, 1H), 8.67 (d, J=2.4 Hz, 1H), 8.14 (d, J=8.0 Hz, 1H), 7.37-7.30 (m, 2H), 6.82-6.63 (m, 4H), 4.94-4.92 (m, 1H), 4.59-4.52 (m, 2H), 3.69 (s, 3H), 3.55-3.47 (m, 1H), 3.26 (s, 3H), 3.19-3.12 (m, 1H), 2.46-2.38 (m, 2H), 1.36 (br d, J=6.0 Hz, 1H), 1.00 (dt, J=1.8, 3.7 Hz, 1H)

### Preparation of Example 62: N-((S)-1-((3P)-3-(4-chloro-1-methyl-3-(methylsulfonamido)-1H-indazol-7-yl)-4-oxo-7-(3-(pentafluoro-16-sulfaneyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

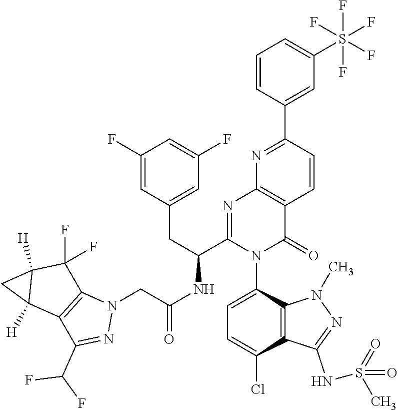

The title compound was prepared according to General Procedure K using 4,4,5,5-tetramethyl-2-(3-(pentafluoro-16-sulfaneyl)phenyl)-1,3,2-dioxaborolane as the coupling partner. The experiment afforded the title compound, N-((S)-1-((3P)-3-(4-chloro-1-methyl-3-(methylsulfonamido)-1H-indazol-7-yl)-4-oxo-7-(3-(pentafluoro-16-sulfaneyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.569 min.; observed ion=1008.4 (M+H). 1H NMR (500 MHz, CD3OD, 303 K) Shift (ppm)=8.87 (s, 1H), 8.82 (d, J=8.3 Hz, 1H), 8.50 (d, J=8.0 Hz, 1H), 8.31 (d, J=8.3 Hz, 1H), 8.07 (dd, J=1.5, 8.3 Hz, 1H), 7.84 (t, J=8.2 Hz, 1H), 7.37-7.31 (m, 2H), 6.83-6.77 (m, 1H), 6.64 (br d, J=6.6 Hz, 2H), 6.67 (br t, J=54.8 Hz, 1H), 4.67-4.57 (m, 3H), 3.66 (s, 3H), 3.55-3.45 (m, 1H), 3.26 (s, 3H), 3.22-3.16 (m, 1H), 2.46-2.39 (m, 2H), 1.35 (s, 1H), 1.00 (br dd, J=1.0, 3.1 Hz, 1H)

### Preparation of Example 63: N-((S)-1-((3P)-3-(4-chloro-1-methyl-3-(methylsulfonamido)-1H-indazol-7-yl)-4-oxo-7-(4-(pentafluoro-l6-sulfaneyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

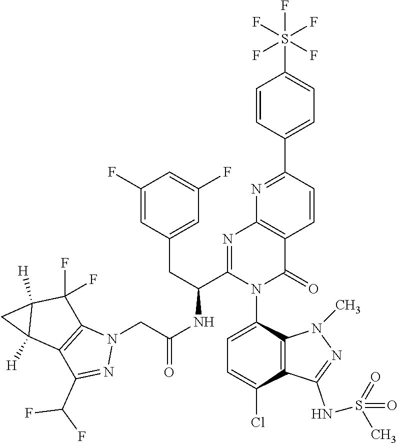

The title compound was prepared according to General Procedure K using 4,4,5,5-tetramethyl-2-(4-(pentafluoro-l6-sulfaneyl)phenyl)-1,3,2-dioxaborolane as the coupling partner. The experiment afforded the title compound, N-((S)-1-((3P)-3-(4-chloro-1-methyl-3-(methylsulfonamido)-1H-indazol-7-yl)-4-oxo-7-(4-(pentafluoro-l6-sulfaneyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.569 min.; observed ion=1008.3 (M+H). 1H NMR (500 MHz, CD3OD, 303 K) Shift (ppm)=8.83 (d, J=8.3 Hz, 1H), 8.48 (d, J=8.6 Hz, 2H), 8.29 (d, J=8.3 Hz, 1H), 8.10 (d, J=7.9 Hz, 2H), 7.35-7.29 (m, 2H), 6.83-6.78 (m, 1H), 6.65 (dd, J=1.9, 7.9 Hz, 2H), 6.68 (t, J=54.8 Hz, 1H), 4.97-4.92 (m, 1H), 4.64-4.55 (m, 2H), 3.67 (s, 3H), 3.53 (dd, J=4.6, 14.5 Hz, 1H), 3.26 (s, 3H), 3.22-3.16 (m, 1H), 2.46-2.39 (m, 2H), 1.36 (br d, J=7.5 Hz, 1H), 1.01 (td, J=2.1, 3.6 Hz, 1H)

### Preparation of Example 64: N-((S)-1-((3P)-3-(4-chloro-1-methyl-3-(methylsulfonamido)-1H-indazol-7-yl)-7-(1H-imidazol-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

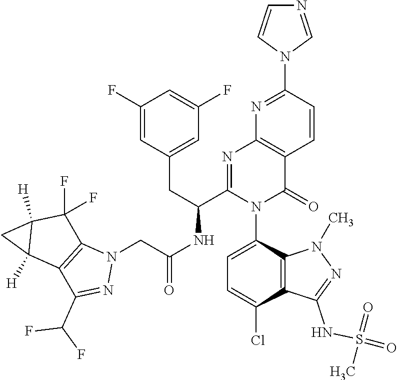

A mixture of 3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (50 mg, 0.050 mmol) and 1H-imidazole (34.2 mg, 0.502 mmol) in 1,2-Dimethoxyethane (DME) (1 mL) was heated at 95° C. for 1 h. LCMS showed a peak with the expected M+H. The mixture was concentrated and the residue was dissolved in DMF, filtered, and the filtrate was subjected to HPLC purification to afford the title compound, N-((S)-1-((3P)-3-(4-chloro-1-methyl-3-(methylsulfonamido)-1H-indazol-7-yl)-7-(1H-imidazol-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.17 min.; observed ion=872.1 (M+H). 1H NMR (500 MHz, METHANOL-d4) δ ppm 8.79-8.88 (m, 2H), 8.16 (t, J=1.49 Hz, 1H), 8.03 (d, J=8.64 Hz, 1 H), 7.26-7.38 (m, 3H), 6.54-6.85 (m, 4H), 4.58 (d, J=10.13 Hz, 2H), 3.67 (s, 3H), 3.47 -3.55 (m, 1H), 3.26 (s, 3H), 3.13-3.21 (m, 1H), 2.37-2.48 (m, 2H), 1.36 (q, J=7.25 Hz, 1 H), 0.98-1.05 (m, 1H).

### Preparation of Example 65: N-((S)-1-((3P)-3-(4-chloro-1-methyl-3-(methylsulfonamido)-1H-indazol-7-yl)-4-oxo-7-(3-(trifluoromethyl)-1H-pyrazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

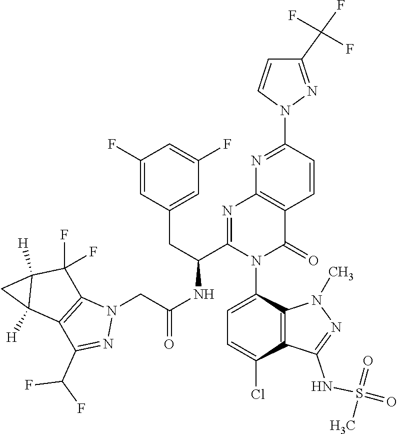

To a solution of 3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (50 mg, 0.050 mmol) and 3-(trifluoromethyl)-1H-pyrazole (10.24 mg, 0.075 mmol) in N,N-Dimethylacetamide (DMA) (1 mL) was added K2CO3 (8.32 mg, 0.060 mmol) and the mixture was heated at 40° C. for 16 h. LCMS showed a peak with the expected M+H. The mixture was filtered and the filtrate was subjected to HPLC purification to afford the title compound, N-((S)-1-((3P)-3-(4-chloro-1-methyl-3-(methylsulfonamido)-1H-indazol-7-yl)-4-oxo-7-(3-(trifluoromethyl)-1H-pyrazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=940.1 (M+H). 1H NMR (500 MHz, METHANOL-d4) δ ppm 8.97 (dt, J=2.76, 1.01 Hz, 1H), 8.88 (d, J=8.34 Hz, 1H), 8.34 (d, J=8.64 Hz, 1H), 7.28-7.37 (m, 2 H), 7.04 (d, J=2.68 Hz, 1H), 6.55-6.85 (m, 4H), 4.56 (d, J=6.85 Hz, 2H), 3.68 (s, 3H), 3.48-3.55 (m, 1H), 3.27 (s, 3H), 3.12-3.21 (m, 1H), 2.39-2.53 (m, 2H), 1.33-1.40 (m, 1 H), 0.97-1.04 (m, 1H).

### Preparation of Example 66: N-((S)-1-((3P)-3-(4-chloro-1-methyl-3-(methylsulfonamido)-1H-indazol-7-yl)-4-oxo-7-(4-(trifluoromethyl)-1H-pyrazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

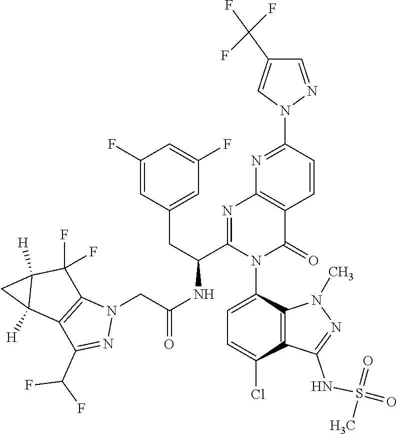

To a solution of 3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (50 mg, 0.050 mmol) and 4-(trifluoromethyl)-1H-pyrazole (10.24 mg, 0.075 mmol) in N,N-Dimethylacetamide (DMA) (1 mL) was added K2CO3 (8.32 mg, 0.060 mmol) and the mixture was heated at 40° C. for 16 h. LCMS showed a peak with the expected M+H. The mixture was filtered and the filtrate was subjected to HPLC purification to afford the title compound, N-((S)-1-((3P)-3-(4-chloro-1-methyl-3-(methylsulfonamido)-1H-indazol-7-yl)-4-oxo-7-(4-(trifluoromethyl)-1H-pyrazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=940.2 (M+H). 1H NMR (500 MHz, METHANOL-d4) δ ppm 9.22 -9.30 (m, 1H), 8.87 (d, J=8.64 Hz, 1H), 8.35 (d, J=8.64 Hz, 1H), 8.23 (s, 1H), 7.25-7.38 (m, 2H), 6.51-6.86 (m, 4H), 4.56 (d, J=7.15 Hz, 2H), 3.68 (s, 3H), 3.48-3.56 (m, 1H), 3.27 (s, 3H), 3.17 (dd, J=14.16, 9.69 Hz, 1H), 2.43 (ddd, J=11.25, 7.67, 4.02 Hz, 2H), 1.33-1.40 (m, 1H), 0.97-1.05 (m, 1H).

### Preparation of Example 67: N-((S)-1-((3P)-3-(4-chloro-1-methyl-3-(methylsulfonamido)-1H-indazol-7-yl)-7-(2-methylbenzo[d]thiazol-6-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

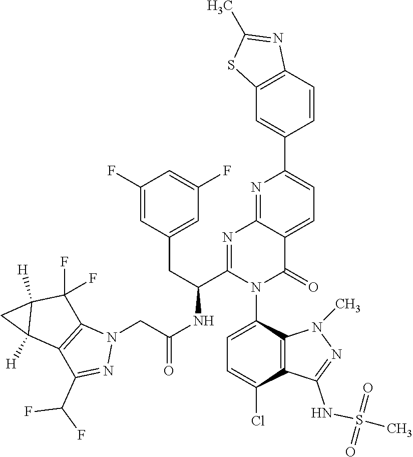

The title compound was prepared according to General Procedure K using (2-methylbenzo[d]thiazol-6-yl)boronic acid as the coupling partner. The experiment afforded the title compound, N-((S)-1-((3P)-3-(4-chloro-1-methyl-3-(methylsulfonamido)-1H-indazol-7-yl)-7-(2-methylbenzo[d]thiazol-6-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.45 min.; observed ion=953.3 (M+H). 1H NMR (500 MHz, METHANOL-d4) δ ppm 8.84-8.92 (m, 1H), 8.75 (d, J=8.35 Hz, 1H), 8.41 (dd, J=8.64, 1.79 Hz, 1H), 8.27 (d, J=8.64 Hz, 1H), 8.09 (d, J=8.34 Hz, 1H), 7.24-7.35 (m, 2H), 6.54-6.85 (m, 4H), 4.53-4.59 (m, 2H), 3.65 (s, 3H), 3.51 (dd, J=14.16, 4.62 Hz, 1H), 3.24 (s, 3H), 3.12-3.20 (m, 1H), 2.91 (s, 3H), 2.37-2.45 (m, 2H), 1.31-1.37 (m, 1H), 0.94-1.02 (m, 1H).

### Preparation of Example 68: N-((S)-1-((3P)-3-(4-chloro-1-methyl-3-(methylsulfonamido)-1H-indazol-7-yl)-7-(1-methyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

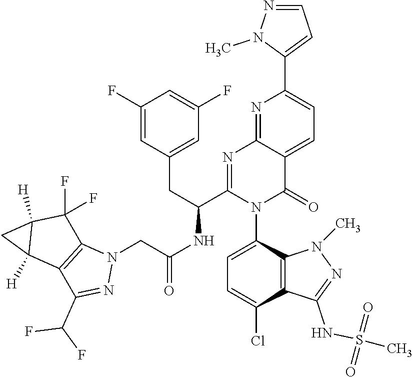

The title compound was prepared according to General Procedure K using (1-methyl-1H-pyrazol-5-yl)boronic acid as the coupling partner. The experiment afforded the title compound, N-((S)-1-((3P)-3-(4-chloro-1-methyl-3-(methylsulfonamido)-1H-indazol-7-yl)-7-(1-methyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.36 min.; observed ion=886.4 (M+H). 1H NMR (500 MHz, METHANOL-d4) δ ppm 8.72 (d, J=8.05 Hz, 1H), 8.04 (d, J=8.34 Hz, 1H), 7.62 (d, J=2.09 Hz, 1H), 7.27-7.36 (m, 2H), 7.05 (d, J=2.09 Hz, 1H), 6.49-6.85 (m, 4H), 4.57 (d, J=8.94 Hz, 2H), 4.40 (s, 3H), 3.64 (s, 3H), 3.49 (dd, J=14.16, 4.32 Hz, 1H), 3.24 (s, 3H), 3.14 (dd, J=14.16, 9.69 Hz, 1H), 2.35-2.45 (m, 2H), 1.30-1.37 (m, 1H), 0.94-1.02 (m, 1H).

### Preparation of Example 69: N-((S)-1-((3P)-3-(4-chloro-1-methyl-3-(methylsulfonamido)-1H-indazol-7-yl)-7-(1-isobutyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

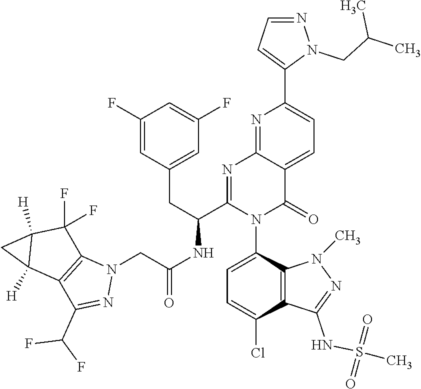

The title compound was prepared according to General Procedure K using (1-isobutyl-1H-pyrazol-5-yl)boronic acid as the coupling partner. The experiment afforded the title compound, N-((S)-1-((3P)-3-(4-chloro-1-methyl-3-(methylsulfonamido)-1H-indazol-7-yl)-7-(1-isobutyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=928.4 (M+H). 1H NMR (500 MHz, METHANOL-d4) δ ppm 8.72 (d, J=8.05 Hz, 1H), 8.03 (d, J=8.34 Hz, 1H), 7.64 (d, J=2.09 Hz, 1H), 7.23-7.33 (m, 2H), 7.04 (d, J=2.09 Hz, 1H), 6.51-6.82 (m, 4H), 4.66 (dd, J=13.26, 7.30 Hz, 1H), 4.45-4.56 (m, 2H), 3.67 (s, 3H), 3.53 (dd, J=14.16, 4.92 Hz, 1H), 3.25-3.27 (m, 2H), 3.24 (s, 3H), 3.11-3.16 (m, 1H), 2.37-2.45 (m, 2H), 2.14-2.23 (m, 1H), 1.31-1.37 (m, 1H), 0.89 (dd, J=6.71, 1.64 Hz, 6H).

### Preparation of Example 70: N-((S)-1-((3P)-3-(4-chloro-1-methyl-3-(methylsulfonamido)-1H-indazol-7-yl)-7-(1,3-dimethyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

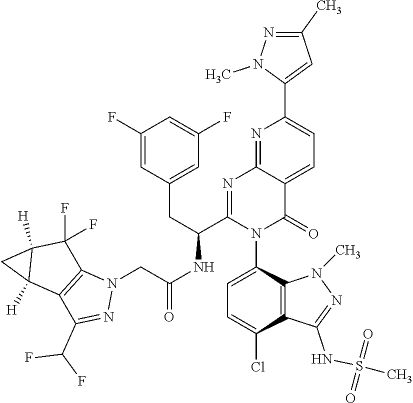

The title compound was prepared according to General Procedure K using (1,3-dimethyl-1H-pyrazol-5-yl)boronic acid as the coupling partner. The experiment afforded the title compound, N-((S)-1-((3P)-3-(4-chloro-1-methyl-3-(methylsulfonamido)-1H-indazol-7-yl)-7-(1,3-dimethyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1 H-cyclopropa[3,4]cyclopenta[1,2-c]pyrazol-1-yl)acetamide. The sample was analyzed using LCMS Method H: retention time=1.37 min.; observed ion=900.4 (M+H). 1H NMR (500 MHz, METHANOL-d4) δ ppm 8.70 (d, J=8.34 Hz, 1H), 7.99 (d, J=8.34 Hz, 1H), 7.29-7.37 (m, 2 H), 6.83 (d, J=0.60 Hz, 1H), 6.49-6.80 (m, 4H), 4.51-4.58 (m, 2H), 4.32 (s, 3H), 3.64 (s, 3H), 3.48 (dd, J=14.31, 4.77 Hz, 1H), 3.24 (s, 3H), 3.13 (dd, J=14.31, 9.84 Hz, 1H), 2.36 -2.44 (m, 2H), 2.33 (s, 3H), 1.30-1.37 (m, 1H), 0.94-1.00 (m, 1H).

### Preparation of Example 71: N-((S)-1-(7-(benzo[d]thiazol-6-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

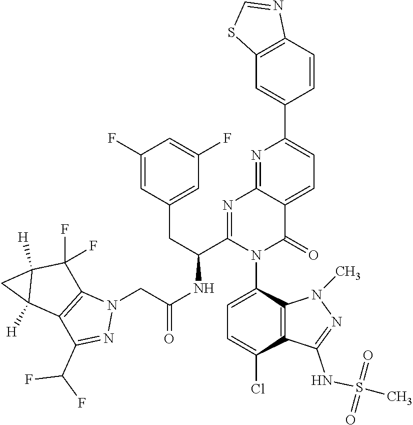

The title compound was prepared according to General Procedure K using benzo[d]thiazol-6-ylboronic acid as the coupling partner. The experiment afforded the title compound, N-((S)-1-(7-(benzo[d]thiazol-6-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=939.1 (M+H). 1H NMR (500 MHz, METHANOL-d4) δ ppm 9.41 (s, 1 H), 9.03 (d, J=1.79 Hz, 1H), 8.77 (d, J=8.05 Hz, 1H), 8.49 (dd, J=8.64, 1.79 Hz, 1H), 8.30 (dd, J=14.90, 8.34 Hz, 2H), 7.23-7.37 (m, 2H), 6.48-6.86 (m, 4H), 4.52-4.59 (m, 2H), 3.66 (s, 3H), 3.52 (dd, J=14.01, 4.47 Hz, 1H), 3.24 (s, 3H), 3.17 (dd, J=14.31, 9.54 Hz, 1 H), 2.35-2.46 (m, 2H), 1.31-1.38 (m, 1H), 0.95-1.03 (m, 1H).

### Preparation of Example 72: N-((S)-1-(7-(benzo[d]thiazol-5-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

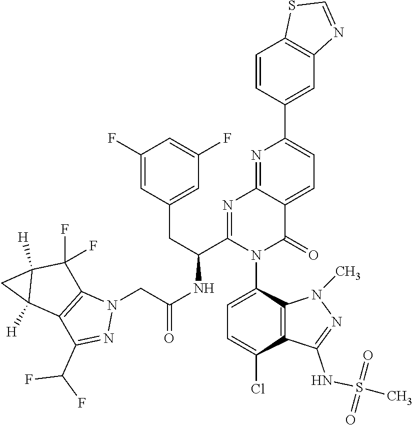

The title compound was prepared according to General Procedure K using benzo[d]thiazol-5-ylboronic acid as the coupling partner. The experiment afforded the title compound, N-((S)-1-(7-(benzo[d]thiazol-5-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.43 min.; observed ion=939.1 (M+H). 1H NMR (500 MHz, METHANOL-d4) δ ppm 9.38 (s, 1 H), 8.98 (d, J=1.49 Hz, 1H), 8.79 (d, J=8.05 Hz, 1H), 8.42 (dd, J=8.64, 1.79 Hz, 1H), 8.25 -8.38 (m, 2H), 7.20-7.44 (m, 2H), 6.47-6.87 (m, 4H), 4.54-4.60 (m, 2H), 3.65 (s, 3H), 3.52 (dd, J=14.45, 4.62 Hz, 1H), 3.24 (s, 3H), 3.17 (dd, J=14.16, 9.69 Hz, 1H), 2.36-2.45 (m, 2H), 1.31-1.38 (m, 1H), 0.97-1.02 (m, 1H).

### Preparation of Example 73: N-((S)-1-((3P)-3-(4-chloro-1-methyl-3-(methylsulfonamido)-1H-indazol-7-yl)-4-oxo-7-(1-(2,2,2-trifluoroethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

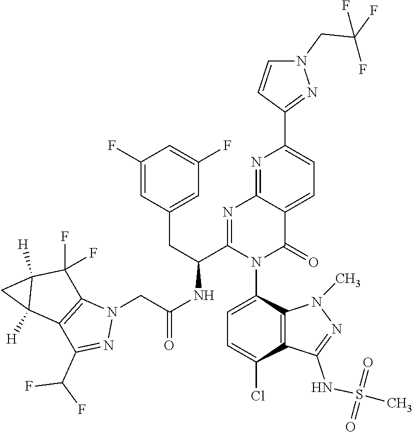

To a mixture of N-((S)-1-(3-(4-chloro-3-(N-(4-methoxybenzyl)methylsulfonamido)-1-methyl-1H-indazol-7-yl)-4-oxo-7-(1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (25 mg, 0.025 mmol) and 2,2,2-trifluoroethyl trifluoromethanesulfonate (29.2 mg, 0.126 mmol) in Acetonitrile (1 mL) was added cesium carbonate (12.31 mg, 0.038 mmol) and the resulting mixture was heated at 60° C. for 1 h. The mixture was cooled to room temperature, filtered, and the filtrate was concentrated in vacuo. The residue was taken up in DCM (0.5 mL) and TFA (1 mL), then to the solution was added triflic acid (0.05 mL). The solution was stirred at rt for 1 h and then concentrated in vacuo. The residue was dissolved in DMF (2 mL), filtered, and the filtrate was subjected to HPLC purification to afford the title compound, N-((S)-1-((3P)-3-(4-chloro-1-methyl-3-(methylsulfonamido)-1H-indazol-7-yl)-4-oxo-7-(1-(2,2,2-trifluoroethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.43 min.; observed ion=954.4 (M+H). 1H NMR (500 MHz, METHANOL-d4) δ ppm 8.71 (d, J=8.35 Hz, 1H), 8.33 (d, J=8.35 Hz, 1H), 7.95 (d, J=2.38 Hz, 1H), 7.29-7.32 (m, 1H), 7.29 (d, J=2.38 Hz, 1H), 7.24-7.27 (m, 1H), 6.45-6.86 (m, 4H), 5.07-5.18 (m, 2H), 4.55 (d, J=5.96 Hz, 2H), 3.64 (s, 3H), 3.50 (dd, J=14.16, 4.62 Hz, 1H), 3.23 (s, 3H), 3.09 -3.19 (m, 1H), 2.34-2.45 (m, 2H), 1.32-1.37 (m, 1H), 0.95-1.03 (m, 1H).

### Preparation of Example 74: N-((S)-1-((3P)-3-(4-chloro-1-methyl-3-(methylsulfonamido)-1H-indazol-7-yl)-7-(1-cyclopropyl-1H-pyrazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

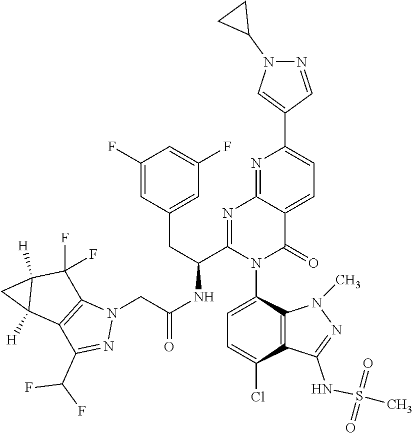

The title compound was prepared according to General Procedure K using 1-cyclopropyl-4-(4,4,5,5-tetramethyl-1,3,2-dioxaborolan-2-yl)-1H-pyrazole as the coupling partner. The experiment afforded the title compound, N-((S)-1-((3P)-3-(4-chloro-1-methyl-3-(methylsulfonamido)-1H-indazol-7-yl)-7-(1-cyclopropyl-1H-pyrazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.36 min.; observed ion=912.4 (M+H). 1H NMR (500 MHz, METHANOL-d4) δ ppm 8.60 (d, J=8.05 Hz, 1H), 8.56 (s, 1H), 8.29 (d, J=0.60 Hz, 1H), 7.94 (d, J=8.34 Hz, 1H), 7.18-7.33 (m, 2H), 6.54-6.83 (m, 4H), 4.55 (d, J=3.87 Hz, 2H), 3.82 (tt, J=7.30, 3.87 Hz, 1H), 3.63 (s, 3H), 3.47-3.53 (m, 1H), 3.23 (s, 3H), 3.09-3.18 (m, 1H), 2.36-2.44 (m, 2H), 1.31 -1.37 (m, 2H), 1.19-1.25 (m, 2H), 1.12-1.18 (m, 2H), 0.96-1.02 (m, 1H).

### Preparation of Example 75: N-((S)-1-((3P)-3-(4-chloro-1-methyl-3-(methylsulfonamido)-1H-indazol-7-yl)-4-oxo-7-(1-(2,2,2-trifluoroethyl)-1H-pyrazol-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

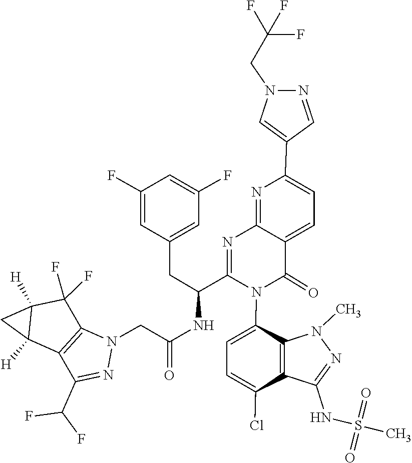

The title compound was prepared according to General Procedure K using 4-(4,4,5,5-tetramethyl-1,3,2-dioxaborolan-2-yl)-1-(2,2,2-trifluoroethyl)-1H-pyrazole as the coupling partner. The experiment afforded the title compound, N-((S)-1-((3P)-3-(4-chloro-1-methyl-3-(methylsulfonamido)-1H-indazol-7-yl)-4-oxo-7-(1-(2,2,2-trifluoroethyl)-1H-pyrazol-4-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.4 min.; observed ion=954.4 (M+H). 1H NMR (500 MHz, METHANOL-d4) δ ppm 8.63-8.66 (m, 2H), 8.40 (s, 1H), 7.98 (d, J=8.34 Hz, 1H), 7.21-7.32 (m, 2H), 6.52-6.82 (m, 4H), 5.11 (q, J=8.54 Hz, 2H), 4.55 (d, J=4.47 Hz, 2H), 3.63 (s, 3H), 3.50 (dd, J=14.45, 4.62 Hz, 1H), 3.23 (s, 3H), 3.13-3.18 (m, 1H), 2.31-2.47 (m, 2H), 1.32-1.37 (m, 1H), 0.95 -1.02 (m, 1H).

### Preparation of Example 76: N-((S)-1-((3P)-3-(4-chloro-1-methyl-3-(methylsulfonamido)-1H-indazol-7-yl)-7-(1-(2-fluoroethyl)-1H-pyrazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

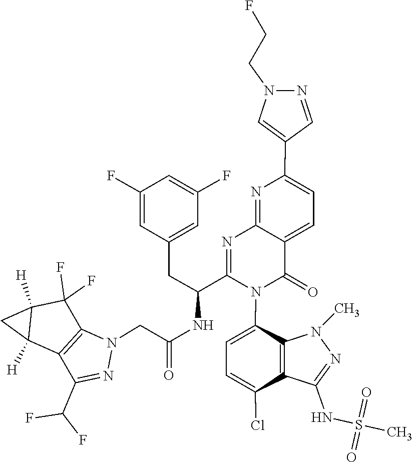

The title compound was prepared according to General Procedure K using 1-(2-fluoroethyl)-4-(4,4,5,5-tetramethyl-1,3,2-dioxaborolan-2-yl)-1H-pyrazole as the coupling partner. The experiment afforded the title compound, N-((S)-1-((3P)-3-(4-chloro-1-methyl-3-(methylsulfonamido)-1H-indazol-7-yl)-7-(1-(2-fluoroethyl)-1H-pyrazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.32 min.; observed ion=918.4 (M+H). 1H NMR (500 MHz, METHANOL-d4) δ ppm 8.62 (d, J=8.34 Hz, 1H), 8.56 (s, 1H), 8.36 (d, J=0.60 Hz, 1H), 7.95 (d, J=8.34 Hz, 1H), 7.35-7.40 (m, 1H), 7.21-7.31 (m, 2H), 6.53-6.81 (m, 4H), 4.78-4.81 (m, 2H), 4.54-4.59 (m, 3 H), 3.63 (s, 3H), 3.49 (dd, J=14.16, 4.32 Hz, 1H), 3.23 (s, 3H), 3.11-3.16 (m, 1H), 2.37 -2.45 (m, 2H), 1.31-1.36 (m, 1H), 0.96-1.01 (m, 1H).

### Preparation of Example 77: N-((S)-1-((3P)-3-(4-chloro-1-methyl-3-(methylsulfonamido)-1H-indazol-7-yl)-7-(1-cyclopropyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

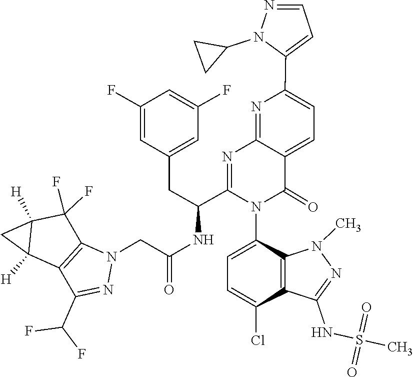

The title compound was prepared according to General Procedure K using 1-cyclopropyl-5-(4,4,5,5-tetramethyl-1,3,2-dioxaborolan-2-yl)-1H-pyrazole as the coupling partner. The experiment afforded the title compound, N-((S)-1-((3P)-3-(4-chloro-1-methyl-3-(methylsulfonamido)-1H-indazol-7-yl)-7-(1-cyclopropyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.41 min.; observed ion=912.4 (M+H). 1H NMR (500 MHz, METHANOL-d4) δ ppm 10.51 -10.57 (m, 1H), 8.76 (d, J=8.34 Hz, 1H), 8.06 (d, J=8.05 Hz, 1H), 7.57 (d, J=2.09 Hz, 1H), 7.27-7.37 (m, 2H), 6.96 (d, J=2.09 Hz, 1H), 6.54-6.82 (m, 4H), 4.55-4.57 (m, 2H), 3.65 (s, 3H), 3.47-3.52 (m, 1H), 3.24 (s, 3H), 3.08-3.14 (m, 1H), 2.35-2.47 (m, 2H), 1.31-1.37 (m, 2H), 1.05-1.15 (m, 4H), 0.98 (td, J=5.89, 4.02 Hz, 1H).

### Preparation of Example 78: N-((S)-1-((3P)-3-(4-chloro-1-methyl-3-(methylsulfonamido)-1H-indazol-7-yl)-7-(1-(cyclopropylmethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

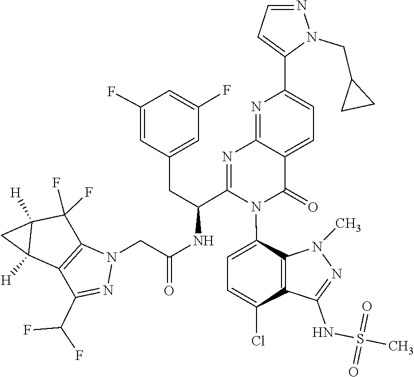

The title compound was prepared according to General Procedure K using 1-(cyclopropylmethyl)-5-(4,4,5,5-tetramethyl-1,3,2-dioxaborolan-2-yl)-1H-pyrazole as the coupling partner. The experiment afforded the title compound, N-((S)-1-((3P)-3-(4-chloro-1-methyl-3-(methylsulfonamido)-1H-indazol-7-yl)-7-(1-(cyclopropylmethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.45 min.; observed ion=926.4 (M+H). 1H NMR (500 MHz, METHANOL-d4) δ ppm 8.72 (d, J=8.34 Hz, 1H), 8.04 (d, J=8.34 Hz, 1H), 7.64 (d, J=2.09 Hz, 1H), 7.25-7.34 (m, 2H), 7.05 (d, J=2.09 Hz, 1H), 6.53-6.80 (m, 4H), 4.53 (s, 2H), 3.66 (s, 3H), 3.51 (dd, J=13.86, 4.92 Hz, 1H), 3.23 (s, 3H), 3.09-3.15 (m, 1H), 2.35-2.45 (m, 2H), 1.31-1.44 (m, 2H), 0.94-1.01 (m, 1H), 0.46-0.53 (m, 2H), 0.36-0.44 (m, 2H).

### Preparation of Example 79: N-((S)-1-((3P)-3-(4-chloro-1-methyl-3-(methylsulfonamido)-1H-indazol-7-yl)-7-(1-(2,2-difluoroethyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

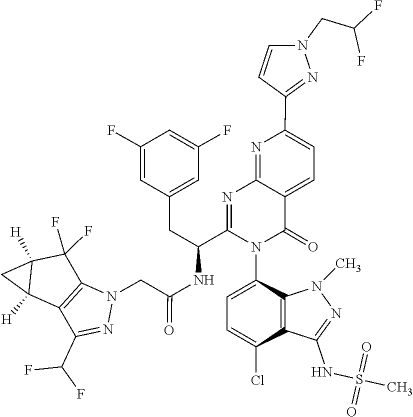

The title compound was prepared according to General Procedure M using 2,2-difluoroethyl trifluoromethanesulfonate as the coupling partner. The experiment afforded the title compound, N-((S)-1-((3P)-3-(4-chloro-1-methyl-3-(methylsulfonamido)-1H-indazol-7-yl)-7-(1-(2,2-difluoroethyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.39 min.; observed ion=936.4 (M+H). 1H NMR (500 MHz, METHANOL-d4) δ ppm 8.69 (d, J=8.34 Hz, 1H), 8.32 (d, J=8.05 Hz, 1H), 7.89 (d, J=2.38 Hz, 1H), 7.31 (br d, J=2.38 Hz, 1H), 7.23-7.26 (m, 2H), 6.61-6.78 (m, 4H), 6.21-6.44 (m, 1H), 4.65-4.72 (m, 2H), 4.56 (d, J=5.96 Hz, 2H), 3.62-3.64 (m, 3H), 3.48-3.52 (m, 1H), 3.23 (s, 3H), 3.13-3.16 (m, 1H), 2.38-2.43 (m, 2H), 1.32-1.37 (m, 1H), 0.95 -1.02 (m, 1H).

### Preparation of Example 80: N-((S)-1-((3P)-3-(4-chloro-1-methyl-3-(methylsulfonamido)-1H-indazol-7-yl)-4-oxo-7-(3-(trifluoromethyl)-1H-1,2,4-triazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

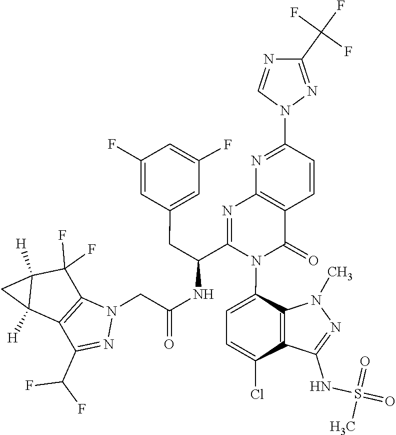

Potassium tert-butoxide (24.70 mg, 0.220 mmol) was added to a stirred solution of phenol (21.70 mg, 0.231 mmol) in 1,4-Dioxane (2 mL) at room temp and the mixture was stirred for 5 min. To the mixture was added 3-(trifluoromethyl)-1H-1,2,4-triazole (43.1 mg, 0.314 mmol), Ruphos Pd G3 (8.77 mg, 10.48 μmol) and 3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (100 mg, 0.105 mmol). The mixture was degassed (brief high vacuum, then refilled with Ar) and the mixture was then stirred at 80° C. for 2 h. The mixture was concentrated and the residue was purified by prep-HPLC to afford the title compound, N-((S)-1-((3P)-3-(4-chloro-1-methyl-3-(methylsulfonamido)-1H-indazol-7-yl)-4-oxo-7-(3-(trifluoromethyl)-1H-1,2,4-triazol-1-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method G: retention time=1.84 min.; observed ion=941.1 (M+H). 1H NMR (500 MHz, METHANOL-d4) δ ppm 9.65 (d, J=0.89 Hz, 1H), 8.93 (d, J=8.64 Hz, 1H), 8.23 (d, J=8.64 Hz, 1H), 7.31 (q, J=7.95 Hz, 2H), 6.49-6.85 (m, 4H), 4.46-4.57 (m, 2H), 3.65 (s, 3H), 3.49 (dd, J=14.16, 4.32 Hz, 1H), 3.24 (s, 3H), 3.15 (dd, J=14.31, 9.84 Hz, 1 H), 2.41 (ddd, J=11.18, 7.60, 4.17 Hz, 2H), 1.32-1.39 (m, 1H), 0.93-1.02 (m, 1H).

### Preparation of Example 81: N-((S)-1-(7-(benzo[d]oxazol-5-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

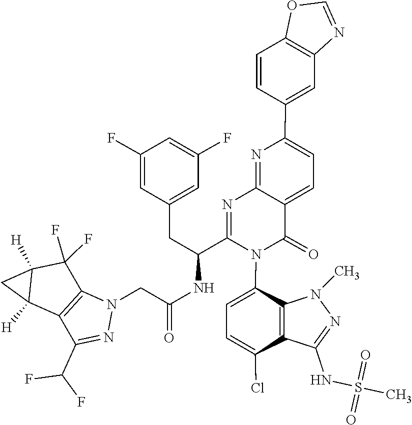

The title compound was prepared according to General Procedure K using benzo[d]oxazol-5-ylboronic acid as the coupling partner. The experiment afforded the title compound, N-((S)-1-(7-(benzo[d]oxazol-5-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.4 min.; observed ion=923.3 (M+H). 1H NMR (500 MHz, METHANOL-d4) δ ppm 8.76 (d, J=8.34 Hz, 1H), 8.69 (d, J=1.49 Hz, 1H), 8.61 (s, 1H), 8.37-8.44 (m, 1H), 8.28 (d, J=8.35 Hz, 1H), 7.91 (dd, J=8.64, 0.60 Hz, 1H), 7.23-7.34 (m, 2H), 6.50-6.83 (m, 4H), 4.52 -4.59 (m, 2H), 3.64 (s, 3H), 3.51 (dd, J=14.45, 4.62 Hz, 1H), 3.23 (s, 3H), 3.15-3.19 (m, 1 H), 2.35-2.46 (m, 2H), 1.31-1.38 (m, 1H), 0.92-1.04 (m, 1H).

### Preparation of Example 82: N-((S)-1-((3P)-3-(4-chloro-1-methyl-3-(methylsulfonamido)-1H-indazol-7-yl)-7-(2-methoxythiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

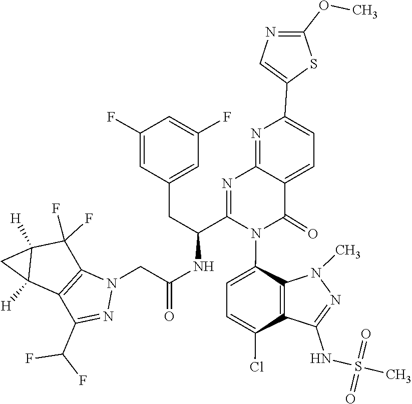

The title compound was prepared according to General Procedure J using 2-methoxy-5-(tributylstannyl)thiazole as the coupling partner. The experiment afforded the title compound, N-((S)-1-((3P)-3-(4-chloro-1-methyl-3-(methylsulfonamido)-1H-indazol-7-yl)-7-(2-methoxythiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.43 min.; observed ion=919.3 (M+H). 1H NMR (500 MHz, METHANOL-d4) δ ppm 8.60 (d, J=8.35 Hz, 1H), 8.12 (s, 1H), 8.06 (d, J=8.34 Hz, 1H), 7.17-7.34 (m, 2H), 6.49-6.82 (m, 4H), 4.50-4.59 (m, 2H), 4.18 (s, 3H), 3.61 (s, 3H), 3.46 (dd, J=14.16, 4.62 Hz, 1H), 3.23 (s, 3H), 3.12 (dd, J=14.16, 9.69 Hz, 1H), 2.35-2.47 (m, 2 H), 1.29-1.40 (m, 1H), 0.91-1.03 (m, 1H).

### Preparation of Example 83: N-((S)-1-((3P)-3-(4-chloro-1-methyl-3-(methylsulfonamido)-1H-indazol-7-yl)-7-(1-methyl-3-(trifluoromethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

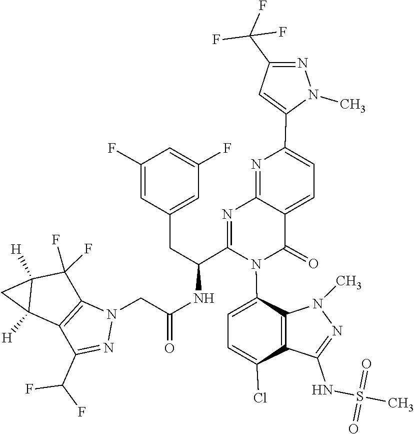

To a mixture of 3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (30 mg, 0.031 mmol), 1-methyl-5-(tributylstannyl)-3-(trifluoromethyl)-1H-pyrazole (20.71 mg, 0.047 mmol) and copper(I) iodide (0.599 mg, 3.14 μmol) in N,N-Dimethylformamide (DMF) (1 mL) was added tetrakis(triphenylphosphine)palladium(0) (3.63 mg, 3.14 μmol). The mixture was then degassed (brief high vacuum, then refilled with Ar) and heated at 100° C. for 16 h. The mixture was cooled to room temperature, filtered, and the filtrate was subjected to prep-HPLC purification to afford the title compound, N-((S)-1-((3P)-3-(4-chloro-1-methyl-3-(methylsulfonamido)-1H-indazol-7-yl)-7-(1-methyl-3-(trifluoromethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=954.2 (M+H). 1H NMR (500 MHz, METHANOL-d4) δ ppm 8.78 (d, J=8.34 Hz, 1H), 8.11 (d, J=8.34 Hz, 1H), 7.43 (s, 1H), 7.30-7.37 (m, 2H), 6.52-6.85 (m, 4H), 4.85-4.88 (m, 1H), 4.53-4.64 (m, 2H), 4.49 (s, 3H), 3.66 (s, 3H), 3.51 (dd, J=14.31,4.47 Hz, 1H), 3.26 (s, 3H), 3.17 (dd, J=14.31, 9.84 Hz, 1H), 2.38-2.47 (m, 2H), 1.33-1.39 (m, 1H), 0.95-1.02 (m, 1H).

### Preparation of Example 84: N-((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

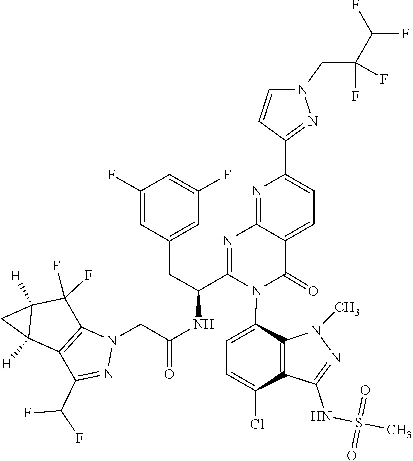

The title compound was prepared according to General Procedure M using 2,2,3,3-tetrafluoropropyl trifluoromethanesulfonate as the coupling partner. The experiment afforded the title compound, N-((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.43 min.; observed ion=986.4 (M+H). 1H NMR (500 MHz, METHANOL-d4) δ ppm 8.73 (d, J=8.34 Hz, 1H), 8.35 (d, J=8.34 Hz, 1H), 7.95 (d, J=2.38 Hz, 1H), 7.26-7.35 (m, 3H), 6.55-6.85 (m, 4H), 6.18-6.46 (m, 1H), 5.02 (br t, J=14.01 Hz, 2H), 4.53-4.62 (m, 2H), 3.66 (s,3H), 3.52 (dd, J=14.16, 4.62 Hz, 1H), 3.26 (s, 3H), 3.16-3.21 (m, 1H), 2.38-2.46 (m, 2H), 1.33-1.39 (m, 1H), 0.97 -1.05 (m, 1H).

### Preparation of Example 85: N-((S)-1-((3P)-3-(4-chloro-1-methyl-3-(methylsulfonamido)-1H-indazol-7-yl)-4-oxo-7-(1-(4,4,4-trifluorobutyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

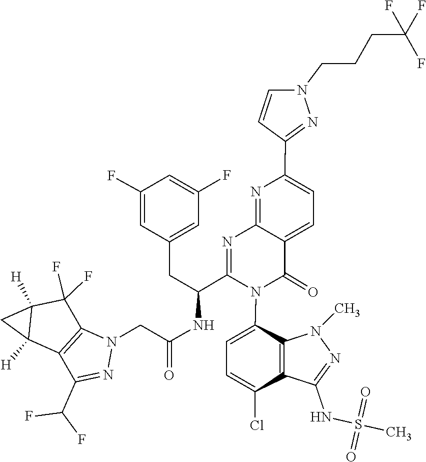

The title compound was prepared according to General Procedure M using 4,4,4-trifluorobutyl trifluoromethanesulfonate as the coupling partner. The experiment afforded the title compound, N-((S)-1-((3P)-3-(4-chloro-1-methyl-3-(methylsuIfonamido)-1H-indazol-7-yl)-4-oxo-7-(1-(4,4,4-trifluorobutyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=982.4 (M+H). 1H NMR (500 MHz, METHANOL-d4) δ ppm 8.71 (d, J=8.05 Hz, 1H), 8.32 (d, J=8.05 Hz, 1H), 7.86 (d, J=2.38 Hz, 1H), 7.32-7.35 (m, 1H), 7.27 (d, J=8.05 Hz, 1H), 7.23 (d, J=2.38 Hz, 1H), 6.56-6.83 (m, 4H), 4.53-4.62 (m, 2H), 4.34-4.44 (m, 2H), 3.66 (s, 3H), 3.52 (dd, J=14.01, 4.77 Hz, 1H), 3.26 (s, 3H), 3.17-3.21 (m, 1H), 2.38-2.45 (m, 2H), 2.16-2.33 (m, 5H), 1.32 -1.37 (m, 1H), 0.95-1.03 (m, 1H).

### Preparation of Example 86: N-((S)-1-((3P)-3-(4-chloro-1-methyl-3-(methylsulfonamido)-1H-indazol-7-yl)-7-(1-(2,2-difluorobutyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

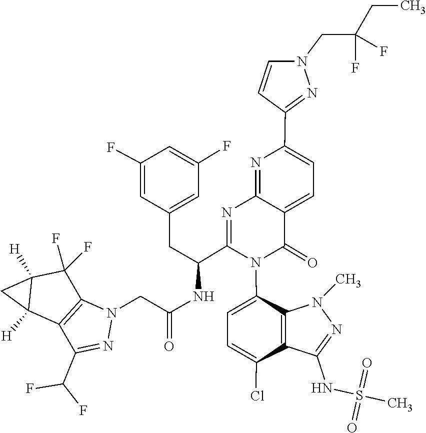

The title compound was prepared according to General Procedure M using 2,2-difluorobutyl trifluoromethanesulfonate as the coupling partner. The experiment afforded the title compound, N-((S)-1-((3P)-3-(4-chloro-1-methyl-3-(methylsulfonamido)-1H-indazol-7-yl)-7-(1-(2,2-difluorobutyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=964.4 (M+H). 1H NMR (500 MHz, METHANOL-d4) δ ppm 8.72 (d, J=8.34 Hz, 1H), 8.34 (d, J=8.34 Hz, 1H), 7.89 (d, J=2.38 Hz, 1H), 7.31-7.36 (m, 1H), 7.27 (dd, J=5.07, 2.68 Hz, 2H), 6.55-6.84 (m, 4H), 4.76 (t, J=13.26 Hz, 2H), 4.61-4.64 (m, 1H), 4.52-4.60 (m, 2H), 3.66 (s, 3H), 3.48-3.56 (m, 1 H), 3.26 (s, 3H), 3.13-3.22 (m, 1H), 2.39-2.46 (m, 2H), 1.90-2.03 (m, 2H), 1.33-1.39 (m, 1H), 1.14 (t, J=7.60 Hz, 3H), 0.98-1.03 (m, 1H).

### Preparation of Example 87: N-((S)-1-((3P)-3-(4-chloro-1-methyl-3-(methylsulfonamido)-1H-indazol-7-yl)-7-(1-(3-fluoropropyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

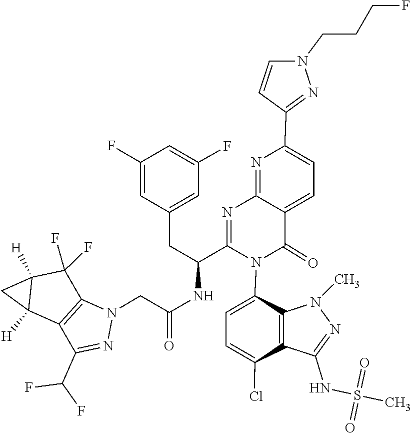

The title compound was prepared according to General Procedure M using 3-fluoropropyl trifluoromethanesulfonate as the coupling partner. The experiment afforded the title compound, N-((S)-1-((3P)-3-(4-chloro-1-methyl-3-(methylsulfonamido)-1H-indazol-7-yl)-7-(1-(3-fluoropropyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.41 min.; observed ion=932.4 (M+H). 1H NMR (500 MHz, METHANOL-d4) δ ppm 8.74 (d, J=8.05 Hz, 0.4H), 8.70 (d, J=8.34 Hz, 0.6H), 8.32 (d, J=8.35 Hz, 0.6H), 8.06 (d, J=8.34 Hz, 0.4H), 7.86 (d, J=2.38 Hz, 0.6H), 7.68 (d, J=2.09 Hz, 0.4H), 7.25-7.36 (m, 2H), 7.22 (d, J=2.38 Hz, 0.6H), 7.09 (d, J=2.09 Hz, 0.4H), 6.53-6.84 (m, 4 H), 4.99-5.13 (m, 1H), 4.55-4.59 (m, 2H), 4.43-4.51 (m, 2H), 3.68 (s, 1.3H), 3.66 (s, 1.7H), 3.53 (ddd, J=14.31, 9.39, 4.92 Hz, 1H), 3.27 (s, 1.3H), 3.26 (s, 1.7H), 3.12-3.21 (m, 1H), 2.27-2.47 (m, 4H), 1.39 (s, 1H), 0.97-1.04 (m, 1H).

### Preparation of Example 88: N-((S)-1-((3P)-3-(4-chloro-1-methyl-3-(methylsulfonamido)-1H-indazol-7-yl)-4-oxo-7-(1-(2,2,3,3,3-pentafluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

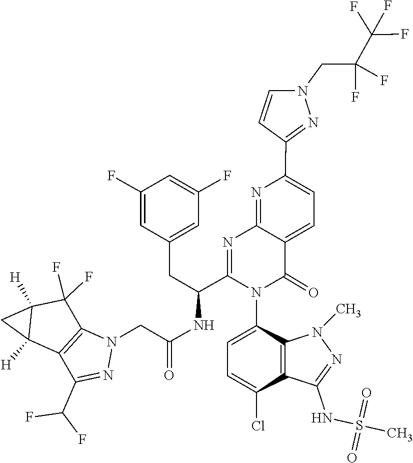

The title compound was prepared according to General Procedure M using 2,2,3,3,3-pentafluoropropyl trifluoromethanesulfonate as the coupling partner. The experiment afforded the title compound, N-((S)-1-((3P)-3-(4-chloro-1-methyl-3-(methylsulfonamido)-1H-indazol-7-yl)-4-oxo-7-(1-(2,2,3,3,3-pentafluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=1004.4 (M+H). 1H NMR (500 MHz, METHANOL-d4) δ ppm 8.73 (d, J=8.34 Hz, 1H), 8.35 (d, J=8.35 Hz, 1H), 7.98 (d, J=2.38 Hz, 1H), 7.24-7.36 (m, 3H), 6.54-6.86 (m, 4H), 5.22 (t, J=14.60 Hz, 2H), 4.55-4.62 (m, 2H), 3.66 (s, 3H), 3.49-3.54 (m, 1H), 3.26 (s, 3H), 3.16-3.20 (m, 1H), 2.37-2.47 (m, 2H), 1.34-1.39 (m, 1H), 1.01 (qd, J=3.78, 2.09 Hz, 1H).

### Preparation of Example 89: N-((S)-1-((3P)-3-(4-chloro-1-methyl-3-(methylsulfonamido)-1H-indazol-7-yl)-4-oxo-7-(1-(3,3,3-trifluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

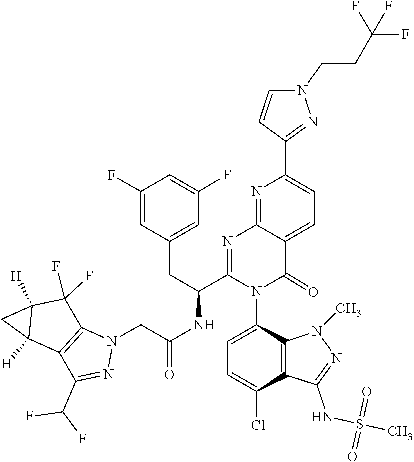

The title compound was prepared according to General Procedure M using 3,3,3-trifluoropropyl trifluoromethanesulfonate as the coupling partner. The experiment afforded the title compound, N-((S)-1-((3P)-3-(4-chloro-1-methyl-3-(methylsulfonamido)-1H-indazol-7-yl)-4-oxo-7-(1-(3,3,3-trifluoropropyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.46 min.; observed ion=968.4 (M+H). 1H NMR (500 MHz, METHANOL-d4) δ ppm 8.74 (d, J=8.34 Hz, 0.3H), 8.71 (d, J=8.05 Hz, 0.7H), 8.33 (d, J=8.34 Hz, 0.7H), 8.08 (d, J=8.34 Hz, 0.3H), 7.89 (d, J=2.09 Hz, 0.7H), 7.71 (s, 0.3H), 7.10-7.37 (m, 3H), 6.56-6.84 (m, 4H), 5.11-5.33 (m, 1H), 4.47-4.64 (m, 4H), 3.69 (s, 1H), 3.66 (s, 2H), 3.48-3.56 (m, 1H), 3.27 (s, 1H), 3.26 (s, 2H), 3.10-3.21 (m, 1H), 2.91-3.02 (m, 2H), 2.38-2.48 (m, 2H), 1.33-1.39 (m, 1H), 0.98-1.03 (m, 1H).

### Preparation of Example 90: N-((S)-1-((3P)-3-(4-chloro-1-methyl-3-(methylsulfonamido)-1H-indazol-7-yl)-4-oxo-7-(5-(trifluoromethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

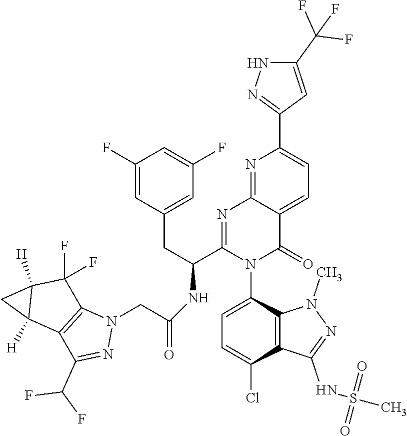

The title compound was prepared according to General Procedure K using (5-(trifluoromethyl)-1H-pyrazol-3-yl)boronic acid as the coupling partner. The experiment afforded the title compound, N-((S)-1-((3P)-3-(4-chloro-1-methyl-3-(methylsulfonamido)-1H-indazol-7-yl)-4-oxo-7-(5-(trifluoromethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.45 min.; observed ion=940.4 (M+H). 1H NMR (500 MHz, METHANOL-d4) δ ppm 8.78 (d, J=8.05 Hz, 1H), 8.13-8.19 (m, 1H), 7.52 (s, 1H), 7.31-7.37 (m, 1H), 7.24-7.29 (m, 1H), 6.51-6.87 (m, 4H), 4.92-4.95 (m, 1H), 4.50 (s, 2H), 3.68 (s, 3H), 3.54 (dd, J=14.31, 4.47 Hz, 1H), 3.26 (s, 3H), 3.18 (dd, J=14.01, 9.54 Hz, 1 H), 2.39-2.48 (m, 2H), 1.40 (s, 1H), 0.96-1.05 (m, 1H).

### Preparation of Example 91: N-((S)-1-((3P)-3-(4-chloro-1-methyl-3-(methylsulfonamido)-1H-indazol-7-yl)-7-(2-(2-hydroxypropan-2-yl)thiazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

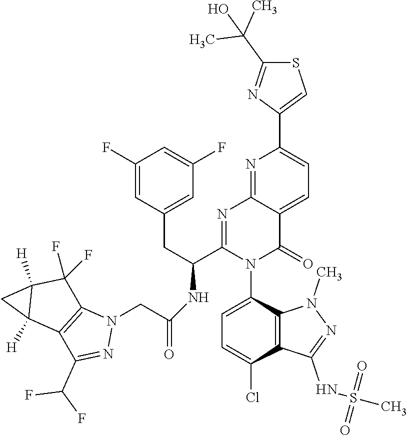

The title compound was prepared according to General Procedure J using 2-(4-(tributylstannyl)thiazol-2-yl)propan-2-ol as the coupling partner. The experiment afforded the title compound, N-((S)-1-((3P)-3-(4-chloro-1-methyl-3-(methylsulfonamido)-1H-indazol-7-yl)-7-(2-(2-hydroxypropan-2-yl)thiazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.39 min.; observed ion=947.4 (M+H). 1H NMR (500 MHz, METHANOL-d4) δ ppm 8.77 (d, J=8.05 Hz, 1H), 8.51 (s, 1H), 8.46 (d, J=8.05 Hz, 1H), 7.31-7.35 (m, 1H), 7.26-7.29 (m, 1H), 6.57-6.84 (m, 4H), 4.52-4.61 (m, 2H), 3.67 (s, 3H), 3.53 (dd, J=14.16, 4.92 Hz, 1H), 3.26 (s, 3H), 3.15-3.22 (m, 1H), 2.38-2.49 (m, 2H), 1.74 (s, 6H), 1.33-1.40 (m, 1H), 0.97-1.06 (m, 1H).

### Preparation of Example 92: N-((S)-1-((3P)-3-(4-chloro-1-methyl-3-(methylsulfonamido)-1H-indazol-7-yl)-7-(2-(2-hydroxypropan-2-yl)thiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

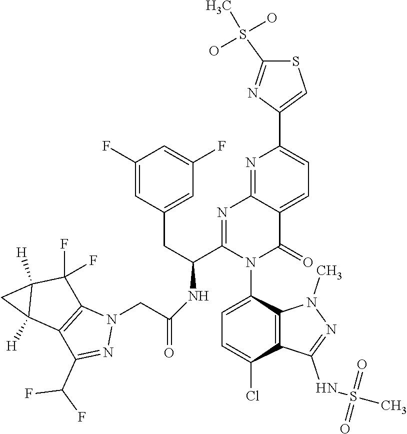

The title compound was prepared according to General Procedure J using 2-(5-(tributylstannyl)thiazol-2-yl)propan-2-ol as the coupling partner. The experiment afforded the title compound, N-((S)-1-((3P)-3-(4-chloro-1-methyl-3-(methylsulfonamido)-1H-indazol-7-yl)-7-(2-(2-hydroxypropan-2-yl)thiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.35 min.; observed ion=947.4 (M+H). 1H NMR (500 MHz, METHANOL-d4) δ ppm 8.69 (d, J=8.34 Hz, 1H), 8.58 (s, 1H), 8.17 (d, J=8.34 Hz, 1H), 7.32-7.36 (m, 1H), 7.24-7.30 (m, 1H), 6.52-6.84 (m, 4H), 4.54-4.62 (m, 2H), 3.65 (s, 3H), 3.50 (dd, J=14.01, 4.47 Hz, 1H), 3.25 (s, 3H), 3.16 (dd, J=14.31, 9.84 Hz, 1H), 2.38-2.46 (m, 2H), 1.70 (s, 6H), 1.34-1.40 (m, 1H), 0.98-1.04 (m, 1H).

### Preparation of Example 93: N-((S)-1-((3P)-3-(4-chloro-1-methyl-3-(methylsulfonamido)-1H-indazol-7-yl)-7-(2-(methylsulfonyl)thiazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

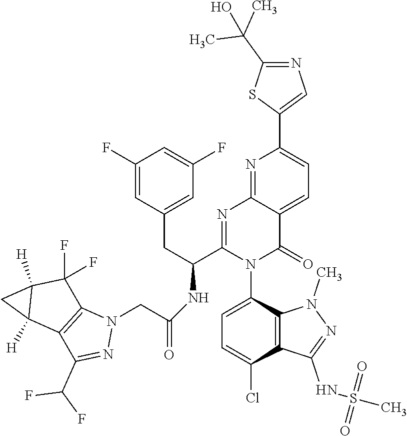

The title compound was prepared according to General Procedure J using 2-(methylsulfonyl)-4-(tributylstannyl)thiazole as the coupling partner. The experiment afforded the title compound, N-((S)-1-((3P)-3-(4-chloro-1-methyl-3-(methylsulfonamido)-1H-indazol-7-yl)-7-(2-(methylsulfonyl)thiazol-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.38 min.; observed ion=967.3 (M+H). 1H NMR (500 MHz, METHANOL-d4) δ ppm 9.01 (s, 1H), 8.84 (d, J=8.05 Hz, 1H), 8.51 (d, J=8.34 Hz, 1H), 7.27-7.37 (m, 2H), 6.54-6.84 (m, 4H), 4.50-4.61 (m, 2H), 3.67 (s, 3H), 3.53-3.56 (m, 1H), 3.52 (s, 3H), 3.26 (s, 3H), 3.14-3.22 (m, 1H), 2.37-2.48 (m, 2H), 1.33-1.40 (m, 1H), 0.97-1.05 (m, 1H).

### Preparation of Example 94: N-((S)-1-((3P)-3-(4-chloro-1-methyl-3-(methylsulfonamido)-1H-indazol-7-yl)-7-(1-(2,2-difluoropropyl)-5-(trifluoromethyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

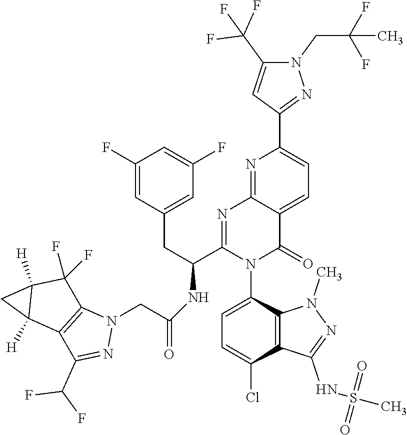

To a mixture of N-((S)-1-(3-(4-chloro-3-(N-(4-methoxybenzyl)methylsulfonamido)-1-methyl-1H-indazol-7-yl)-4-oxo-7-(5-(trifluoromethyl)-1H-pyrazol-3-yl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (80 mg, 0.075 mmol) and 2,2-difluoropropyl trifluoromethanesulfonate (51.6 mg, 0.226 mmol) in Acetonitrile (1 mL) was added cesium carbonate (36.9 mg, 0.113 mmol) and the resulting mixture was heated at 60° C. for 1 h. The mixture was then cooled to room temperature, filtered, and the filtrate was concentrated in vacuo. The residue was taken up in DCM (0.5 mL) and TFA (1 mL), and to the solution was added triflic acid (0.05 mL). The solution was stirred at rt for 1 h and then concentrated in vacuo. The residue was then dissolved in DMF (2 mL) and purified by prep HPLC to afford two isolates containing the target mass:

The first peak to elute was the title compound, N-((S)-1-((3P)-3-(4-chloro-1-methyl-3-(methylsulfonamido)-1H-indazol-7-yl)-7-(1-(2,2-difluoropropyl)-5-(trifluoromethyl)-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.3 min.; observed ion=1018 (M+H). 1H NMR (500 MHz, METHANOL-d4) δ ppm 8.77 (d, J=8.05 Hz, 1H), 8.37 (d, J=8.05 Hz, 1H), 7.70 (d, J=0.60 , 1H), 7.28-7.35 (m, 2H), 6.57-6.82 (m, 4H), 4.90-4.92 (m, 1H), 4.85-4.88 (m, 2H), 4.54-4.61 (m, 2H), 3.66 (s, 3H), 3.52 (dd, J=14.31, 4.47Hz, 1H), 3.26 (s, 3H), 3.15-3.20 (m, 1H), 2.39-2.46 (m, 2H), 1.82 (t, J=18.78 Hz, 3H), 1.33-1.39 (m, 1H), 0.97-1.04 (m, 1H).

The second peak to elute was Example 95.

### Preparation of Example 95: N-((S)-1-((3P)-3-(4-chloro-1-methyl-3-(methylsulfonamido)-1H-indazol-7-yl)-7-(1-(2,2-difluoropropyl)-3-(trifluoromethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

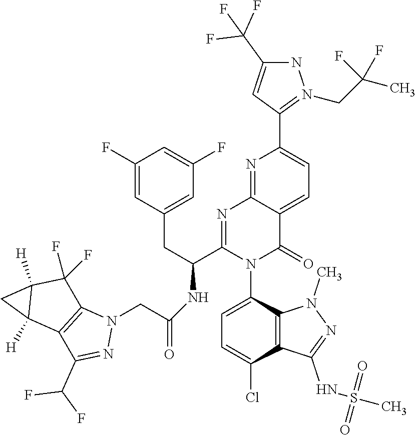

See preparation of Example 94 for the procedure which afforded the title compound, N-((S)-1-((3P)-3-(4-chloro-1-methyl-3-(methylsulfonamido)-1H-indazol-7-yl)-7-(1-(2,2-difluoropropyl)-3-(trifluoromethyl)-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.34 min.; observed ion=1018 (M+H). 1H NMR (500 MHz, METHANOL-d4) δ ppm 8.79 (d, J=8.05Hz, 1H), 8.11 (d, J=8.05 Hz, 1H), 7.50 (s, 1H), 7.31-7.36 (m, 2H), 6.55-6.83 (m, 4H), 5.67-5.78 (m, 1H), 5.47-5.58 (m, 1H), 4.91-4.94 (m, 1H), 4.54 (s, 2H), 3.69 (s, 3H), 3.54 (dd, J=14.16, 4.62 Hz, 1H), 3.26 (s, 3H), 3.16 (dd, J=14.01, 9.54 Hz, 1H), 2.39-2.46 (m, 2H), 1.66 (t, J=18.78 Hz, 3H), 1.33-1.39 (m, 1 H), 0.97-1.02 (m, 1H).

### Preparation of Example 96: N-((S)-1-((3P)-3-(4-chloro-1-methyl-3-(methylsulfonamido)-1H-indazol-7-yl)-7-(1-(2,2-difluoropropyl)-5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

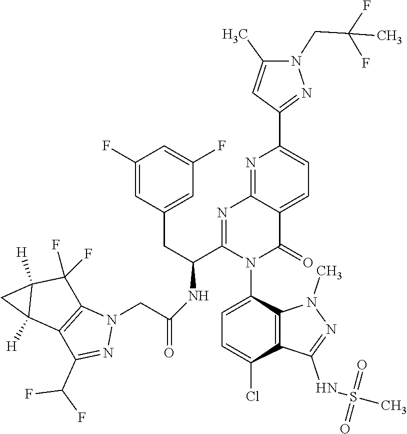

To a mixture of N-((S)-1-(3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (85 mg, 0.084 mmol) and 2,2-difluoropropyl trifluoromethanesulfonate (57.8 mg, 0.253 mmol) in Acetonitrile (2 mL) was added cesium carbonate (41.3 mg, 0.127 mmol) and the resulting mixture was heated at 60° C. for 1 h. The mixture was then cooled to room temperature, filtered, and the filtrate was concentrated in vacuo. The residue was dissolved in DCM (0.5 mL) and TFA (1 mL), then to the solution was added triflic acid (0.05 mL). The solution was stirred at rt for 1 h and then was concentrated in vacuo. The residue was dissolved in DMF (2 mL) and then was subjected to prep-HPLC purification to afford two isolates containing the target mass:

The first peak to elute was Example 97.

The second peak to elute was the title compound, N-((S)-1-((3P)-3-(4-chloro-1-methyl-3-(methylsulfonamido)-1H-indazol-7-yl)-7-(1-(2,2-difluoropropyl)-5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.13 min.; observed ion=964 (M+H). 1H NMR (500 MHz, METHANOL-d4) δ ppm 8.67 (d, J=8.35 Hz, 1H), 8.28 (d, J=8.34 Hz, 1H), 7.31-7.33 (m, 1H), 7.23-7.26 (m, 1H), 7.02 (s, 1H), 6.58-6.83 (m, 5H), 4.90-4.94 (m, 1H), 4.68 (t, J=12.52 Hz, 2H), 4.54-4.63 (m, 2 H), 3.65 (s, 3H), 3.50 (dd, J=14.31, 4.77 Hz, 1H), 3.26 (s, 3H), 3.16 (dd, J=14.16, 9.69 Hz, 1H), 2.47 (s, 3H), 2.40-2.45 (m, 2H), 1.74 (t, J=18.93 Hz, 3H), 1.36 (q, J=7.05 Hz, 1H), 1.00 (br d, J=2.68Hz, 1H).

### Preparation of Example 97: N-((S)-1-((3P)-3-(4-chloro-1-methyl-3-(methylsulfonamido)-1H-indazol-7-yl)-7-(1-(2,2-difluoropropyl)-3-methyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

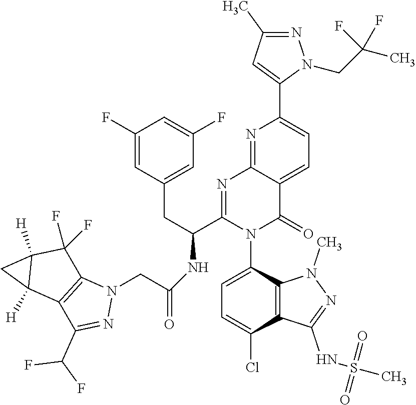

See preparation of Example 96 for the procedure which afforded the title compound, N-((S)-1-((3P)-3-(4-chloro-1-methyl-3-(methylsulfonamido)-1H-indazol-7-yl)-7-(1-(2,2-difluoropropyl)-3-methyl-1H-pyrazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.06 min.; observed ion=964.05 (M+H). 1H NMR (500 MHz, METHANOL-d4) δ ppm 8.68 (d, J=8.35 Hz, 1H), 8.31 (d, J=8.35 Hz, 1H), 7.40 (d, J=7.75 Hz, 1H), 7.27 (d, J=7.75 Hz, 1H), 7.05 (d, J=0.89 Hz, 1H), 6.54-6.79 (m, 4H), 4.91-4.95 (m, 1H), 4.77-4.83 (m, 2H), 4.69 (t, J=12.67 Hz, 2H), 3.41-3.46 (m, 1H), 3.29 (s, 3H), 3.20 (s, 3H), 3.00 (dd, J=13.41, 6.26 Hz, 1H), 2.50-2.54 (m, 1H), 2.48 (s, 3 H), 1.74 (t, J=18.93 Hz, 3H), 1.38-1.44 (m, 1H), 1.08-1.13 (m, 1H).

### Preparation of Example 98: N-((S)-1-((3P)-3-(4-chloro-1-methyl-3-(methylsulfonamido)-1H-indazol-7-yl)-7-(5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

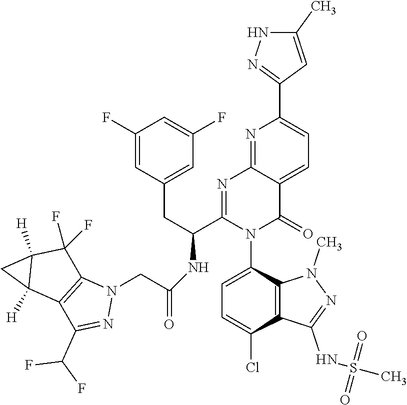

The title compound was prepared according to General Procedure K using (5-methyl-1H-pyrazol-3-yl)boronic acid as the coupling partner. The experiment afforded the title compound, N-((S)-1-((3P)-3-(4-chloro-1-methyl-3-(methylsulfonamido)-1H-indazol-7-yl)-7-(5-methyl-1H-pyrazol-3-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.31 min.; observed ion=886.4 (M+H). 1H NMR (500 MHz, METHANOL-d4) δ ppm 8.69 (br d, J=8.35 Hz, 1H), 8.27 (ddd, J=5.44, 3.65, 1.94 Hz, 1H), 7.19-7.34 (m, 2H), 6.98 (s, 1H), 6.51-6.84 (m, 4H), 4.50-4.67 (m, 3H), 3.65 (s, 3H), 3.52 (br dd, J=13.71, 4.17 Hz, 1H), 3.24 (s, 3H), 3.14-3.20 (m, 1H), 2.31-2.54 (m, 5H), 1.33-1.43 (m, 1H), 0.96-1.01 (m, 1H).

### Preparation of Example 99: N-((S)-1-((3P)-3-(4-chloro-1-methyl-3-(methylsulfonamido)-1H-indazol-7-yl)-7-(2-(1,1-difluoroethyl)thiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

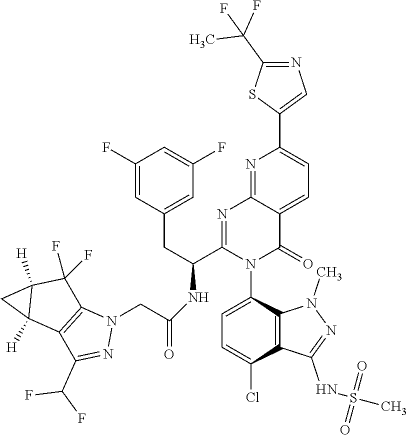

The title compound was prepared according to General Procedure J using 2-(1,1-difluoroethyl)-5-(tributylstannyl)thiazole as the coupling partner. The experiment afforded the title compound, N-((S)-1-((3P)-3-(4-chloro-1-methyl-3-(methylsulfonamido)-1H-indazol-7-yl)-7-(2-(1,1-difluoroethyl)thiazol-5-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=953.3 (M+H). 1H NMR (500 MHz, METHANOL-d4) δ ppm 8.71-8.78 (m, 2H), 8.26 (d, J=8.35 Hz, 1H), 7.27-7.36 (m, 2H), 6.56-6.83 (m, 4H), 4.60 (d, J=14.31 Hz, 2H), 3.65 (s, 3H), 3.50 (dd, J=14.01, 4.47 Hz, 1 H), 3.25 (s, 3H), 3.15-3.20 (m, 1H), 2.39-2.46 (m, 2H), 2.19 (t, J=18.63 Hz, 3H), 1.34 -1.39 (m, 1H), 0.97-1.04 (m, 1H).

### Preparation of Example 100: N-((S)-1-((3P)-3-(4-chloro-1-methyl-3-(methylsulfonamido)-1H-indazol-7-yl)-7-(3-methyl-1H-1,2,4-triazol-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

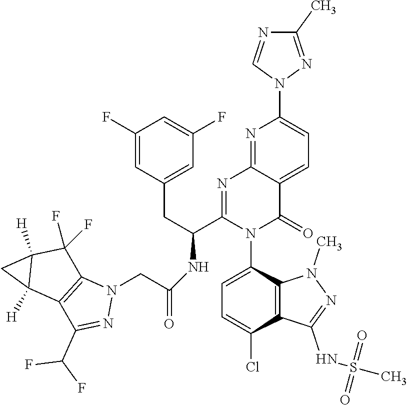

To a stirred solution of phenol (21.70 mg, 0.231 mmol) in 1,4-Dioxane (2 mL) at room temp was added potassium tert-butoxide (24.70 mg, 0.220 mmol) and the mixture was stirred for 5 min. To the mixture was added 3-methyl-1H-1,2,4-triazole (26.1 mg, 0.314 mmol), Ruphos Pd G3 (8.77 mg, 10.48 μmol) and 3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (100 mg, 0.105 mmol). The mixture was degassed (brief high vacuum, then refilled with Ar) and then stirred at 100° C. for 2 h. The mixture was concentrated in vacuo and the resulting residue was subjected to HPLC purification to afford the title compound, N-((S)-1-((3P)-3-(4-chloro-1-methyl-3-(methylsulfonamido)-1H-indazol-7-yl)-7-(3-methyl-1H-1,2,4-triazol-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=2.88 min.; observed ion=887.0 (M+H). ¹H NMR (500 MHz, METHANOL-d₄) δ ppm 9.43 (s, 1 H), 8.88 (d, J=8.64 Hz, 1H), 8.18 (d, J=8.64 Hz, 1H), 7.28-7.37 (m, 2H), 6.57-6.84 (m, 4 H), 4.50-4.60 (m, 2H), 3.67 (s, 3H), 3.51 (dd, J=14.16, 4.32 Hz, 1H), 3.27 (s, 3H), 3.16 (dd, J=14.01, 9.84 Hz, 1H), 2.54 (s, 3H), 2.43 (ddd, J=11.40, 7.67, 4.17 Hz, 2H), 1.34 -1.39 (m, 1H), 0.98-1.04 (m, 1H).

### IUPAC Chemical Names:

The IUPAC chemical names for each example are listed below. At this time these names are not recognized by common software such tools such as ChemDraw or JChem. Therefore, the chemical names used throughout the Examples section above were generated with ChemDraw with P/M nomenclature manually inserted. The chemical names can be converted to chemical structures using ChemDraw after the P/M nomenclature—e.g., “(3P)-”—is removed.

| Example | IUPAC Name |
|---|---|
| Example 1 | N-[(1S)-1-[(3P)-3-(4-chloro-3-methanesulfonamido- |
| | 1-methyl-1H-indazol-7-yl)-4-oxo-6-[6-(trifluoromethyl) |
| | pyridin-2-yl]-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 2 | N-[(1S)-1-[(3P)-3-(4-chloro-3-methanesulfonamido- |
| | 1-methyl-1H-indazol-7-yl)-6-[4-(difluoromethyl) |
| | pyrimidin-2-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 3 | N-[(1S)-1-[(3P)-3-(4-chloro-3-methanesulfonamido- |
| | 1-methyl-1H-indazol-7-yl)-6-[6-(difluoromethyl) |
| | pyridin-2-yl]-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 4 | N-[(1S)-1-[(3P)-3-(4-chloro-3-methanesulfonamido- |
| | 1-methyl-1H-indazol-7-yl)-4-oxo-7-[2- |
| | (trifluoromethyl)-1,3-thiazol-4-yl]-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 5 | N-[(1S)-1-[(3P)-3-(4-chloro-3-methanesulfonamido- |
| | 1-methyl-1H-indazol-7-yl)-4-oxo-7-[4-(trifluoromethyl) |
| | pyrimidin-2-yl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 6 | N-[(1S)-1-[(3P)-3-(4-chloro-3-methanesulfonamido- |
| | 1-methyl-1H-indazol-7-yl)-7-[4-(difluoromethyl) |
| | pyrimidin-2-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 7 | N-[(1S)-1-[(3P)-3-(4-chloro-3-methanesulfonamido- |
| | 1-methyl-1H-indazol-7-yl)-7-(3-methylpyrazin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 8 | N-[(1S)-1-[(3P)-3-(4-chloro-3-methanesulfonamido- |
| | 1-methyl-1H-indazol-7-yl)-4-oxo-7-(pyrazin-2-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 9 | N-[(1S)-1-[(3P)-3-(4-chloro-3-methanesulfonamido- |
| | 1-methyl-1H-indazol-7-yl)-4-oxo-7-[6-(trifluoromethyl) |
| | pyridin-2-yl]-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 10 | N-[(1S)-1-[(3P)-3-(4-chloro-3-methanesulfonamido- |
| | 1-methyl-1H-indazol-7-yl)-7-(4-methylpyrimidin-2-yl)- |
| | 4-oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 11 | N-[(1S)-1-[(3P)-3-(4-chloro-3-methanesulfonamido-1- |
| | methyl-1H-indazol-7-yl)-7-(4,6-dimethylpyrimidin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona- |
| | 1(6),8-dien-7-yl]acetamide |
| Example 12 | N-[(1S)-1-[(3P)-3-(4-chloro-3-methanesulfonamido- |
| | 1-methyl-1H-indazol-7-yl)-7-[6-(1,1-difluoroethyl) |
| | pyridin-2-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 13 | N-[(1S)-1-[(3P)-3-(4-chloro-3-methanesulfonamido- |
| | 1-methyl-1H-indazol-7-yl)-4-oxo-7-[6- |
| | (trifluoromethyl)pyrazin-2-yl]-3H,4H-pyrido |
| | [2,3-d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 14 | N-[(1S)-1-[(3P)-7-[4,6-bis(trifluoromethyl)pyridin-2- |
| | yl]-3-(4-chloro-3-methanesulfonamido-1-methyl- |
| | 1H-indazol-7-yl)-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 15 | N-[(1S)-1-[(3P)-3-(4-chloro-3-methanesulfonamido- |
| | 1-methyl-1H-indazol-7-yl)-4-oxo-7-[6-(trifluoromethyl) |
| | pyrimidin-4-yl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 16 | N-[(1S)-1-[(3P)-3-(4-chloro-3-methanesulfonamido- |
| | 1-methyl-1H-indazol-7-yl)-4-oxo-7-[4-(trifluoromethyl) |
| | pyridin-2-yl]-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 17 | N-[(1S)-1-[(3P)-3-(4-chloro-3-methanesulfonamido- |
| | 1-methyl-1H-indazol-7-yl)-4-oxo-7-[5-(trifluoromethyl) |
| | pyrimidin-4-yl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 18 | N-[(1S)-1-[(3P)-3-(4-chloro-3-methanesulfonamido- |
| | 1-methyl-1H-indazol-7-yl)-7-[6-methyl-2- |
| | (trifluoromethyl)pyrimidin-4-yl]-4-oxo-3H,4H-pyrido |
| | [2,3-d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 19 | N-[(1S)-1-[(3P)-3-(4-chloro-3-methanesulfonamido- |
| | 1-methyl-1H-indazol-7-yl)-7-[4-fluoro-3- |
| | (trifluoromethyl)phenyl]-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 20 | N-[(1S)-1-[(3P)-3-(4-chloro-3-methanesulfonamido- |
| | 1-methyl-1H-indazol-7-yl)-7-[3-(difluoromethoxy) |
| | phenyl]-4-oxo-3H,4H-pyrido[2,3-d]pyrimidin-2-yl]- |
| | 2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 21 | N-[(1S)-1-[(3P)-3-(4-chloro-3-methanesulfonamido-1- |
| | methyl-1H-indazol-7-yl)-4-oxo-7-[3-(trifluoromethoxy) |
| | phenyl]-3H,4H-pyrido[2,3-d]pyrimidin-2-yl]- |
| | 2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 22 | N-[(1S)-1-[(3P)-3-(4-chloro-3-methanesulfonamido-1- |
| | methyl-1H-indazol-7-yl)-7-[4-fluoro-2-(trifluoromethyl) |
| | phenyl]-4-oxo-3H,4H-pyrido[2,3-d]pyrimidin-2-yl]- |
| | 2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 23 | N-[(1S)-1-[(3P)-3-(4-chloro-3-methanesulfonamido-1- |
| | methyl-1H-indazol-7-yl)-7-[3-(difluoromethyl) |
| | phenyl]-4-oxo-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 24 | N-[(1S)-1-[(3P)-3-(4-chloro-3-methanesulfonamido-1- |
| | methyl-1H-indazol-7-yl)-4-oxo-7-[3-(trifluoromethyl) |
| | phenyl]-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 25 | N-[(1S)-1-[(3P)-3-(4-chloro-3-methanesulfonamido-1- |
| | methyl-1H-indazol-7-yl)-4-oxo-7-[2-(trifluoromethyl) |
| | phenyl]-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 26 | N-[(1S)-1-[(3P)-3-(4-chloro-3-methanesulfonamido- |
| | 1-methyl-1H-indazol-7-yl)-4-oxo-7-[4-(trifluoromethoxy) |
| | phenyl]-3H,4H-pyrido[2,3-d]pyrimidin-2-yl]- |
| | 2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 27 | N-[(1S)-1-[(3P)-3-(4-chloro-3-methanesulfonamido-1- |
| | methyl-1H-indazol-7-yl)-7-[4-(difluoromethoxy) |
| | phenyl]-4-oxo-3H,4H-pyrido[2,3-d]pyrimidin-2-yl]- |
| | 2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 28 | N-[(1S)-1-[(3P)-3-(4-chloro-3-methanesulfonamido-1- |
| | methyl-1H-indazol-7-yl)-4-oxo-7-(pyrimidin-4- |
| | yl)-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 29 | N-[(1S)-1-[(3P)-3-(4-chloro-3-methanesulfonamido-1- |
| | methyl-1H-indazol-7-yl)-7-(5-chloropyridin-3-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 30 | N-[(1S)-1-[(3P)-3-(4-chloro-3-methanesulfonamido-1- |
| | methyl-1H-indazol-7-yl)-7-(6-fluoropyridin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 31 | N-[(1S)-1-[(3P)-3-(4-chloro-3-methanesulfonamido-1- |
| | methyl-1H-indazol-7-yl)-7-(6-methylpyridin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 32 | N-[(1S)-1-[(3P)-3-(4-chloro-3-methanesulfonamido-1- |
| | methyl-1H-indazol-7-yl)-7-(2-fluoro-4- |
| | methanesulfonylphenyl)-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 33 | N-[(1S)-1-[(3P)-3-(4-chloro-3-methanesulfonamido-1- |
| | methyl-1H-indazol-7-yl)-4-oxo-7-(pyridazin-4-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 34 | N-[(1S)-1-[(3P)-3-(4-chloro-3-methanesulfonamido-1- |
| | methyl-1H-indazol-7-yl)-4-oxo-7-(pyridin-4-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 35 | N-[(1S)-1-[(3P)-3-(4-chloro-3-methanesulfonamido-1- |
| | methyl-1H-indazol-7-yl)-7-(4-methanesulfonylphenyl)- |
| | 4-oxo-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 36 | N-[(1S)-1-[(3P)-3-(4-chloro-3-methanesulfonamido-1- |
| | methyl-1H-indazol-7-yl)-7-[3-fluoro-6-(trifluoromethyl) |
| | pyridin-2-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 37 | N-[(1S)-1-[(3P)-3-(4-chloro-3-methanesulfonamido- |
| | 1-methyl-1H-indazol-7-yl)-4-oxo-7-[2- |
| | (trifluoromethyl)pyrimidin-4-yl]-3H,4H-pyrido[2,3-d] |
| | pyrimidin-2-yl]-2-(3,5-difluorophenyl)ethyl]- |
| | 2-[(2S,4R)-9-(difluoromethyl)-5,5-difluoro- |
| | 7,8-diazatricyclo[4.3.0.0²,⁴]nona-1(6),8-dien-7-yl] |
| | acetamide |
| Example 38 | N-[(1S)-1-[(3P)-3-(4-chloro-3-methanesulfonamido- |
| | 1-methyl-1H-indazol-7-yl)-7-[6-(difluoromethyl) |
| | pyridin-2-yl]-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 39 | N-[(1S)-1-[(3P)-3-(4-chloro-3-methanesulfonamido- |
| | 1-methyl-1H-indazol-7-yl)-7-[4-(difluoromethyl) |
| | pyridin-2-yl]-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 47 | N-[(1S)-1-[(3P)-3-(4-chloro-3-methanesulfonamido- |
| | 1-methyl-1H-indazol-7-yl)-4-oxo-7-(pyrimidin-2-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴] |
| | nona-1(6),8-dien-7-yl]acetamide |
| Example 48 | N-[(1S)-1-[(3P)-3-(4-chloro-3-methanesulfonamido- |
| | 1-methyl-1H-indazol-7-yl)-7-(2-methylpyridin-4-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 49 | N-[(1S)-1-[(3P)-3-(4-chloro-3-methanesulfonamido- |
| | 1-methyl-1H-indazol-7-yl)-7-(4-methylpyridin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 50 | N-[(1S)-1-[(3P)-3-(4-chloro-3-methanesulfonamido- |
| | 1-methyl-1H-indazol-7-yl)-7-(6-chloropyridin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 51 | N-[(1S)-1-[(3P)-3-(4-chloro-3-methanesulfonamido- |
| | 1-methyl-1H-indazol-7-yl)-7-(5-methoxypyridin-3-yl)- |
| | 4-oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 52 | N-[(1S)-1-[(3P)-3-(4-chloro-3-methanesulfonamido-1- |
| | methyl-1H-indazol-7-yl)-4-oxo-7-(pyridin-3-yl)-3H, |
| | 4H-pyrido[2,3-d]pyrimidin-2-yl]-2-(3,5-difluorophenyl) |
| | ethyl]-2-[(2S,4R)-9-(difluoromethyl)-5,5-difluoro-7,8- |
| | diazatricyclo[4.3.0.0²,⁴]nona-1(6),8-dien-7-yl]acetamide |
| Example 54 | N-[(1S)-1-[(3P)-3-(4-chloro-3-methanesulfonamido-1- |
| | methyl-1H-indazol-7-yl)-7-(6-methoxypyrazin-2-yl)-4- |
| | oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 55 | N-[(1S)-1-[(3P)-3-(4-chloro-3-methanesulfonamido-1- |
| | methyl-1H-indazol-7-yl)-4-oxo-7-(pyrimidin-5-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 56 | N-[(1S)-1-[(3P)-3-(4-chloro-3-methanesulfonamido- |
| | 1-methyl-1H-indazol-7-yl)-7-(4-methoxypyrimidin-2- |
| | yl)-4-oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 57 | N-[(1S)-1-[(3P)-3-(4-chloro-3-methanesulfonamido-1- |
| | methyl-1H-indazol-7-yl)-7-(3,5-dichloropyrazin-2-yl)- |
| | 4-oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 58 | N-[(1S)-1-[(3P)-3-(4-chloro-3-methanesulfonamido-1- |
| | methyl-1H-indazol-7-yl)-7-(2-methanesulfonylpyrimidin- |
| | 5-yl)-4-oxo-3H,4H-pyrido[2,3-d]pyrimidin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 59 | N-[(1S)-1-[(3P)-3-(4-chloro-3-methanesulfonamido-1- |
| | methyl-1H-indazol-7-yl)-7-(2-methoxypyridin-4-yl)-4- |
| | oxo-3H,4H-pyrido[2,3-d]pyrimidin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 60 | N-[(1S)-1-[(3P)-3-(4-chloro-3-methanesulfonamido-1- |
| | methyl-1H-indazol-7-yl)-4-oxo-7-(pyridin-2-yl)- |
| | 3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 61 | N-[(1S)-1-[(3P)-3-(4-chloro-3-methanesulfonamido-1- |
| | methyl-1H-indazol-7-yl)-7-(3-chloropyrazin-2-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 62 | N-[(1S)-1-[(3P)-3-(4-chloro-3-methanesulfonamido-1- |
| | methyl-1H-indazol-7-yl)-4-oxo-7-[3-(pentafluoro- |
| | A6-sulfanyl)phenyl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 63 | N-[(1S)-1-[(3P)-3-(4-chloro-3-methanesulfonamido-1- |
| | methyl-1H-indazol-7-yl)-4-oxo-7-[4-(pentafluoro-A6- |
| | sulfanyl)phenyl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 64 | N-[(1S)-1-[(3P)-3-(4-chloro-3-methanesulfonamido-1- |
| | methyl-1H-indazol-7-yl)-7-(1H-imidazol-1-yl)- |
| | 4-oxo-3H,4H-pyrido[2,3-d]pyrimidin-2-yl]-2-(3,5- |
| | difluorophenyl)ethyl]-2-[(2S,4R)-9-(difluoromethyl)- |
| | 5,5-difluoro-7,8-diazatricyclo[4.3.0.0²,⁴]nona-1 |
| | (6),8-dien-7-yl]acetamide |
| Example 65 | N-[(1S)-1-[(3P)-3-(4-chloro-3-methanesulfonamido- |
| | 1-methyl-1H-indazol-7-yl)-4-oxo-7-[3-(trifluoromethyl)- |
| | 1H-pyrazol-1-yl]-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 66 | N-[(1S)-1-[(3P)-3-(4-chloro-3-methanesulfonamido- |
| | 1-methyl-1H-indazol-7-yl)-4-oxo-7-[4-(trifluoromethyl)- |
| | 1H-pyrazol-1-yl]-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 67 | N-[(1S)-1-[(3P)-3-(4-chloro-3-methanesulfonamido- |
| | 1-methyl-1H-indazol-7-yl)-7-(2-methyl-1,3- |
| | benzothiazol-6-yl)-4-oxo-3H,4H-pyrido[2,3-d] |
| | pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl] |
| | acetamide |
| Example 68 | N-[(1S)-1-[(3P)-3-(4-chloro-3-methanesulfonamido- |
| | 1-methyl-1H-indazol-7-yl)-7-(1-methyl-1H-pyrazol- |
| | 5-yl)-4-oxo-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 69 | N-[(1S)-1-[(3P)-3-(4-chloro-3-methanesulfonamido- |
| | 1-methyl-1H-indazol-7-yl)-7-[1-(2-methylpropyl)- |
| | 1H-pyrazol-5-yl]-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 70 | N-[(1S)-1-[(3P)-3-(4-chloro-3-methanesulfonamido- |
| | 1-methyl-1H-indazol-7-yl)-7-(1,3-dimethyl-1H- |
| | pyrazol-5-yl)-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 71 | N-[(1S)-1-[(3P)-7-(1,3-benzothiazol-6-yl)-3- |
| | (4-chloro-3-methanesulfonamido-1-methyl- |
| | 1H-indazol-7-yl)-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 72 | N-[(1S)-1-[(3P)-7-(1,3-benzothiazol-5-yl)-3-(4- |
| | chloro-3-methanesulfonamido-1-methyl-1H- |
| | indazol-7-yl)-4-oxo-3H,4H-pyrido[2,3-d] |
| | pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 73 | N-[(1S)-1-[(3P)-3-(4-chloro-3-methanesulfonamido- |
| | 1-methyl-1H-indazol-7-yl)-4-oxo-7-[1-(2,2,2- |
| | trifluoroethyl)-1H-pyrazol-3-yl]-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 74 | N-[(1S)-1-[(3P)-3-(4-chloro-3-methanesulfonamido- |
| | 1-methyl-1H-indazol-7-yl)-7-(1-cyclopropyl-1H- |
| | pyrazol-4-yl)-4-oxo-3H,4H-pyrido[2,3-d] |
| | pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 75 | N-[(1S)-1-[(3P)-3-(4-chloro-3-methanesulfonamido- |
| | 1-methyl-1H-indazol-7-yl)-4-oxo-7-[1-(2,2,2- |
| | trifluoroethyl)-1H-pyrazol-4-yl]-3H,4H- |
| | pyrido[2,3-d]pyrimidin-2-yl]-2-(3,5-difluorophenyl) |
| | ethyl]-2-[(2S,4R)-9-(difluoromethyl)-5,5-difluoro-7,8- |
| | diazatricyclo[4.3.0.0²,⁴]nona-1(6),8-dien-7-yl]acetamide |
| Example 76 | N-[(1S)-1-[(3P)-3-(4-chloro-3-methanesulfonamido- |
| | 1-methyl-1H-indazol-7-yl)-7-[1-(2-fluoroethyl)-1H- |
| | pyrazol-4-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 77 | N-[(1S)-1-[(3P)-3-(4-chloro-3-methanesulfonamido- |
| | 1-methyl-1H-indazol-7-yl)-7-(1-cyclopropyl-1H- |
| | pyrazol-5-yl)-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 78 | N-[(1S)-1-[(3P)-3-(4-chloro-3-methanesulfonamido- |
| | 1-methyl-1H-indazol-7-yl)-7-[1-(cyclopropylmethyl)- |
| | 1H-pyrazol-5-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 79 | N-[(1S)-1-[(3P)-3-(4-chloro-3-methanesulfonamido- |
| | 1-methyl-1H-indazol-7-yl)-7-[1-(2,2-difluoroethyl)- |
| | 1H-pyrazol-3-yl]-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien- |
| | 7-yl]acetamide |
| Example 80 | N-[(1S)-1-[(3P)-3-(4-chloro-3-methanesulfonamido- |
| | 1-methyl-1H-indazol-7-yl)-4-oxo-7-[3-(trifluoromethyl)- |
| | 1H-1,2,4-triazol-1-yl]-3H,4H-pyrido[2,3- |
| | d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 81 | N-[(1S)-1-[(3P)-7-(1,3-benzoxazol-5-yl)-3-(4- |
| | chloro-3-methanesulfonamido-1-methyl-1H- |
| | indazol-7-yl)-4-oxo-3H,4H-pyrido[2,3-d]pyrimidin- |
| | 2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 82 | N-[(1S)-1-[(3P)-3-(4-chloro-3-methanesulfonamido- |
| | 1-methyl-1H-indazol-7-yl)-7-(2-methoxy-1,3- |
| | thiazol-5-yl)-4-oxo-3H,4H-pyrido[2,3-d] |
| | pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 83 | N-[(1S)-1-[(3P)-3-(4-chloro-3-methanesulfonamido-1- |
| | methyl-1H-indazol-7-yl)-7-[1-methyl-3-(trifluoromethyl)- |
| | 1H-pyrazol-5-yl]-4-oxo-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 84 | N-[(1S)-1-[(3P)-3-(4-chloro-3-methanesulfonamido- |
| | 1-methyl-1H-indazol-7-yl)-4-oxo-7-[1-(2,2,3,3- |
| | tetrafluoropropyl)-1H-pyrazol-3-yl]-3H,4H-pyrido |
| | [2,3-d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 85 | N-[(1S)-1-[(3P)-3-(4-chloro-3-methanesulfonamido- |
| | 1-methyl-1H-indazol-7-yl)-4-oxo-7-[1-(4,4,4- |
| | trifluorobutyl)-1H-pyrazol-3-yl]-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 86 | N-[(1S)-1-[(3P)-3-(4-chloro-3-methanesulfonamido- |
| | 1-methyl-1H-indazol-7-yl)-7-[1-(2,2-difluorobutyl)- |
| | 1H-pyrazol-3-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 87 | N-[(1S)-1-[(3P)-3-(4-chloro-3-methanesulfonamido- |
| | 1-methyl-1H-indazol-7-yl)-7-[1-(3-fluoropropyl)-1H- |
| | pyrazol-3-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 88 | N-[(1S)-1-[(3P)-3-(4-chloro-3-methanesulfonamido- |
| | 1-methyl-1H-indazol-7-yl)-4-oxo-7-[1-(2,2,3,3,3- |
| | pentafluoropropyl)-1H-pyrazol-3-yl]-3H,4H- |
| | pyrido[2,3-d]pyrimidin-2-yl]-2-(3,5-difluorophenyl) |
| | ethyl]-2-[(2S,4R)-9-(difluoromethyl)-5,5-difluoro- |
| | 7,8-diazatricyclo[4.3.0.02,4]nona-1(6),8-dien-7- |
| | yl]acetamide |
| Example 89 | N-[(1S)-1-[(3P)-3-(4-chloro-3-methanesulfonamido- |
| | 1-methyl-1H-indazol-7-yl)-4-oxo-7-[1-(3,3,3- |
| | trifluoropropyl)-1H-pyrazol-3-yl]-3H,4H-pyrido[2,3- |
| | d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 90 | N-[(1S)-1-[(3P)-3-(4-chloro-3-methanesulfonamido- |
| | 1-methyl-1H-indazol-7-yl)-4-oxo-7-[5-(trifluoromethyl)- |
| | 1H-pyrazol-3-yl]-3H,4H-pyrido[2,3- |
| | d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 91 | N-[(1S)-1-[(3P)-3-(4-chloro-3-methanesulfonamido- |
| | 1-methyl-1H-indazol-7-yl)-7-[2-(2-hydroxypropan-2-yl)- |
| | 1,3-thiazol-4-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo[4.3.0.0²,⁴] |
| | nona-1(6),8-dien-7-yl]acetamide |
| Example 92 | N-[(1S)-1-[(3P)-3-(4-chloro-3-methanesulfonamido-1- |
| | methyl-1H-indazol-7-yl)-7-[2-(2-hydroxypropan-2-yl)- |
| | 1,3-thiazol-5-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo[4.3.0.0²,⁴] |
| | nona-1(6),8-dien-7-yl]acetamide |
| Example 93 | N-[(1S)-1-[(3P)-3-(4-chloro-3-methanesulfonamido-1- |
| | methyl-1H-indazol-7-yl)-7-(2-methanesulfonyl-1,3- |
| | thiazol-4-yl)-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 94 | N-[(1S)-1-[(3P)-3-(4-chloro-3-methanesulfonamido- |
| | 1-methyl-1H-indazol-7-yl)-7-[1-(2,2-difluoropropyl)- |
| | 5-(trifluoromethyl)-1H-pyrazol-3-yl]-4-oxo-3H,4H- |
| | pyrido[2,3-d]pyrimidin-2-yl]-2-(3,5-difluorophenyl) |
| | ethyl]-2-[(2S,4R)-9-(difluoromethyl)-5,5-difluoro-7,8- |
| | diazatricyclo[4.3.0.02,4]nona-1(6),8-dien-7-yl]acetamide |
| Example 95 | N-[(1S)-1-[(3P)-3-(4-chloro-3-methanesulfonamido-1- |
| | methyl-1H-indazol-7-yl)-7-[1-(2,2-difluoropropyl)- |
| | 3-(trifluoromethyl)-1H-pyrazol-5-yl]-4-oxo-3H,4H-pyrido |
| | [2,3-d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.02,4]nona-1(6),8-dien-7-yl]acetamide |
| Example 96 | N-[(1S)-1-[(3P)-3-(4-chloro-3-methanesulfonamido-1- |
| | methyl-1H-indazol-7-yl)-7-[1-(2,2-difluoropropyl)- |
| | 5-methyl-1H-pyrazol-3-yl]-4-oxo-3H,4H-pyrido[2,3-d] |
| | pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.02,4]nona-1(6),8-dien-7-yl]acetamide |
| Example 97 | N-[(1S)-1-[(3P)-3-(4-chloro-3-methanesulfonamido- |
| | 1-methyl-1H-indazol-7-yl)-7-[1-(2,2-difluoropropyl)-3- |
| | methyl-1H-pyrazol-5-yl]-4-oxo-3H,4H-pyrido[2,3-d] |
| | pyrimidin-2-yl]-2-(3,5-difluorophenyl)ethyl]-2-[(2S, |
| | 4R)-9-(difluoromethyl)-5,5-difluoro-7,8-diazatricyclo |
| | [4.3.0.02,4]nona-1(6),8-dien-7-yl]acetamide |
| Example 98 | N-[(1S)-1-[(3P)-3-(4-chloro-3-methanesulfonamido-1- |
| | methyl-1H-indazol-7-yl)-7-(5-methyl-1H-pyrazol-3- |
| | yl)-4-oxo-3H,4H-pyrido[2,3-d]pyrimidin-2-yl]-2- |
| | (3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example 99 | N-[(1S)-1-[(3P)-3-(4-chloro-3-methanesulfonamido- |
| | 1-methyl-1H-indazol-7-yl)-7-[2-(1,1-difluoroethyl)- |
| | 1,3-thiazol-5-yl]-4-oxo-3H,4H-pyrido[2,3-d]pyrimidin- |
| | 2-yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |
| Example | N-[(1S)-1-[(3P)-3-(4-chloro-3-methanesulfonamido-1- |
| 100 | methyl-1H-indazol-7-yl)-7-[2-(1,1-difluoroethyl)-1,3- |
| | thiazol-5-yl]-4-oxo-3H,4H-pyrido[2,3-d]pyrimidin-2- |
| | yl]-2-(3,5-difluorophenyl)ethyl]-2-[(2S,4R)-9- |
| | (difluoromethyl)-5,5-difluoro-7,8-diazatricyclo |
| | [4.3.0.0²,⁴]nona-1(6),8-dien-7-yl]acetamide |

### Biological Methods:

HIV cell culture assay—MT-2 cells, 293T cells and the proviral DNA clone of NL₄₋₃ virus were obtained from the NIH AIDS Research and Reference Reagent Program. MT-2 cells were propagated in RPMI 1640 media supplemented with 10% heat inactivated fetal bovine serum (FBS), 100 mg/ml penicillin G and up to 100 units/mL streptomycin. The 293T cells were propagated in DMEM media supplemented with 10% heat inactivated FBS, 100 mg/mL penicillin G and 100 mg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, WI). Supernatent was harvested after 2-3 days and the amount of virus present was titered in MT-2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT-2 cells infected for 4-5 days with the recombinant virus in the presence of serial dilutions of the compound.

The 50% effective concentration (EC₅₀) was calculated by using the exponential form of the median effect equation where (Fa)=1/[1+(ED₅₀/drug conc.)m] (Johnson VA, Byington RT. Infectivity Assay. In Techniques in HIV Research. ed. Aldovini A, Walker BD. 71-76. New York: Stockton Press. 1990). The 50% inhibitory concentration (EC₅₀) was calculated by using the exponential form of the median effect equation where percent inhibition=1/[1+(EC₅₀/drug concentration)m], where m is a parameter that reflects the slope of the concentration-response curve.

Compound cytotoxicity and the corresponding CC₅₀ values were determined using the same protocol as described in the antiviral assay except that uninfected cells were used. Cytotoxicity was assessed on day 4 in uninfected MT2 cells by using a XTT (2,3-bis[2-Methoxy-4-nitro-5-sulfophenyl]-2H-tetrazolium-5-carboxyanilide inner salt)-based colorimetric assay (Sigma-Aldrich, St Louis, Mo.).

| Example | EC₅₀ (nM) | CC₅₀ (uM) |
|---|---|---|
| Example 1 | 0.055 | >0.1 |
| Example 2 | 0.031 | >0.1 |
| Example 3 | 0.051 | >0.1 |
| Example 4 | 0.055 | >0.1 |
| Example 5 | 0.021 | >0.1 |
| Example 6 | 0.023 | >0.1 |
| Example 7 | 0.020 | >0.1 |
| Example 8 | 0.019 | >0.1 |
| Example 9 | 0.065 | >0.1 |
| Example 10 | 0.030 | >0.1 |
| Example 11 | 0.036 | >0.1 |
| Example 12 | 0.060 | >0.1 |
| Example 13 | 0.040 | >0.1 |
| Example 14 | 0.28 | >0.1 |
| Example 15 | 0.025 | >0.1 |
| Example 16 | 0.052 | >0.1 |
| Example 17 | 0.038 | >0.1 |
| Example 18 | 0.068 | >0.1 |
| Example 19 | 0.23 | >0.1 |
| Example 20 | 0.20 | >0.1 |
| Example 21 | 0.39 | >0.1 |
| Example 22 | 0.052 | >0.1 |
| Example 23 | 0.11 | >0.1 |
| Example 24 | 0.16 | >0.1 |
| Example 25 | 0.060 | >0.1 |
| Example 26 | 0.51 | >0.1 |
| Example 27 | 0.34 | >0.1 |
| Example 28 | 0.040 | >0.1 |
| Example 29 | 0.057 | >0.1 |
| Example 30 | 0.034 | >0.1 |
| Example 31 | 0.031 | >0.1 |
| Example 32 | 0.075 | >0.1 |
| Example 33 | 0.45 | >0.1 |
| Example 34 | 0.070 | >0.1 |
| Example 35 | 0.14 | >0.1 |
| Example 36 | 0.045 | >0.1 |
| Example 37 | 0.11 | >0.1 |
| Example 38 | 0.051 | >0.1 |
| Example 39 | 0.046 | >0.1 |
| Example 47 | 0.067 | >0.1 |
| Example 48 | 0.053 | >0.1 |
| Example 49 | 0.018 | >0.1 |
| Example 50 | 0.056 | >0.1 |
| Example 51 | 0.085 | >0.1 |
| Example 52 | 0.055 | >0.1 |
| Example 54 | 0.039 | >0.1 |
| Example 55 | 0.090 | >0.1 |
| Example 56 | 0.051 | >0.1 |
| Example 57 | 0.073 | >0.1 |
| Example 59 | 0.050 | >0.1 |
| Example 60 | 0.032 | >0.1 |
| Example 61 | 0.025 | >0.1 |
| Example 64 | 0.53 | >0.1 |
| Example 65 | 0.062 | >0.1 |
| Example 66 | 0.070 | >0.1 |
| Example 67 | 0.55 | >0.1 |
| Example 68 | 0.049 | >0.1 |
| Example 69 | 0.099 | >0.1 |
| Example 70 | 0.041 | >0.1 |
| Example 71 | 0.097 | >0.1 |
| Example 72 | 0.11 | >0.1 |
| Example 73 | 0.029 | >0.1 |
| Example 74 | 0.054 | >0.1 |
| Example 75 | 0.090 | >0.1 |
| Example 76 | 0.11 | >0.1 |
| Example 77 | 0.073 | >0.1 |
| Example 78 | 0.090 | >0.1 |
| Example 79 | 0.047 | >0.1 |
| Example 80 | 0.028 | >0.1 |
| Example 81 | 0.082 | >0.1 |
| Example 82 | 0.069 | >0.1 |
| Example 83 | 0.070 | >0.1 |
| Example 84 | 0.041 | >0.1 |
| Example 85 | 0.096 | >0.1 |
| Example 86 | 0.084 | >0.1 |
| Example 87 | 0.036 | >0.1 |
| Example 88 | 0.079 | >0.1 |
| Example 89 | 0.056 | >0.1 |
| Example 90 | 0.098 | >0.1 |
| Example 91 | 0.037 | >0.1 |
| Example 92 | 0.065 | >0.1 |
| Example 93 | 0.056 | >0.1 |
| Example 94 | 0.16 | >0.1 |
| Example 95 | 0.25 | >0.1 |
| Example 96 | 0.044 | >0.1 |
| Example 97 | 12 | >0.1 |
| Example 98 | 0.20 | >0.1 |
| Example 99 | 0.062 | >0.1 |

The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

1. A compound of Formula I, or a pharmaceutically acceptable salt thereof: 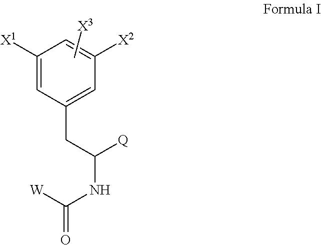
wherein:
X¹ and X² are independently selected from H, F, Cl or —CH₃ and X³ is H, F, Cl, —CH₃, —OCH₃, —OCHF₂, or —OCF₃ with the proviso that within the group X¹, X², and X³ the substituent Cl is not used more than twice and the substituent —CH₃ is not used more than twice;
Q is selected from: 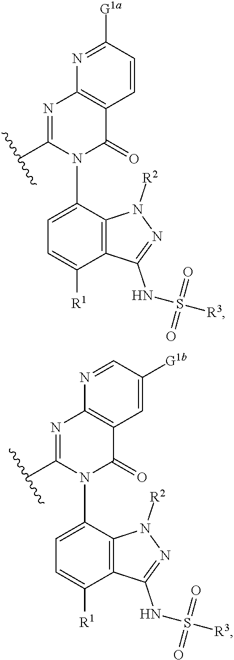
R¹ is H, Cl, or CH₃;
R² is H, C₁-C₃alkyl optionally substituted with 1-3 fluorines, or C₃-C₆cycloalkyl optionally substituted with 1-2 fluorines;
R³ is C₁-C₃alkyl or C₃-C₄cycloalkyl;
G^{1a} is phenyl, pyridine, pyrazine or pyrimidine, each of which is substituted with —SF_{5,} or G^{1a} is selected from: 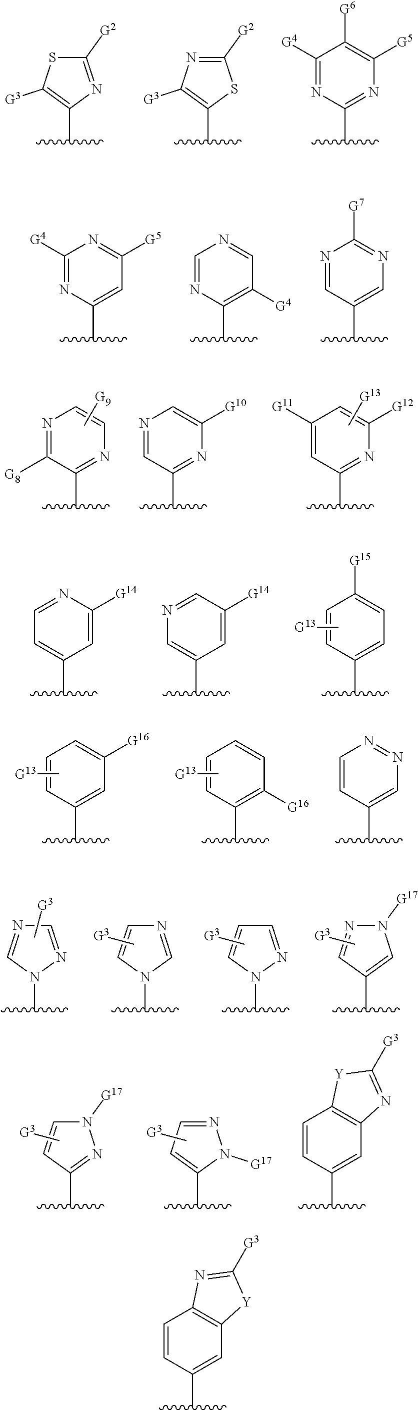
G² is C₁-C₃alkyl, —O(C₁-C₃alkyl), —S(O₂)CH₃, or —C(CH₃)₂OH wherein C₁-C₃alkyl is optionally substituted with 1-3 fluorines;
G³ is H, or methyl optionally substituted with 1-3 fluorines;
G⁴ and G⁵ are independently selected from H, —O(C₁-C₃alkyl), or C₁-C₂alkyl optionally substituted with 1-3 fluorines;
G⁶ is H, Cl, or F;
G⁷ is H, —OCH₃, or —S(O₂)CH₃;
G⁸ is H, methyl, ethyl, or Cl;
G⁹ is H or Cl;
G¹⁰ is H, C₁-C₂alkyl, —OCH₃, or —SF5 where C₁-C₂alkyl is optionally substituted with 1-3 fluorines;
G¹¹ and G¹² are independently selected from H, F, Cl or C₁—C₂alkyl wherein C₁-C₂alkyl is optionally substituted with 1-3 fluorines;
G¹³ is H or F;
G¹⁴ is H, methyl, Cl, —OCH₃;
G¹⁵ is —O(C₁-C₂alkyl) substituted with 1-3 fluorines, or —S(O₂)CH₃;
G¹⁶ is C₁-C₂alkyl or —O(C₁-C₂alkyl) wherein C₁-C₂alkyl is substituted with 1-3 fluorines;
G¹⁷ is H, cPr, —CH2cPr, or C₁-C₄alkyl wherein C₁-C₄alkyl is optionally substituted with 1-5 fluorines;
Y is O, S or N;
G^{1b} is pyridine, pyrimidine, pyrazine, or phenyl, each of which is substituted once from the group F, Cl, or C₁-C₂alkyl wherein C₁-C₂alkyl is optionally substituted with 1-3 fluorines;
W is selected from: 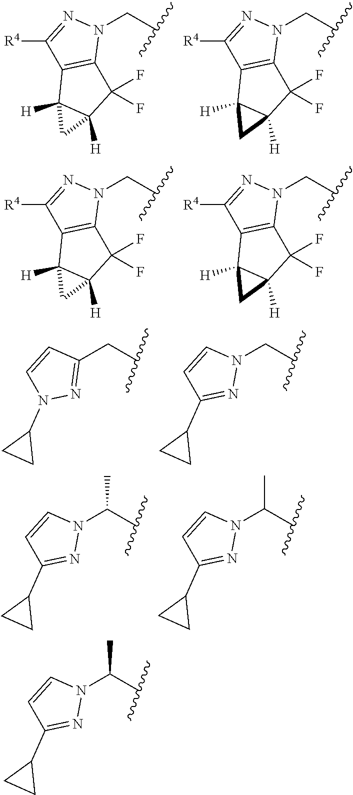
wherein R⁴ is methyl optionally substituted with 1-3 fluorines.

2. A compound or salt according to claim 1 wherein Q is the following:

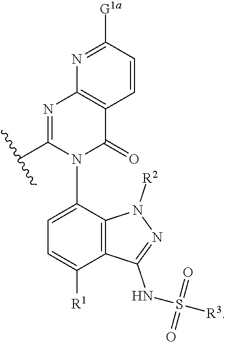

3. A compound or salt according to claim 1 wherein Q is the following:

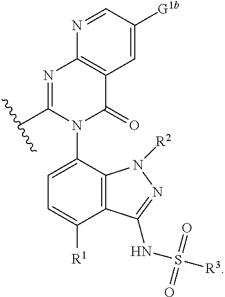

4. A compound or salt according to claim 1 wherein W is the following:

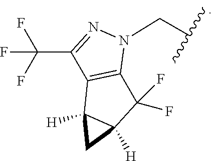

5. A compound or salt according to claim 1 wherein W is the following:

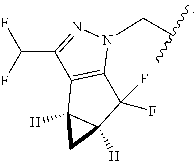

6. A compound or salt according to claim 1 wherein W is the following: 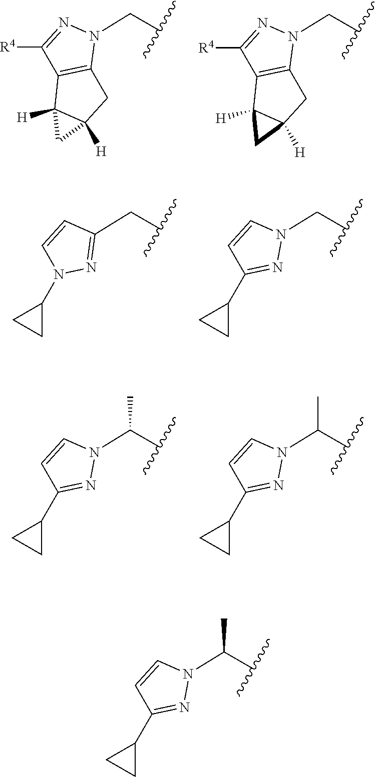
wherein R⁴ is methyl optionally substituted with 1-3 fluorines.

7. A compound or salt according to claim 1 wherein R¹ is Cl; R² is methyl, 2,2-difluoroethyl, or 2,2,2-trifluoroethyl; and R³ is methyl or cyclopropyl.

8. A compound or salt according to claim 1 wherein R¹ is Cl; R² is methyl; and R³ is methyl.

9. A compound or salt according to claim 1 wherein X³ is H.

10. A compound or salt according to claim 1 wherein X¹ is F, X² is F, and X³ is H.

11. A compound or salt according to claim 1 wherein if X³ is H then at least one of X¹ and X² is other than F.

12. A compound or salt according to claim 1 wherein G^{1a} is one of the following:

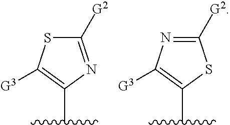

13. A compound or salt according to claim 1 wherein G^{1a} is one of the following:

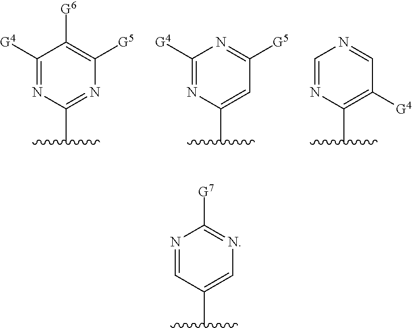

14. A compound or salt according to claim 1 wherein G^{1a} is one of the following:

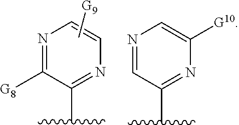

15. A compound or salt according to claim 1 wherein G^{1a} is one of the following:

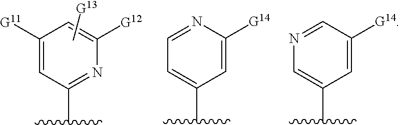

16. A compound or salt according to claim 1 wherein G^{1a} is one of the following:

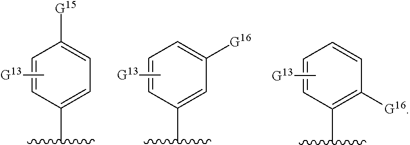

17. A compound or salt according to claim 1 wherein G^{1a} is:

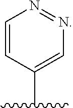 [SMILES: *c1ccnnc1]

18. A compound or salt according to claim 1 wherein G^{1a} is one of the following:

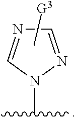

19. A compound or salt according to claim 1 wherein G^{1a} is one of the following:

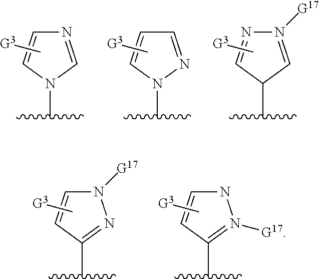

20. A compound or salt according to claim 1 wherein G^{1a} is one of the following:

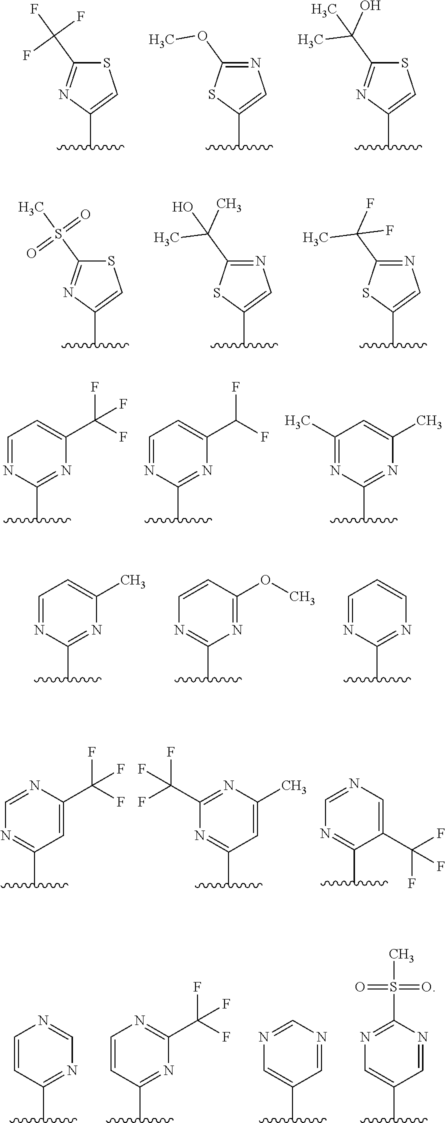

21. A compound or salt according to claim 1 wherein G^{1a} is one of the following:

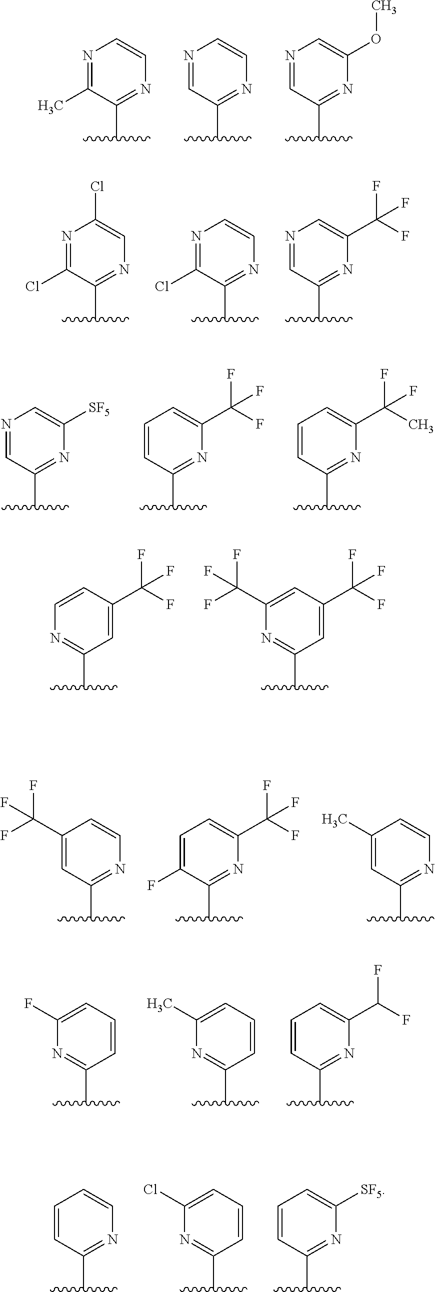

22. A compound or salt according to claim 1 wherein G^{1a} is one of the following:

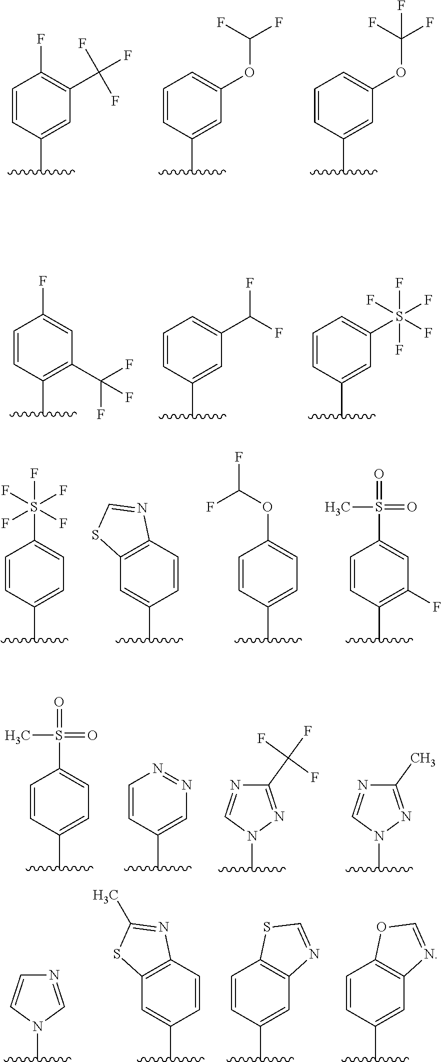

23. A compound or salt according to claim 1 wherein G^{1a} is one of the following:

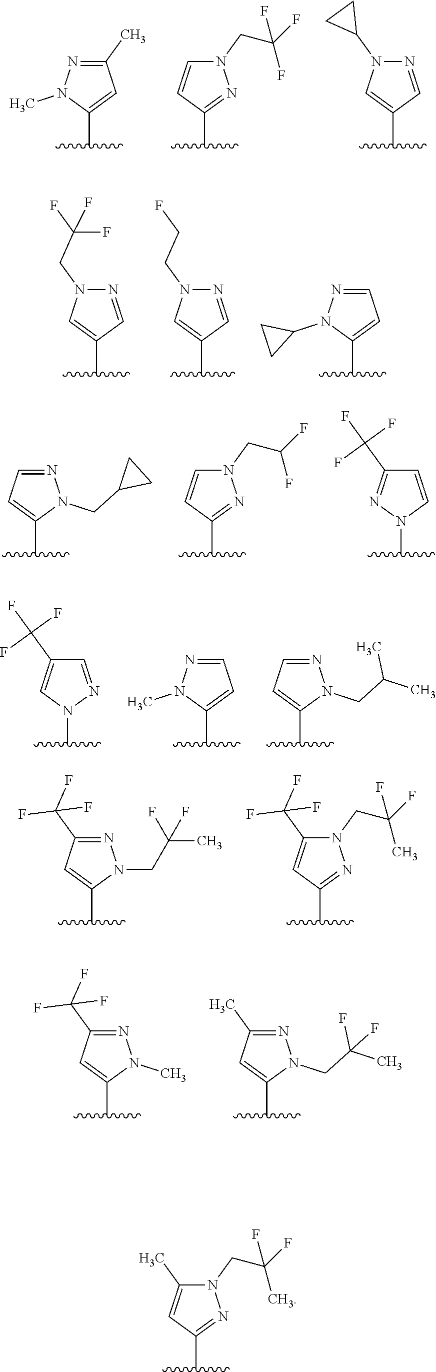

24. A compound or salt according to claim 1 wherein G^{1b} is one of the following:

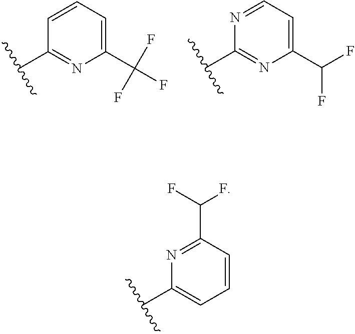

25. A compound or salt according to claim 1 wherein G^{1a} or G^{1b} contains 2-3 fluorines.

26. A compound or salt according to claim 1 wherein the chemical formula of G^{1a} or G^{1b} is C₍₄₋₆₎H₍₂₋₃₎F₍₂₋₃₎N₍₁₋₂₎.

27. A compound or salt according to claim 1 wherein the stereochemistry is as depicted below:

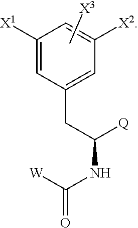

28. A compound or salt according to claim 1 wherein the stereochemistry is as depicted below:

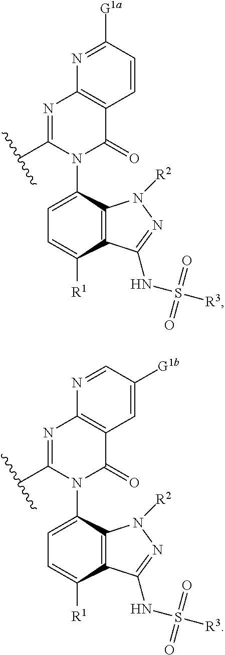

29. A compound or salt according to claim 1, selected from the group consisting of: 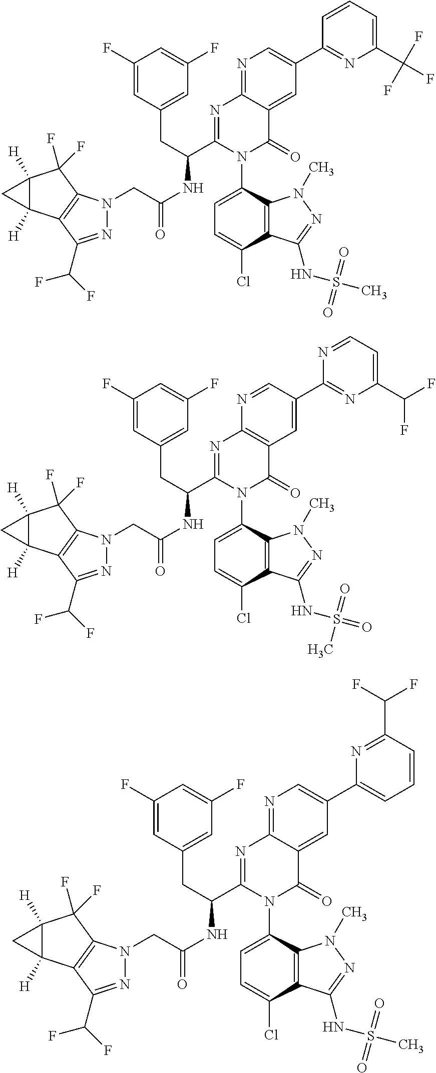 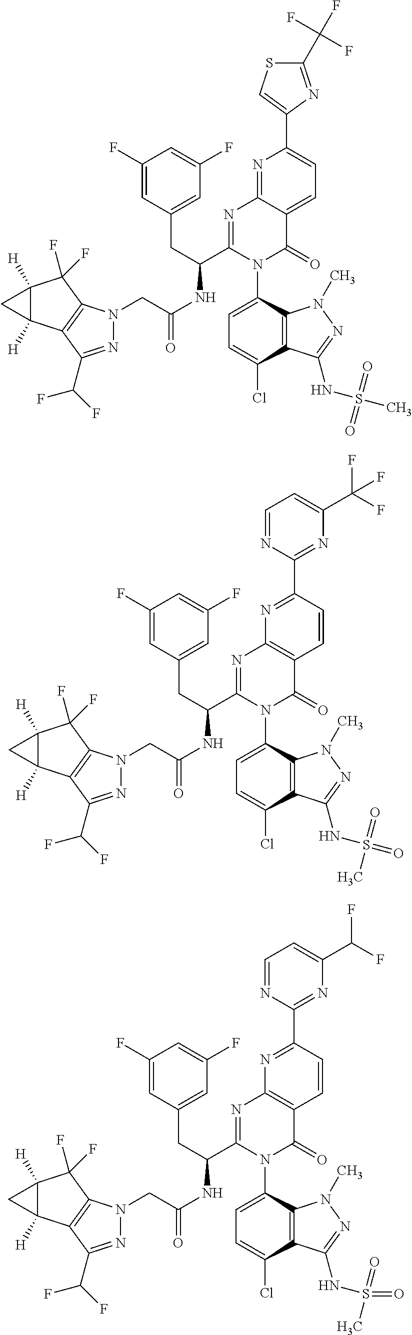 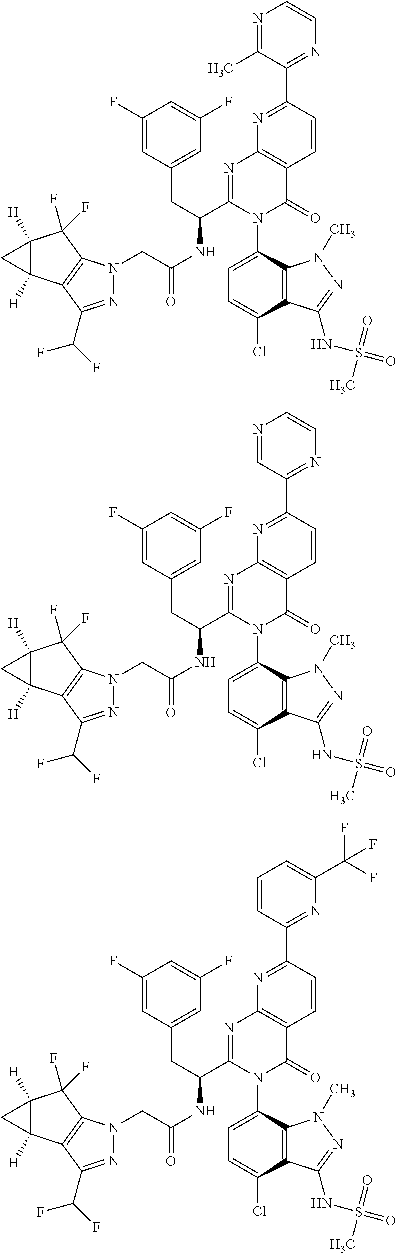 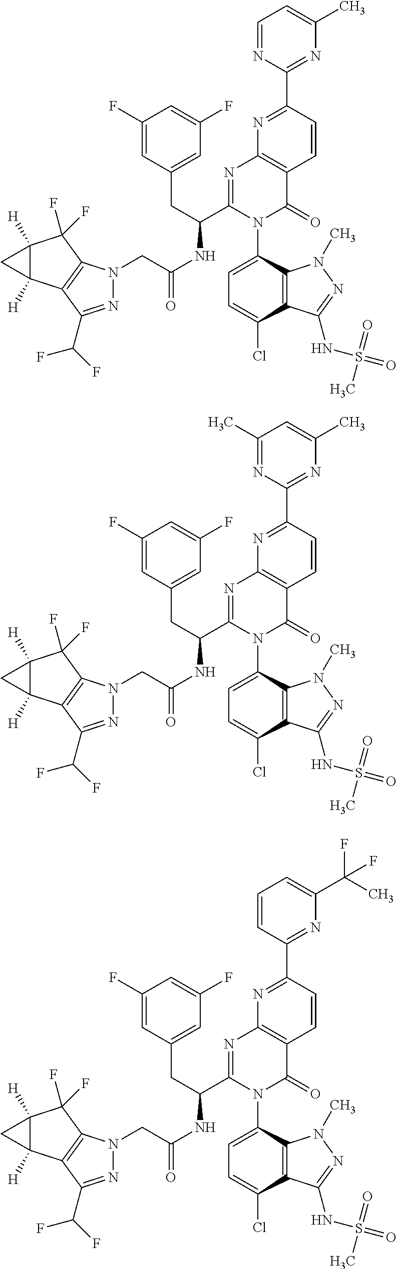 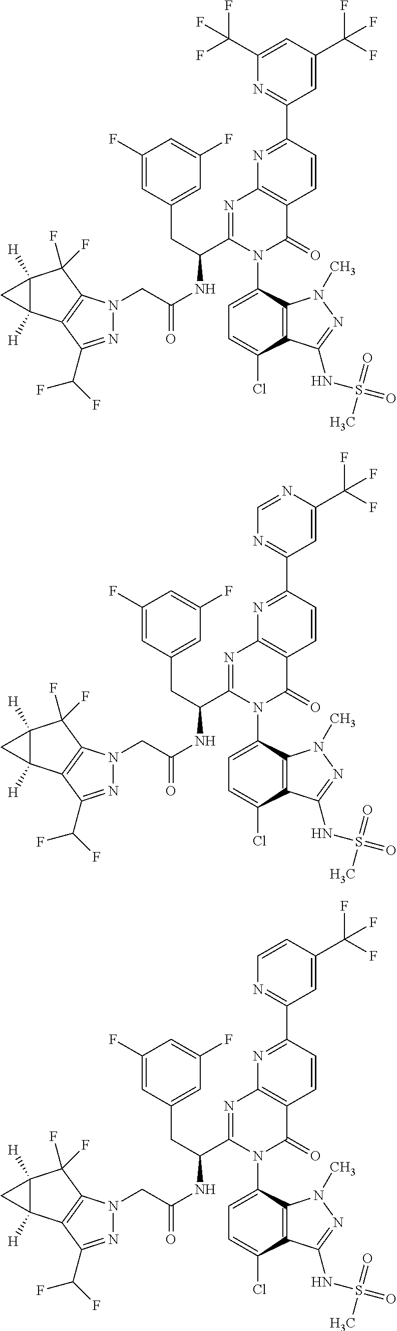 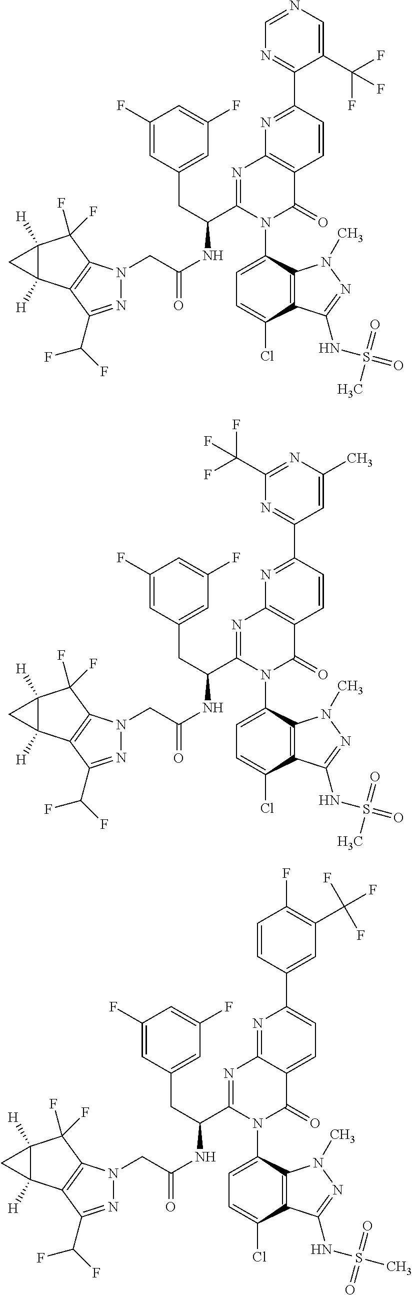 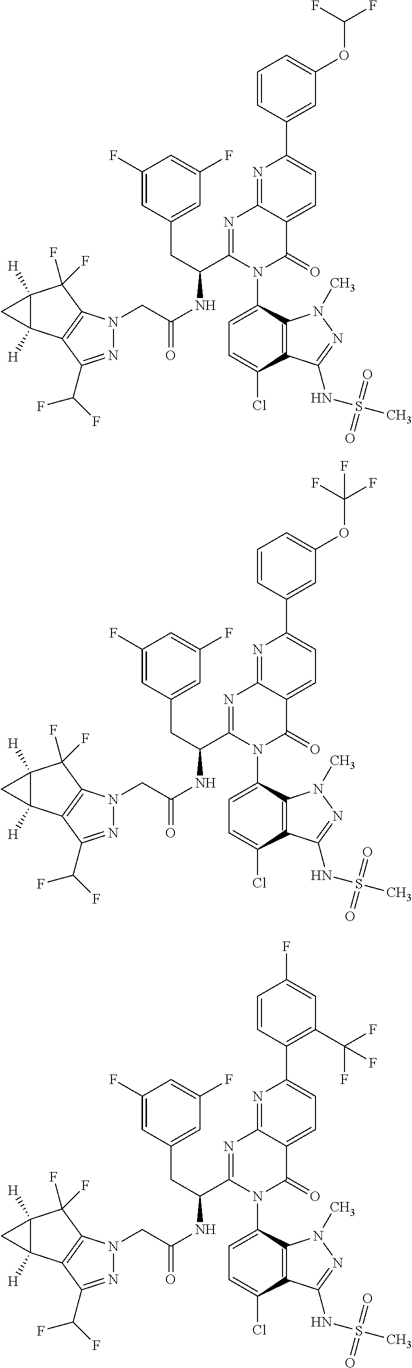
and pharmaceutically acceptable salts thereof.

30. A compound or salt according to claim 1, selected from the group consisting of: 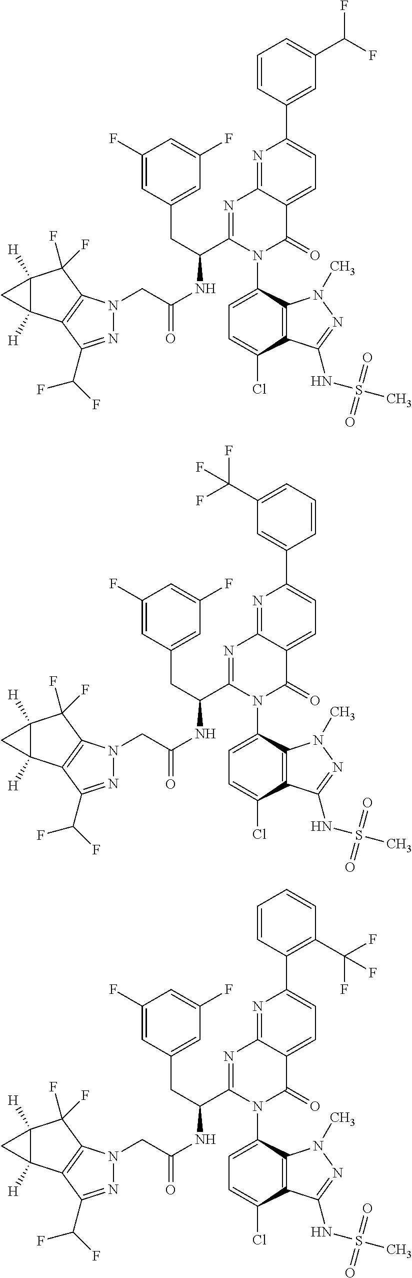 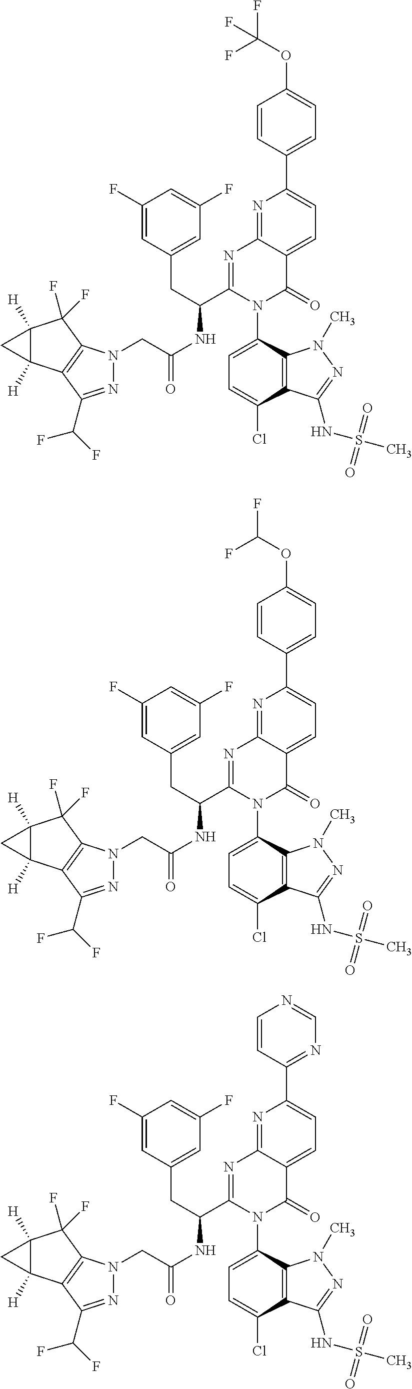 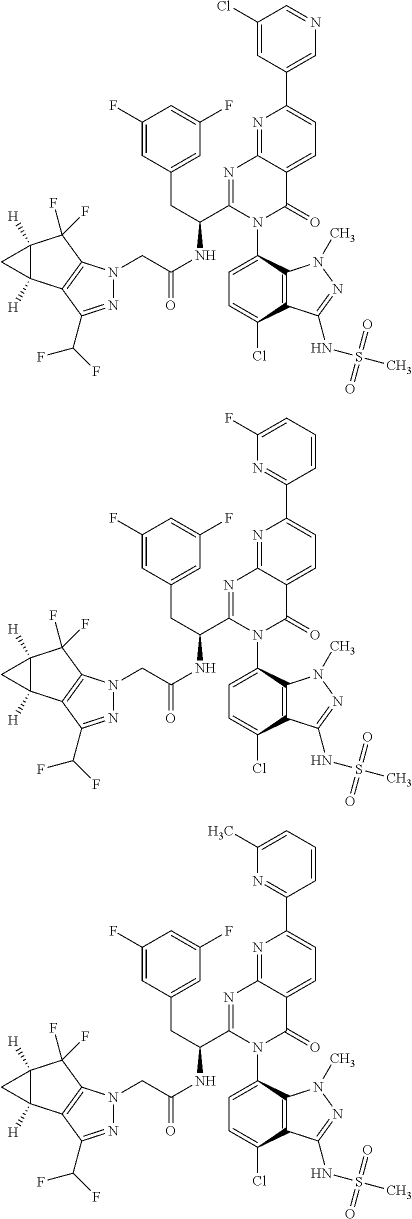 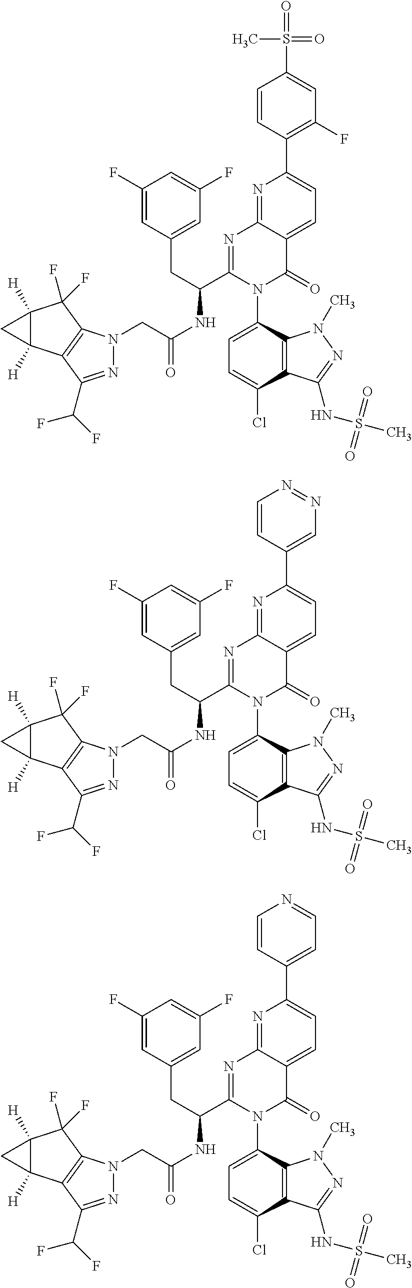 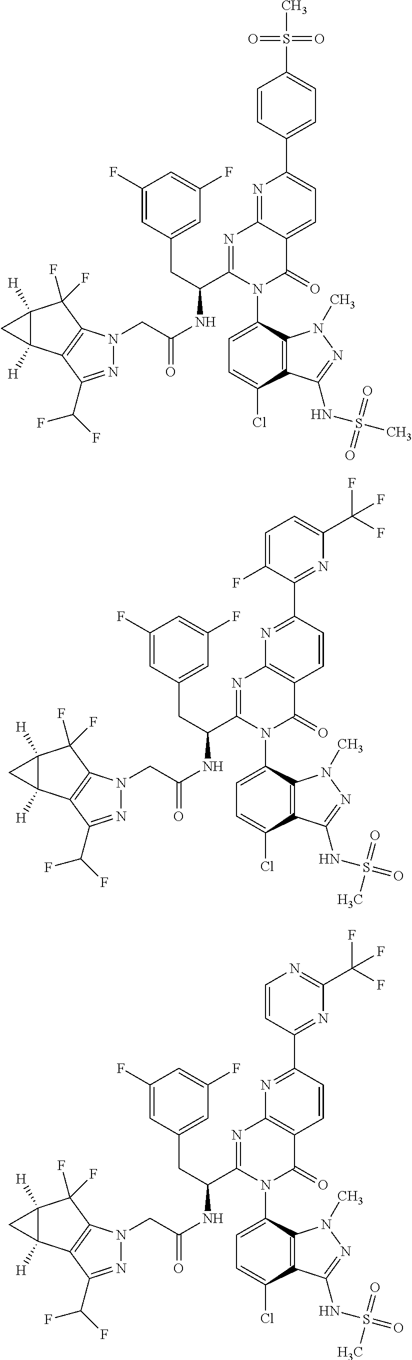 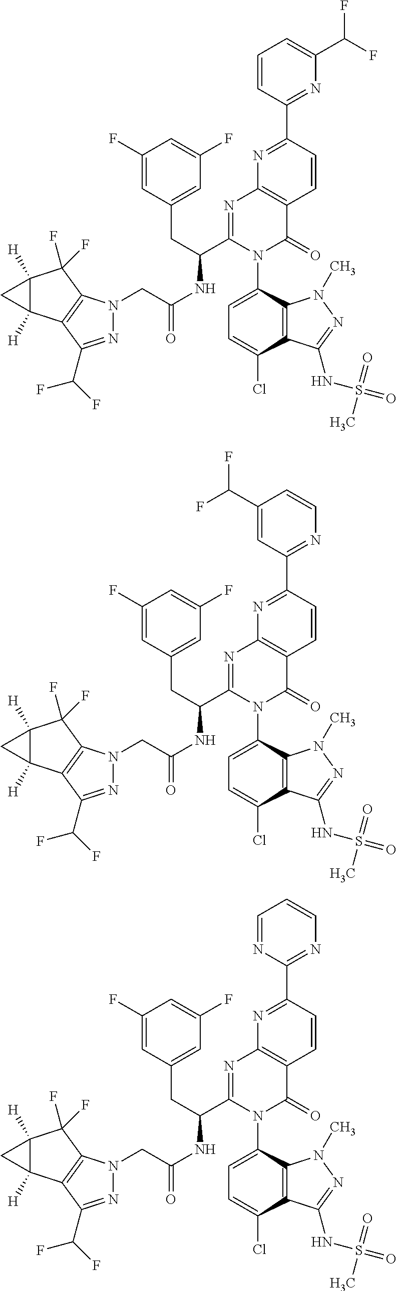 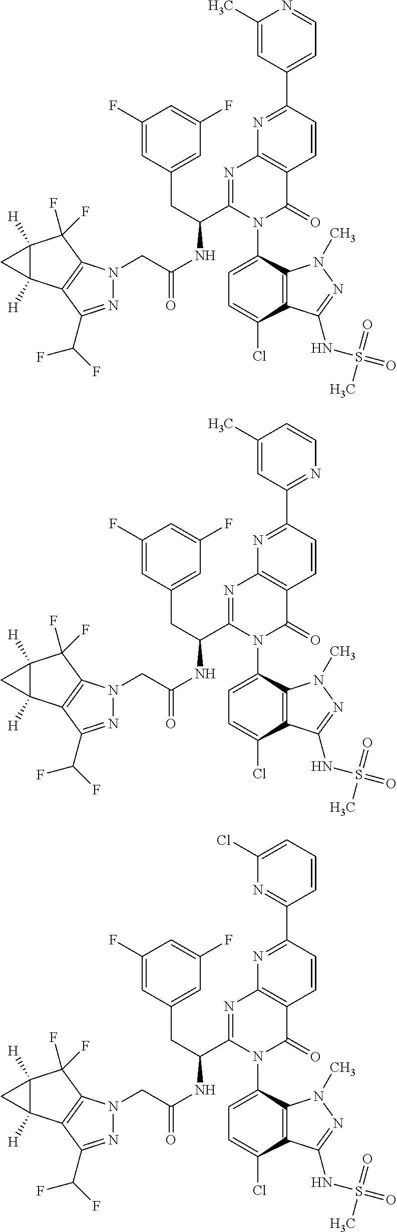
and pharmaceutically acceptable salts thereof.

31. A compound or salt according to claim 1, selected from the group consisting of: 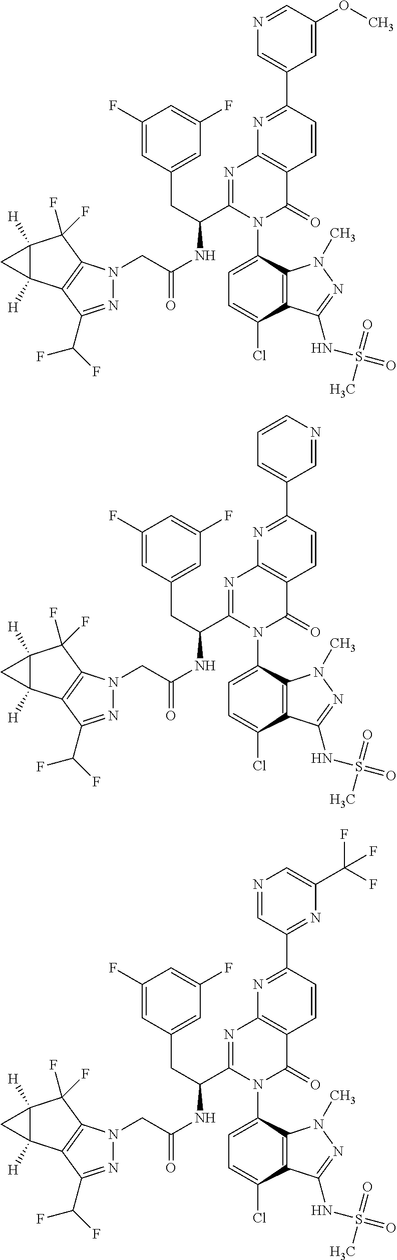 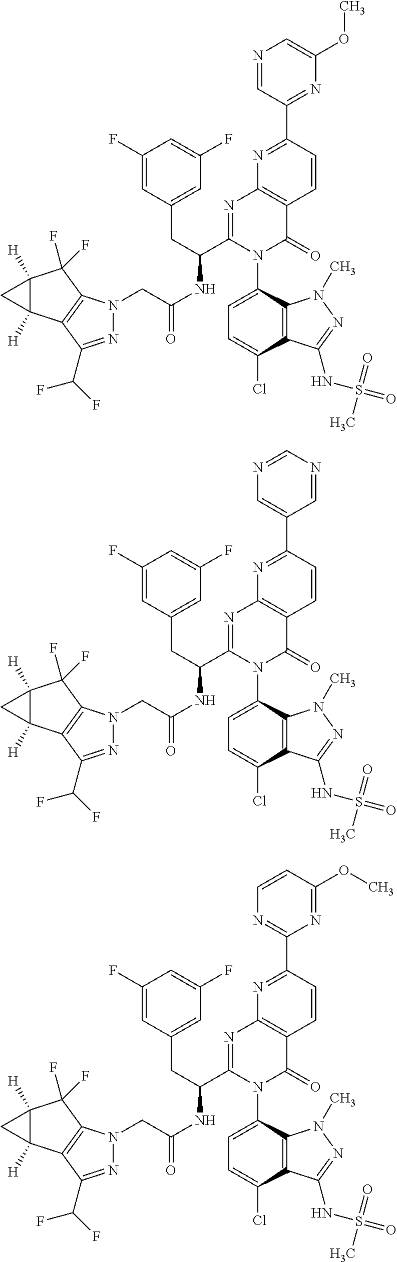 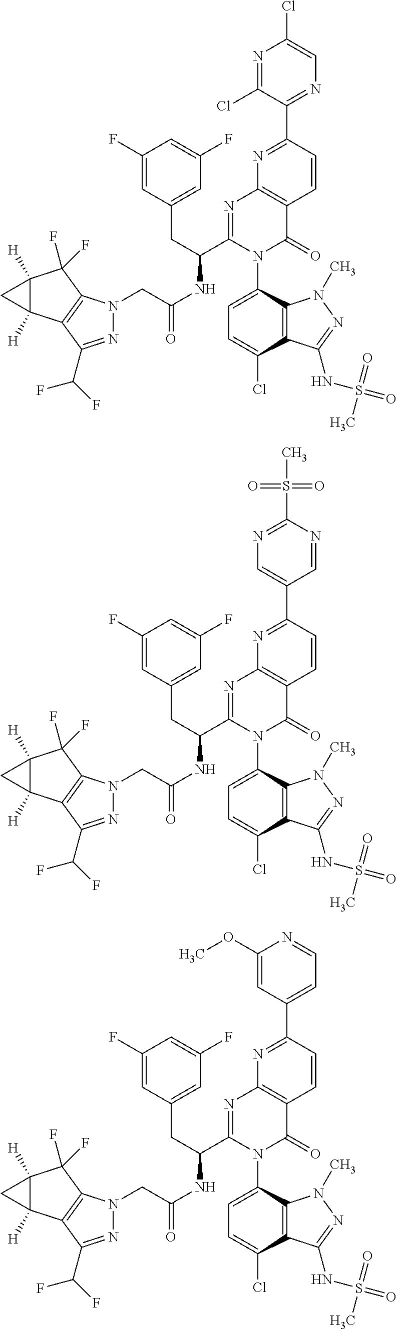 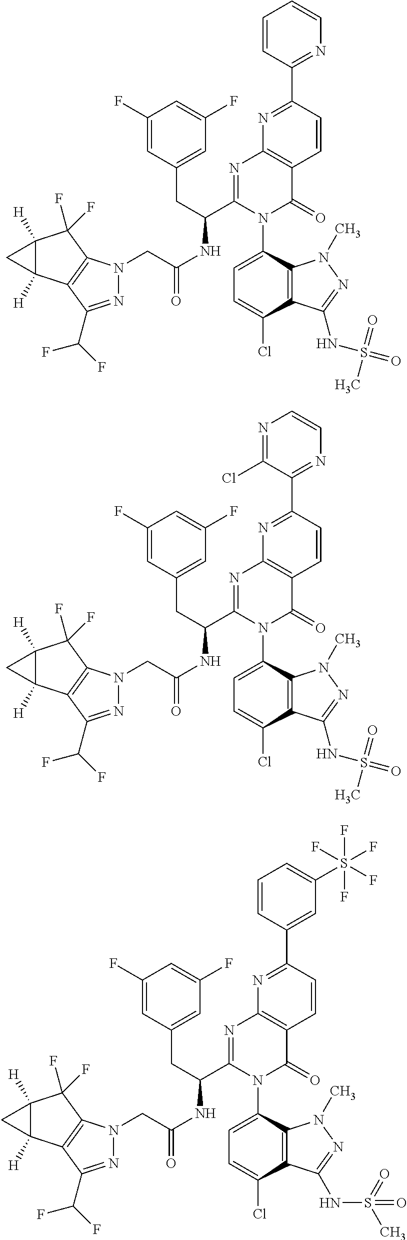 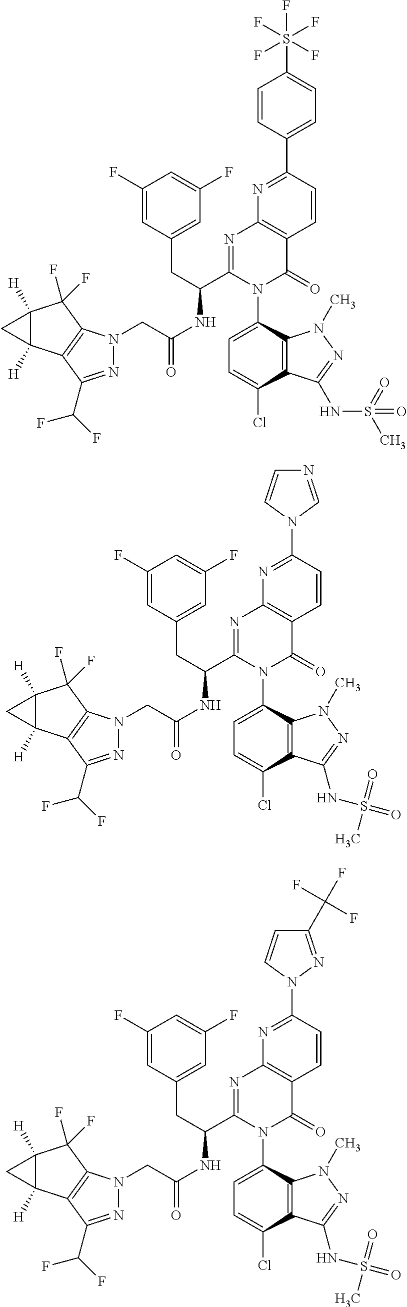 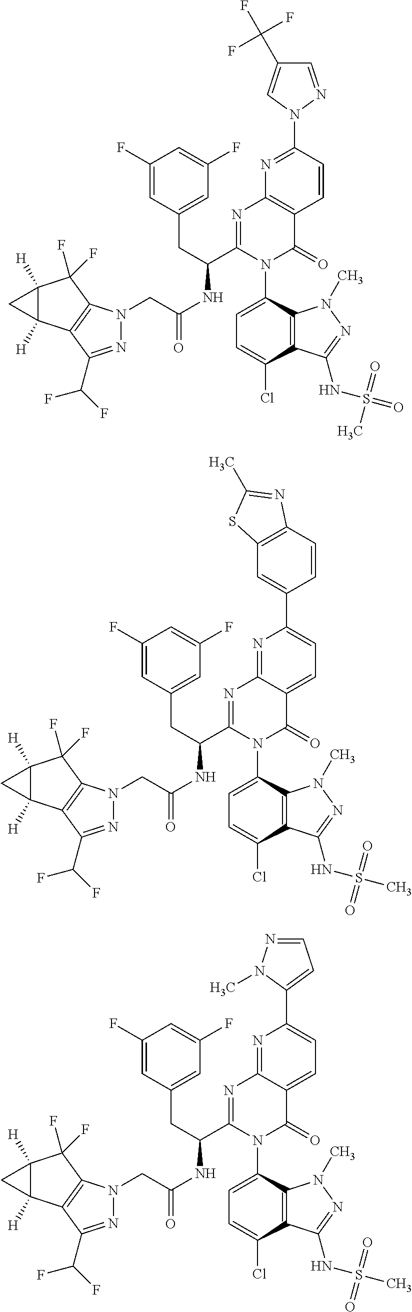
and pharmaceutically acceptable salts thereof.

32. A compound or salt according to claim 1, selected from the group consisting of: 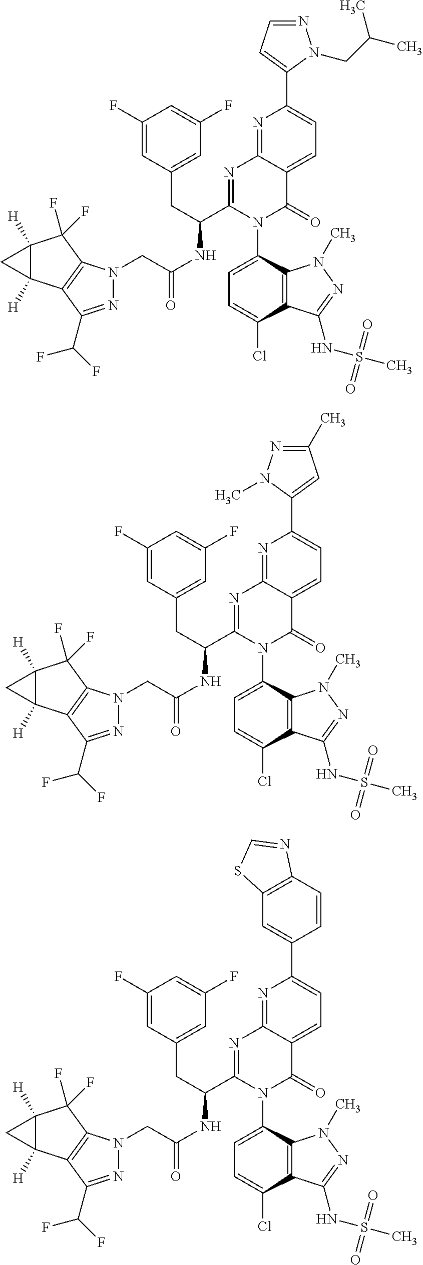 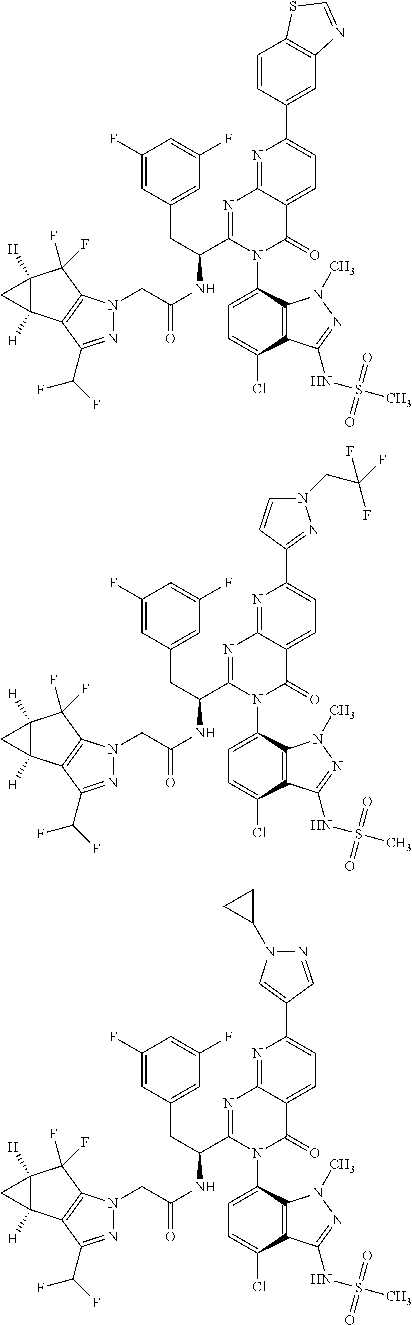 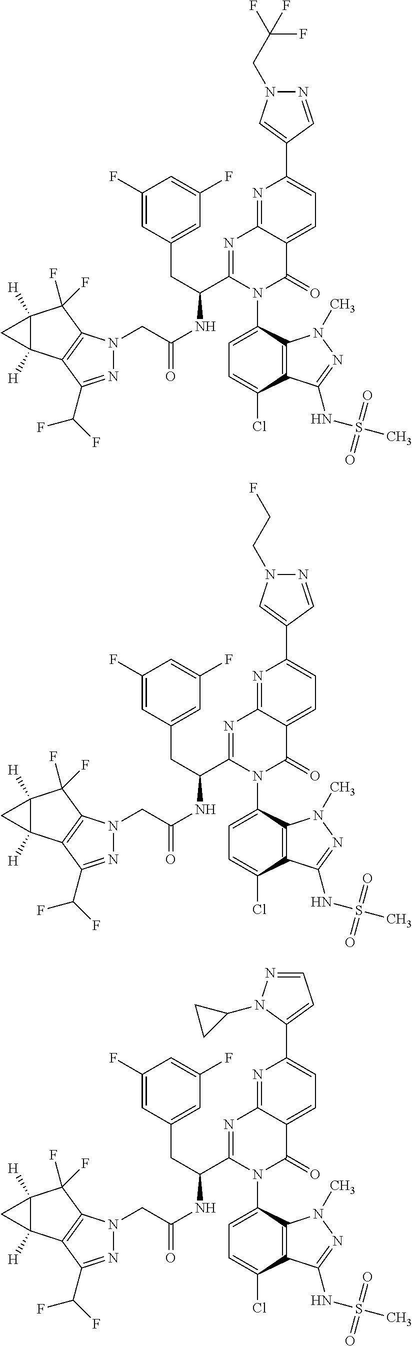 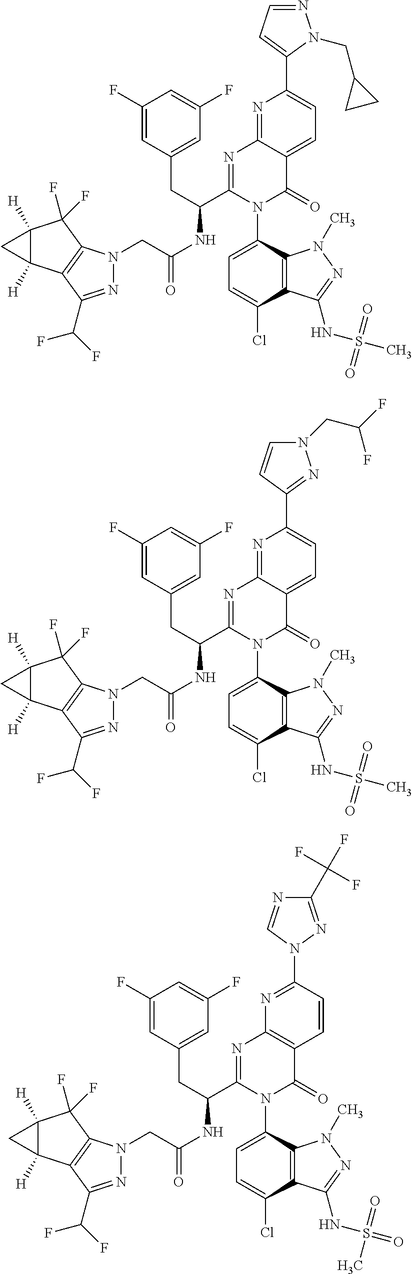 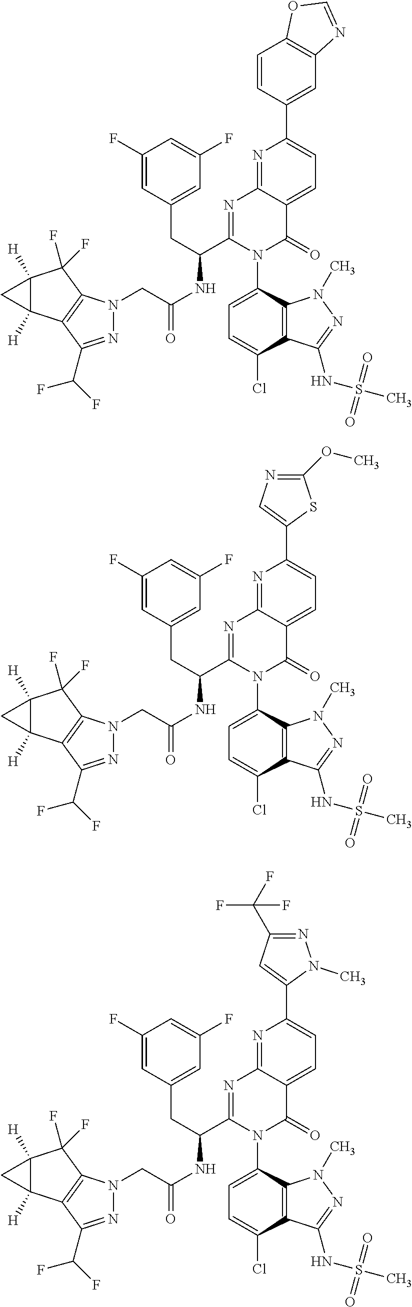 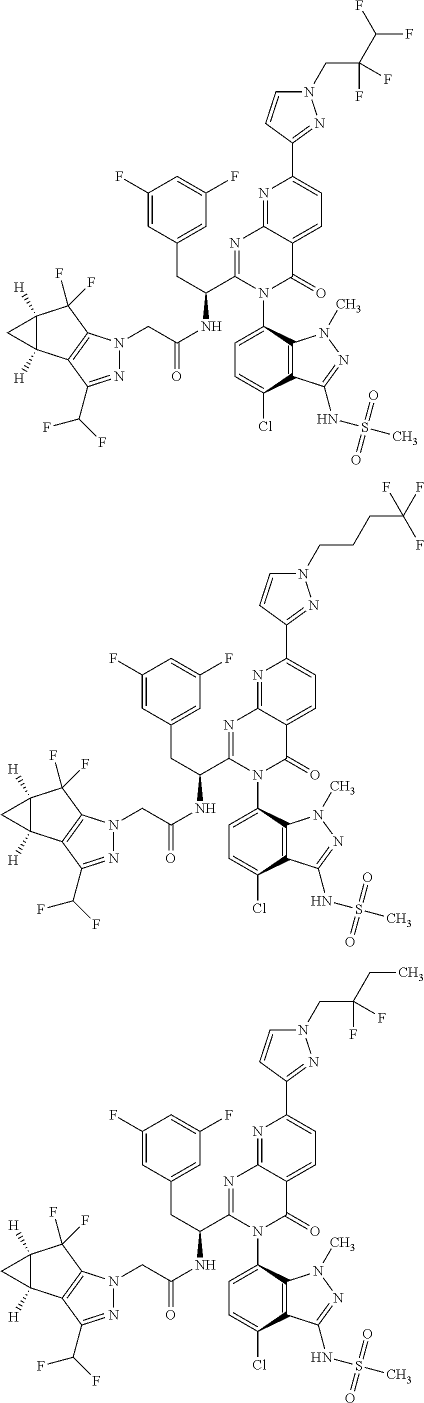 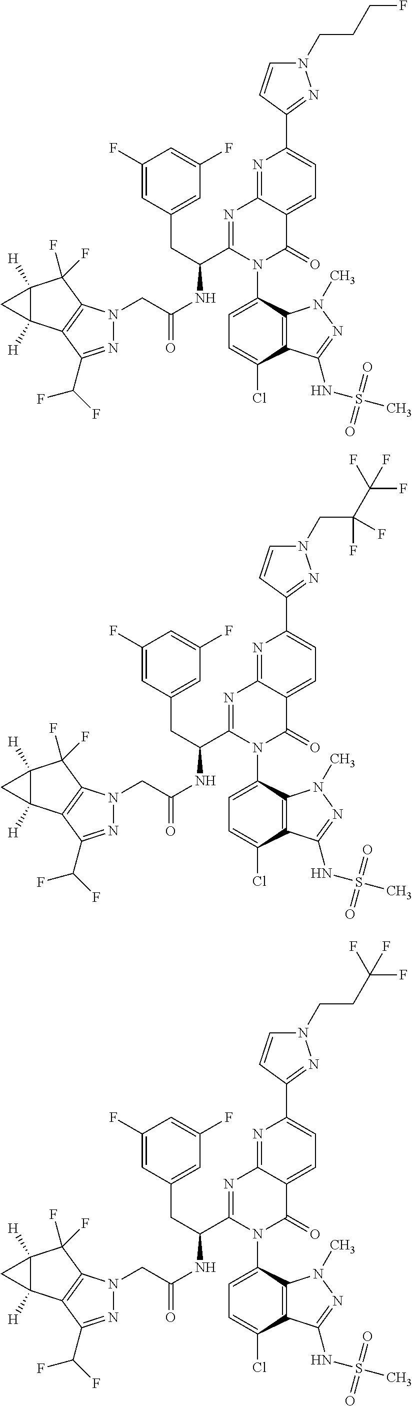
and pharmaceutically acceptable salts thereof.

33. A compound or salt according to claim 1, selected from the group consisting of: 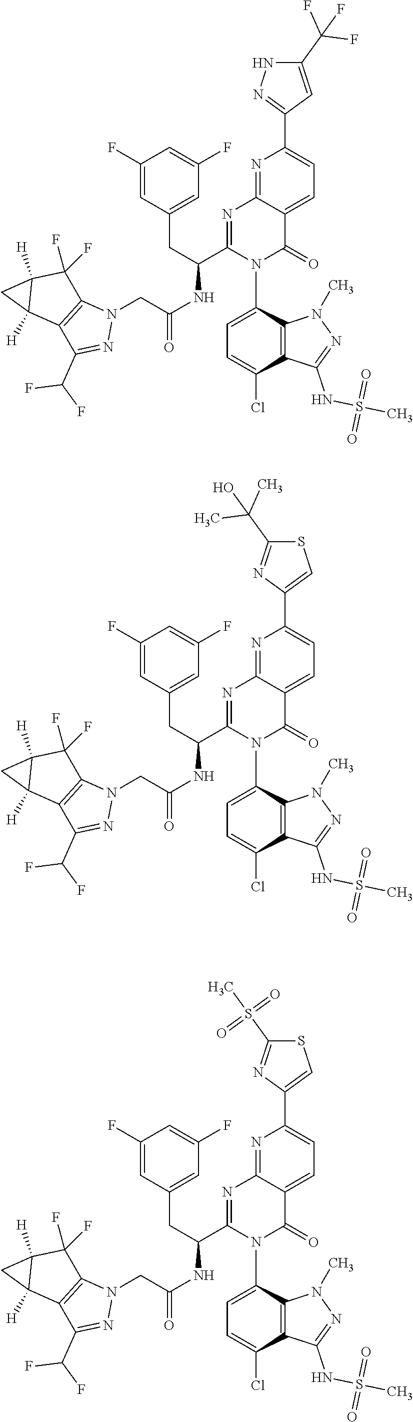 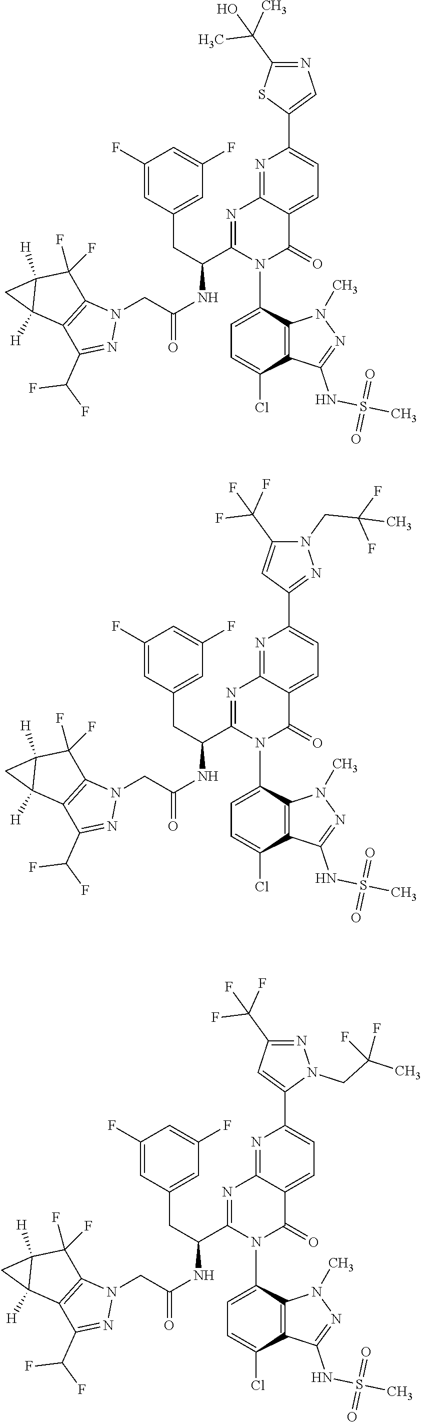 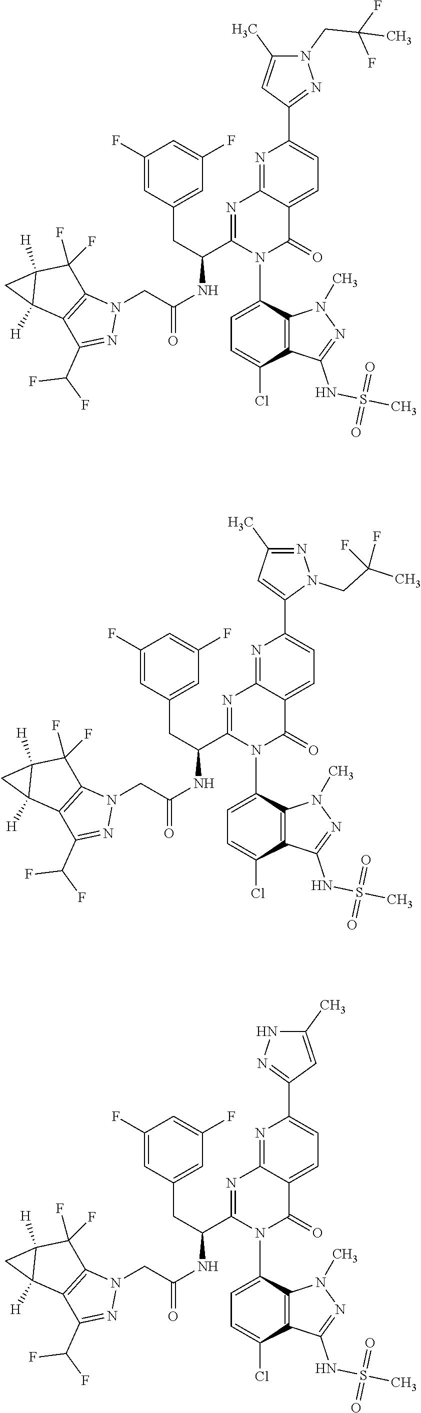 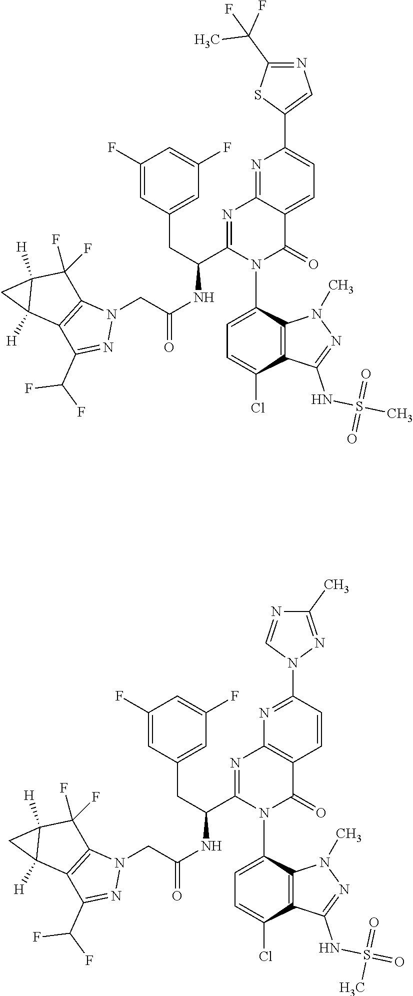
and pharmaceutically acceptable salts thereof.

34. A pharmaceutical composition comprising a compound or salt according to claim 1.

35. A composition according to claim 34 further comprising a pharmaceutically acceptable excipient.

36. A composition according to claim 34 suitable for oral administration, for intramuscular injection, or for subcutaneous injection.

37. A method of treating HIV infection in a human comprising administration of a compound or salt according to claim 1.

38. The method of claim 37 wherein said administration is oral.

39. The method of claim 37 wherein said administration is intramuscular injection or subcutaneous injection.

40. The method of claim 37 wherein said method further comprises administration of at least one other agent used for treatment of HIV infection in a human.

41. The method of claim 40 wherein said at least one other agent is selected from the group consisting of dolutegravir, bictegravir, lamivudine, fostemsavir, and cabotegravir.

42-44. (canceled)
